SEQUENCE LISTING (154 sequences)

SEQ ID NO: 1 - protein, 5 aa, synthetic construct
DYAMH

SEQ ID NO: 2 - protein, 17 aa, synthetic construct
AITWNSGHIDYADSVEG

SEQ ID NO: 3 - protein, 12 aa, synthetic construct
VSYLSTASSLDY

SEQ ID NO: 4 - protein, 121 aa, synthetic construct [sequence omitted]

SEQ ID NO: 5 - protein, 451 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 11 aa, synthetic construct
RASQGIRNYLA

SEQ ID NO: 7 - protein, 7 aa, synthetic construct
AASTLQS

SEQ ID NO: 8 - protein, 9 aa, synthetic construct
QRYNRAPYT

SEQ ID NO: 9 - protein, 107 aa, synthetic construct [sequence omitted]

SEQ ID NO: 10 - protein, 214 aa, synthetic construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 8 aa, synthetic construct
GYTFTNYG

SEQ ID NO: 12 - protein, 8 aa, synthetic construct
INTYTGEP

SEQ ID NO: 13 - protein, 16 aa, synthetic construct
AKYPHYYGSSHWYFDV

SEQ ID NO: 14 - protein, 123 aa, synthetic construct [sequence omitted]

SEQ ID NO: 15 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16-17 - skipped (2 entries)

SEQ ID NO: 18 - protein, 9 aa, synthetic construct
QQYSTVPWT

SEQ ID NO: 19 - protein, 110 aa, synthetic construct
DIQAATMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIK

SEQ ID NO: 20 - protein, 217 aa, synthetic construct
DIQAATMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 8 aa, synthetic construct
GFTFSSYT

SEQ ID NO: 22 - protein, 11 aa, synthetic construct
TFISYDGNNKY

SEQ ID NO: 23 - protein, 11 aa, synthetic construct
ARTGWLGPFDY

SEQ ID NO: 24 - protein, 118 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 25 - protein, 448 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 7 aa, synthetic construct
QSVGSSY

SEQ ID NO: 27 - skipped

SEQ ID NO: 28 - protein, 9 aa, synthetic construct
QQYGSSPWT

SEQ ID NO: 29 - protein, 108 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 30 - protein, 215 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 5 aa, synthetic construct
SYIMM

SEQ ID NO: 32 - protein, 10 aa, synthetic construct
SIYPSGGITF

SEQ ID NO: 33 - protein, 9 aa, synthetic construct
IKLGTVTTV

SEQ ID NO: 34 - protein, 120 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 35 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 8 aa, synthetic construct
VGGYNYVS

SEQ ID NO: 37 - protein, 6 aa, synthetic construct
DVSNRP

SEQ ID NO: 38 - protein, 10 aa, synthetic construct
SSYTSSSTRV

SEQ ID NO: 39 - protein, 110 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 40 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 41 - protein, 5 aa, synthetic construct
RYWMS

SEQ ID NO: 42 - protein, 10 aa, synthetic construct
NIKQDGSEKY

SEQ ID NO: 43 - protein, 10 aa, synthetic construct
EGGWFGELAF

SEQ ID NO: 44 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 45 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 8 aa, synthetic construct
RVSSSYLA

SEQ ID NO: 47 - protein, 6 aa, synthetic construct
DASSRA

SEQ ID NO: 48 - protein, 9 aa, synthetic construct
QQYGSLPWT

SEQ ID NO: 49 - protein, 108 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 50 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 13 aa, synthetic construct
KASGYTFTNYYMY

SEQ ID NO: 52 - protein, 10 aa, synthetic construct
GINPSNGGTN

SEQ ID NO: 53 - protein, 13 aa, synthetic construct
ARRDYRFDMGFDY

SEQ ID NO: 54 - protein, 120 aa, synthetic construct [sequence omitted]

SEQ ID NO: 55 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 15 aa, synthetic construct
RASKGVSTSGYSYLH

SEQ ID NO: 57 - protein, 8 aa, synthetic construct
YLASYLES

SEQ ID NO: 58 - protein, 9 aa, synthetic construct
QHSRDLPLT

SEQ ID NO: 59 - protein, 111 aa, synthetic construct [sequence omitted]

SEQ ID NO: 60 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 7 aa, synthetic construct
ITFSNSG

SEQ ID NO: 62 - protein, 8 aa, synthetic construct
WYDGSKRY

SEQ ID NO: 63 - skipped

SEQ ID NO: 64 - protein, 113 aa, synthetic construct [sequence omitted]

SEQ ID NO: 65 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 66 - protein, 6 aa, synthetic construct
QSVSSY

SEQ ID NO: 67 - protein, 7 aa, synthetic construct
DASNRAT

SEQ ID NO: 68 - protein, 6 aa, synthetic construct
SSNWPR

SEQ ID NO: 69 - protein, 107 aa, synthetic construct [sequence omitted]

SEQ ID NO: 70 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 235 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 72 - protein, 467 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 10 aa, synthetic construct
GFNIKDTYIH

SEQ ID NO: 74 - protein, 17 aa, synthetic construct
RIYPTNGYTRYADSVKG

SEQ ID NO: 75 - protein, 11 aa, synthetic construct
WGGDGFYAMDY

SEQ ID NO: 76 - protein, 120 aa, synthetic construct [sequence omitted]

SEQ ID NO: 77 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 11 aa, synthetic construct
RASQDVNTAVA

SEQ ID NO: 79 - protein, 7 aa, synthetic construct
SASFLYS

SEQ ID NO: 80 - protein, 9 aa, synthetic construct
QQHYTTPPT

SEQ ID NO: 81 - protein, 107 aa, synthetic construct [sequence omitted]

SEQ ID NO: 82 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 8 aa, synthetic construct
GFTFTDYT

SEQ ID NO: 84 - protein, 8 aa, synthetic construct
VNPNSGGS

SEQ ID NO: 85 - protein, 12 aa, synthetic construct
ARNLGPSFYFDY

SEQ ID NO: 86 - protein, 119 aa, synthetic construct [sequence omitted]

SEQ ID NO: 87 - protein, 448 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 6 aa, synthetic construct
QDVSIG

SEQ ID NO: 89 - skipped

SEQ ID NO: 90 - protein, 9 aa, synthetic construct
QQYYIYPYT

SEQ ID NO: 91 - protein, 107 aa, synthetic construct [sequence omitted]

SEQ ID NO: 92 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 10 aa, synthetic construct
GFSLTNYGVH

SEQ ID NO: 94 - protein, 10 aa, synthetic construct
VIWSGGNTDY

SEQ ID NO: 95 - protein, 13 aa, synthetic construct
ARALTYYDYEFAY

SEQ ID NO: 96 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 97 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 11 aa, synthetic construct
RASQSIGTNIH

SEQ ID NO: 99 - protein, 7 aa, synthetic construct
YASESIS

SEQ ID NO: 100 - protein, 10 aa, synthetic construct
QQNNNWPTTF

SEQ ID NO: 101 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 102 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 10 aa, synthetic construct
GYTFTSYNMH

SEQ ID NO: 104 - protein, 17 aa, synthetic construct
AIYPGNGDTSYNQKFKG

SEQ ID NO: 105 - protein, 14 aa, synthetic construct
ARSTYYGGDWYFNV

SEQ ID NO: 106 - protein, 121 aa, synthetic construct [sequence omitted]

SEQ ID NO: 107 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 10 aa, synthetic construct
RASSSVSYIH

SEQ ID NO: 109 - protein, 9 aa, synthetic construct
ATSNLASGV

SEQ ID NO: 110 - protein, 9 aa, synthetic construct
QQWTSNPPT

SEQ ID NO: 111 - protein, 106 aa, synthetic construct [sequence omitted]

SEQ ID NO: 112 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 10 aa, synthetic construct
GYAFSYSWIN

SEQ ID NO: 114 - protein, 17 aa, synthetic construct
RIFPGDGDTDYNGKFKG

SEQ ID NO: 115 - protein, 12 aa, synthetic construct
ARNVFDGYWLVY

SEQ ID NO: 116 - protein, 20 aa, synthetic construct
OBINUTUZUMABGAZYVAVH

SEQ ID NO: 117 - protein, 449 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 16 aa, synthetic construct
RSSKSLLHSNGITYLY

SEQ ID NO: 119 - protein, 9 aa, synthetic construct
QMSNLVSGV

SEQ ID NO: 120 - protein, 9 aa, synthetic construct
AQNLELPYT

SEQ ID NO: 121 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 122 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 406 aa, synthetic construct
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 124 - protein, 401 aa, synthetic construct
NVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSMEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWGGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVC

SEQ ID NO: 125 - protein, 925 aa, synthetic construct
MIHTNLKKKFSCCVLVFLLFAVICVWKEKKKGSYYDSFKLQTKEFQVLKSLGKLAMGSDSQSVSSSSTQDPHRGRQTLGSLRGLAKAKPGGGGSGGGGSGGGGSNVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSMEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWGGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVCGSGATNFSLLKQAGDVEENPGPMRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 126 - protein, 934 aa, synthetic construct
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGGGGGSGGGGSGGGGSNVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSMEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWGGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVCGSGATNFSLLKQAGDVEENPGPMRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 127 - protein, 398 aa, synthetic construct
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 128 - protein, 22 aa, synthetic construct
GSGATNFSLLKQAGDVEENPGP

SEQ ID NO: 129 - protein, 21 aa, synthetic construct
GSGEGRGSLLTCGDVEENPGP

SEQ ID NO: 130 - protein, 23 aa, synthetic construct
GSGQCTNYALLKLAGDVESNPGP

SEQ ID NO: 131 - protein, 25 aa, synthetic construct
GSGVKQTLNFDLLKLAGDVESNPGP

SEQ ID NO: 132 - DNA, 1221 bp, synthetic construct
  source 1..1221: mol_type = other DNA
atgatccacaccaatctgaagaagaagtttagctgttgcgtgctggtgttcctgctgttcgctgtgatctgcgtgtggaaggagaagaagaagggcagctattatgactcctttaagctgcagaccaaggagttccaggtgctgaagtccctgggcaagctggccatgggcagcgactcccagtccgtgagctcctccagcacccaggacccccaccggggaaggcagaccctgggatccctgaggggcctggctaaggccaagcctgaggcttccttccaggtgtggaataaggatagctccagcaagaacctgatccctaggctgcagaagatctggaagaactatctgagcatgaacaagtataaggtgagctataagggccctggccctggcatcaagttctccgccgaggccctgaggtgccacctgagagatcacgtgaatgtgagcatggtggaggtgaccgacttcccttttaacacctccgagtgggagggctacctgcccaaggagagcatccggaccaaggccggcccttggggcagatgtgccgtggtgtccagcgccggctccctgaagagctcccagctgggccgggagatcgatgaccacgatgctgtgctgaggttcaatggcgctcctaccgctaactttcagcaggatgtgggcaccaagaccaccatccggctgatgaatagccagctggtgaccaccgagaagcggtttctgaaggactccctgtacaacgagggcatcctgatcgtgtgggacccctccgtgtaccacagcgacatccccaagtggtatcagaaccccgactacaacttcttcaataattacaagacctaccggaagctgcaccccaaccagcccttttatatcctgaagccccagatgccttgggagctgtgggacatcctgcaggagatctcccccgaggagatccagcctaacccccctagcagcggcatgctgggcatcatcatcatgatgaccctgtgcgatcaggtggatatctacgagttcctgcccagcaagcggaagaccgacgtgtgttattattatcagaagttcttcgattccgcctgcaccatgggcgcctatcaccctctgctgtacgagaagaatctggtgaagcacctgaaccagggcaccgacgaggacatctatctgctgggcaaggccaccctgcccggctttaggaccatccactgctga

SEQ ID NO: 133 - DNA, 1545 bp, synthetic construct
  source 1..1545: mol_type = other DNA
atgaaaaactttttattattaactttaatattacttactgcttgtaataattcagaagaaaatacacaatctattattaaaaatgatattaataaaactattattgatgaggagtatgttaatttagagccaattaatcaatcaaacatctcttttacaaaacactcttgggtacaaacttgtggtacgcaacaactattaacagaacaaaataaagagtcaatatcattatctgtagtggcgccacgattagatgacgatgaaaagtactgctttgattttaatggtgttagtaataaaggtgaaaaatatataacaaaagtaacattaaacgtagtggctccatctttagaggtttatgttgatcatgcatctcttccaactcttcagcagctaatggatattattaaatcggaagaagaaaatcctacagcacaaagatatatagcttgggggagaatagttccgactgatgagcaaatgaaagagttaaatattacatcgtttgcattgataaataaccatacaccagctgacttagtacaagaaattgttaagcaagcacaaacaaagcatagattgaatgttaaacttagctctaacactgctcattcatttgataatttagtgccaatactaaaagaattaaattcgtttaataacgttacggtaacaaatatagatttatatgatgatggttcagcagaatatgtaaatttatataactggagagatacattaaataaaacagataatttaaaaattggtaaagattatcttgaggatgtcattaatggtatcaatgaagacacttcaaatacaggaacatcatctgtttataactggcaaaaactatatccagctaactaccattttttaagaaaagattatttaactttagaaccatcattacatgagttacgagactatattggtgatagtttaaagcaaatgcaatgggatggtttcaaaaaattcaatagcaaacaacaagaattattcttatcgattgttaattttgacaaacaaaaattacaaaatgaatataattcatctaatttaccaaactttgtgtttacaggtacgactgtatgggctggtaaccatgaaagagagtattatgcgaaacaacaaattaatgtcattaataatgcaattaatgaatcgagcccacattatttaggcaatagttatgatttgttcttcaaaggtcaccctggtggcggtatcattaatacattaataatgcaaaactatccttcaatggttgatattccatcaaaaatatcatttgaagttttgatgatgacagatatgcttcctgatgcagttgctggtatagcgagctctttatatttcacgataccagctgaaaaaattaaatttatagtttttacatcgacagaaactataactgatcgtgaaactgctttgagaagtcctttagttcaagtaatgataaaactaggtattgtaaaagaagagaatgtacttttttgggctgatctgccaaattgtgaaacaggtgtttgtattgcagtctag

SEQ ID NO: 134 - DNA, 2778 bp, synthetic construct
  source 1..2778: mol_type = other DNA
atgatccacaccaacctgaagaagaagttctcctgctgtgtgctggtgtttctgctgttcgctgtgatctgcgtgtggaaggagaagaagaagggcagctattatgacagctttaagctgcagaccaaggagtttcaggtgctgaagtccctgggcaagctggccatgggctccgactcccagtccgtgtccagctccagcacccaggatcctcaccggggcaggcagaccctgggctctctgagaggcctggctaaggctaagcccggcggcggaggcagcggaggaggaggatctggcggcggaggatccaatgtggtggccccttccctggaggtgtatgtggaccacgcctccctgcccaccctgcagcagctgatggatatcatcaagagcgaggaggagaatcctaccgcccagcggtatatcgcctggggccggatcgtgcctaccgacgagcagatgaaggagctgaacatcacctccttcgctctgatcaacaatcacacccccgccgacctggtgcaggagatcgtgaagcaggcccagaccaagcaccggctgaacgtgaagctgtcctccaacaccgcccactcctttgacaacctggtgcctatcctgaaggagctgaattcctttaataacgtgaccgtgaccaacatcgacctgtacgacgacggctccatggagtatgtgaacctgtataactggagggacaccctgaataagaccgacaatctgaagatcggcaaggactatctggaggacgtgatcaatggcatcaatgaggacacctccaacaccggcaccagcagcgtgtataactggcagaagctgtaccctgctaattaccactttctgaggaaggattatctgaccctggagccttccctgcacgagctgagggactacatcggcgatagcctgaagcagatgcagtgggatggctttaagaagttcaacagcaagcagcaggagctgtttctgagcatcgtgaattttgacaagcagaagctgcagaacgagtataactcctccaatctgcctaactttgtgttcaccggcaccaccgtgtggggcggcaatcacgagcgggagtactatgccaagcagcagatcaacgtgatcaataatgccatcaatgagagcagcccccactacctgggcaatagctacgatctgttcttcaagggccaccctggcggcggcatcatcaacaccctgatcatgcagaactatccttccatggtggatatccctagcaagatctccttcgaggtgctgatgatgaccgacatgctgcccgacgccgtggctggcatcgcctctagcctgtattttaccatccccgctgagaagatcaagttcatcgtgtttaccagcaccgagaccatcaccgaccgggagaccgccctgaggtccccactggtgcaggtgatgatcaagctgggcatcgtgaaggaggagaacgtgctgttttgggccgacctgcccaactgtgagaccggcgtgtgtggctccggcgctaccaacttctccctgctgaagcaggctggcgatgtggaggagaatcccggccctatgcggctgcgggagcccctgttgtccggctctgccgctatgcccggcgcttccctgcagagagcctgtcggctgctggtggctgtgtgcgctctgcacctgggcgtgaccctggtgtactatctggctggccgggatctgagccggctgcctcagctggtgggcgtgagcacccccctgcagggaggatccaactccgccgctgccatcggccagtcctccggagagctgcggaccggaggcgctaggcctccaccaccactgggcgcttcttcccagcctcggcccggaggagatagcagccccgtggtggactccggccctggacctgcttccaacctgaccagcgtgcccgtgcctcacaccaccgctctgtccctgcccgcctgtcccgaggagtcccctctgctggtgggccctatgctgatcgagtttaatatgcctgtggacctggagctggtggctaagcagaatcctaacgtgaagatgggcggcaggtacgctcccagggactgcgtgagccctcacaaggtggccatcatcatccctttccggaaccggcaggagcacctgaagtactggctgtactacctgcaccctgtgctgcagaggcagcagctggactatggcatctacgtgatcaaccaggccggcgacaccatctttaaccgggccaagctgctgaatgtgggctttcaggaggccctgaaggattatgactacacctgctttgtgtttagcgatgtggatctgatccctatgaacgatcacaacgcctaccggtgcttcagccagcctcggcacatcagcgtggccatggacaagttcggcttctccctgccttacgtgcagtacttcggcggcgtgtccgccctgtccaagcagcagttcctgaccatcaacggctttcctaataactactggggctggggcggcgaggatgatgacatctttaatcggctggtgtttaggggcatgagcatcagccggcctaacgccgtggtgggcaggtgcaggatgatcaggcactcccgggacaagaagaacgagcctaatcctcagaggtttgacaggatcgctcacaccaaggagaccatgctgagcgacggcctgaatagcctgacctaccaggtgctggacgtgcagaggtaccctctgtatacccagatcaccgtggatatcggcacccctagctga

SEQ ID NO: 135 - DNA, 1431 bp, synthetic construct
  source 1..1431: mol_type = other DNA
atgaggctgcgggagccattgctgagcggctccgctgccatgcctggcgcttctctgcagagggcttgtaggctgctggtggccgtgtgcgccctgcacctgggagtgaccctggtgtactacctggctggccgggacctgtcccggctgcctcagttggtgggcgtgtccacccctctgcagggcggcggaggcagcggaggaggaggatctggcggcggaggatccaatgtggtggccccttccctggaggtgtatgtggaccacgcctccctgcccaccctgcagcagctgatggatatcatcaagagcgaggaggagaatcctaccgcccagcggtatatcgcctggggccggatcgtgcctaccgacgagcagatgaaggagctgaacatcacctccttcgctctgatcaacaatcacacccccgccgacctggtgcaggagatcgtgaagcaggcccagaccaagcaccggctgaacgtgaagctgtcctccaacaccgcccactcctttgacaacctggtgcctatcctgaaggagctgaattcctttaataacgtgaccgtgaccaacatcgacctgtacgacgacggctccatggagtatgtgaacctgtataactggagggacaccctgaataagaccgacaatctgaagatcggcaaggactatctggaggacgtgatcaatggcatcaatgaggacacctccaacaccggcaccagcagcgtgtataactggcagaagctgtaccctgctaattaccactttctgaggaaggattatctgaccctggagccttccctgcacgagctgagggactacatcggcgatagcctgaagcagatgcagtgggatggctttaagaagttcaacagcaagcagcaggagctgtttctgagcatcgtgaattttgacaagcagaagctgcagaacgagtataactcctccaatctgcctaactttgtgttcaccggcaccaccgtgtggggcggcaatcacgagcgggagtactatgccaagcagcagatcaacgtgatcaataatgccatcaatgagagcagcccccactacctgggcaatagctacgatctgttcttcaagggccaccctggcggcggcatcatcaacaccctgatcatgcagaactatccttccatggtggatatccctagcaagatctccttcgaggtgctgatgatgaccgacatgctgcccgacgccgtggctggcatcgcctctagcctgtattttaccatccccgctgagaagatcaagttcatcgtgtttaccagcaccgagaccatcaccgaccgggagaccgccctgaggtccccactggtgcaggtgatgatcaagctgggcatcgtgaaggaggagaacgtgctgttttgggccgacctgcccaactgtgagaccggcgtgtgt

SEQ ID NO: 136 - DNA, 1197 bp, synthetic construct
  source 1..1197: mol_type = other DNA
atgaggctgcgggagccattgctgagcggctccgctgccatgcctggcgcttctctgcagagggcttgtaggctgctggtggccgtgtgcgccctgcacctgggagtgaccctggtgtactacctggctggccgggacctgtcccggctgcctcagttggtgggcgtgtccacccctctgcagggcggaagcaatagcgccgctgccatcggccagtcctccggagagctgcggaccggaggcgctaggccacctccacctctgggcgcttcctcccagcctcggcctggaggagatagcagccccgtggtggatagcggccctggcccagcttctaacctgaccagcgtgcctgtgccccacaccaccgccctgagcctgcctgcttgccccgaggagtcccccctgctggtgggaccaatgctgatcgagttcaatatgcctgtggatctggagctggtggccaagcagaatcccaatgtgaagatgggcggccggtacgctccccgggattgtgtgtcccctcacaaggtggctatcatcatccctttccggaaccggcaggagcacctgaagtactggctgtattacctgcaccccgtgctgcagaggcagcagctggactatggcatctacgtgatcaatcaggctggcgacaccatcttcaatcgggctaagctgctgaacgtgggctttcaggaggctctgaaggactacgactacacctgctttgtgttcagcgatgtggacctgatccccatgaacgaccacaacgcttataggtgcttctcccagcctaggcacatctccgtggctatggacaagtttggcttctccctgccctacgtgcagtattttggcggcgtgtccgccctgagcaagcagcagtttctgaccatcaatggctttcctaacaattactggggctggggcggcgaggatgatgacatcttcaaccggctggtgttcaggggcatgtccatcagcaggcctaacgctgtggtgggccggtgtaggatgatcaggcactcccgggacaagaagaatgagcccaatcctcagcggtttgaccggatcgcccacaccaaggagaccatgctgtccgacggcctgaattccctgacctaccaggtgctggacgtgcagaggtatcctctgtacacccagatcaccgtggatatcggcaccccttcctga

SEQ ID NO: 137 - protein, 313 aa, synthetic construct
MFTPVRRRVRTAALALSAAAALVLGSTAASGASATPSPAPAPAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTP

SEQ ID NO: 138 - protein, 843 aa, synthetic construct
MDKHLLVKRTLGCVCAATLMGAALATHHDSLNTVKAEEKTVQTGKTDQQVGAKLVQEIREGKRGPLYAGYFRTWHDRASTGIDGKQQHPENTMAEVPKEVDILFVFHDHTASDSPFWSELKDSYVHKLHQQGTALVQTIGVNELNGRTGLSKDYPDTPEGNKALAAAIVKAFVTDRGVDGLDIDIEHEFTNKRTPEEDARALNVFKEIAQLIGKNGSDKSKLLIMDTTLSVENNPIFKGIAEDLDYLLRQYYGSQGGEAEVDTINSDWNQYQNYIDASQFMIGFSFFEESASKGNLWFDVNEYDPNNPEKGKDIEGTRAKKYAEWQPSTGGLKAGIFSYAIDRDGVAHVPSTYKNRTSTNLQRHEVDNISHTDYTVSRKLKTLMTEDKRYDVIDQKDIPDPALREQIIQQVGQYKGDLERYNKTLVLTGDKIQNLKGLEKLSKLQKLELRQLSNVKEITPELLPESMKKDAELVMVGMTGLEKLNLSGLNRQTLDGIDVNSITHLTSFDISHNSLDLSEKSEDRKLLMTLMEQVSNHQKITVKNTAFENQKPKGYYPQTYDTKEGHYDVDNAEHDILTDFVFGTVTKRNTFIGDEEAFAIYKEGAVDGRQYVSKDYTYEAFRKDYKGYKVHLTASNLGETVTSKVTATTDETYLVDVSDGEKVVHHMKLNIGSGAIMMENLAKGAKVIGTSGDFEQAKKIFDGEKSDRFFTWGQTNWIAFDLGEINLAKEWRLFNAETNTEIKTDSSLNVAKGRLQILKDTTIDLEKMDIKNRKEYLSNDENWTDVAQMDDAKAIFNSKLSNVLSRYWRFCVDGGASSYYPQYTELQILGQRLSNDVANTLKD

SEQ ID NO: 139 - DNA, 1104 bp, synthetic construct
  source 1..1104: mol_type = other DNA
gactcgaagcactcgctcggcgcttccgactggcggggcgccgaccatcccgttgggagaccatcagccatgttcactccggttcgcagaagggtgcggacggctgcgctcgcgctctcggccgccgcggccctcgtcctcggttccaccgccgcgagcggcgcgtcagcgaccccctcacccgctccggccccggccccggccccggtgaagcaggggccgacctcggtggcctacgtcgaggtgaacaacaacagcatgctcaacgtcggcaagtacaccctggcggacggaggcggcaacgccttcgacgtagccgtgatcttcgcggcgaacatcaactacgacaccggcacgaagacggcctacctgcacttcaacgagaacgtgcagcgcgtccttgacaacgctgtcacgcagatacggccgttgcagcaacagggcatcaaggtcctcctctcggtgctcggcaaccaccagggcgccgggttcgcgaacttcccctcacagcaggcggcttcggcgttcgcgaagcagctctcggacgccgtggcgaagtacggcctcgacggcgtcgacttcgacgacgaatacgccgagtacggcaacaacggcaccgcgcagcccaacgacagttcgttcgtgcacctggtgacggcactgcgcgcgaacatgcccgacaagatcatcagcctctacaacatcggcccggccgcgtcccgcctgtcgtacggcggtgtcgacgtctccgacaagttcgactacgcctggaatccctactacggcacctggcaggtccccggcatcgcactgcccaaggcgcagctgtcgccggcggccgtcgagatcggccggacctcacggagcaccgtcgccgacctcgcccgtcgcaccgtcgacgaggggtacggcgtctatctgacgtacaacctcgacggcggcgatcgcaccgccgacgtctccgcgttcaccagggagctgtacggcagcgaggcggtccggacgccgtaggggcgtcggggcctgccgtcagtccagtacgaaggtgccgccggcggtggtcgcctggccgtgcccgaaagcggccgccggcgtccaggatcc

SEQ ID NO: 140 - DNA, 2776 bp, synthetic construct
  source 1..2776: mol_type = other DNA
ggcgaactataggaatgcgcttacatagatggtatatcagatgggaagccatgacttagtaccaaaaataaggagtgtccaaatggataaacatttgttggtaaaaagaacactagggtgtgtttgtgctgcaacgttgatgggagctgccttagcgacccaccatgattcactcaatactgtaaaagcggaggagaagactgttcaaacaggaaagacagatcagcaggttggtgctaaattggtacaggaaatccgtgaaggaaaacgcggaccactatatgctggttattttaggacatggcatgatcgtgcttcaacaggaatagatggtaaacagcaacatccagaaaatactatggctgaggtcccaaaagaagttgatatcttatttgtttttcatgaccatacagcttcagatagtccattttggtctgaattaaaggacagttatgtccataaattacatcaacagggaacggcacttgttcagacaattggtgttaacgaattaaatggacgtacaggtttatctaaagattatcctgatactcctgaggggaacaaagctttagcagcagccattgtcaaggcatttgtaactgatcgtggtgtcgatggactagatattgatattgagcacgaatttacgaacaaaagaacacctgaagaagatgctcgtgctctaaatgtttttaaagagattgcgcagttaataggtaaaaatggtagtgataaatctaaattgctcatcatggacactaccctaagtgttgaaaataatccaatatttaaagggatagcggaagatcttgattatcttcttagacaatattatggttcacaaggtggagaagctgaagtggatactataaactctgattggaaccaatatcagaattatattgatgctagccagttcatgattggattctccttttttgaagaatctgcgtccaaagggaatttatggtttgatgttaacgaatacgaccctaacaatcctgaaaaagggaaagatattgaaggaacacgtgctaaaaaatatgcagagtggcaacctagtacaggtggtttaaaagcaggtatattctcttatgctattgatcgtgatggagtggctcatgttccttcaacatataaaaataggactagtacaaatttacaacggcatgaagtcgataatatctcacatactgactacaccgtatctcgaaaattaaaaacattgatgaccgaagacaaacgctatgatgtcattgatcaaaaagacattcctgacccagcattaagagaacaaatcattcaacaagttggacagtataaaggcgatttggaacgttataacaagacattggtgcttacaggagataagattcaaaatcttaaaggactagaaaaattaagcaagttacaaaaattagagttgcgccagctatctaacgttaaagaaattactccagaacttttgccggaaagcatgaaaaaagatgctgagcttgttatggtaggcatgactggtttagaaaaactaaaccttagtggtctaaatcgtcaaactttagacggtatagacgtgaatagtattacgcatttgacatcatttgatatttcacataatagtttggacttgtcggaaaagagtgaagaccgtaaactattaatgactttgatggagcaggtttcaaatcatcaaaaaataacggtgaaaaatacggcttttgaaaatcaaaaaccgaaaggttattatcctcagacgtatgataccaaagaaggtcattatgatgttgataatgcagaacatgatattttaactgattttgtttttggaactgttactaaacgtaatacctttattggagacgaagaagcatttgctatctataaagaaggagctgtcgatggtcgacaatatgtgtctaaagactatacttatgaagcttttcgtaaagactataaaggttacaaggttcatttaactgcttctaacctaggagaaacagttacttctaaggtaactgctactactgatgaaacttacttagtagatgtttctgatggggaaaaagttgttcaccacatgaaactcaatataggatctggtgccatcatgatggaaaatctggcaaaaggggctaaagtgattggtacatctggggactttgagcaagcaaagaagattttcgatggtgaaaagtcagatagattcttcacttggggacaaactaactggatagcttttgatctaggagaaattaatcttgcgaaggaatggcgtttatttaatgcagagacaaatactgaaataaagacagatagtagcttaaacgtggctaaaggacgtcttcagattttaaaagatacaactattgatttagaaaaaatggacataaaaaatcgtaaagagtatctgtcgaatgatgaaaattggactgatgttgctcagatggatgatgcaaaagcgatatttaatagtaaattatccaatgttttatctcggtattggcggttttgtgtagatggtggagctagctcttattaccctcaatataccgaacttcaaatcctcggacaacgtttatcaaatgatgtcgctaatacgctgaaggattgatattaaagtcctacgattacaaagataaacagtttctcaaagttgatttaagcactttaaaacctaactaaaaatctgagatgaatagtcccagatttttagtcttttataggttttgatgacataaagctaaataatcgttagactaccagaaagtggcgc

SEQ ID NO: 141 - DNA, 66 bp, synthetic construct
  source 1..66: mol_type = other DNA
ggctccggcgctaccaacttctccctgctgaagcaggctggcgatgtggaggagaatcccggccct

SEQ ID NO: 142 - DNA, 63 bp, synthetic construct
  source 1..63: mol_type = other DNA
ggttcaggtgagggccgcggcagtctgctgacttgcggcgacgtcgaggagaaccccggtcct

SEQ ID NO: 143 - DNA, 69 bp, synthetic construct
  source 1..69: mol_type = other DNA
gggagtgggcaatgcactaactatgcattgcttaagttggctggtgatgtagaatctaatcctgggccc

SEQ ID NO: 144 - DNA, 75 bp, synthetic construct
  source 1..75: mol_type = other DNA
ggcagtggggtgaagcagaccctcaattttgacttgttgaaactcgcaggcgacgtagaatctaaccccggacct

SEQ ID NO: 145 - DNA, 7840 bp, synthetic construct
  source 1..7840: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccaccatgatccacaccaatctgaagaagaagtttagctgttgcgtgctggtgttcctgctgttcgctgtgatctgcgtgtggaaggagaagaagaagggcagctattatgactcctttaagctgcagaccaaggagttccaggtgctgaagtccctgggcaagctggccatgggcagcgactcccagtccgtgagctcctccagcacccaggacccccaccggggaaggcagaccctgggatccctgaggggcctggctaaggccaagcctgaggcttccttccaggtgtggaataaggatagctccagcaagaacctgatccctaggctgcagaagatctggaagaactatctgagcatgaacaagtataaggtgagctataagggccctggccctggcatcaagttctccgccgaggccctgaggtgccacctgagagatcacgtgaatgtgagcatggtggaggtgaccgacttcccttttaacacctccgagtgggagggctacctgcccaaggagagcatccggaccaaggccggcccttggggcagatgtgccgtggtgtccagcgccggctccctgaagagctcccagctgggccgggagatcgatgaccacgatgctgtgctgaggttcaatggcgctcctaccgctaactttcagcaggatgtgggcaccaagaccaccatccggctgatgaatagccagctggtgaccaccgagaagcggtttctgaaggactccctgtacaacgagggcatcctgatcgtgtgggacccctccgtgtaccacagcgacatccccaagtggtatcagaaccccgactacaacttcttcaataattacaagacctaccggaagctgcaccccaaccagcccttttatatcctgaagccccagatgccttgggagctgtgggacatcctgcaggagatctcccccgaggagatccagcctaacccccctagcagcggcatgctgggcatcatcatcatgatgaccctgtgcgatcaggtggatatctacgagttcctgcccagcaagcggaagaccgacgtgtgttattattatcagaagttcttcgattccgcctgcaccatgggcgcctatcaccctctgctgtacgagaagaatctggtgaagcacctgaaccagggcaccgacgaggacatctatctgctgggcaaggccaccctgcccggctttaggaccatccactgcggcagcggcgctaccaatttcagcctgctgaagcaggctggcgatgtggaggagaaccccggccccatgaggctgcgggagccattgctgagcggctccgctgccatgcctggcgcttctctgcagagggcttgtaggctgctggtggccgtgtgcgccctgcacctgggagtgaccctggtgtactacctggctggccgggacctgtcccggctgcctcagttggtgggcgtgtccacccctctgcagggcggaagcaatagcgccgctgccatcggccagtcctccggagagctgcggaccggaggcgctaggccacctccacctctgggcgcttcctcccagcctcggcctggaggagatagcagccccgtggtggatagcggccctggcccagcttctaacctgaccagcgtgcctgtgccccacaccaccgccctgagcctgcctgcttgccccgaggagtcccccctgctggtgggaccaatgctgatcgagttcaatatgcctgtggatctggagctggtggccaagcagaatcccaatgtgaagatgggcggccggtacgctccccgggattgtgtgtcccctcacaaggtggctatcatcatccctttccggaaccggcaggagcacctgaagtactggctgtattacctgcaccccgtgctgcagaggcagcagctggactatggcatctacgtgatcaatcaggctggcgacaccatcttcaatcgggctaagctgctgaacgtgggctttcaggaggctctgaaggactacgactacacctgctttgtgttcagcgatgtggacctgatccccatgaacgaccacaacgcttataggtgcttctcccagcctaggcacatctccgtggctatggacaagtttggcttctccctgccctacgtgcagtattttggcggcgtgtccgccctgagcaagcagcagtttctgaccatcaatggctttcctaacaattactggggctggggcggcgaggatgatgacatcttcaaccggctggtgttcaggggcatgtccatcagcaggcctaacgctgtggtgggccggtgtaggatgatcaggcactcccgggacaagaagaatgagcccaatcctcagcggtttgaccggatcgcccacaccaaggagaccatgctgtccgacggcctgaattccctgacctaccaggtgctggacgtgcagaggtatcctctgtacacccagatcaccgtggatatcggcaccccttcctgatgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 146 - DNA, 8131 bp, synthetic construct
  source 1..8131: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccaccatgatccacaccaacctgaagaagaagttctcctgctgtgtgctggtgtttctgctgttcgctgtgatctgcgtgtggaaggagaagaagaagggcagctattatgacagctttaagctgcagaccaaggagtttcaggtgctgaagtccctgggcaagctggccatgggctccgactcccagtccgtgtccagctccagcacccaggatcctcaccggggcaggcagaccctgggctctctgagaggcctggctaaggctaagcccggcggcggaggcagcggaggaggaggatctggcggcggaggatccaatgtggtggccccttccctggaggtgtatgtggaccacgcctccctgcccaccctgcagcagctgatggatatcatcaagagcgaggaggagaatcctaccgcccagcggtatatcgcctggggccggatcgtgcctaccgacgagcagatgaaggagctgaacatcacctccttcgctctgatcaacaatcacacccccgccgacctggtgcaggagatcgtgaagcaggcccagaccaagcaccggctgaacgtgaagctgtcctccaacaccgcccactcctttgacaacctggtgcctatcctgaaggagctgaattcctttaataacgtgaccgtgaccaacatcgacctgtacgacgacggctccatggagtatgtgaacctgtataactggagggacaccctgaataagaccgacaatctgaagatcggcaaggactatctggaggacgtgatcaatggcatcaatgaggacacctccaacaccggcaccagcagcgtgtataactggcagaagctgtaccctgctaattaccactttctgaggaaggattatctgaccctggagccttccctgcacgagctgagggactacatcggcgatagcctgaagcagatgcagtgggatggctttaagaagttcaacagcaagcagcaggagctgtttctgagcatcgtgaattttgacaagcagaagctgcagaacgagtataactcctccaatctgcctaactttgtgttcaccggcaccaccgtgtggggcggcaatcacgagcgggagtactatgccaagcagcagatcaacgtgatcaataatgccatcaatgagagcagcccccactacctgggcaatagctacgatctgttcttcaagggccaccctggcggcggcatcatcaacaccctgatcatgcagaactatccttccatggtggatatccctagcaagatctccttcgaggtgctgatgatgaccgacatgctgcccgacgccgtggctggcatcgcctctagcctgtattttaccatccccgctgagaagatcaagttcatcgtgtttaccagcaccgagaccatcaccgaccgggagaccgccctgaggtccccactggtgcaggtgatgatcaagctgggcatcgtgaaggaggagaacgtgctgttttgggccgacctgcccaactgtgagaccggcgtgtgtggctccggcgctaccaacttctccctgctgaagcaggctggcgatgtggaggagaatcccggccctatgcggctgcgggagcccctgttgtccggctctgccgctatgcccggcgcttccctgcagagagcctgtcggctgctggtggctgtgtgcgctctgcacctgggcgtgaccctggtgtactatctggctggccgggatctgagccggctgcctcagctggtgggcgtgagcacccccctgcagggaggatccaactccgccgctgccatcggccagtcctccggagagctgcggaccggaggcgctaggcctccaccaccactgggcgcttcttcccagcctcggcccggaggagatagcagccccgtggtggactccggccctggacctgcttccaacctgaccagcgtgcccgtgcctcacaccaccgctctgtccctgcccgcctgtcccgaggagtcccctctgctggtgggccctatgctgatcgagtttaatatgcctgtggacctggagctggtggctaagcagaatcctaacgtgaagatgggcggcaggtacgctcccagggactgcgtgagccctcacaaggtggccatcatcatccctttccggaaccggcaggagcacctgaagtactggctgtactacctgcaccctgtgctgcagaggcagcagctggactatggcatctacgtgatcaaccaggccggcgacaccatctttaaccgggccaagctgctgaatgtgggctttcaggaggccctgaaggattatgactacacctgctttgtgtttagcgatgtggatctgatccctatgaacgatcacaacgcctaccggtgcttcagccagcctcggcacatcagcgtggccatggacaagttcggcttctccctgccttacgtgcagtacttcggcggcgtgtccgccctgtccaagcagcagttcctgaccatcaacggctttcctaataactactggggctggggcggcgaggatgatgacatctttaatcggctggtgtttaggggcatgagcatcagccggcctaacgccgtggtgggcaggtgcaggatgatcaggcactcccgggacaagaagaacgagcctaatcctcagaggtttgacaggatcgctcacaccaaggagaccatgctgagcgacggcctgaatagcctgacctaccaggtgctggacgtgcagaggtaccctctgtatacccagatcaccgtggatatcggcacccctagctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 147 - DNA, 6573 bp, synthetic construct
  source 1..6573: mol_type = other DNA
gacggatcgggagatctcccgatcccctatggtgcactctcagtacaatctgctcgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccaccatgatccacaccaatctgaagaagaagtttagctgttgcgtgctggtgttcctgctgttcgctgtgatctgcgtgtggaaggagaagaagaagggcagctattatgactcctttaagctgcagaccaaggagttccaggtgctgaagtccctgggcaagctggccatgggcagcgactcccagtccgtgagctcctccagcacccaggacccccaccggggaaggcagaccctgggatccctgaggggcctggctaaggccaagcctgaggcttccttccaggtgtggaataaggatagctccagcaagaacctgatccctaggctgcagaagatctggaagaactatctgagcatgaacaagtataaggtgagctataagggccctggccctggcatcaagttctccgccgaggccctgaggtgccacctgagagatcacgtgaatgtgagcatggtggaggtgaccgacttcccttttaacacctccgagtgggagggctacctgcccaaggagagcatccggaccaaggccggcccttggggcagatgtgccgtggtgtccagcgccggctccctgaagagctcccagctgggccgggagatcgatgaccacgatgctgtgctgaggttcaatggcgctcctaccgctaactttcagcaggatgtgggcaccaagaccaccatccggctgatgaatagccagctggtgaccaccgagaagcggtttctgaaggactccctgtacaacgagggcatcctgatcgtgtgggacccctccgtgtaccacagcgacatccccaagtggtatcagaaccccgactacaacttcttcaataattacaagacctaccggaagctgcaccccaaccagcccttttatatcctgaagccccagatgccttgggagctgtgggacatcctgcaggagatctcccccgaggagatccagcctaacccccctagcagcggcatgctgggcatcatcatcatgatgaccctgtgcgatcaggtggatatctacgagttcctgcccagcaagcggaagaccgacgtgtgttattattatcagaagttcttcgattccgcctgcaccatgggcgcctatcaccctctgctgtacgagaagaatctggtgaagcacctgaaccagggcaccgacgaggacatctatctgctgggcaaggccaccctgcccggctttaggaccatccactgctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 148 - DNA, 6550 bp, synthetic construct
  source 1..6550: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccaccatgaggctgcgggagccattgctgagcggctccgctgccatgcctggcgcttctctgcagagggcttgtaggctgctggtggccgtgtgcgccctgcacctgggagtgaccctggtgtactacctggctggccgggacctgtcccggctgcctcagttggtgggcgtgtccacccctctgcagggcggaagcaatagcgccgctgccatcggccagtcctccggagagctgcggaccggaggcgctaggccacctccacctctgggcgcttcctcccagcctcggcctggaggagatagcagccccgtggtggatagcggccctggcccagcttctaacctgaccagcgtgcctgtgccccacaccaccgccctgagcctgcctgcttgccccgaggagtcccccctgctggtgggaccaatgctgatcgagttcaatatgcctgtggatctggagctggtggccaagcagaatcccaatgtgaagatgggcggccggtacgctccccgggattgtgtgtcccctcacaaggtggctatcatcatccctttccggaaccggcaggagcacctgaagtactggctgtattacctgcaccccgtgctgcagaggcagcagctggactatggcatctacgtgatcaatcaggctggcgacaccatcttcaatcgggctaagctgctgaacgtgggctttcaggaggctctgaaggactacgactacacctgctttgtgttcagcgatgtggacctgatccccatgaacgaccacaacgcttataggtgcttctcccagcctaggcacatctccgtggctatggacaagtttggcttctccctgccctacgtgcagtattttggcggcgtgtccgccctgagcaagcagcagtttctgaccatcaatggctttcctaacaattactggggctggggcggcgaggatgatgacatcttcaaccggctggtgttcaggggcatgtccatcagcaggcctaacgctgtggtgggccggtgtaggatgatcaggcactcccgggacaagaagaatgagcccaatcctcagcggtttgaccggatcgcccacaccaaggagaccatgctgtccgacggcctgaattccctgacctaccaggtgctggacgtgcagaggtatcctctgtacacccagatcaccgtggatatcggcaccccttcctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 149 - DNA, 8201 bp, synthetic construct
  source 1..8201: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctaagcttgccgccaccatgagggtccccgctcagctcctggggctcctgctgctctggctcccaggtgcacgatgtgatatccaaatgactcaaagtccaagtagtctgtccgcaagcgtcggcgatcgcgtgaccatcacatgtagagcttctcaaggcatccggaactacctggcttggtaccagcagaagcctggcaaggcccccaagctgctgatctatgccgcttctaccctccagtccggcgtgcctagcagattttccggctccggatctggaacagacttcaccctgaccatctcctctctgcagcctgaggacgtggccacctactactgccagcggtacaacagagccccatacaccttcggccagggcaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgaggatccactagtccagtgtggtggaattcagatccgttaacggttaccaactacctagactggattcgtgacaacatgcggccgtgatatctacgtatgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgacagctatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgggtacgtcctcacattcagtgatcagcactgaacacagacccgcggccgctcgaggccgccaccatgggttggagcctcatcttgctcttccttgtcgctgttgctacgcgtgtcgaagtgcaactagtggaaagtggtggtggtctcgtgcagcccggaaggtctctgcggctgtcctgtgctgcttccggcttcaccttcgacgattatgccatgcactgggtccggcaagctcctggcaagggcctggagtgggtctctgccatcacctggaattctggccacatcgactacgccgactccgtggaaggcagatttaccatctccagagacaacgccaagaacagcctgtacctgcagatgaactccctgagagccgaggatacagccgtgtactactgcgccaaagtgtcctacctgtctaccgcttctagcctggactactggggccagggcaccctggtgaccgtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 150 - RNA, 23 nt, synthetic construct
  source 1..23: mol_type = other RNA
catggactggttcctggcgttgg

SEQ ID NO: 151 - RNA, 23 nt, synthetic construct
  source 1..23: mol_type = other RNA
ctccagctttgcaagaatcttgg

SEQ ID NO: 152 - RNA, 23 nt, synthetic construct
  source 1..23: mol_type = other RNA
ataaaacaataaggtcccccagg

SEQ ID NO: 153 - RNA, 4158 nt, synthetic construct
  source 1..4158: mol_type = other RNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagggctccgga

SEQ ID NO: 154 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

(19) United States
(12) Patent Application Publication
(10) Pub. No.: US 2025/0333518 A1
(43) Pub. Date: Oct. 30, 2025

(54) ENGINEERED GLYCOPROTEIN POPULATION AND USES THEREOF

(71) Applicant: Rock BioMedical, Inc., Taipei City (TW)

(72) Inventors: Jeng-Shin LEE, Lincoln, MA (US); Szu-Wen WANG, Taipei City (TW); Chi-Huey WONG, Rancho Santa Fe, CA (US)

(21) Appl. No.: 19/072,276

(22) Filed: Mar. 6, 2025

Related U.S. Application Data
(63) Continuation-in-part of application No. PCT/US24/60715, filed on Dec. 18, 2024.
(63) Continuation-in-part of application No. PCT/US24/34588, filed on Jun. 19, 2024.
(60) Provisional application No. 63/639,286, filed on Apr. 26, 2024.

(51) Int. Cl. C07K 16/28; C07K 16/24; C07K 16/32; C12N 5/071; C12N 9/10; C12N 9/22; C12N 15/11; C12N 15/85

(52) U.S. Cl. CPC C07K 16/2863; C07K 16/241; C07K 16/2887; C07K 16/32; C12N 5/0682; C12N 5/0686; C12N 9/1051; C12N 9/1081; C12N 9/22; C12N 15/111; C12N 15/85; C07K 2317/14; C07K 2317/41; C07K 2317/732; C07K 2317/734; C07K 2317/94; C12N 2310/20; C12Y 204/01; C12Y 204/99

(57) ABSTRACT

The present disclosure relates to enriched engineered glycoprotein populations with a therapeutically optimized heterogeneous glycan profile. The heterogeneous glycan profile comprises sialylated complex type glycans, which are associated with improved efficacy of the glycoprotein. The present disclosure also relates to compositions and uses of the engineered glycoprotein populations and cell-based method of producing them.

### CROSS-REFERENCE TO RELATED APPLICATIONS

This application claims the benefit of priority of U.S. Provisional Patent Applications No. 63/639,286, filed on Apr. 26, 2024, PCT Patent Application No. PCT/US24/34588, filed on Jun. 19, 2024, and PCT Patent Application No. PCT/US24/60715, filed on Dec. 18, 2024. The entireties of the aforementioned applications are incorporated herein by reference.

### SEQUENCE LISTING

The instant disclosure contains a Sequence Listing which is submitted electronically in.xml format and is hereby incorporated by reference in its entirety. The .xml copy, created on Mar. 5, 2025, is named “A1000-02000PCT_20250305_SeqListing.xml” and is 200,005 bytes in size.

### FIELD

The present disclosure is related generally to methods and compositions for generating therapeutic antibodies and related glycoproteins with enriched and optimized engineered glycan profiles to optimize biological activity and/or efficacy; particularly, the present disclosure is related to cell-based pathway engineering methods and compositions for enriching populations of therapeutic antibodies with specific optimized glycan profiles with increased/improved relative percent sialylation in various glycoforms thereby improving biological and/or therapeutic efficacy and treatment methods of using the same.

### BACKGROUND OF THE INVENTION

Over the past few years, protein therapeutics, including therapeutic antibodies, antibody conjugates, or fusion proteins with target-binding abilities, have gained increasing prominence in almost every field of medicine. Most of these protein therapeutics are glycoproteins. Taking therapeutic antibodies as examples, the glycans on the Fc region of the antibodies have been proven to be essential and determinative of therapeutic efficacy. Therefore, glycoprotein's post-translational modifications, particularly towards their glycan profile, have drawn increased attention in academic and industrial sectors. Previous generations of glycoengineering technology based on in vitro chemoenzymatic reactions have met with mixed success due to the inherent challenges in the multi-step process and the need for stringent CMC control; rendering vast difficulties in terms of cost and time in the subsequent regulatory processes and clinical development processes. Thus, the development of mammalian cell expression platforms that are capable of directly producing engineering glycoproteins with desired/optimized glycan profiles is one of the important focuses in the field of developing/producing the next generation of therapeutic antibodies.

### BRIEF SUMMARY OF THE INVENTION

Accordingly, the present disclosure provides methods and compositions directed to cell-based pathway engineering platforms for generating the next generation of therapeutic antibodies with optimized and highly sialylated glycan profiles exhibiting surprising and unexpected improved and/or optimized therapeutic characteristics compared to their existing commercial counterparts.

In one aspect, the disclosure provides an enriched but not homogeneous population of therapeutic antibodies with optimized glycan and/or sialylation profiles directly via cell-based pathway engineering. Particularly, in one aspect, the disclosure provides an engineered glycoprotein population with a heterogeneous glycan profile, comprising a plurality of glycoproteins, wherein the heterogenous glycan profile collectively comprises at least 20%, 25%, 30%, 35%, 40%, 45%, 50%, 55%, 60%, 65%, 70%, 72%, 75%, 77%, 80%, 85%, 86%, 87%, 88%, 89%, 90%, 91%, 92%, 93%, 94%, or 95% of sialylated complex type (SCT) glycans, and the SCT glycans include two or more types of sialylated glycans. Particularly, in an example of this aspect, the disclosure provides improved therapeutic proteins (e.g., an antibody population) comprising an optimized glycan profile and/or optimized sialylation profile. Particularly, in an example of this aspect, the optimized sialylation profile is characterized by a high relative percent of sialylation. In such aspect, the engineered glycoprotein population of the present disclosure is characterized by improved ADCC, CDC, ADCP, vaccinal effect, and/or extended half-life compared to their reference product counterpart (e.g., commercial version of the therapeutic antibody), which is without the desired heterogeneous glycan profile.

In one aspect, the present disclosure provides a pharmaceutically acceptable composition comprising an engineered glycoprotein population of the present disclosure and a pharmaceutically acceptable excipient. In one embodiment, the engineered glycoprotein population is generated/produced by cell-based pathway engineering.

In one aspect, the present disclosure provides a method of increasing binding between an Fc gamma receptor (FcγR) and a glycoprotein, comprising: engineering the glycoprotein to obtain an engineered glycoprotein population, wherein the engineered glycoprotein population has a desired glycan profile in accordance with the present disclosure.

In one aspect, the present disclosure provides a method of treating a disease caused by a dysfunctional cell, comprising administering to a subject in need thereof a pharmaceutically effective amount of the engineered glycoprotein population of the present disclosure, wherein the glycoprotein is configured to target the dysfunctional cell and thereby improving therapeutic endpoints.

In one aspect, the present disclosure provides a cell for expressing a sialylated glycoprotein, wherein the cell constitutively and/or controllably expresses an exogenous sialyltransferase catalytic peptide and an exogenous galactosyltransferase catalytic peptide, wherein the exogenous sialyltransferase catalytic peptide and the exogenous galactosyltransferase catalytic peptide are translated in close proximity; and wherein the cell is deficient in endogenous fucosyltransferase activity; and deficiency in endogenous fucosyltransferase activity is made by introducing a vector into a parent cell of the cell, wherein the vector comprises: a nucleic acid encoding a Clustered Regularly Interspaced Short Palindromic Repeat (CRISPR) crRNA comprising a nucleotide sequence as set forth in SEQ ID NO: 150, SEQ ID NO: 151, or SEQ ID NO: 152; and a nucleic acid encoding a tracrRNA.

In one aspect, the present disclosure provides a cell for expressing a sialylated glycoprotein, comprising: a first nucleic acid, encoding an exogenous sialyltransferase catalytic peptide; a second nucleic acid, encoding an exogenous galactosyltransferase catalytic peptide; and a vector comprising: a nucleic acid encoding a Clustered Regularly Interspaced Short Palindromic Repeat (CRISPR) crRNA comprising a nucleotide sequence as set forth in SEQ ID NO: 150, SEQ ID NO: 151, or SEQ ID NO: 152; and a nucleic acid encoding a tracrRNA.

In one aspect, the present disclosure provides a cell culture medium, comprising a basal medium and a peracetylated 4-fluoro N-Acetylmannosamine (4-F-ManNAc), wherein the basal medium comprises a carbon source, amino acids, and minerals, and the cell culture medium is devoid of sialyltransferase.

In one aspect, the present disclosure provides a cell culture medium, comprising a complex culture medium and a peracetylated 4-fluoro N-Acetylmannosamine (4-F-ManNAc), wherein the cell culture medium is devoid of sialyltransferase.

### BRIEF DESCRIPTION OF THE DRAWINGS

FIG. 1 is a graphic representation of a flow cytometry assay showing the exemplary results of an SNA staining assay conducted to demonstrate the exemplary glycoengineered Adalimumab produced by using the exemplary CHO cell (SWG-006) of the present disclosure. A comparative cell transfected with B4GALT1 and ST6GAL1 configured to be expressed in separate transcriptions, and an untransfected cell (i.e., a parental cell, as a negative control). In this particular exemplary embodiment, the glycoengineered Adalimumab antibodies produced by the exemplary cell (SWG-006) of the present disclosure had a 5.44% SNA-positive population. The Adalimumab antibodies produced by the comparative cell had a 4.58% SNA-positive population. The parental cell expressed 0.022% SNA-positive Adalimumab antibodies.

FIG. 2 is a graphic representation of a flow cytometry assay showing the results of an SNA staining assay conducted to examine the glycoengineered Adalimumab produced by using the exemplary SWG-006 CHO cell of the present disclosure, the exemplary SWG-015 cell of the present disclosure, and untransfected cells (i.e., a parental cell, as a negative control). The glycoengineered Adalimumab antibodies produced by the SWG-006 cells of the present disclosure had a 39.8% SNA-positive population. The glycoengineered Adalimumab antibodies produced by the SWG-015 cells of the present disclosure had a 28.5% SNA-positive population. The parental cell expressed 0.25% SNA-positive Adalimumab antibodies.

FIG. 3A is a graphic representation of an LS/MS-MS spectrum conducted to demonstrate the glycoforms of the glycoengineered Adalimumab antibodies produced by an exemplary stabilized clone SAII-A3 of the SWG-006 CHO cell according to the present disclosure. The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 3B is a graphic representation of an LS/MS-MS spectrum conducted to demonstrate the glycoforms of the exemplary glycoengineered Adalimumab antibodies produced by an exemplary stabilized clone SAI-G1 of the SWG-006 CHO cell according to the present disclosure. The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 3C is a graphic representation of an LS/MS-MS spectrum conducted to demonstrate the glycoforms of the exemplary glycoengineered Adalimumab antibodies produced by an exemplary stabilized clone SAI-D4 of the SWG-006 CHO cell according to the present disclosure. The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 3D is a graphic representation of an LS/MS-MS spectrum conducted to demonstrate the glycoforms of the exemplary glycoengineered Adalimumab antibodies produced by an exemplary stabilized clone SAI-F12 of the CHO SWG-006 cell according to the present disclosure. The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 3E is a graphic representation of an LS/MS-MS spectrum conducted to demonstrate the glycoforms of the exemplary glycoengineered Adalimumab antibodies produced by a parental CHO cell. No sialylated glycans were observed. The Diamond shape represents sialic acid; the open circle shape represents galactose; the square shape represents GlcNAc; the solid circle shape represents mannose; the triangle shape represents fucose.

FIG. 4A is a graphic representation of an LS/MS-MS spectrum conducted to investigate the glycoforms of a commercial product of Adalimumab (Humira®). No sialylated glycans were observed. The Diamond shape represents sialic acid; the open circle shape represents galactose; the square shape represents GlcNAc; the solid circle shape represents mannose; the triangle shape represents fucose.

FIG. 4B is a graphic representation of an LS/MS-MS spectrum conducted to demonstrate the glycoforms of the glycoengineered Adalimumab antibodies produced by a parental CHO cell. No sialylated glycans were observed. The Diamond shape represents sialic acid; the open circle shape represents galactose; the square shape represents GlcNAc; the solid circle shape represents mannose; the triangle shape represents fucose.

FIG. 4C is a graphic representation of an LS/MS-MS spectrum conducted to demonstrate the glycoforms of the glycoengineered Adalimumab antibodies produced by an exemplary stabilized clone SAII-A3 of the SWG-006 CHO cell according to the present disclosure. The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 5A is a graphic representation of the data of an intact protein mass (IPM) analysis conducted to investigate the glycoforms of the chMC81370 antibodies produced by a parental HEK293T cell. No sialylated glycans were observed. The Diamond shape represents sialic acid; the open circle shape represents galactose; the square shape represents GlcNAc; the solid circle shape represents mannose; the triangle shape represents fucose.

FIG. 5B is a graphic representation of the data of an intact protein mass (IPM) analysis conducted to demonstrate the glycoforms of the chMC81370 antibodies produced by an exemplary HEK293 cell according to the present disclosure. The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 6 is a graphic representation of the data of a glycoform analysis conducted to investigate the chMC81370 antibodies produced by a wild-type cell (i.e. a parental cell) and an exemplary HEK293 cell according to the present disclosure (an “engineered” cell). Left: percentage of fucosylated glycan; Middle: percentage of galactosylated glycan; Right: percentage of sialylated glycan.

FIG. 7 is a graphic representation of the data of an intact protein mass (IPM) analysis conducted to demonstrate the glycoforms of the chMC81370 antibodies produced by an exemplary HEK293 cell according to the present disclosure, untreated (top), treated with Streptococcus pneumonia a2-3 neuraminidase (middle), or treated with Clostridium perfringens neuraminidase (bottom). The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 8 is a graphic representation of the data of an intact protein mass (IPM) analysis conducted to demonstrate the glycoforms of the chMC81370 antibodies produced by a comparative cell transfected with a hB4GalT1 gene but not hST6GAL1 gene. The data shows that the antibodies had increased galactosylation generated from cells with knock-in of hB4GalT1 only but did not show terminal sialylation. The Diamond shape represents sialic acid; the open circle shape represents galactose; the square shape represents GlcNAc; the solid circle shape represents mannose; the triangle shape represents fucose.

FIG. 9A is a graphic representation of the data of an intact protein mass (IPM) analysis conducted to investigate the changes in glycoforms of the chMC81370 antibodies produced by an exemplary HEK293 cell according to the present disclosure over five days. Top: Day 0 to Day 3 (samples collected on Day 3); Bottom: Day 4 to Day 5 (samples collected on Day 5). The sialylated glycans are labeled with solid arrows. The Diamond shape represents sialic acid; the open circle shape represents galactose; the square shape represents GlcNAc; the solid circle shape represents mannose; the triangle shape represents fucose.

FIG. 9B is a graphic representation of the data of an intact protein mass (IPM) analysis conducted to investigate the changes in the glycoforms of the chMC81370 antibodies produced by a comparative FUT8 knock-out cell over five days. Top: Day 0 to Day 3 (samples collected on Day 3); Bottom: Day 4 to Day 5 (samples collected on Day 5). The Diamond shape represents sialic acid; the open circle shape represents galactose; the square shape represents GlcNAc; the solid circle shape represents mannose; the triangle shape represents fucose.

FIG. 10 is a graphic representation of the data of an intact protein mass (IPM) analysis conducted to investigate the changes in the glycoforms of the chMC81370 antibodies produced by an exemplary HEK293 cell according to the present disclosure over three days. Samples were collected on Day 1 (Top), Day 2 (Middle), and Day 3 (Bottom) respectively. The Diamond shape represents sialic acid; the open circle shape represents galactose; the square shape represents GlcNAc; the solid circle shape represents mannose; the triangle shape represents fucose.

FIG. 11 is a graphic representation of ELISA data showing the binding affinity of the antibodies produced by a wild-type cell (i.e. a parental cell) and an exemplary HEK293 cell according to the present disclosure (an “SCT-enriched” cell) to FcγIIA receptors (Left), FcγIIB receptors (Middle), and FcγIIIA receptors (Right). The binding of the antibody was measured by using an anti-human IgG Fc antibody conjugated with HRP.

FIG. 12A presents a result of protein electrophoresis showing that the chMC18370 antibodies expressed by an exemplary HEK293 cell according to the present disclosure (Endo H KI “+”) and a parental GnT1-KO EXPI293 cell (Endo H KI “−”) are different in molecular weight as band shifting was observed.

FIG. 12B presents a result of a transglycosylation assay showing that the chMC18370 antibodies expressed by an exemplary Endo H knock-in cell, according to the present disclosure, had a high efficiency of transglycosylation. Transglycosylation was observed within 15 minutes.

FIG. 12C is a graphic representation of the data of an intact protein mass (IPM) analysis conducted to investigate the glycoforms of the chMC81370 antibodies produced by a parental cell (GnT1-KO EXPI293 cell). The square shape represents GlcNAc, and the solid circle shape represents mannose.

FIG. 12D is a graphic representation of the data of an intact protein mass (IPM) analysis conducted to investigate the glycoforms of the chMC81370 antibodies produced by an exemplary Endo H knock-in cell according to the present disclosure. The square shape represents GlcNAc, and the solid circle shape represents mannose.

FIG. 12E is a graphic representation of the data of a glycoform analysis conducted to investigate the chMC81370 antibodies produced by a GnT1-KO EXPI293 cell, an exemplary Endo H knock-in according to the present disclosure, and an exemplary Endo S2 knock-in cell according to the present disclosure. Left: Percentage of mannose type glycan; Right: percentage of mono-GlcNAc glycan.

FIG. 13A presents a result of protein electrophoresis showing that the chMC18370 antibodies expressed by an exemplary HEK293 cell according to the present disclosure (Endo S2 KI “+”) and a parental GnT1-KO EXPI293 cell (Endo S2 KI “−”) are different in molecular weight as band shifting was observed.

FIG. 13B presents a result of a transglycosylation assay showing that the chMC18370 antibodies expressed by an exemplary Endo S2 knock-in cell, according to the present disclosure, had a high efficiency of transglycosylation. Transglycosylation was observed within 15 minutes.

FIG. 13C is a graphic representation of the data of an intact protein mass (IPM) analysis conducted to investigate the glycoforms of the chMC81370 antibodies produced by an exemplary Endo S2 knock-in cell according to the present disclosure. The square shape represents GlcNAc.

FIG. 14A is a graphic representation of ELISA data showing the binding affinity of the antibodies produced by the exemplary ExpiCHO (ST6GAL1+/B4GALT1+/FUT8-) cells according to the present disclosure (“SCT-enriched”; see Example 4), and the exemplary Endo H knock-in cells with transglycosylation according to the present disclosure (“SCT homogeneous”; see Example 9).

FIG. 14B is a graphic representation of the data of an ADCC reporter assay showing the comparison between the antibodies produced by a wild-type cell (i.e. a parental cell), the exemplary ExpiCHO (ST6GAL1+/B4GALT1+/FUT8-) cells according to the present disclosure (“SCT-enriched””; see Example 4), and the exemplary Endo H knock-in cells with transglycosylation according to the present disclosure (“SCT homogeneous”; see Example 9).

FIG. 15 is an electrophoresis photo of PCR products. Lane 1, Lane 2, and Lane 3 were loaded with PCR products obtained from cells of low LCA signal, cells of intermediate signal, and cells of intermediate signal and further added with 20 μg/mL LCA. Lane 4 was loaded with the PCR products from parental cells.

FIG. 16 shows graphic representations of FACS spectrums. The left shows cells of low LCA signals, the Middle shows cells of intermediate LCA signals, and the right shows parental cells.

FIG. 17 is a graphic representation of an intact protein mass (IPM) analysis (LC/MASS) conducted to demonstrate the glycoengineered Herceptin® protein population's glycan profile produced according to the present disclosure. The peaks representing heterogeneous sialylated glycans are numbered. Peak 1 is SiazGal₂GlcNAc₂Man₃GlcNAc₂; Peak 2 is SiaHexGal₂GlcNAC₂Man₃GlcNAc₂; Peak 3 is SiaGal₂GlcNAc₂Man₃GlcNAc₂; Peak 4 is SiaGalGlcNAcMansGlcNAc₂; Peak 5 is SiaGalGlcNAc₂Man₃GlcNAc₂; Peak 6 is SiaGalGlcNAcMan₄GlcNAc₂; and Peak 7 is SiaGalGlcNAcMan₃GlcNAc₂.

FIG. 18 is a graphic representation of an intact protein mass (IPM) analysis (LC/MASS) conducted to demonstrate the glycan profile of a commercial Herceptin® product.

FIG. 19 is a graphic representation of an intact protein mass (IPM) analysis (LC/MASS) conducted to demonstrate the Herceptin® protein population's glycan profile produced by CHO-S cells.

FIG. 20 is a graphic representation of an intact protein mass (IPM) analysis (LC/MASS) conducted to demonstrate the glycoengineered MabThera® protein population's glycan profile produced according to the present disclosure. The peaks representing heterogeneous sialylated glycans are numbered. Peak 1 is SiazGal₂GlcNAczMan₃GlcNAc₂; Peak 2 is SiaHexGal₂GlcNAczMan₃GlcNAc₂; Peak 3 is SiaGal₂GlcNAc₂Man₃GlcNAc₂; Peak 4 is SiaGalGlcNAcMansGlcNAc₂; Peak 5 is SiaGalGlcNAc₂Man₃GlcNAc₂; Peak 6 is SiaGalGlcNAcMan₄GlcNAc₂; and Peak 7 is SiaGalGlcNAcMan₃GlcNAc₂.

FIG. 21 is a graphic representation of an intact protein mass (IPM) analysis (LC/MASS) conducted to demonstrate the glycan profile of a commercial MabThera® product.

FIG. 22 is a graphic representation of an intact protein mass (IPM) analysis (LC/MASS) conducted to demonstrate the MabThera® protein population's glycan profile produced by CHO-S cells.

FIG. 23 is a graphic representation of an intact protein mass (IPM) analysis (LC/MASS) conducted to demonstrate the glycoengineered Erbitux® protein population's glycan profile produced according to the present disclosure. The peaks representing heterogeneous sialylated glycans are numbered. Peak 1 is SiazGal2GlcNAczMan3GlcNAc2; Peak 2 is SiaHexGal2GlcNAC2Man3GlcNAc2; Peak 3 is SiaGal2GlcNAc2Man3GlcNAc2; Peak 4 is SiaGalGlcNAcMansGlcNAc2; Peak 5 is SiaGalGlcNAc2Man3GlcNAc2; Peak 6 is SiaGalGlcNAcMan4GlcNAc2; and Peak 7 is SiaGalGlcNAcMan3GlcNAc2.

FIG. 24 is a graphic representation of an intact protein mass (IPM) analysis (LC/MASS) conducted to demonstrate the glycan profile of a commercial Erbitux® product.

FIG. 25 is a graphic representation of an intact protein mass (IPM) analysis (LC/MASS) conducted to demonstrate the Erbitux® protein population's glycan profile produced by CHO-S cells.

FIG. 26 is a graphic representation of an intact protein mass (IPM) analysis (LC/MASS) conducted to demonstrate the glycoengineered Humira® protein population's glycan profile produced according to the present disclosure. The peaks representing heterogeneous sialylated glycans are numbered. Peak 1 is SiazGal₂GlcNAczMan₃GlcNAC₂Fuc, and Peak 3 is SiaGal₂GlcNAC₂Man₃GlcNAcz or SiaGal₂GlcNAc₂Man₃GlcNAc₂Fuc.

FIG. 27 is a graphic representation of an intact protein mass (IPM) analysis (LC/MASS) conducted to demonstrate the glycan profile of a commercial Humira® product.

FIG. 28 is a graphic representation of an intact protein mass (IPM) analysis (LC/MASS) conducted to demonstrate the Humira® protein population's glycan profile produced by CHO-S cells.

FIG. 29 is a graphic representation of an intact protein mass (IPM) analysis (LC/MASS) conducted to demonstrate the glycoengineered Avastin® protein population's glycan profile produced according to the present disclosure. The peaks representing heterogeneous sialylated glycans are numbered. Peak 1 is SiazGal₂GlcNAc₂Man₃GlcNAc₂Fuc; Peak 2 is SiaHexGal₂GlcNAc₂Man₃GlcNAc₂; Peak 3 is SiaGal₂GlcNAczMan₃GlcNAc₂; Peak 4 is SiaGalGlcNAcMansGlcNAc₂Fuc; Peak 5 is SiaGalGlcNAc₂Man₃GlcNAc₂Fuc; Peak 6 is SiaGalGlcNAcMan₄GlcNAc₂Fuc; and Peak 7 is SiaGalGlcNAcMan₃GlcNAc₂Fuc.

FIG. 30 is a graphic representation of an intact protein mass (IPM) analysis (LC/MASS) conducted to demonstrate the glycan profile of a commercial Avastin® product.

FIG. 31 is a graphic representation of an intact protein mass (IPM) analysis (LC/MASS) conducted to demonstrate the Avastin® protein population's glycan profile produced by CHO-S cells.

FIG. 32 is a graphic representation of an intact protein mass (IPM) analysis (LC/MASS) conducted to demonstrate the glycoengineered Yervoy® protein population's glycan profile produced according to the present disclosure. The peaks representing heterogeneous sialylated glycans are numbered. Peak 1 is SiazGal₂GlcNAC₂Man₃GlcNAc₂Fuc; Peak 2 is SiaHexGal₂GlcNAc₂Man₃GlcNAc₂Fuc; Peak 3 is SiaGal₂GlcNAc₂Man₃GlcNAc₂Fuc; Peak 4 is SSiaGalGlcNAcMansGlcNAc₂Fuc; Peak 5 is SiaGalGlcNAC₂Man₃GlcNAc₂Fuc; Peak 6 is SiaGalGlcNAcMan₄GlcNAc₂Fuc; and Peak 7 is SiaGalGlcNAcMan₃GlcNAc₂Fuc.

FIG. 33 is a graphic representation of an intact protein mass (IPM) analysis (LC/MASS) conducted to demonstrate the glycan profile of a commercial Yervoy® product.

FIG. 34 is a graphic representation of an intact protein mass (IPM) analysis (LC/MASS) conducted to demonstrate the Yervoy® protein population's glycan profile produced by CHO-S cells.

FIG. 35 is a graphic representation of an intact protein mass (IPM) analysis (LC/MASS) conducted to demonstrate the glycoengineered Keytruda® protein population's glycan profile produced according to the present disclosure. The peaks representing heterogeneous sialylated glycans are numbered. Peak 1 is Sia₂Gal₂GlcNAc₂Man₃GlcNAc₂Fuc; Peak 3 is SiaGal₂GlcNAc₂Man₃GlcNAc₂Fuc; Peak 4 is SiaGalGlcNAcMansGlcNAc₂Fuc; Peak 5 is SiaGalGlcNAC₂Man₃GlcNAc₂Fuc; Peak 6 is SiaGalGlcNAcMan₄GlcNAc₂Fuc; and Peak 7 is SiaGalGlcNAcMan₃GlcNAc₂Fuc.

FIG. 36 is a graphic representation of an intact protein mass (IPM) analysis (LC/MASS) conducted to demonstrate the glycan profile of a commercial Keytruda® product.

FIG. 37 is a graphic representation of an intact protein mass (IPM) analysis (LC/MASS) conducted to demonstrate the Keytruda® protein population's glycan profile produced by CHO-S cells.

FIG. 38 is a graphic representation of an intact protein mass (IPM) analysis (LC/MASS) conducted to demonstrate the glycoengineered Opdivo® protein population's glycan profile produced according to the present disclosure. The peaks representing heterogeneous sialylated glycans are numbered. Peak 1 is SiazGal₂GlcNAC₂Man₃GlcNAc₂Fuc; Peak 2 is SiaHexGal₂GlcNAC₂Man₃GlcNAc₂Fuc; Peak 3 is SiaGal₂GlcNAc₂Man₃GlcNAc₂Fuc; Peak 4 is SiaGalGlcNAcMansGlcNAc₂Fuc; Peak 5 is SiaGalGlcNAc₂Man₃GlcNAc₂Fuc; Peak 6 is SiaGalGlcNAcMan₄GlcNAc₂Fuc; and Peak 7 is SiaGalGlcNAcMan₃GlcNAc₂Fuc.

FIG. 39 is a graphic representation of an intact protein mass (IPM) analysis (LC/MASS) conducted to demonstrate the Opdivo® protein population's glycan profile produced by CHO-S cells.

FIG. 40 is a graphic representation of an intact protein mass (IPM) analysis (LC/MASS) conducted to demonstrate the glycoengineered Bavencio® protein population's glycan profile produced according to the present disclosure. The peaks representing heterogeneous sialylated glycans are numbered. Peak 1 is SiazGal₂GlcNAC₂Man₃GlcNAc2; Peak 2 is SiaHexGal₂GlcNAc₂Man₃GlcNAc₂; Peak 3 is SiaGal₂GlcNAc₂Man₃GlcNAc₂; Peak 4 is SiaGalGlcNAcMansGlcNAc₂; Peak 5 is SiaGalGlcNAc₂Man₃GlcNAc₂; Peak 6 is SiaGalGlcNAcMan₄GlcNAc₂; and Peak 7 is SiaGalGlcNAcMan₃GlcNAc₂.

FIG. 41 is a graphic representation of an intact protein mass (IPM) analysis (LC/MASS) conducted to demonstrate the Bavencio® protein population's glycan profile produced by CHO-S cells.

FIG. 42 is a graphic representation of an intact protein mass (IPM) analysis (LC/MASS) conducted to demonstrate the glycoengineered Imfinzi® protein population's glycan profile produced according to the present disclosure. The peaks representing heterogeneous sialylated glycans are numbered. Peak 1 is SiazGal₂GlcNAC₂Man₃GlcNAc₂Fuc; Peak 2 is SiaHexGal₂GlcNAc₂Man₃GlcNAc₂Fuc; Peak 3 is SiaGal₂GlcNAc₂Man₃GlcNAc₂; Peak 4 is SiaGalGlcNAcMansGlcNAc₂Fuc; Peak 5 is SiaGalGlcNAc₂Man₃GlcNAc₂Fuc; Peak 6 is SiaGalGlcNAcMan₄GlcNAc₂Fuc; and Peak 7 is SiaGalGlcNAcMan₃GlcNAc₂Fuc.

FIG. 43 is a graphic representation of an intact protein mass (IPM) analysis (LC/MASS) conducted to demonstrate the glycan profile of a commercial Imfinzi® product.

FIG. 44 is a graphic representation of an intact protein mass (IPM) analysis (LC/MASS) conducted to demonstrate the Imfinzi® protein population's glycan profile produced by CHO-S cells.

FIG. 45 is a graphic representation of an intact protein mass (IPM) analysis (LC/MASS) conducted to demonstrate the glycoengineered Perjeta® protein population's glycan profile produced according to the present disclosure. The peaks representing heterogeneous sialylated glycans are numbered. Peak 1 is SiazGal₂GlcNAC₂Man₃GlcNAc₂Fuc; Peak 2 is SiaHexGal₂GlcNAc₂Man₃GlcNAc₂Fuc; Peak 3 is SiaGal₂GlcNAc₂Man₃GlcNAc₂; Peak 4 is SiaGalGlcNAcMansGlcNAc₂Fuc; Peak 5 is SiaGalGlcNAC₂Man₃GlcNAc₂Fuc; Peak 6 is SiaGalGlcNAcMan₄GlcNAc₂Fuc; and Peak 7 is SiaGalGlcNAcMan₃GlcNAc₂Fuc.

FIG. 46 is a graphic representation of an intact protein mass (IPM) analysis (LC/MASS) conducted to demonstrate the glycan profile of a commercial Perjeta® product.

FIG. 47 is a graphic representation of an intact protein mass (IPM) analysis (LC/MASS) conducted to demonstrate the Perjeta® protein population's glycan profile produced by CHO-S cells.

FIG. 48 is a graphic representation of an intact protein mass (IPM) analysis (LC/MASS) conducted to demonstrate the glycoengineered Gazyva® protein population's glycan profile produced according to the present disclosure. The peaks representing heterogeneous sialylated glycans are numbered. Peak 1 is SiazGal₂GlcNAczMan₃GlcNAc₂Fuc; Peak 2 is SiaHexGal₂GlcNAc₂Man₃GlcNAc₂Fuc; Peak 3 is SiaGal₂GlcNAc₂Man₃GlcNAc₂; Peak 4 is SiaGalGlcNAcMansGlcNAc₂Fuc; Peak 5 is SiaGalGlcNAc₂Man₃GlcNAc₂Fuc; Peak 6 is SiaGalGlcNAcMan₄GlcNAc₂Fuc; and Peak 7 is SiaGalGlcNAcMan₃GlcNAc₂Fuc.

FIG. 49 is a graphic representation of an intact protein mass (IPM) analysis (LC/MASS) conducted to demonstrate the glycan profile of a commercial Gazyva® product.

FIG. 50 is a graphic representation of an intact protein mass (IPM) analysis (LC/MASS) conducted to demonstrate the Gazyva® protein population's glycan profile produced by CHO-S cells.

FIG. 51 is a graphic representation of an LC/MS-MS analysis conducted to demonstrate the glycoengineered Adalimumab (Humira®) protein population's glycan profile produced with the presence of peracetylated 4-F-ManNAc, according to the present disclosure. The peaks representing heterogeneous sialylated glycans are numbered. Peak 1 is SiazGal₂GlcNAC₂Man₃GlcNAc₂Fuc; Peak 3 is SiaGal₂GlcNAc₂Man₃GlcNAc₂Fuc; Peak 4 is SiaGalGlcNAcMansGlcNAc₂Fuc; Peak 6 is SiaGalGlcNAcMan₄GlcNAc₂Fuc; Peak 7 is SiaGalGlcNAcMan₃GlcNAc₂Fuc; and Peak 8 is SiaGal₃GlcNAc₃Man₃GlcNAc₂Fuc. The glycans with modified terminal sialic acids (7-fluoro sialic acid) are indicated by solid arrows.

FIG. 52 is a graphic representation of an ADCC assay's result showing the comparison between an exemplary Trastuzumab population of the present disclosure (Biobetter) and a commercially available Trastuzumab product (Herceptin).

FIG. 53 is a graphic representation of an ADCC assay's result showing the comparison between an exemplary Pertuzumab population of the present disclosure (Biobetter) and a commercially available Pertuzumab product (Perjeta).

FIG. 54 is a graphic representation of an ADCC assay's result showing the comparison between an exemplary Rituximab population of the present disclosure (Biobetter) and a commercially available Rituximab product (MabThera).

FIG. 55 is a graphic representation of an ADCC assay's result showing the comparison between an exemplary Cetuximab population of the present disclosure (Biobetter) and a commercially available Cetuximab product (Erbitux).

FIG. 56 is a graphic representation of an ELISA's result showing the target binding affinity by comparing an exemplary Trastuzumab population of the present disclosure (Biobetter) and a commercially available Trastuzumab product (Herceptin).

FIG. 57 is a graphic representation of an ELISA's result showing the target binding affinity by comparing an exemplary Rituximab population of the present disclosure (Biobetter) and a commercially available Rituximab product (MabThera).

FIG. 58 is a graphic representation of an ELISA's result showing the target binding affinity by comparing an exemplary Cetuximab population of the present disclosure (Biobetter) and a commercially available Cetuximab product (Erbitux).

FIG. 59 is a graphic representation of an ELISA's result showing the target binding affinity by comparing an exemplary Adalimumab population of the present disclosure (Biobetter) and a commercially available Adalimumab product (Humira).

FIG. 60 is a graphic representation of an ELISA's result showing the target binding affinity by comparing an exemplary Bevacizumab population of the present disclosure (Biobetter) and a commercially available Bevacizumab product (Avastin).

FIG. 61 is a graphic representation of an ELISA's result showing the target binding affinity by comparing an exemplary Ipilimumab population of the present disclosure (Biobetter) and a commercially available Ipilimumab product (Yervoy).

FIG. 62 is a graphic representation of an ELISA's result showing the target binding affinity by comparing an exemplary Pembrolizumab population of the present disclosure (Biobetter) and a commercially available Pembrolizumab product (Keytruda).

FIG. 63 is a graphic representation of an ELISA's result showing the target binding affinity by comparing an exemplary Nivolumab population of the present disclosure (Biobetter) and a commercially available Nivolumab product (Opdivo).

FIG. 64 is a graphic representation of an ELISA's result showing the target binding affinity by comparing an exemplary Avelumab population of the present disclosure (Biobetter) and a commercially available Avelumab product (Bavencio).

FIG. 65 is a graphic representation of an ELISA's result showing the target binding affinity by comparing an exemplary Durvalumab population of the present disclosure (Biobetter) and a commercially available Durvalumab product (IMFINZI).

FIG. 66 is a graphic representation of an ELISA's result showing the target binding affinity by comparing an exemplary Pertuzumab population of the present disclosure (Biobetter) and a commercially available Pertuzumab product (Perjeta).

FIG. 67 is a graphic representation of an ELISA's result showing the target binding affinity by comparing an exemplary Obinutuzumab population of the present disclosure (Biobetter) and a commercially available Obinutuzumab product (Gazyva).

FIG. 68 is a graphic representation of an ADCP assay conducted to demonstrate the phagocytosis ability of an exemplary glycoprotein population of the present disclosure. CHOS represents a control glycoprotein population produced by CHO-S cells, and GO denotes Glyco-Optimization, which represents a glycoprotein population produced according to the present disclosure. Contril Ab represents an IgG1 antibody (PGT145), targeting HIV, which was used in this experiment as a negative control to establish a benchmark signal for phagocytosis determination.

FIG. 69A is a graphic representation of an ADCC organoid assay conducted to demonstrate the ADCC effects of a BioBetter Herceptin® sample according to an example of the present disclosure and a commercial Herceptin® product against a HER3+ organoid at a concentration of 0.08 μg/mL and a E: T ratio of 1:1.

FIG. 69B is a graphic representation of an ADCC organoid assay conducted to demonstrate the ADCC effects of a BioBetter Herceptin® sample according to an example of the present disclosure and a commercial Herceptin® product against a HER 1+ organoid at a concentration of 0.08 μg/mL and a E: T ratio of 1:1.

FIG. 70A is a graphic representation of an ADCC organoid assay conducted to demonstrate the ADCC effects of a BioBetter Herceptin® sample according to an example of the present disclosure and a commercial Herceptin® product against a HER3+ organoid at given concentration (0, 0.08, 0.4, 2, and 10 μm/mL) and E: T ratios (1:1, 3:1, and 0:1).

FIG. 70B is a graphic representation of an ADCC organoid assay conducted to demonstrate the ADCC effects of a BioBetter Herceptin® sample according to an example of the present disclosure and a commercial Herceptin® product against a HER 1+ organoid at given concentration (0, 0.08, 0.4, 2, and 10 μm/mL) and E: T ratios (1:1, 3:1, and 0:1).

### DETAILED DESCRIPTION OF THE INVENTION

Glycosylation is a common post- or co-translational modification found in most cell proteins. The importance of a glycoprotein's glycoform on therapeutic effects has been recognized. Taking therapeutic antibodies as an example, binding between an antibody and a target cell or a pathogen triggers a variety of downstream immune functions, including antibody-dependent cellular cytotoxicity (ADCC), complement-dependent cytotoxicity (CDC), antibody-dependent cellular phagocytosis (ADCP), antibody-dependent vaccinal effect (ADVE), phagocytosis, complement activation, etc. These immune cell-based responses require the binding of the antibody Fc region to specific Fc receptors (e.g., FcγRIIA or FcγRIIIA) on immune cells, in which the glycoside composition and the structure of the glycan conjugated on the antibody Fc region have been proved to be critical. The glycoforms are also known to affect antibody circulation in human bodies. Therefore, it has been a goal in the art to develop methodologies to optimize the glycoform of a therapeutic protein in order to improve the biological activities and efficacy thereof.

To that end, the effects of sialylated complex type (SCT) glycans and core fucoses of a Fc glycan have drawn attention. SCT glycan refers to oligosaccharides, which comprise a glycan chain having at least one mannose, sugar residues other than mannoses, and at least one terminal sialic acid. A SCT glycan can be monoantennary, biantennary, or triantennary, and typically comprises a common trimannose core, which consists of three mannose and two N-acetylglucosamine (GlcNAc) residues (i.e., “Man₃GlcNAc₂”, wherein “Man” refers to mannose). The terminal sialic acid can be formed on at least one of the arms of a biantennary (e.g., a1,6 or a1,3 arm) or triantennary structure connecting to a preceding sugar residue (e.g., a galactose) of the glycan chain via an a2,6 or a2,3 linkage. SCT glycans have been shown to be associated with enhanced effector functions, including ADCC, CDC, ADCP, and ADVE, especially ADCP and ADVE, which can be enhanced through the conformation changes of the immunoglobins triggered by sialylation and enabling better interaction between the Fc region and FcγRIIA receptors. It is also recognized that Fc domain sialylation shields the otherwise terminal galactose on the Fc-glycan, which can be recognized, if unmasked, by the hepatic asialoglycoprotein receptor (ASGPR), resulting in clearance of the biotherapeutic from the circulation. On the other hand, the absence of core fucoses on the Fc-glycan is correlated with enhancing binding between Fc regions and FcγRIIIA receptors, which is directly associated with ADCC.

Accordingly, improving biological efficacy of glycoprotein therapeutics have focused on the two desired features, including sialylated glycans and the absence of core fucoses. Many adopt approaches that entail chemoenzymatic reactions to modify the Fc-glycans of produced therapeutic proteins (e.g., therapeutic antibodies) with terminal sialic acids. The enzymatic process generates sialylated glycans with high homogeneity (e.g., the generated therapeutic protein population substantially has only one glycoform); however, it is costly and time-consuming. Furthermore, unlike the in vivo synthesis of protein and DNA, glycosylation is template-independent. It is, therefore, also unknown whether a glycoprotein population with such a homogeneous glycan profile is beneficial to their biological activities in vivo. Some developed cell line platforms to directly produce therapeutic proteins with desired Fc-glycans. Nevertheless, cell lines are different from one another in their performance. Hence, proper sialylation levels need to be determined in order to identify the most suitable cell line and the most efficacious enrichment/homogeneity for the sialylated Fc-glycan of therapeutic proteins.

In light of the foregoing, the present disclosure's approach to focus on taking advantage of the human immune system to maximize the Fc-mediated killing activity through balanced effector functions without possible immunogenicity generated from the antibody. More importantly, the antibody-induced immune cells, according to the present disclosure, are capable of differentiating self and non-self invaders with a significant increase in safety and efficacy and a decrease in induction of immunogenicity.

Furthermore, the cell lines used in current commercial antibody development do not express appropriate human glycosyltransferases. CHO cells, one of the most common cell types used in the field, do not have the sialylation activity, and HEK293 cells only express a very low level of 2,3-sialyltransferase, which has a negative impact on the effector functions. Besides, while FcγRIIIA-mediated ADCC is important, it is mainly exerted by NK cells, which are not typically around tumor environment. In contrast, FcγRIIA-mediated ADCP and vaccinal effect (ADVE) have been overlooked as both are difficult to optimize, and therapeutic antibodies that provide high ADCC often have low ADCP and ADVE. The present disclosure provides the glycoproteins (e.g., antibody) with heterogeneous glycan profile comprises at least about 20% (preferably about 60%) induce similar ADCC as glycoproteins with homogeneous glycan profile but better ADCP and ADVE with the potential of inducing memory CD8 T-cell response. In addition, the manufacturing process of the antibodies according to the present disclosure is much more cost-effective compared to other manufacturing processes that require enzymatic reactions.

Antibody Population with Desired Glycan Profile

One aspect of the present disclosure provides an engineered glycoprotein population comprising a plurality of glycoproteins. The engineered glycoprotein population comprises a heterogeneous glycan profile, wherein the heterogeneous glycan profile comprises at least 20%, 25%, 30%, 35%, 40%, 45%, 50%, 55%, 60%, 65%, 70%, 72%, 75%, 77%, 80%, 85%, 86%, 87%, 88%, 89%, 90%, 91%, 92%, 93%, 94%, 90%, or 95% of sialylated complex type (SCT) glycans, and the SCT glycans comprises two or more types of sialylated glycans (e.g., more than one type of sialylated glycans). In some embodiments of the present disclosure, the SCT glycan comprises a trimannose core, which comprises three mammose and two N-acetylglucosamine (GlcNAc) residues with a biantennary (e.g., a1,6 or a1,3 arm) or a triantennary branch. In certain embodiments, the sialylated glycans comprise a terminal sialic acid, which optionally is connected to a preceding sugar residue (e.g., a galactose) of the glycan via an a2,6 or a2,3 linkage (preferably an a2,6 linkage). In some embodiments, the sialylated glycans do not have a core fucose. In some embodiments, the engineered glycoprotein population is produced by a cell of the present disclosure, which will be described below in further detail.

In some embodiments, the engineered glycoprotein population of the present disclosure is an isolated glycoprotein population. As used herein, “isolated” means that a subject protein or polypeptide (1) is free of at least some other proteins or polypeptides with which it would typically be found in nature, (2) is essentially free of other proteins or polypeptides from the same source, e.g., from the same species, (3) is expressed by a cell from a different species or an engineered cell that does not exist in nature, (4) has been separated from at least about 50 percent of polynucleotides, lipids, carbohydrates, or other materials with which it is associated in nature, (5) is not associated (by covalent or noncovalent interaction) with portions of a protein or polypeptide with which the “isolated protein” or “isolated polypeptide” may be associated in nature, (6) is operably associated (by covalent or noncovalent interaction) with a polypeptide with which it is not associated in nature, or (7) does not occur in nature. Such an isolated protein or polypeptide can be encoded by genomic DNA, cDNA, mRNA or other RNA, of may be of synthetic origin according to any of a number of well-known chemistries for artificial peptide and protein synthesis, or any combination thereof. In certain embodiments, the isolated protein or polypeptide is substantially free from proteins or polypeptides or other contaminants that are found in its natural environment that would interfere with its use (therapeutic, diagnostic, prophylactic, research or otherwise).

Furthermore, in some embodiments, the engineered glycoprotein population comprises a plurality of recombinant glycoproteins. As used herein, “recombinant” means that the glycoprotein is produced by introducing an engineered nucleic acid into a host organism, like bacteria, yeast, or mammalian cells using laboratory or industrial processes, and in some situations, isolated or purified for clinical or industrial uses. The recombinant glycoprotein is different from its naturally occurring counterpart, and the difference can be shown at least from the glycan conjugated/post-translation modified on the glycoprotein considered from an individual protein basis or a protein population basis. For example, the recombinant glycoprotein of the present disclosure exhibits SCT enriched glycoform compared to its naturally occurring counterpart.

### Heterogeneity

As used herein, “glycoform” refers to the type of glycan conjugated with a glycoprotein. A “glycan profile” refers to the collective types of glycoforms observed from a glycoprotein population, and in some specific embodiments, a glycan profile refers to the characteristic “fingerprint” of the N-glycan species that have been released from a glycoprotein or antibody, cither enzymatically or chemically, and then analyzed for their carbohydrate structure, for example, using LC-HPLC, or MALDI-TOF MS, and the like. See, for example, the review in Current Analytical Chemistry, Vol. 1, No. 1 (2005), pp. 28-57; herein incorporated by reference in its entirety. As used herein, “heterogeneous,” used to describe a glycan profile, means that the glycan profile comprises two or more types of glycoforms (e.g., two or more types of sialylated glycans, meaning the glycan profile or the sialylated glycans of the engineered glycoprotein population is not homogeneous). In contrast, if a glycoprotein population is described as having a homogeneous glycan profile, the glycoprotein population comprises substantially only one glycoform. In other words, substantially, a homogeneous glycan profile comprises a glycoprotein population where each glycoprotein of the population comprises the same glycoform.

Without wishing to be bound by theories, the present disclosure believes that the heterogeneous glycan profile comprising two or more types (e.g., more than one type) of sialylated glycans benefits the glycoprotein's efficacy in vivo. The researchers in the field have been suggesting a homogeneous glycan profile comprising basically only one kind of sialylated glycoform or glycan. Despite the fact that the benefit of sialylation has been established in the field, little has been known as to each different kind of sialylated glycans' role in interacting with different activation and inhibition Fcγ receptors and cell types, let alone that any glycoprotein populations in nature rarely have a homogeneous glycan profile. Therefore, the present disclosure contemplates that a heterogeneous glycan profile of the present disclosure is beneficial in ensuring diversity, which can be important in vivo and is compromised in a homogeneous glycan profile. Accordingly, in some embodiments, the engineered glycoprotein population of the present disclosure improved the ADCC, CDC, ADCP, ADVE (vaccinal effect), and/or extended half-live, especially ADCP and vaccinal effect, of the glycoprotein population, compared to their reference product counterpart which is without the desired heterogenous glycan profile.

In some embodiments, the SCT glycans of the glycan profile is of a homogeneity, which is lower than 100%, 99%, 95%, 90%, 88%, 85%, 80%, 75%, 70%, 65%, 60%, 55%, 50%, 45%, 40%, 35%, 30%, 25%, 20%, 15%, 10%, or 5%, (meaning any kind of SCT glycans of the glycan profile comprises no more than 99%, 95%, 90%, 88%, 85%, 80%, 75%, 70%, 65%, 60%, 55%, 50%, 45%, 40%, 35%, 30%, 25%, 20%, 15%, 10%, or 5% of the glycans of the glycan profile) or any range defined by the foregoing endpoints, such as 5% to 99%, 5%, to 90%, 5% to 80%, 5% to 70%, 5% to 60%, 5% to 50%, 5% to 40%, 5% to 30%, 5% to 20%, 5% to 10%, 10% to 99%, 10%, to 90%, 10% to 80%, 10% to 70%, 10% to 60%, 10% to 50%, 10% to 40%, 10% to 30%, 10% to 20%, 20% to 99%, 20%, to 90%, 20% to 80%, 20% to 70%, 20% to 60%, 20% to 50%, 20% to 40%, 20% to 30%, 40% to 99%, 40%, to 90%, 40% to 80%, 40% to 70%, 40% to 60%, or 40% to 50%, including or excluding any foregoing numbers. In some embodiments, the heterogeneous glycan profile comprises no more than 95%, 90%, or 88% of SCT glycans. In some embodiments, the heterogenous glycan profile comprises 20% to 95%, 20% to 90%, 20% to 88%, 20% to 86%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 30% to 95%, 30% to 90%, 30% to 88%, 30% to 86%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 40% to 95%, 40% to 90%, 40% to 88%, 40% to 86%, 40% to 85%, 40% to 80%, 40% to 75%, 40% to 70%, 40% to 65%, 40% to 60%, 40% to 55%, 40% to 50%, 40% to 45%, 50% to 95%, 50% to 90%, 50% to 88%, 50% to 86%, 50% to 85%, 50% to 80%, 50% to 75%, 50% to 70%, 50% to 65%, 50% to 60%, 50% to 55%, 60% to 95%, 60% to 90%, 60% to 88%, 60% to 86%, 60% to 85%, 60% to 80%, 60% to 75%, 60% to 70%, or 60% to 65% of SCT glycans. All numbers can be modified by “about,” which is defined herein.

In some embodiments, the SCT glycans of the glycan profile can be monoantennary, biantennary, or triantennary. In certain embodiments, the SCT glycans comprise biantennary glycans, which comprise at least 5%, 10%, 15%, 20%, 25%, 30%, 35%, 40%, 45%, 50%, 55%, 60%, 65%, 70%, 75%, 80%, 85%, 90%, 95%, or 100% of the glycans of the engineered glycoprotein population's glycan profile, or any range defined by the foregoing endpoints, such as 5 to 100%, 5 to 95%, 5 to 90%, 5 to 85%, 5 to 80%, 5 to 70%, 5 to 60%, 5 to 50%, 5 to 40%, 5 to 30%, 5 to 20%, 5 to 10%, 10 to 100%, 10 to 95%, 10 to 90%, 10 to 85%, 10 to 80%, 10 to 70%, 10 to 60%, 10 to 50%, 10 to 40%, 10 to 30%, 10 to 20%, 20 to 100%, 20 to 90%, 20 to 80%, 20 to 70%, 20 to 60%, 20 to 50%, 20 to 40%, 20 to 30%, 50 to 100%, 50 to 95%, 50 to 90%, 50 to 85%, 50 to 80%, 50 to 70%, or 50 to 60%, including or excluding any foregoing numbers. All numbers can be modified by “about,” which is defined herein. In certain embodiments, the SCT glycans comprise triantennary glycans, which comprise at least 0.1%, 0.5%, 1%, 2%, 3%, 4%, 5%, 6%, 7%, 8%, 9%, 10%, 11%, 12%, 13%, 14%, or 15% of the glycans of the engineered glycoprotein population's glycan profile, or any range defined by the foregoing endpoints, such as 0.1 to 15%, 0.1 to 14%, 0.1 to 12%, 0.1 to 10%, 0.1 to 8%, 0.1 to 5%, 0.1 to 3%, 0.1 to 1%, 1 to 15%, 1 to 14%, 1 to 12%, 1 to 10%, 1 to 8%, 1 to 5%, 1 to 3%, 3 to 15%, 3 to 14%, 3 to 12%, 3 to 10%, 3 to 8%, 3 to 5%, 5 to 15%, 5 to 14%, 5 to 12%, 5 to 10%, 5 to 8%, including or excluding any foregoing numbers. All numbers can be modified by “about,” which is defined herein. In some embodiments, the glycan profile has no monoantennary SCT glycan; or the monoantennary SCT glycan thereof is at an amount that is not detectable.

In certain embodiments, the SCT glycans of the engineered glycoprotein population's glycan profile comprises a first SCT glycan and a second SCT glycan, and each or any of the first SCT glycan and the second SCT glycan comprises independently no more than 70%, 65%, 60%, 55%, 50%, 45%, 40%, 35%, 30%, 25%, 20%, 15%, 10%, 9%, 8%, 7%, 6%, 5%, 4%, 3.5%, 3%, 2.5%, 2%, 1.5%, 1%, 0.5%, 0.1%, 0.05%, or 0.01% of the glycans of the engineered glycoprotein population's glycan profile, or any range defined by the foregoing endpoints, such as 0.01% to 70%, 0.05% to 70%, 0.1% to 70%, 0.5% to 70%, 1% to 70%, 2% to 70%, 3% to 70%, 5% to 70%, 8% to 70%, 10% to 70%, 15% to 70%, 20% to 70%, 25% to 70%, 30% to 70%, 35% to 70%, 40% to 70%, 45% to 70%, 0.01% to 60%, 0.05% to 60%, 0.1% to 60%, 0.5% to 60%, 1% to 60%, 2% to 60%, 3% to 60%, 5% to 60%, 8% to 60%, 10% to 60%, 15% to 60%, 20% to 60%, 25% to 60%, 30% to 60%, 35% to 60%, 40% to 60%, 45% to 60%, 0.01% to 50%, 0.05% to 50%, 0.1% to 50%, 0.5% to 50%, 1% to 50%, 2% to 50%, 3% to 50%, 5% to 50%, 8% to 50%, 10% to 50%, 15% to 50%, 20% to 50%, 25% to 50%, 30% to 50%, 35% to 50%, 40% to 50%, 45% to 50%, 0.01% to 45%, 0.05% to 45%, 0.1% to 45%, 0.5% to 45%, 1% to 45%, 2% to 45%, 3% to 45%, 5% to 45%, 8% to 45%, 10% to 45%, 15% to 45%, 20% to 45%, 25% to 45%, 30% to 45%, 35% to 45%, 40% to 45%, 0.01% to 40%, 0.05% to 40%, 0.1% to 40%, 0.5% to 40%, 1% to 40%, 2% to 40%, 3% to 40%, 5% to 40%, 8% to 40%, 10% to 40%, 15% to 40%, 20% to 40%, 25% to 40%, 30% to 40%, 35% to 40%, 0.01% to 30%, 0.05% to 30%, 0.1% to 30%, 0.5% to 30%, 1% to 30%, 2% to 30%, 3% to 30%, 5% to 30%, 8% to 30%, 10% to 30%, 15% to 30%, 20% to 30%, 25% to 30%, 0.01% to 25%, 0.05% to 25%, 0.1% to 25%, 0.5% to 25%, 1% to 25%, 2% to 25%, 3% to 25%, 5% to 25%, 8% to 25%, 10% to 25%, 15% to 25%, 20% to 25%, 0.01% to 15%, 0.05% to 15%, 0.1% to 15%, 0.5% to 15%, 1% to 15%, 2% to 15%, 3% to 15%, 5% to 15%, 8% to 15%, 10% to 15%, 0.01% to 10%, 0.05% to 10%, 0.1% to 10%, 0.5% to 10%, 1% to 10%, 5% to 10%, 0.01% to 5%, 0.05% to 5%, 0.1% to 5%, 0.5% to 5%, 1% to 5%, or 3% to 5%, including or excluding any foregoing numbers. All numbers can be modified by “about,” which is defined herein.

In certain embodiments, the first SCT glycan is SiaGal₂GlcNAc₂Man₃GlcNAc₂ or SiaGal₂GlcNAc₂Man₃GlcNAc₂Fuc (Peak 3 glycan as labeled herein), and the second glycan is SiazGal₂GlcNAC₂Man₃GlcNAc₂ or SiazGal₂GlcNAc₂Man₃GlcNAc₂Fuc (Peak 1 glycan as labeled herein). In some embodiments, the first SCT glycan and the second SCT glycan are selected from a group consisting of:

| Peak No. | Sialylated complex type glycans |
|---|---|
| 1 | Sia₂Gal₂GlcNAc₂Man₃GlcNAc₂ or Sia₂Gal₂GlcNAc₂Man₃GlcNAc₂Fuc |
| 2 | SiaHexGal₂GlcNAc₂Man₃GlcNAc₂ or SiaHexGal₂GlcNAc₂Man₃GlcNAc₂Fuc |
| 3 | SiaGal₂GlcNAc₂Man₃GlcNAc₂ or SiaGal₂GlcNAc₂Man₃GlcNAc₂Fuc |
| 4 | SiaGalGlcNAcMan₅GlcNAc₂ or SiaGalGlcNAcMan₅GlcNAc₂Fuc |
| 5 | SiaGalGlcNAc₂Man₃GlcNAc₂ or SiaGalGlcNAc₂Man₃GlcNAc₂Fuc |
| 6 | SiaGalGlcNAcMan₄GlcNAc₂ or SiaGalGlcNAcMan₄GlcNAc₂Fuc |
| 7 | SiaGalGlcNAcMan₃GlcNAc₂ or SiaGalGlcNAcMan₃GlcNAc₂Fuc |
| 8 | SiaGal₃GlcNAc₃Man₃GlcNAc₂ or SiaGal₃GlcNAc₃Man₃GlcNAc₂Fuc |

provided that the first SCT glycan and the second SCT glycan are not the same.

In some embodiments, the first SCT glycan is SiaGal₂GlcNAC₂Man₃GlcNAc₂ or SiaGal₂GlcNAC₂Man₃GlcNAc₂Fuc (Peak 3 glycan as labeled herein), the second glycan is SiazGal₂GlcNAc₂Man₃GlcNAC₂ or SiazGal₂GlcNAc₂Man₃GlcNAc₂Fuc (Peak 1 glycan as labeled herein), and the glycan profile further comprises one or all of SiaHexGal₂GlcNAczMan₃GlcNAc₂ or SiaHexGal₂GlcNAc₂Man₃GlcNAc₂Fuc, SiaGalGlcNAcMansGlcNAc₂ or SiaGalGlcNAcMansGlcNAc₂Fuc, SiaGalGlcNAc₂Man₃GlcNAc₂ or SiaGalGlcNAc₂Man₃GlcNAC₂Fuc, SiaGalGlcNAcMan₄GlcNAc₂ or SiaGalGlcNAcMan₄GlcNAc₂Fuc, SiaGalGlcNAcMan₃GlcNAc₂ or SiaGalGlcNAcMan₃GlcNAc₂Fuc, and SiaGal₃GlcNAc₃Man₃GlcNAc₂ or SiaGal₃GlcNAc₃Man₃GlcNAc₂Fuc.

The percentage of sialylated glycans or SCT described herein represents a relative abundance. The percentage can be determined by using analytical tools developed and accepted in the field. In some embodiments, the percentage can be determined by using intact protein mass (IPM) with LC/MASS, which reduces the glycoproteins to be examined and identifies the glycans originally conjugated on the glycoproteins before reduction through their molecular weight. The relative abundance of the identified glycan can then be determined by calculating the area under the curve (AUC) of the individual glycoform as a proportion of the total AUC. Some other exemplary tools for examining the percentages of glycoforms include, but not limited to, fluorescent tags labeling HILIC-HPLC analysis, Lectin hybridization, or Lectin ELISA.

In some embodiments, the SiaGal₂GlcNAc₂Man₃GlcNAc₂ or SiaGal₂GlcNAc₂Man₃GlcNAc₂Fuc glycan (the Peak 1 glycan) comprises no more than 70%, 65%, 60%, 6%, 50%, 45%, 40%, 35%, 30%, 25%, 20%, 15%, 10%, 9%, 8%, 7%, 6%, 5%, 4%, 3.5%, 3%, 2.5%, 2%, 1.5%, 1%, 0.5%, or 0.1% of the glycans of the engineered glycoprotein population's glycan profile, or any range defined by the foregoing endpoints, such as 0.1% to 70%, 0.5% to 70%, 1% to 70%, 2% to 70%, 3% to 70%, 5% to 70%, 8% to 70%, 10% to 70%, 15% to 70%, 20% to 70%, 25% to 70%, 30% to 70%, 35% to 70%, 40% to 70%, 45% to 70%, 0.1% to 60%, 0.5% to 60%, 1% to 60%, 2% to 60%, 3% to 60%, 5% to 60%, 8% to 60%, 10% to 60%, 15% to 60%, 20% to 60%, 25% to 60%, 30% to 60%, 35% to 60%, 40% to 60%, 45% to 60%, 0.1% to 50%, 0.5% to 50%, 1% to 50%, 2% to 50%, 3% to 50%, 5% to 50%, 8% to 50%, 10% to 50%, 15% to 50%, 20% to 50%, 25% to 50%, 30% to 50%, 35% to 50%, 40% to 50%, 45% to 50%, 0.1% to 45%, 0.5% to 45%, 1% to 45%, 2% to 45%, 3% to 45%, 5% to 45%, 8% to 45%, 10% to 45%, 15% to 45%, 20% to 45%, 25% to 45%, 30% to 45%, 35% to 45%, 40% to 45%, 0.1% to 40%, 0.5% to 40%, 1% to 40%, 2% to 40%, 3% to 40%, 5% to 40%, 8% to 40%, 10% to 40%, 15% to 40%, 20% to 40%, 25% to 40%, 30% to 40%, 35% to 40%, 0.1% to 30%, 0.5% to 30%, 1% to 30%, 2% to 30%, 3% to 30%, 5% to 30%, 8% to 30%, 10% to 30%, 15% to 30%, 20% to 30%, 25% to 30%, 0.1% to 25%, 0.5% to 25%, 1% to 25%, 2% to 25%, 3% to 25%, 5% to 25%, 8% to 25%, 10% to 25%, 15% to 25%, 20% to 25%, 0.1% to 15%, 0.5% to 15%, 1% to 15%, 2% to 15%, 3% to 15%, 5% to 15%, 8% to 15%, 10% to 15%, 0.1% to 10%, 0.5% to 10%, 1% to 10%, 5% to 10%, 0.1% to 5%, 0.5% to 5%, 1% to 5%, or 3% to 5%, including or excluding any foregoing numbers. All numbers can be modified by “about,” which is defined herein.

In some embodiments, the SiazGal₂GlcNAC₂Man₃GlcNAc₂ or SiazGal₂GlcNAc₂Man₃GlcNAc₂Fuc glycan (the Peak 2 glycan) comprises no more than 10%, 9%, 8%, 7%, 6%, 5%, 4%, 3.5%, 3%, 2.5%, 2%, 1.5%, 1%, 0.5%, or 0.1% of the glycans of the engineered glycoprotein population's glycan profile, or any range defined by the foregoing endpoints, such as 0.1% to 10%, 0.5% to 10%, 1% to 10%, 5% to 10%, 0.1% to 5%, 0.5% to 5%, 1% to 5%, 3% to 5%, 0.1% to 1%, 0.5% to 1%, or 0.1% to 0.5%, including or excluding any foregoing numbers. All numbers can be modified by “about,” which is defined herein.

In some embodiments, the SiaGalGlcNAcMansGlcNAc₂ or SiaGalGlcNAcMansGlcNAc₂Fuc glycan (the Peak 3 glycan) comprises no more than 70%, 65%, 60%, 6%, 50%, 45%, 40%, 35%, 30%, 25%, 20%, 15%, 10%, 9%, 8%, 7%, 6%, 5%, 4%, 3.5%, 3%, 2.5%, 2%, 1.5%, 1%, 0.5%, or 0.1% of the glycans of the engineered glycoprotein population's glycan profile, or any range defined by the foregoing endpoints, such as 0.1% to 70%, 0.5% to 70%, 1% to 70%, 2% to 70%, 3% to 70%, 5% to 70%, 8% to 70%, 10% to 70%, 15% to 70%, 20% to 70%, 25% to 70%, 30% to 70%, 35% to 70%, 40% to 70%, 45% to 70%, 0.1% to 60%, 0.5% to 60%, 1% to 60%, 2% to 60%, 3% to 60%, 5% to 60%, 8% to 60%, 10% to 60%, 15% to 60%, 20% to 60%, 25% to 60%, 30% to 60%, 35% to 60%, 40% to 60%, 45% to 60%, 0.1% to 50%, 0.5% to 50%, 1% to 50%, 2% to 50%, 3% to 50%, 5% to 50%, 8% to 50%, 10% to 50%, 15% to 50%, 20% to 50%, 25% to 50%, 30% to 50%, 35% to 50%, 40% to 50%, 45% to 50%, 0.1% to 45%, 0.5% to 45%, 1% to 45%, 2% to 45%, 3% to 45%, 5% to 45%, 8% to 45%, 10% to 45%, 15% to 45%, 20% to 45%, 25% to 45%, 30% to 45%, 35% to 45%, 40% to 45%, 0.1% to 40%, 0.5% to 40%, 1% to 40%, 2% to 40%, 3% to 40%, 5% to 40%, 8% to 40%, 10% to 40%, 15% to 40%, 20% to 40%, 25% to 40%, 30% to 40%, 35% to 40%, 0.1% to 30%, 0.5% to 30%, 1% to 30%, 2% to 30%, 3% to 30%, 5% to 30%, 8% to 30%, 10% to 30%, 15% to 30%, 20% to 30%, 25% to 30%, 0.1% to 25%, 0.5% to 25%, 1% to 25%, 2% to 25%, 3% to 25%, 5% to 25%, 8% to 25%, 10% to 25%, 15% to 25%, 20% to 25%, 0.1% to 15%, 0.5% to 15%, 1% to 15%, 2% to 15%, 3% to 15%, 5% to 15%, 8% to 15%, 10% to 15%, 0.1% to 10%, 0.5% to 10%, 1% to 10%, 5% to 10%, 0.1% to 5%, 0.5% to 5%, 1% to 5%, or 3% to 5%, including or excluding any foregoing numbers. All numbers can be modified by “about,” which is defined herein.

In some embodiments, the SiaGalGlcNAC₂Man₃GlcNAc₂ or SiaGalGlcNAc₂Man₃GlcNAc₂Fuc glycan (the Peak 4 glycan) comprises no more than 20%, 15%, 10%, 9%, 8%, 7%, 6%, 5%, 4%, 3.5%, 3%, 2.5%, 2%, 1.5%, 1%, 0.5%, or 0.1% of the glycans of the engineered glycoprotein population's glycan profile, or any range defined by the foregoing endpoints, such as 0.1% to 20%, 0.5% to 20%, 1% to 20%, 2% to 20%, 3% to 20%, 5% to 20%, 8% to 20%, 10% to 20%, 15% to 20%, 0.1% to 15%, 0.5% to 15%, 1% to 15%, 2% to 15%, 3% to 15%, 5% to 15%, 8% to 15%, 10% to 15%, 0.1% to 10%, 0.5% to 10%, 1% to 10%, 5% to 10%, 0.1% to 5%, 0.5% to 5%, 1% to 5%, 3% to 5%, 0.1% to 1%, 0.1% to 0.5%, or 0.5% to 1%, including or excluding any foregoing numbers. All numbers can be modified by “about,” which is defined herein.

In some embodiments, the SiaGalGlcNAcMan₄GlcNAc₂ or SiaGalGlcNAcMan₄GlcNAc₂Fuc glycan (the Peak 5 glycan) comprises no more than 20%, 15%, 10%, 9%, 8%, 7%, 6%, 5%, 4%, 3.5%, 3%, 2.5%, 2%, 1.5%, 1%, 0.5%, or 0.1% of the glycans of the engineered glycoprotein population's glycan profile, or any range defined by the foregoing endpoints, such as 0.1% to 20%, 0.5% to 20%, 1% to 20%, 2% to 20%, 3% to 20%, 5% to 20%, 8% to 20%, 10% to 20%, 15% to 20%, 0.1% to 15%, 0.5% to 15%, 1% to 15%, 2% to 15%, 3% to 15%, 5% to 15%, 8% to 15%, 10% to 15%, 0.1% to 10%, 0.5% to 10%, 1% to 10%, 5% to 10%, 0.1% to 5%, 0.5% to 5%, 1% to 5%, 3% to 5%, 0.1% to 1%, 0.1% to 0.5%, or 0.5% to 1%, including or excluding any foregoing numbers. All numbers can be modified by “about,” which is defined herein.

In some embodiments, the SiaHexGal₂GlcNAczMan₃GlcNAc₂ or SiaHexGal₂GlcNAczMan₃GlcNAc₂Fuc glycan (the Peak 6 glycan) comprises no more than 10%, 9%, 8%, 7%, 6%, 5%, 4%, 3.5%, 3%, 2.5%, 2%, 1.5%, 1%, 0.5%, or 0.1% of the glycans of the engineered glycoprotein population's glycan profile, or any range defined by the foregoing endpoints, such as 0.1% to 10%, 0.5% to 10%, 1% to 10%, 5% to 10%, 0.1% to 5%, 0.5% to 5%, 1% to 5%, 3% to 5%, 0.1% to 1%, 0.5% to 1%, or 0.1% to 0.5%, including or excluding any foregoing numbers. All numbers can be modified by “about,” which is defined herein.

In some embodiments, SiaGalGlcNAcMan₃GlcNAc₂ or SiaGalGlcNAcMan₃GlcNAc₂Fuc glycan (the Peak 7 glycan) comprises no more than 10%, 9%, 8%, 7%, 6%, 5%, 4%, 3.5%, 3%, 2.5%, 2%, 1.5%, 1%, 0.5%, or 0.1% of the glycans of the engineered glycoprotein population's glycan profile, or any range defined by the foregoing endpoints, such as 0.1% to 10%, 0.5% to 10%, 1% to 10%, 5% to 10%, 0.1% to 5%, 0.5% to 5%, 1% to 5%, 3% to 5%, 0.1% to 1%, 0.5% to 1%, or 0.1% to 0.5%, including or excluding any foregoing numbers. All numbers can be modified by “about,” which is defined herein.

In some embodiments, SiaGal₃GlcNAc₃Man₃GlcNAc₂ or SiaGal₃GlcNAc₃Man₃GlcNAc₂Fuc glycan (the Peak 8 glycan) comprises no more than 10%, 9%, 8%, 7%, 6%, 5%, 4%, 3.5%, 3%, 2.5%, 2%, 1.5%, 1%, 0.5%, 0.1%, 0.05%, or 0.01% of the glycans of the engineered glycoprotein population's glycan profile, or any range defined by the foregoing endpoints, such as 0.01% to 10%, 0.05% to 10%, 0.1% to 10%, 0.5% to 10%, 1% to 10%, 5% to 10%, 0.01% to 5%, 0.05% to 5%, 0.1% to 5%, 0.5% to 5%, 1% to 5%, 3% to 5%, 0.01% to 1%, 0.05% to 1%, 0.1% to 1%, 0.5% to 1%, 0.01% to 0.5%, 0.05% to 0.5%, 0.1% to 0.5%, or 0.01% to 0.05%, including or excluding any foregoing numbers. All numbers can be modified by “about,” which is defined herein.

### Modified Terminal Sialic Acid

It is believed that terminal sialic acids can improve circulatory half-lives of the therapeutic proteins by shielding the galactose on the glycan. That is to say, the integrity of the terminal sialic acids of the engineered glycoprotein population is associated with circulatory clearance thereof. Therefore, in some embodiments, the sialylated glycans comprises a terminal 7-fluoro sialic acid. Without wishing to be bound by theories, the modified sialic acids increase circulatory half-lives and improved pharmacokinetic profiles of the engineered glycoprotein population.

In some embodiments, the SCT glycans terminated with a 7-fluoro sialic acid comprises about 0.1%, 0.2%, 0.3%, 0.4% 0.5%, 1%, 1.5%, 2%, 5%, 6%, 7%, 8%, 9%, 10%, 12%, 15%, or 20% of the SCT glycans, or any range defined by the foregoing endpoints, such as 0.1 to 20%, 0.1 to 15%, 0.1 to 10%, 0.1 to 9%, 0.1 to 8%, 0.1 to 7%, 0.1 to 6%, 0.1 to 5%, 0.1 to 2%, 0.1 to 1%, 0.1 to 0.5%, 0.3 to 20%, 0.3 to 15%, 0.3 to 10%, 0.3 to 9%, 0.3 to 8%, 0.3 to 7%, 0.3 to 6%, 0.3 to 5%, 0.3 to 2%, 0.3 to 1%, 1 to 20%, 1 to 15%, 1 to 10%, 1 to 9%, 1 to 8%, 1 to 7%, 1 to 6%, 1 to 5%, 1 to 2%, 5 to 20%, 5 to 15%, 5 to 10%, 5 to 9%, 5 to 8%, 5 to 7%, or 5 to 6%, including or excluding any foregoing numbers. All numbers can be modified by “about,” which is defined herein.

### Glycoprotein

In some embodiments, the glycoprotein of the present disclosure is a therapeutic protein. As used here, “therapeutic protein” refers to biologically-derived or synthetic polypeptide designed to treat, prevent, or cure diseases and medical conditions, which can include, but not limited to, antibodies, enzymes, cytokines, protein receptors, and fusion proteins thereof. In some embodiments, the glycoprotein comprises a target-binding site and a glycosylation site. The target can be a surface protein of a dysfunctional cell, which can be a cell that has lost some or all of its normal function, has gained abnormal function, or has been inhibited or activated by another dysfunctional cell, thereby failing to perform its normal function. In some examples, the dysfunctional cell is a tumor cell or an immune cell. In some embodiments, the target can be, but not limited to, tumor necrosis factor-alpha (TNFα), Vascular Endothelial Growth Factor (VEGF), Cytotoxic T-Lymphocyte Associated protein 4 (CTLA-4), PD-L1, PD-1, Human Epidermal Growth Factor Receptor 2 (HER2), Epidermal Growth Factor Receptor (EGFR), CD38, CD52, CD20, or tumor-associated carbohydrate antigens.

In some embodiments, the therapeutic protein comprises a fragment crystallizable (Fc) region, which comprises the glycosylation site. In some embodiments, the target-binding site is an antigen-binding site, and wherein the glycoprotein comprises a first peptide and a second peptide, and the first peptide and the second peptide form the antigen-binding site. In certain embodiments, the first peptide is a heavy chain of an antibody, and the second peptide is a light chain of the antibody. In some specific embodiments, the glycoprotein is an antibody or antigen-binding fragment thereof. Yet in some embodiments, the glycoprotein is an immunogenic protein, such as a viral envelop protein, or a spike protein of a virus. For example, the immunogenic protein can be but is not limited to an influenza hemagglutinin or a SARS-CoV-2 spike protein.

In the embodiments that the glycoprotein is an antibody (e.g., a glycoantibody) or an antigen-binding fragment thereof, the antibody can be a monoclonal antibody. In some embodiments, the antibody can be a bispecific, tri-specific, or multi-specific antibody. In the embodiments that the glycoprotein is an antibody or an antigen-binding fragment thereof, the glycan can be located on a heavy chain or an Fc region. It is important to note that the glycosylation site of an antibody that regulates the antibody functions can differ from subtype to subtype. For example, the preBCR assembly is important for B cell development and is critically regulated by the N-glycan at N46 on μHC. The N-glycan at N402 on μHC has been linked to antibody oligomerization and complement activation. Besides, IgG N-glycosylation at N297 on γHC plays a critical role in complement activation and Fcγ receptor activation leading to various effector functions. Therefore, in some embodiments, the glycan can be located on N46, N402, and/or N297 of a heavy chain.

In some embodiments, each glycoprotein of the engineered glycoprotein population targets the same molecule. In certain embodiments, each glycoprotein of the engineered glycoprotein population targets the same epitope. In some specific embodiments, each glycoprotein of the engineered glycoprotein population is a monoclonal antibody targeting the same epitope.

In some embodiments, the glycoprotein is a therapeutic antibody. In certain embodiments, the glycoprotein is Adalimumab (Humira®), Adalimumab-atto (Amjevita®), Bevacizumab (Avastin®), Alemtuzumab (Campath®), Ipilimumab (Yervoy®), Avelumab (Bavencio®), Durvalumab (IMFINZI®), Pembrolizumab (Keytruda®), Nivolumab (Opdivo®), Etanercept (Enbrel®), Trastuzumab (Herceptin®), Pertuzumab (Perjeta®), Cetuximab (Erbitux®), Rituximab (Rituxan®), Rituximab-atto (Truxima®), Obinutuzumab (Gazyva®), Infliximab (Remicade®), Ofatumumab (Arzerra®), Golimumab (Simponi®), Atezolizumab (Tecentriq®), Ocrelizumab (OCREVUS®), Gazyvaro Infliximab (Remicade®), Zanidatamab (Ziihera®), Daratumumab (Darzalex®), or Trastuzumab emtansine (Kadcyla®).

Without wishing to be bound by theories, the present disclosure contemplates that for therapeutic proteins whose therapeutic mechanisms aim on blocking the interaction between a normal cell and a dysfunctional cell by targeting the normal cell, the desired glycan profiles thereof, in some embodiments, would exhibit the desired heterogeneous SCT glycans as described herein while maintain the core fucose. In this category, the exemplary therapeutic proteins include, but are not limited to, Adalimumab (Humira®), Bevacizumab (Avastin®), Ipilimumab (Yervoy®), Avelumab (Bavencio®), Durvalumab (IMFINZI®), Pembrolizumab (Keytruda®), Nivolumab (Opdivo®), and Etanercept (Enbrel®).

On the other hand, for therapeutic proteins whose therapeutic mechanisms entail targeting the dysfunctional cells (e.g., a cancer cell) directly, the desired glycan profiles thereof, in some embodiments, would exhibit the desired heterogeneous SCT glycans without the core fucose, thereby increasing the ADCC effects thereof. In this category, the exemplary therapeutic proteins include, but are not limited to, Trastuzumab (Herceptin®), Pertuzumab (Perjeta®), Cetuximab (Erbitux®), Rituximab (Rituxan®), and Obinutuzumab (Gazyva®).

In certain embodiments, the glycoprotein is a monoclonal antibody targeting TNF-α. In some embodiments, the glycoprotein is a monoclonal antibody competing antigen-binding with Adalimumab (Humira®). In certain embodiments, the glycoprotein comprises a heavy chain variable complementarity-determining region 1 (VHCDR1) comprising an amino acid sequence as set forth in SEQ ID NO: 01, a VHCDR2 comprising an amino acid sequence as set forth in SEQ ID NO: 02, a VHCDR3 comprising an amino acid sequence as set forth in SEQ ID NO: 03, a light chain variable complementarity-determining region 1 (VLCDR1) comprising an amino acid sequence as set forth in SEQ ID NO: 06, a VLCDR2 comprising an amino acid sequence as set forth in SEQ ID NO: 07, and a VLCDR3 comprising an amino acid sequence as set forth in SEQ ID NO: 08. In certain embodiments, the glycoprotein comprises a heavy chain variable region comprising an amino acid sequence having at least 70%, 80%, 90%, 95% or 100% identity as the amino acid sequence set forth in SEQ ID NO: 04; and/or the glycoprotein comprises a light chain variable region comprising an amino acid sequence having at least 70%, 80%, 90%, 95% or 100% identity as the amino acid sequence set forth in SEQ ID NO: 09. Yet in certain embodiments, the glycoprotein comprises a heavy chain comprising an amino acid sequence having at least 70%, 80%, 90%, 95% or 100% identity as the amino acid sequence set forth in SEQ ID NO: 05; and/or the glycoprotein comprises a light chain comprising an amino acid sequence having at least 70%, 80%, 90%, 95% or 100% identity as the amino acid sequence set forth in SEQ ID NO: 10. All numbers can be modified by “about,” which is defined herein.

In certain embodiments, the glycoprotein is a monoclonal antibody targeting VEGF-A. In some embodiments, the glycoprotein is a monoclonal antibody competing antigen-binding with Bevacizumab (Avastin®). In certain embodiments, the glycoprotein comprises a heavy chain variable complementarity-determining region 1 (VHCDR1) comprising an amino acid sequence as set forth in SEQ ID NO: 11, a VHCDR2 comprising an amino acid sequence as set forth in SEQ ID NO: 12, a VHCDR3 comprising an amino acid sequence as set forth in SEQ ID NO: 13, a light chain variable complementarity-determining region 1 (VLCDR1) comprising an amino acid sequence as set forth in SEQ ID NO: 16, a VLCDR2 comprising an amino acid sequence as set forth in SEQ ID NO: 17, and a VLCDR3 comprising an amino acid sequence as set forth in SEQ ID NO: 18. In certain embodiments, the glycoprotein comprises a heavy chain variable region comprising an amino acid sequence having at least 70%, 80%, 90%, 95% or 100% identity as the amino acid sequence set forth in SEQ ID NO: 14; and/or the glycoprotein comprises a light chain variable region comprising an amino acid sequence having at least 70%, 80%, 90%, 95% or 100% identity as the amino acid sequence set forth in SEQ ID NO: 19. Yet in certain embodiments, the glycoprotein comprises a heavy chain comprising an amino acid sequence having at least 70%, 80%, 90%, 95% or 100% identity as the amino acid sequence set forth in SEQ ID NO: 15; and/or the glycoprotein comprises a light chain comprising an amino acid sequence having at least 70%, 80%, 90%, 95% or 100% identity as the amino acid sequence set forth in SEQ ID NO: 20. All numbers can be modified by “about,” which is defined herein.

In certain embodiments, the glycoprotein is a monoclonal antibody targeting CTLA-4. In some embodiments, the glycoprotein is a monoclonal antibody competing antigen-binding with Ipilimumab (Yervoy®). In certain embodiments, the glycoprotein comprises a heavy chain variable complementarity-determining region 1 (VHCDR1) comprising an amino acid sequence as set forth in SEQ ID NO: 21, a VHCDR2 comprising an amino acid sequence as set forth in SEQ ID NO: 22, a VHCDR3 comprising an amino acid sequence as set forth in SEQ ID NO: 23, a light chain variable complementarity-determining region 1 (VLCDR1) comprising an amino acid sequence as set forth in SEQ ID NO: 26, a VLCDR2 comprising an amino acid sequence as set forth in SEQ ID NO: 27, and a VLCDR3 comprising an amino acid sequence as set forth in SEQ ID NO: 28. In certain embodiments, the glycoprotein comprises a heavy chain variable region comprising an amino acid sequence having at least 70%, 80%, 90%, 95% or 100% identity as the amino acid sequence set forth in SEQ ID NO: 24; and/or the glycoprotein comprises a light chain variable region comprising an amino acid sequence having at least 70%, 80%, 90%, 95% or 100% identity as the amino acid sequence set forth in SEQ ID NO: 29. Yet in some embodiments, the glycoprotein comprises a heavy chain comprising an amino acid sequence having at least 70%, 80%, 90%, 95% or 100% identity as the amino acid sequence set forth in SEQ ID NO: 25; and/or the glycoprotein comprises a light chain comprising an amino acid sequence having at least 70%, 80%, 90%, 95% or 100% identity as the amino acid sequence set forth in SEQ ID NO: 30. All numbers can be modified by “about,” which is defined herein.

In certain embodiments, the glycoprotein is a monoclonal antibody targeting PD-L1. In some embodiments, the glycoprotein is a monoclonal antibody competing antigen-binding with Avelumab (Bavencio®) or Durvalumab (IMFINZI®).

In certain embodiments, the glycoprotein comprises a heavy chain variable complementarity-determining region 1 (VHCDR1) comprising an amino acid sequence as set forth in SEQ ID NO: 31, a VHCDR2 comprising an amino acid sequence as set forth in SEQ ID NO: 32, a VHCDR3 comprising an amino acid sequence as set forth in SEQ ID NO: 33, a light chain variable complementarity-determining region 1 (VLCDR1) comprising an amino acid sequence as set forth in SEQ ID NO: 36, a VLCDR2 comprising an amino acid sequence as set forth in SEQ ID NO: 37, and a VLCDR3 comprising an amino acid sequence as set forth in SEQ ID NO: 38. In certain embodiments, the glycoprotein comprises a heavy chain variable region comprising an amino acid sequence having at least 70%, 80%, 90%, 95% or 100% identity as the amino acid sequence set forth in SEQ ID NO: 34; and/or the glycoprotein comprises a light chain variable region comprising an amino acid sequence having at least 70%, 80%, 90%, 95% or 100% identity as the amino acid sequence set forth in SEQ ID NO: 39. Yet in some embodiments, the glycoprotein comprises a heavy chain comprising an amino acid sequence having at least 70%, 80%, 90%, 95% or 100% identity as the amino acid sequence set forth in SEQ ID NO: 35; and/or the glycoprotein comprises a light chain comprising an amino acid sequence having at least 70%, 80%, 90%, 95% or 100% identity as the amino acid sequence set forth in SEQ ID NO: 40. All numbers can be modified by “about,” which is defined herein.

In certain embodiments, the glycoprotein comprises a heavy chain variable complementarity-determining region 1 (VHCDR1) comprising an amino acid sequence as set forth in SEQ ID NO: 41, a VHCDR2 comprising an amino acid sequence as set forth in SEQ ID NO: 42, a VHCDR3 comprising an amino acid sequence as set forth in SEQ ID NO: 43, a light chain variable complementarity-determining region 1 (VLCDR1) comprising an amino acid sequence as set forth in SEQ ID NO: 46, a VLCDR2 comprising an amino acid sequence as set forth in SEQ ID NO: 47, and a VLCDR3 comprising an amino acid sequence as set forth in SEQ ID NO: 48. In certain embodiments, the glycoprotein comprises a heavy chain variable region comprising an amino acid sequence having at least 70%, 80%, 90%, 95% or 100% identity as the amino acid sequence set forth in SEQ ID NO: 44; and/or the glycoprotein comprises a light chain variable region comprising an amino acid sequence having at least 70%, 80%, 90%, 95% or 100% identity as the amino acid sequence set forth in SEQ ID NO: 49. In certain embodiments, the glycoprotein comprises a heavy chain comprising an amino acid sequence having at least 70%, 80%, 90%, 95% or 100% identity as the amino acid sequence set forth in SEQ ID NO: 45; and/or the glycoprotein comprises a light chain comprising an amino acid sequence having at least 70%, 80%, 90%, 95% or 100% identity as the amino acid sequence set forth in SEQ ID NO: 50. All numbers can be modified by “about,” which is defined herein.

In certain embodiments, the glycoprotein is a monoclonal antibody targeting PD-1. In some embodiments, the glycoprotein is a monoclonal antibody competing antigen-binding with Pembrolizumab (Keytruda®) or Nivolumab (Opdivo®).

In certain embodiments, the glycoprotein comprises a heavy chain variable complementarity-determining region 1 (VHCDR1) comprising an amino acid sequence as set forth in SEQ ID NO: 51, a VHCDR2 comprising an amino acid sequence as set forth in SEQ ID NO: 52, a VHCDR3 comprising an amino acid sequence as set forth in SEQ ID NO: 53, a light chain variable complementarity-determining region 1 (VLCDR1) comprising an amino acid sequence as set forth in SEQ ID NO: 56, a VLCDR2 comprising an amino acid sequence as set forth in SEQ ID NO: 57, and a VLCDR3 comprising an amino acid sequence as set forth in SEQ ID NO: 58. In certain embodiments, the glycoprotein comprises a heavy chain variable region comprising an amino acid sequence having at least 70%, 80%, 90%, 95% or 100% identity as the amino acid sequence set forth in SEQ ID NO: 54; and/or the glycoprotein comprises a light chain variable region comprising an amino acid sequence having at least 70%, 80%, 90%, 95% or 100% identity as the amino acid sequence set forth in SEQ ID NO: 59. Yet in certain embodiments, the glycoprotein comprises a heavy chain comprising an amino acid sequence having at least 70%, 80%, 90%, 95% or 100% identity as the amino acid sequence set forth in SEQ ID NO: 55; and/or the glycoprotein comprises a light chain comprising an amino acid sequence having at least 70%, 80%, 90%, 95% or 100% identity as the amino acid sequence set forth in SEQ ID NO: 60. All numbers can be modified by “about,” which is defined herein.

In certain embodiments, the glycoprotein comprises a heavy chain variable complementarity-determining region 1 (VHCDR1) comprising an amino acid sequence as set forth in SEQ ID NO: 61, a VHCDR2 comprising an amino acid sequence as set forth in SEQ ID NO: 62, a VHCDR3 comprising an amino acid sequence as set forth in SEQ ID NO: 63, a light chain variable complementarity-determining region 1 (VLCDR1) comprising an amino acid sequence as set forth in SEQ ID NO: 66, a VLCDR2 comprising an amino acid sequence as set forth in SEQ ID NO: 67, and a VLCDR3 comprising an amino acid sequence as set forth in SEQ ID NO: 68. In certain embodiments, the glycoprotein comprises a heavy chain variable region comprising an amino acid sequence having at least 70%, 80%, 90%, 95% or 100% identity as the amino acid sequence set forth in SEQ ID NO: 64; and/or the glycoprotein comprises a light chain variable region comprising an amino acid sequence having at least 70%, 80%, 90%, 95% or 100% identity as the amino acid sequence set forth in SEQ ID NO: 69. Yet in certain embodiments, the glycoprotein comprises a heavy chain comprising an amino acid sequence having at least 70%, 80%, 90%, 95% or 100% identity as the amino acid sequence set forth in SEQ ID NO: 65; and/or the glycoprotein comprises a light chain comprising an amino acid sequence having at least 70%, 80%, 90%, 95% or 100% identity as the amino acid sequence set forth in SEQ ID NO: 70. All numbers can be modified by “about,” which is defined herein.

In certain embodiments, the glycoprotein is a fusion protein comprising a TNFα receptor domain and an Fc domain. In some embodiments, the glycoprotein is a fusion protein comprising a target-binding site competing with Etanercept (Enbrel®). In certain embodiments, the glycoprotein comprises an amino acid sequence having at least 70%, 80%, 90%, 95% or 100% identity as the amino acid sequence set forth in SEQ ID NO: 71 or SEQ ID NO: 72.

In certain embodiments, the glycoprotein is a monoclonal antibody targeting HER-2. In some embodiments, the glycoprotein is a monoclonal antibody competing antigen-binding with Trastuzumab (Herceptin®) or Pertuzumab (Perjeta®).

In certain embodiments, the glycoprotein comprises a heavy chain variable complementarity-determining region 1 (VHCDR1) comprising an amino acid sequence as set forth in SEQ ID NO: 73, a VHCDR2 comprising an amino acid sequence as set forth in SEQ ID NO: 74, a VHCDR3 comprising an amino acid sequence as set forth in SEQ ID NO: 75, a light chain variable complementarity-determining region 1 (VLCDR1) comprising an amino acid sequence as set forth in SEQ ID NO: 78, a VLCDR2 comprising an amino acid sequence as set forth in SEQ ID NO: 79, and a VLCDR3 comprising an amino acid sequence as set forth in SEQ ID NO: 80. In certain embodiments, the glycoprotein comprises a heavy chain variable region comprising an amino acid sequence having at least 70%, 80%, 90%, 95% or 100% identity as the amino acid sequence set forth in SEQ ID NO: 76; and/or a light chain variable region comprising an amino acid sequence having at least 70%, 80%, 90%, 95% or 100% identity as the amino acid sequence set forth in SEQ ID NO: 81. In certain embodiments, the glycoprotein comprises a heavy chain comprising an amino acid sequence having at least 70%, 80%, 90%, 95% or 100% identity as the amino acid sequence set forth in SEQ ID NO: 77; and/or a light chain comprising an amino acid sequence having at least 70%, 80%, 90%, 95% or 100% identity as the amino acid sequence set forth in SEQ ID NO: 82. All numbers can be modified by “about,” which is defined herein.

In certain embodiments, the glycoprotein comprises a heavy chain variable complementarity-determining region 1 (VHCDR1) comprising an amino acid sequence as set forth in SEQ ID NO: 83, a VHCDR2 comprising an amino acid sequence as set forth in SEQ ID NO: 84, a VHCDR3 comprising an amino acid sequence as set forth in SEQ ID NO: 85, a light chain variable complementarity-determining region 1 (VLCDR1) comprising an amino acid sequence as set forth in SEQ ID NO: 88, a VLCDR2 comprising an amino acid sequence as set forth in SEQ ID NO: 89, and a VLCDR3 comprising an amino acid sequence as set forth in SEQ ID NO: 90. In certain embodiments, the glycoprotein comprises a heavy chain variable region comprising an amino acid sequence having at least 70%, 80%, 90%, 95% or 100% identity as the amino acid sequence set forth in SEQ ID NO: 86; and/or the glycoprotein comprises a light chain variable region comprising an amino acid sequence having at least 70%, 80%, 90%, 95% or 100% identity as the amino acid sequence set forth in SEQ ID NO: 91. In certain embodiments, the glycoprotein comprises a heavy chain comprising an amino acid sequence having at least 70%, 80%, 90%, 95% or 100% identity as the amino acid sequence set forth in SEQ ID NO: 87; and/or the glycoprotein comprises a light chain comprising an amino acid sequence having at least 70%, 80%, 90%, 95% or 100% identity as the amino acid sequence set forth in SEQ ID NO: 92. All numbers can be modified by “about,” which is defined herein.

In certain embodiments, the glycoprotein is a monoclonal antibody targeting EGFR. In some embodiments, the glycoprotein is a monoclonal antibody competing antigen-binding with Cetuximab (Erbitux®). In certain embodiments, the glycoprotein comprises a heavy chain variable complementarity-determining region 1 (VHCDR1) comprising an amino acid sequence as set forth in SEQ ID NO: 93, a VHCDR2 comprising an amino acid sequence as set forth in SEQ ID NO: 94, a VHCDR3 comprising an amino acid sequence as set forth in SEQ ID NO: 95, a light chain variable complementarity-determining region 1 (VLCDR1) comprising an amino acid sequence as set forth in SEQ ID NO: 98, a VLCDR2 comprising an amino acid sequence as set forth in SEQ ID NO: 99, and a VLCDR3 comprising an amino acid sequence as set forth in SEQ ID NO: 100. In certain embodiments, the heavy chain variable region comprising an amino acid sequence having at least 70%, 80%, 90%, 95% or 100% identity as the amino acid sequence set forth in SEQ ID NO: 96; and/or the glycoprotein comprises a light chain variable region comprising an amino acid sequence having at least 70%, 80%, 90%, 95% or 100% identity as the amino acid sequence set forth in SEQ ID NO: 101. In certain embodiments, the glycoprotein comprises a heavy chain comprising an amino acid sequence having at least 70%, 80%, 90%, 95% or 100% identity as the amino acid sequence set forth in SEQ ID NO: 97; and/or the glycoprotein comprises a light chain comprising an amino acid sequence having at least 70%, 80%, 90%, 95% or 100% identity as the amino acid sequence set forth in SEQ ID NO: 102. All numbers can be modified by “about,” which is defined herein.

In certain embodiments, the glycoprotein is a monoclonal antibody targeting CD20. In some embodiments, the glycoprotein is a monoclonal antibody competing antigen-binding with Rituximab (Rituxan®) or Obinutuzumab (Gazyva®).

In certain embodiments, the glycoprotein comprises a heavy chain variable complementarity-determining region 1 (VHCDR1) comprising an amino acid sequence as set forth in SEQ ID NO: 103, a VHCDR2 comprising an amino acid sequence as set forth in SEQ ID NO: 104, a VHCDR3 comprising an amino acid sequence as set forth in SEQ ID NO: 105, a light chain variable complementarity-determining region 1 (VLCDR1) comprising an amino acid sequence as set forth in SEQ ID NO: 108, a VLCDR2 comprising an amino acid sequence as set forth in SEQ ID NO: 109, and a VLCDR3 comprising an amino acid sequence as set forth in SEQ ID NO: 110. In certain embodiments, the glycoprotein comprises a heavy chain variable region comprising an amino acid sequence having at least 70%, 80%, 90%, 95% or 100% identity as the amino acid sequence set forth in SEQ ID NO: 106; and/or the glycoprotein comprises a light chain variable region comprising an amino acid sequence having at least 70%, 80%, 90%, 95% or 100% identity as the amino acid sequence set forth in SEQ ID NO: 111. In certain embodiments, the glycoprotein comprises a heavy chain comprising an amino acid sequence having at least 70%, 80%, 90%, 95% or 100% identity as the amino acid sequence set forth in SEQ ID NO: 107; and/or the glycoprotein comprises a light chain comprising an amino acid sequence having at least 70%, 80%, 90%, 95% or 100% identity as the amino acid sequence set forth in SEQ ID NO: 112. All numbers can be modified by “about,” which is defined herein.

In certain embodiments, the glycoprotein comprises a heavy chain variable complementarity-determining region 1 (VHCDR1) comprising an amino acid sequence as set forth in SEQ ID NO: 13, a VHCDR2 comprising an amino acid sequence as set forth in SEQ ID NO: 114, a VHCDR3 comprising an amino acid sequence as set forth in SEQ ID NO: 115, a light chain variable complementarity-determining region 1 (VLCDR1) comprising an amino acid sequence as set forth in SEQ ID NO: 118, a VLCDR2 comprising an amino acid sequence as set forth in SEQ ID NO: 119, and a VLCDR3 comprising an amino acid sequence as set forth in SEQ ID NO: 120. In certain embodiments, a heavy chain variable region comprising an amino acid sequence having at least 70%, 80%, 90%, 95% or 100% identity as the amino acid sequence set forth in SEQ ID NO: 116; and/or a light chain variable region comprising an amino acid sequence having at least 70%, 80%, 90%, 95% or 100% identity as the amino acid sequence set forth in SEQ ID NO: 121. In certain embodiments, a heavy chain comprising an amino acid sequence having at least 70%, 80%, 90%, 95% or 100% identity as the amino acid sequence set forth in SEQ ID NO: 117; and/or a light chain comprising an amino acid sequence having at least 70%, 80%, 90%, 95% or 100% identity as the amino acid sequence set forth in SEQ ID NO: 122. All numbers can be modified by “about,” which is defined herein.

Yet in some embodiments, the glycoproteins described herein target pathogenic protein or toxin and are useful for treating an infectious disease. Exemplary monoclonal antibodies for infectious disease include, but are not limited to, anti-Ebola antibodies such as MB-003 (c13C6, h13F6 and c6D8), ZMab (m1H3, m2G4 and m4G7) and ZMapp (c13C6, c2G4, c4G7), anti-HIV antibodies such as VRC01, VRC02, VRC03, VRC06, b12, HJ16, 8ANC131, 8ANC134, CH103, NIH45, NIH46, NIH45G54W, NIH46G54W, 3BNC117, 3BNC60, VRC-PG04, INC9, 12A12, 12A21, VRC23, PG9, PGT145, PGDM1400, PG16, 2G12, PGT121, PGT128, PGT135, 4E10, 10E8, Z13 and 2F5, anti-influenza antibodies such as C179, CR6261, F10, FI6, CR8020, CH65, C05, TCN-032, D005, CR9114 and S139/1, anti-coronavirus antibody, anti-Pan-sarbecovirus antibody, anti-Pan-betacoronavirus antibody, and anti-SARS antibody.

### Composition

One aspect of the present disclosure provides a composition comprising an engineered glycoprotein population described herein and a pharmaceutically acceptable excipient. In some embodiments, the composition can be a pharmaceutic or therapeutic composition for treating a disease caused by a dysfunctional cell, wherein the glycoprotein is configured to target the dysfunctional cell.

In some embodiments, the pharmaceutically acceptable excipient comprises a solvent, dispersion media, diluent, dispersion, suspension aid, surface active agent, isotonic agent, thickening or emulsifying agent, preservative, polymer, peptide, protein, cell, hyaluronidase, or mixtures thereof. The types of pharmaceutically acceptable excipient can be chosen depending on the formulation of the composition. In certain embodiments, the composition is formulated in a form of tablets (including chewable tablets), capsules, strips, syrups, powders, pastilles, sachets, effervescent compositions, pills, coated bead systems, granules, microspheres, dragées, films, orally administrable films, liquid, solutions, solids, suspensions, or emulsions.

Other pharmaceutically acceptable carriers, excipients or stabilizers such as those described in Remington's Pharmaceutical Sciences 16th edition, Osol, A. Ed. (1980) may be included in the pre-lyophilized formulation (and/or the lyophilized formulation and/or the reconstituted formulation) provided that they do not adversely affect the desired characteristics of the formulation. Acceptable carriers, excipients or stabilizers are nontoxic to recipients at the dosages and concentrations employed, including but not limited to additional buffering agents, preservatives, co-solvents, antioxidants including ascorbic acid and methionine, chelating agents such as EDTA, metal complexes (e.g. Zn-protein complexes), biodegradable polymers such as polyesters, and/or salt-forming counterions such as sodium.

In some embodiments, the composition comprises at least 5, 10, 20, 25, 30, 40,50, 60, 70,80, 90, 100, 120, 150, 175, 200, 220, 250, 275, 300, 350, 450, 500, 550, 600, 650, 700, 750, 800, 850, 900, 950, or 1000 mg/mL of the engineered glycoprotein population of the present disclosure. The concentration of the engineered glycoprotein population can be a final concentration for use or a concentration suitable for storage. In some embodiments, the concentration is designed to be a pharmaceutically effective amount, or the concentration is so designed that multiple administrations of the composition will accumulatively reach the pharmaceutically effective amount. In some embodiments, the composition comprises 5 to 1000 mg/mL, 5 to 900 mg/mL, 5 to 800 mg/mL, 5 to 700 mg/mL, 5 to 600 mg/mL, 5 to 500 mg/mL, 5 to 400 mg/mL, 5 to 300 mg/mL, 5 to 200 mg/mL, 5 to 150 mg/mL, 5 to 100 mg/mL, 5 to 50 mg/mL, 25 to 1000 mg/mL, 25 to 900 mg/mL, 25 to 800 mg/mL, 25 to 700 mg/mL, 25 to 600 mg/mL, 25 to 500 mg/mL, 25 to 400 mg/mL, 25 to 300 mg/mL, 25 to 200 mg/mL, 25 to 150 mg/mL, 25 to 100 mg/mL, 25 to 50 mg/mL, 50 to 1000 mg/mL, 50 to 900 mg/mL, 50 to 800 mg/mL, 50 to 700 mg/mL, 50 to 600 mg/mL, 50 to 500 mg/mL, 50 to 400 mg/mL, 50 to 300 mg/mL, 50 to 200 mg/mL, 50 to 150 mg/mL, 50 to 100 mg/mL, 100 to 1000 mg/mL, 100 to 900 mg/mL, 100 to 800 mg/mL, 100 to 700 mg/mL, 100 to 600 mg/mL, 100 to 500 mg/mL, 100 to 400 mg/mL, 100 to 300 mg/mL, 100 to 200 mg/mL, or 100 to 150 mg/mL of the engineered glycoprotein population of the present disclosure. All numbers can be modified by “about,” which is defined herein.

After preparation of the glycoprotein as described herein by the cell of the present disclosure, a “pre-lyophilized formulation” can be produced. The glycoproteins for preparing the composition/formulation of the present disclosure are preferably essentially pure and desirably essentially homogeneous (i.e. free from contaminating proteins etc) Noted that the “homogeneous” here in this paragraphs describes the type of glycoproteins, meaning the glycoproteins of the engineered protein population for preparing the composition/formulation target the same molecule (e.g., targeting the same antigen or epitope); the “homogeneous” here in this paragraph does not refer to the glycoforms. “Essentially pure” protein means a composition comprising at least about 90% by weight of the protein, based on total weight of the composition, preferably at least about 95% by weight. “Essentially homogeneous” protein means a composition comprising at least about 99% by weight of protein, based on total weight of the composition. In certain embodiments, the protein is an antibody.

The amount of glycoproteins in the pre-lyophilized formulation is determined taking into account the desired dose volumes, mode(s) of administration etc. Where the protein of choice is an intact antibody (a full-length antibody), from about 2 mg/mL to about 50 mg/mL, preferably from about 5 mg/mL to about 40 mg/mL and most preferably from about 20-30 mg/mL is an exemplary starting protein concentration. The protein is generally present in solution. For example, the protein may be present in a pH-buffered solution at a pH from about 4 to 8, and preferably from about 5 to 7. Exemplary buffers include histidine, phosphate, Tris, citrate, succinate and other organic acids. The buffer concentration can be from about 1 mM to about 20 mM, or from about 3 mM to about 15 mM, depending, for example, on the buffer and the desired isotonicity of the formulation (e.g. of the reconstituted formulation). The preferred buffer is histidine in that, as demonstrated below, this can have lyoprotective properties. Succinate was shown to be another useful buffer.

The lyoprotectant is added to the pre-lyophilized formulation. In preferred embodiments, the lyoprotectant is a non-reducing sugar such as sucrose or trehalose. The amount of lyoprotectant in the pre-lyophilized formulation is generally such that, upon reconstitution, the resulting formulation will be isotonic. However, hypertonic reconstituted formulations may also be suitable. In addition, the amount of lyoprotectant must not be too low such that an unacceptable amount of degradation/aggregation of the protein occurs upon lyophilization. Where the lyoprotectant is a sugar (such as sucrose or trehalose) and the protein is an antibody, exemplary lyoprotectant concentrations in the pre-lyophilized formulation are from about 10 mM to about 400 mM, and preferably from about 30 mM to about 300 mM, and most preferably from about 50 mM to about 100 mM.

The ratio of protein to lyoprotectant is selected for each protein and lyoprotectant combination. In the case of an antibody as the protein of choice and a sugar (e.g., sucrose or trehalose) as the lyoprotectant for generating an isotonic reconstituted formulation with a high protein concentration, the molar ratio of lyoprotectant to antibody may be from about 100 to about 1500 moles lyoprotectant to 1 mole glycoprotein, and preferably from about 200 to about 1000 moles of lyoprotectant to 1 mole glycoprotein, for example from about 200 to about 600 moles of lyoprotectant to 1 mole glycoprotein.

In preferred embodiments of the present disclosure, it has been found to be desirable to add a surfactant to the pre-lyophilized formulation. Alternatively, or in addition, the surfactant may be added to the lyophilized formulation and/or the reconstituted formulation. Exemplary surfactants include nonionic surfactants such as polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linolcamidopropyl-, myristamidopropyl-, palmidopropyl-, or isostearamidopropyl-betaine (e.g lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethyl glycol, polypropyl glycol, and copolymers of ethylene and propylene glycol (e.g. Pluronics, PF68 etc). The amount of surfactant added is such that it reduces aggregation of the reconstituted protein and minimizes the formation of particulates after reconstitution. For example, the surfactant may be present in the pre-lyophilized formulation in an amount from about 0.001 to 0.5%, and preferably from about 0.005 to 0.05%.

In certain embodiments of the present disclosure, a mixture of the lyoprotectant (such as sucrose or trehalose) and a bulking agent (e.g. mannitol or glycine) is used in the preparation of the pre-lyophilization formulation. The bulking agent may allow for the production of a uniform lyophilized cake without excessive pockets therein etc.

The pharmaceutical compositions and formulations described herein are preferably stable. A “stable” formulation/composition is one in which the antibody therein essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N. Y., Pubs. (1991) and Jones, A. Adv. Drug Delivery Rev. 10:29-90 (1993). Stability can be measured at a selected temperature for a selected time period.

The formulations to be used for in vivo administration must be sterile. This is readily accomplished by filtration through sterile filtration membranes, prior to, or following, lyophilization and reconstitution. Alternatively, sterility of the entire mixture may be accomplished by autoclaving the ingredients, except for protein, at about 120° C. for about 30 minutes, for example.

After the protein, lyoprotectant and other optional components are mixed together, the formulation is lyophilized. Many different freeze-dryers are available for this purpose such as Hull50® (Hull, USA) or GT20® (Leybold-Heraeus, Germany) freeze-dryers. Freeze-drying is accomplished by freezing the formulation and subsequently subliming ice from the frozen content at a temperature suitable for primary drying. Under this condition, the product temperature is below the eutectic point or the collapse temperature of the formulation. Typically, the shelf temperature for the primary drying will range from about-30 to 25° C. (provided the product remains frozen during primary drying) at a suitable pressure, ranging typically from about 50 to 250 mTorr. The formulation, size and type of the container holding the sample (e.g., glass vial) and the volume of liquid will mainly dictate the time required for drying, which can range from a few hours to several days (e.g. 40-60 hrs). A secondary drying stage may be carried out at about 0-40° C., depending primarily on the type and size of container and the type of protein employed. However, it was found herein that a secondary drying step may not be necessary. For example, the shelf temperature throughout the entire water removal phase of lyophilization may be from about 15-30° C. (e.g., about 20° C.). The time and pressure required for secondary drying will be that which produces a suitable lyophilized cake, dependent, e.g., on the temperature and other parameters. The secondary drying time is dictated by the desired residual moisture level in the product and typically takes at least about 5 hours (e.g. 10-15 hours). The pressure may be the same as that employed during the primary drying step. Frecze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial. As a general proposition, lyophilization will result in a lyophilized formulation in which the moisture content thereof is less than about 5%, and preferably less than about 3%.

At the desired stage, typically when it is time to administer the protein to the patient, the lyophilized formulation may be reconstituted with a diluent such that the protein concentration in the reconstituted formulation is at least 50 mg/mL, for example from about 50 mg/mL to about 400 mg/mL, more preferably from about 80 mg/mL to about 300 mg/mL, and most preferably from about 90 mg/mL to about 150 mg/mL. Such high protein concentrations in the reconstituted formulation are considered to be particularly useful where subcutaneous delivery of the reconstituted formulation is intended. However, for other routes of administration, such as intravenous administration, lower concentrations of the protein in the reconstituted formulation may be desired (for example from about 5-50 mg/mL, or from about 10-40 mg/mL protein in the reconstituted formulation). In certain embodiments, the protein concentration in the reconstituted formulation is significantly higher than that in the pre-lyophilized formulation. For example, the protein concentration in the reconstituted formulation may be about 2-40 times, preferably 3-10 times and most preferably 3-6 times (e.g. at least three fold or at least four fold) that of the pre-lyophilized formulation.

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution. The diluent optionally contains a preservative. Exemplary preservatives have been described above, with aromatic alcohols such as benzyl or phenol alcohol being the preferred preservatives. The amount of preservative employed is determined by assessing different preservative concentrations for compatibility with the protein and preservative efficacy testing. For example, if the preservative is an aromatic alcohol (such as benzyl alcohol), it can be present in an amount from about 0.1-2.0% and preferably from about 0.5-1.5%, but most preferably about 1.0-1.2%. Preferably, the reconstituted formulation has less than 6000 particles per vial which are >10 mm size.

### Methods of Treating a Disease Caused by a Dysfunctional Cell

One aspect of the present disclosure provides a method of treating a disease caused by a dysfunctional cell. The method comprises administering a subject in need the engineered glycoprotein population of the present disclosure, wherein the glycoprotein is configured to target the dysfunctional cell. As used herein and described above, a dysfunctional cell is a cell that has lost some or all of its normal function, has gained abnormal function, or has been inhibited or activated by another dysfunctional cell, thereby failing to perform its normal function. In some embodiments, the glycoprotein targets a surface marker (e.g., a protein or a glycan) of the dysfunctional cell, and such surface protein includes, but not limited to, tumor necrosis factor-alpha (TNFα), Vascular Endothelial Growth Factor (VEGF), Cytotoxic T-Lymphocyte Associated protein 4 (CTLA-4), PD-L1, PD-1, Human Epidermal Growth Factor Receptor 2 (HER2), Epidermal Growth Factor Receptor (EGFR), CD38, CD52, CD20, or tumor-associated carbohydrate antigens. In certain embodiments, the subject in need is a mammalian (e.g. a human).

In some examples, the dysfunctional cell is a tumor cell or an immune cell. In certain embodiments, the disease is an autoimmune disease or cancer, including, but not limited to, arthritis (including, rheumatoid arthritis, juvenile rheumatoid arthritis, juvenile idiopathic arthritis, psoriatic arthritis, polyarthritis, oligoarthritis, enthesitis-related arthritis, axial spondyloarthritis), ankylosing spondylitis, Crohn's disease, ulcerative colitis, plaque psoriasis, hidradenitis suppurativa, uveitis, ankylosing spondylitis, granulomatosis with polyangiitis (GPA), microscopic polyangiitis (MPA), pemphigus vulgaris, idiopathic thrombocytopeni purpura, myasthenia gravis, Epstein-Barr virus-positive mucocutaneous ulcers, macular degeneration, breast cancer, colorectal cancer, lung cancer (e.g., non-small cell lung cancer), kidney cancer, brain cancer, bladder cancer, ovarian cancer, cervical cancer, melanoma, prostate cancer, urothelial cancer, skin cancer (e.g., Merkel-cell carcinoma), head and neck cancer, Hodgkin lymphoma, Non-Hodgkin's lymphoma (NHL; e.g., follicular lymphoma), chronic lymphocytic leukemia (CLL), and mature B-cell acute leukemia (B-AL), gastric cancer, malignant pleural mesothelioma, urothelial carcinoma, colon cancer, esophageal squamous cell carcinoma, liver cancer, gastric cancer, esophageal cancer, or gastroesophageal junction cancer.

In some embodiments, the glycoprotein is Adalimumab (Humira®), and the disease is rheumatoid arthritis (including, rheumatoid arthritis, juvenile rheumatoid arthritis, juvenile idiopathic arthritis, psoriatic arthritis, polyarthritis, oligoarthritis, enthesitis-related arthritis, axial spondyloarthritis), ankylosing spondylitis, Crohn's disease, ulcerative colitis, plaque psoriasis, hidradenitis suppurativa, uveitis, ankylosing spondylitis, granulomatosis with polyangiitis (GPA), microscopic polyangiitis (MPA), pemphigus vulgaris, idiopathic thrombocytopeni purpura, myasthenia gravis, or Epstein-Barr virus-positive mucocutaneous ulcers.

In certain embodiments, when the glycoprotein is or competes target-binding with Adalimumab (Humira®), the disease is rheumatoid arthritis (including, rheumatoid arthritis, juvenile rheumatoid arthritis, juvenile idiopathic arthritis, psoriatic arthritis, polyarthritis, oligoarthritis, enthesitis-related arthritis, axial spondyloarthritis), ankylosing spondylitis, Crohn's disease, ulcerative colitis, plaque psoriasis, hidradenitis suppurativa, uveitis, ankylosing spondylitis, granulomatosis with polyangiitis (GPA), microscopic polyangiitis (MPA), pemphigus vulgaris, idiopathic thrombocytopenia purpura, myasthenia gravis, or Epstein-Barr virus-positive mucocutaneous ulcers.

In certain embodiments, when the glycoprotein is or competes target-binding with Bevacizumab (Avastin®), the disease is breast cancer, colorectal cancer, kidney cancer, lung cancer, brain cancer, ovarian cancer, cervical cancer, or macular degeneration.

In certain embodiments, when the glycoprotein is or competes target-binding with Ipilimumab (Yervoy®), the disease is melanoma, prostate cancer, lung cancer, or urothelial cancer.

In certain embodiments, when the glycoprotein is or competes target-binding with Avelumab (Bavencio®), the disease is bladder cancer, kidney cancer, or skin cancer.

In certain embodiments, when the glycoprotein is or competes target-binding with Durvalumab (IMFINZI®), the disease is non-small cell lung cancer.

In certain embodiments, when the glycoprotein is or competes target-binding with Pembrolizumab (Keytruda®), the disease is Non-Hodgkin's lymphoma (NHL) or chronic lymphocytic leukemia (CLL).

In certain embodiments, when the glycoprotein is or competes target-binding with Nivolumab (Opdivo®), the disease is melanoma, non-small cell lung cancer, malignant pleural mesothelioma, kidney cancer, Hodgkin lymphoma, head and neck cancer, urothelial cancer, colorectal cancer, liver cancer, esophageal cancer, or gastric cancer.

In certain embodiments, when the glycoprotein is or competes target-binding with Etanercept (Enbrel®), and the disease is arthritis (including, rheumatoid arthritis, juvenile rheumatoid arthritis, juvenile idiopathic arthritis, psoriatic arthritis, polyarthritis, oligoarthritis, enthesitis-related arthritis, axial spondyloarthritis), ankylosing spondylitis, Crohn's disease, ulcerative colitis, plaque psoriasis, hidradenitis suppurativa, uveitis, ankylosing spondylitis, granulomatosis with polyangiitis (GPA), microscopic polyangiitis (MPA), pemphigus vulgaris, idiopathic thrombocytopenia purpura, myasthenia gravis, or Epstein-Barr virus-positive mucocutaneous ulcers.

In certain embodiments, when the glycoprotein is or competes target-binding with Etanercept (Enbrel®), and the disease is breast cancer or gastric cancer.

In certain embodiments, when the glycoprotein is or competes target-binding with Pertuzumab (Perjeta®), the disease is breast cancer.

In certain embodiments, when the glycoprotein is or competes target-binding with Rituximab (Rituxan®), the disease is non-Hodgkin lymphoma (NHL), chronic lymphocytic leukemia (CLL), rheumatoid arthritis, granulomatosis with polyangiitis, idiopathic thrombocytopenia purpura, pemphigus vulgaris, myasthenia gravis, or Epstein-Barr virus-positive mucocutaneous ulcers.

In certain embodiments, when the glycoprotein is or competes target-binding with Obinutuzumab (Gazyva®), the disease is chronic lymphocytic leukemia (CLL) or follicular lymphoma.

In some embodiments, the engineered glycoprotein population is administered together with a pharmaceutically acceptable excipient. The pharmaceutically acceptable excipient can be a solvent, dispersion media, diluent, dispersion, suspension aid, surface active agent, isotonic agent, thickening or emulsifying agent, preservative, polymer, peptide, protein, cell, hyaluronidase, or mixtures thereof. The administering, in some embodiments, is made or via orally, intravenously (IV) intramuscularly (IM), intrathecally, subcutaneously, sublingually, buccally, rectally, vaginally, ocular route, otic route, nasally, inhalation, nebulization, cutaneously (topical or systemic), or transdermally. To facilitate the efficacy of the engineered glycoprotein population or the administration, in certain embodiments, the engineered glycoprotein population is formulated as the composition of the present disclosure, which can be in a form of tablets (including chewable tablets), capsules, strips, syrups, powders, pastilles, sachets, effervescent compositions, pills, coated bead systems, granules, microspheres, dragées, films, orally administrable films, liquid, solutions, solids, suspensions, or emulsions.

In some embodiments, the method comprises administering a subject in need a pharmaceutically effect amount of the engineered glycoprotein population of the present disclosure, and the pharmaceutically effective amount is at least 5, 10, 20, 25, 30, 40,50, 60, 70,80, 90, 100, 120, 150, 175, 200, 220, 250, 275, 300, 350, 450, 500, 550, 600, 650, 700, 750, 800, 850, 900, 950, or 1000 mg per day, or any range defined by the foregoing endpoints, such as 5 to 1000 mg per day, 5 to 900 mg per day, 5 to 800 mg per day, 5 to 700 mg per day, 5 to 600 mg per day, 5 to 500 mg per day, 5 to 400 mg per day, 5 to 300 mg per day, 5 to 200 mg per day, 5 to 150 mg per day, 5 to 100 mg per day, 5 to 50 mg per day, 25 to 1000 mg per day, 25 to 900 mg per day, 25 to 800 mg per day, 25 to 700 mg per day, 25 to 600 mg per day, 25 to 500 mg per day, 25 to 400 mg per day, 25 to 300 mg per day, 25 to 200 mg per day, 25 to 150 mg per day, 25 to 100 mg per day, 25 to 50 mg per day, 50 to 1000 mg per day, 50 to 900 mg per day, 50 to 800 mg per day, 50 to 700 mg per day, 50 to 600 mg per day, 50 to 500 mg per day, 50 to 400 mg per day, 50 to 300 mg per day, 50 to 200 mg per day, 50 to 150 mg per day, 50 to 100 mg per day, 100 to 1000 mg per day, 100 to 900 mg per day, 100 to 800 mg per day, 100 to 700 mg per day, 100 to 600 mg per day, 100 to 500 mg per day, 100 to 400 mg per day, 100 to 300 mg per day, 100 to 200 mg per day, or 100 to 150 mg per day, including or excluding any foregoing numbers. All numbers can be modified by “about,” which is defined herein.

In some embodiments, the pharmaceutically effective amount is achieved by multiple administrations. In such embodiments, the multiple administrations is performed at an interval of at least 0.5, 1, 2, 3, 4, 5, 6, 7, 8, 9, 10, 11, 12, 13, 14, 15, 16, 17, 18, 19, 20, 21, 22, 23, or 24 hours, or any range defined by the foregoing endpoints, such as 0.5 to 24 hours, 0.5 to 20 hours, 0.5 to 16 hours, 0.5 to 12 hours, 0.5 to 8 hours, 0.5 to 4 hours, 0.5 to 2 hours, 2 to 24 hours, 2 to 20 hours, 2 to 16 hours, 2 to 12 hours, 2 to 8 hours, 2 to 4 hours, 4 to 24 hours, 4 to 20 hours, 4 to 16 hours, 4 to 12 hours, 4 to 8 hours, 12 to 24 hours, 12 to 20 hours, or 12 to 16 hours, including or excluding any foregoing numbers. All numbers can be modified by “about,” which is defined herein.

### Cell

### Cells for Expressing a Sialylated Glycoprotein

One aspect of the present disclosure provides a cell for expressing a sialylated glycoprotein, which expresses constitutively and/or controllably an exogenous sialyltransferase catalytic peptide and an exogenous galactosyltransferase catalytic peptide, wherein the exogenous sialyltransferase catalytic peptide and the exogenous galactosyltransferase catalytic peptide are translated in close proximity. As used herein, “translated in close proximity” describes the translation event of the exogenous sialyltransferase catalytic peptide and the translation event of the transcription of the exogenous galactosyltransferase catalytic peptide happen closely to each other both temporally and spatially.

In some embodiments, the exogenous sialyltransferase catalytic peptide and the exogenous galactosyltransferase catalytic peptide are expressed in a single transcript. As used herein, “in a single transcript” means the transcription of the exogenous sialyltransferase catalytic peptide and the transcription of the exogenous galactosyltransferase catalytic peptide are performed transcriptionally under the same promoter or the DNA encoding the exogenous sialyltransferase catalytic peptide and the DNA encoding the exogenous galactosyltransferase catalytic peptide are transcribed into a single mRNA molecule.

The present disclosure contemplates that translating the exogenous sialyltransferase catalytic peptide and the exogenous galactosyltransferase catalytic peptide in proximity to each other or expressing the two peptides in a single transcription provides higher sialylation to the glycoprotein. Without wishing to be bound by any theories, since the catalytic reaction exerted by a galactosyltransferase generates a galactosylated glycan, which is the substrate of a sialyltransferase, translating the two enzymes in proximity or expressing them in a single transcription ensures the two catalytic reactions happen closely or nearly simultaneously, thereby higher sialylation efficiency.

Ribosomal shifting approach. In some embodiments, the cell of the present disclosure comprises a first nucleic acid configured to express the exogenous sialyltransferase catalytic peptide; and a second nucleic acid configured to express the exogenous galactosyltransferase catalytic peptide, wherein the first nucleic and the second nucleic acid are controlled under the same promoter. In some embodiments, the first nucleic acid and the second nucleic acid are connected to each other via a connecting nucleic acid, which is configured to encode a ribosomal shifting peptide. A ribosomal shifting peptide is configured to render ribosomal skipping, where the ribosomal shifting peptide prevents the ribosome from covalently linking a newly inserted amino acid while continuing the translation, resulting in co-translational cleavage of a polyprotein into separate peptides/proteins. In some embodiments, the ribosomal shifting peptide comprises an amino acid sequence of DxExNPGP, wherein x denotes any amino acid, D denotes aspartic acid, E donates glutamic acid, N denotes asparagine, P denotes proline, and G denotes glycine. In certain embodiments, the ribosomal shifting peptide comprises an amino acid sequence as set forth in SEQ ID NO: 128, SEQ ID NO: 129, SEQ ID NO: 130, or SEQ ID NO: 131. In certain embodiments, the connecting nucleic acid comprises a sequence as set forth in SEQ ID NO: 141, SEQ ID NO: 142, SEQ ID NO: 143, or SEQ ID NO: 144.

Fusion protein approach. Alternatively, the exogenous sialyltransferase catalytic peptide and the exogenous galactosyltransferase catalytic peptide are, in some embodiments, configured to be expressed into a fusion protein. In some embodiments, the fusion protein comprises a first portion having the sialyltransferase catalytic peptide and a second portion having the galactosyltransferase catalytic peptide, wherein the first portion and the second portion are connected to each other via a non-cleavable linker or a cleavable linker, and the cleavable linker is configured to be cleavable post-translation of the fusion protein, thereby upon cleavage releasing the sialyltransferase catalytic peptide and the galactosyltransferase catalytic peptide as separate proteins.

In some embodiments, the sialyltransferase catalytic peptide is an alpha-2,6-sialyltransferase. In some embodiments, the sialyltransferase is a beta-galactoside alpha-2,6-sialyltransferase 1. In certain embodiments, the sialyltransferase catalytic peptide comprises a product of an ST6Gall gene or a PspST gene. In certain embodiments, the sialyltransferase catalytic peptide comprises an amino acid sequence as the sequence set forth in SEQ ID NO: 123 or SEQ ID NO: 124. Yet in certain embodiments, the sialyltransferase catalytic peptide comprises an amino acid sequence as set forth in SEQ ID NO: 125 or SEQ ID NO: 126.

In some embodiments, the galactosyltransferase catalytic peptide is a beta-1,4-galactosyltransferase 1. In certain embodiments, the galactosyltransferase catalytic peptide comprises a product of a B4GALTI gene. In certain embodiments, the galactosyltransferase catalytic peptide comprises an amino acid sequence as set forth in SEQ ID NO: 127.

In some embodiments, the cell comprises a first nucleic acid configured to express the exogenous sialyltransferase catalytic peptide, wherein the first nucleic acid is derived from an ST6Gall gene or a PspST gene. As used herein, “derived from” describes that the first nucleic acid comprises a nucleotide sequence exactly the same as a reference gene; for example, the first nucleic acid might comprise a nucleotide sequence as set forth in SEQ ID NO: 132 or SEQ ID NO: 133. In some other situations, the “derived from” describes that the first nucleic acid comprises a nucleotide sequence modified from the reference gene, provided that the product of the modified nucleotide sequence exerts the same or similar catalytic functionality as the product of the reference gene. The modification might be made for accommodating codon usage or optimizing transcription/translation efficiency or accuracy in a host cell. In other examples, the modification adds a signal peptide directing the gene product to a certain place within or out of the cells. For example, the first nucleic acid can be derived from a PspST gene that the first nucleic acid comprises a nucleotide sequence encoding a product of the PspST gene and a nucleotide sequence encoding a signal peptide that directs the gene product to the Golgi body where glycosylation takes place. The signal peptide, in certain embodiments, can be the signal peptide of an ST6Gall gene or a B4GALTI gene. In such embodiments, the first nucleic acid comprises a nucleotide sequence as set forth in SEQ ID NO: 134 or SEQ ID NO: 135.

In some embodiments, the cell comprises a second nucleic acid configured to express the exogenous galactosyltransferase catalytic peptide, wherein the second nucleic acid is derived from a B4GALT1 gene. In certain embodiments, the second nucleic acid comprises a nucleotide sequence as set forth in SEQ ID NO: 136.

In some embodiments, the first nucleic acid and the second nucleic acid are transcriptionally controlled under the same promoter, which can be a constitutive promoter or an activable promoter. The constitutive promoter can be but is not limited to a CMV promoter, a T7 promoter, a Human Elongation Factor 1 Alpha (EFlα) promoter, a Chicken β-Actin (CAG) promoter, or an SV40 promoter. The activable promoter provides a controllable expression of the exogenous sialyltransferase and the exogenous galactosyltransferase. The activable promoter can be but is not limited to a Tetracycline-Inducible Promoter (activable by doxycycline) or a dihydrofolate reductase (DHFR) gene promoter (used to select and amplify gene expression).

Fucosyltransferase knockout. In some embodiments, the cell of the present disclosure is deficient in endogenous fucosyltransferase activity. As used herein, “deficient in endogenous fucosyltransferase activity” means the cell does not have an endogenous gene encoding the fucosyltransferase, the gene encoding the fucosyltransferase is deleted or at least partially deleted so the gene cannot be translated into a functioning fucosyltransferase, or the gene encoding the fucosyltransferase is deleted or at least partially deleted so even if the gene can be translated into a fucosyltransferase, the encoded fucosyltransferase has defect and cannot perform catalytic reaction as it should be in a normal status. In some embodiments, the cell of the present disclosure is deficient in fucosyltransferase activity.

The deficiency in fucosyltransferase activity can be obtained by gene editing tools known in the field, including, but not limited to, such as Sleeping Beauty transposon or I-Scel meganuclease-mediated transposon, or targeted gene editing, such as using transcription activator-like effector nuclease (TALEN) or clustered regularly interspaced short palindromic repeats (CRISPR)/CRISPR-associated protein. Those tools are described below in more details.

Accordingly, in one aspect, the present disclosure provides a cell for expressing a sialylated glycoprotein. The cell comprises a first nucleic acid, encoding an exogenous sialyltransferase catalytic peptide; a second nucleic acid, encoding an exogenous galactosyltransferase catalytic peptide; and a vector comprising: a nucleic acid encoding a Clustered Regularly Interspaced Short Palindromic Repeat (CRISPR) crRNA comprising a nucleotide sequence as set forth in SEQ ID NO: 150, SEQ ID NO: 151, or SEQ ID NO: 152; and a nucleic acid encoding a tracrRNA.

Yet in another aspect, the present disclosure provides a cell for expressing a sialylated glycoprotein. The cell constitutively and/or controllably expresses an exogenous sialyltransferase catalytic peptide and an exogenous galactosyltransferase catalytic peptide, wherein the exogenous sialyltransferase catalytic peptide and the exogenous galactosyltransferase catalytic peptide are translated in close proximity; and the cell is deficient in endogenous fucosyltransferase activity, and deficiency in endogenous fucosyltransferase activity is made by introducing a vector into a parent cell of the cell, wherein the vector comprises a nucleic acid encoding a Clustered Regularly Interspaced Short Palindromic Repeat (CRISPR) crRNA comprising a nucleotide sequence as set forth in SEQ ID NO: 150, SEQ ID NO: 151, or SEQ ID NO: 152; and a nucleic acid encoding a tracrRNA.

The tracrRNA, in some embodiments, might comprise a nucleotide sequence having at least 70%, 80%, 90%, 95%, or 100% identity as the nucleotide sequence set forth in SEQ ID NO: 154. In some embodiments, the vector further comprises a nucleic acid encoding a CRISPR-associated (Cas) protein, such as a Cas9, Cas 12a, Cas13, Cpf1, Cas8, or Cas5. In certain embodiments, the Cas protein is a Cas9 nuclease. In such embodiments, the nucleic acid encoding the Cas9 nuclease might comprise a nucleotide sequence having at least 70%, 80%, 90%, 95% or 100% identity as the nucleotide sequence set forth in SEQ ID NO: 153. All numbers can be modified by “about,” which is defined herein.

Parental cell. In some embodiments, the cell of the present disclosure is derived from a parental cell (e.g., a wild-type cell), which can be a mammalian cell. In some embodiments, the parental cell does not have an endogenous sialyltransferase and/or an endogenous galactosyltransferase. In some other embodiments, the parental cell might have an endogenous sialyltransferase and/or an endogenous galactosyltransferase. Nevertheless, the present disclosure surprisedly discovers that overexpression of the endogenous sialyltransferase and/or the endogenous galactosyltransferase does not increase the sialylation as expressing exogenous ones can achieve. In some embodiments, the parental cell and/or the cell of the present disclosure are deficient in fucosyltransferase 8 activity. In certain embodiments, the parental cell and/or the cell of the present disclosure are deficient in a FUT8 gene encoding a product of the fucosyltransferase activity. In some embodiments, the parental cell and/or the cell of the present disclosure are or are derived from a Chinese hamster ovary (CHO) cell or a HEK293 cell. The CHO cell can be but is not limited to ExpiCHO, CHO-K1, CHO-C, CHOZN, and CHOKIQ. In some embodiments, “cell” used herein is interchangeably with “cell line.”

Cells for expressing a recombinant glycoprotein. In some embodiments, the cell further comprises a payload nucleic acid configured to encode a recombinant glycoprotein, and the expression of the payload nucleic acid is transcriptionally controlled by a constitutive or an activable promoter. The constitutive promoter and the activable promoter are as described above and herein. In some embodiments, the recombinant glycoprotein expressed or produced by the cell is conjugated with a glycan. In certain embodiments, the glycan can be an N-linked glycan (i.e., N-glycan) or an O-linked glycan (i.e., O-glycan).

Glycan and glycoprotein. The cell of the present disclosure is capable of expressing a sialylated glycoprotein. In some embodiments, the sialylated glycoprotein comprises a sialyl complex type (SCT) glycan, which can be conjugated to the protein via a lysine residue (i.e., O-glycan) or an asparagine residue (i.e., N-glycan). In some embodiments, the cell of the present disclosure is configured to express an SCT-enriched protein. As used herein, “SCT-enriched” describes that the proteins expressed by the cell of the present disclosure have a higher SCT glycan percentage compared to the same proteins expressed by a parental cell used to produce the cells of the present disclosure. In some embodiments, the SCT glycan percentage is 10%, 20%, 30%, 40%, 50%, 60%, 70%, 80%, 90%, 100%, 200%, 300%, 400%, 500% higher than that of the proteins expressed by a parental cell, or any range defined by the foregoing endpoints, such as 10% to 500%, 10% to 400%, 10% to 300%, 10% to 200%, 10% to 100%, 10% to 90%, 10% to 80%, 10% to 70%, 10% to 60%, 10% to 50%, 10% to 40%, 10% to 30%, 10% to 20%, 30% to 500%, 30% to 400%, 30% to 300%, 30% to 200%, 30% to 100%, 30% to 90%, 30% to 80%, 30% to 70%, 30% to 60%, 30% to 50%, or 30% to 40%. In certain embodiments, the SCT glycan percentage is enriched to 1.2, 1.5, 2, 2.5, 3, 3.5, 4, 4.5, 5, 5.5, 6, 6.5, 7, 7.5, 8, 8.5, 9, 9.5, or 10 times the SCT glycan percentage of the proteins expressed by a parental cell, or any range defined by the foregoing endpoints, such as 1.2 to 10 times, 1.2 to 9 times, 1.2 to 8 times, 1.2 to 7 times, 1.2 to 6 times, 1.2 to 5 times, 1.2 to 4 times, 1.2 to 3 times, 1.2 to 2 times, 2 to 10 times, 2 to 9 times, 2 to 8 times, 2 to 7 times, 2 to 6 times, 2 to 5 times, 2 to 4 times, 2 to 3 times, 5 to 10 times, 5 to 9 times, 5 to 8 times, 5 to 7 times, or 5 to 6 times. In certain embodiments, the glycan is a mono-antennary or bi-antennary a2-6 sialyl complex type (SCT) glycan. In some embodiments, the glycan is a galactose-rich SCT glycan with or without core-fucose. In some other embodiments, the glycan is a fully galactosylated SCT glycan with or without core-fucose. All numbers can be modified by “about,” which is defined herein.

In certain embodiments, the glycoprotein is Adalimumab (Humira®), Adalimumab-atto (Amjevita®), Bevacizumab (Avastin®), Alemtuzumab (Campath®) Ipilimumab (Yervoy®), Avelumab (Bavencio®), Durvalumab (IMFINZI®), Pembrolizumab (Keytruda®), Nivolumab (Opdivo®), Etanercept (Enbrel®), Trastuzumab (Herceptin®), Pertuzumab (Perjeta®), Cetuximab (Erbitux®), Rituximab (Rituxan®), Rituximab-atto (Truxima®), Obinutuzumab (Gazyva®), Infliximab (Remicade®), Ofatumumab (Arzerra®), Golimumab (Simponi®), Atezolizumab (Tecentriq®), Ocrelizumab (OCREVUS®), Gazyvaro Infliximab (Remicade®), Zanidatamab (Ziihera®), Daratumumab (Darzalex®), or Trastuzumab emtansine (Kadcyla®). In certain embodiments, the glycoprotein is an immunogenic protein, such as a viral envelop protein, or a spike protein of a virus. For example, the immunogenic protein can be but is not limited to an influenza hemagglutinin or a SARS-COV-2 spike protein.

### Cells for Expressing a GlcNAc Glycoprotein

One aspect of the present disclosure provides a cell for expressing a GlcNAc glycoprotein, wherein the cell is deficient in N-acetylglucosaminyltransferase 1 (GnT1) activity and constitutively or controllably expresses an exogenous endoglycosidase. As described herein, “GlcNAc glycoprotein” describes that the glycan conjugated on the glycoprotein is composed of mainly GlcNAc. In some embodiments, the GlcNAc glycan can be a mono-GlcNAc glycan or a GlcNAc-Fuc glycan. In some embodiments, the glycoprotein is as described above and herein.

The exogenous endoglycosidase can be but is not limited to an endoglycosidase H (Endo H) or an endoglycosidase S2 (Endo S2). In certain embodiments, the exogenous endoglycosidase comprises an amino acid sequence as set forth in SEQ ID NO: 137 or SEQ ID NO: 138. In some certain embodiments, the cell comprises a nucleic acid configured to encode the exogenous endoglycosidase, wherein the nucleic acid might comprise a nucleotide sequence as set forth in SEQ ID NO: 139 or SEQ ID NO: 140. The nucleic acid can be controlled under a constitutive or an activable promoter. The constitutive promoter can be but is not limited to a CMV promoter, a T7 promoter, an EFIA promoter, a CAG promoter, or an SV40 promoter. The activable promoter provides a controllable expression of the exogenous sialyltransferase and the exogenous galactosyltransferase. The activable promoter can be but is not limited to a Tetracycline-Inducible Promoter (activable by doxycycline) or a dihydrofolate reductase (DHFR) gene promoter (used to select and amplify gene expression).

As described herein, “deficient in N-acetylglucosaminyltransferase I (GnT1) activity” means that the cell does not have a functioning N-acetylglucosaminyltransferase I, which can result from knocking out an endogenous gene encoding the N-acetylglucosaminyltransferase I or a mutation at the gene causing missense, nonsense, or frameshift silent of the gene. Without wishing to be bound by any theories, a cell deficient in GnT1 activity produces a mannose-rich glycan, which is preferentially cleaved by an endoglycosidase, thereby generating a GlcNAc glycoprotein.

Parent cell. In some embodiments, the cell of the present disclosure is derived from a parent cell, which can be a mammalian cell. In some embodiments, the parent cell is deficient in N-acetylglucosaminyltransferase I (GnT1) activity. In some other embodiments, the parent cell is not deficient in acetylglucosaminyltransferase I (GnT1) activity, while the cell of the present disclosure is genetically engineered to be deficient in GnT1 activity. In some embodiments, the parent cell and/or the cell of the present disclosure are deficient in fucosyltransferase 8 activity. In certain embodiments, the parent cell and/or the cell of the present disclosure are deficient in a FUT8 gene encoding a product of the fucosyltransferase activity. In some embodiments, the parent cell and/or the cell of the present disclosure are or are derived from a Chinese hamster ovary (CHO) cell or a HEK293 cell. The CHO cell can be but is not limited to ExpiCHO, CHO-S, CHO-K1, CHO-C, CHOZN, and CHOKIQ, and the HEK293 cell can be Expi293F™ GnT1 KO.

Cells for expressing a recombinant glycoprotein. In some embodiments, the cell further comprises a payload nucleic acid configured to encode a recombinant glycoprotein, and the expression of the payload nucleic acid is controlled by a constitutive or an activable promoter. The constitutive promoter and the activable promoter are as described above and herein. In some embodiments, the recombinant glycoprotein expressed or produced by the cell is conjugated with a glycan. In certain embodiments, the glycan can be an N-linked glycan.

### Production of the Cells of the Present Disclosure

The production of the cells according to an embodiment of the present disclosure entails engineering a parent cell so that the engineered cell expresses the exogenous enzymes. In some embodiments, the production of the cells entails engineering a parent cell to knock in and/or knock out a gene of interest. In some embodiments, the engineering can be performed using transfection approaches or gene editing approaches, which can be either stable or transient.

### Transfection

The transfection can be conducted using any conventional methodologies with the consideration of high transfection efficiency, minimal cell toxicity, low or no significant effects on normal physiology, and case of operation.

In some embodiments, the transfection can be performed using virus-mediated methods. The virus-mediated methods use a viral vector to bring a nucleic acid configured to encode a desired gene product into a host cell. Some commonly used viral vectors include but are not limited to Adenovirus, Adeno-associated virus, retrovirus murine leukemia virus, Herpes simplex virus, Vaccinia virus, and Sindbis virus. Virus-mediated methods are highly effective, given the infectious nature of the viral particles. The downsides of these methods, however, lie in the concerns of immunogenicity and cytotoxicity. Nevertheless, many virus-mediated methods have been well-studied and developed to ease the concerns, and in some embodiments, the cell of the present disclosure is used to produce a desired glycoprotein in which uses, the transfection is not performed on a patient or a human subject directly. Therefore, the immunogenicity and cytotoxicity concerns are less critical for the present disclosure.

In some embodiments, the transfection can be performed using chemical methods, such as using a cationic polymer, calcium phosphate, a cationic lipid, or a cationic amino acid. Specific examples of chemical methods include but are not limited to using DEAE-dextran, polyethylencimine, dendrimer, polybrene, calcium phosphate, lipofectin, DOTAP, lipofectamine, CTAB/DOPE, or DOTMA. The underlying principle of those methods is using those positively charged chemicals to make nucleic acid/chemical complexes with negatively charged nucleic acids. The nucleic acid/chemical complexes are believed to be attracted to the negatively charged cell membrane and pass through it eventually via endocytosis, phagocytosis, or both. The efficiency of those chemical methods can be affected by several factors including the nucleic acid/chemical ratio, pH value of the environment, and cell membrane conditions.

In some embodiments, the transfection can be performed using physical methods, such as direct microinjection, biolistic particle delivery, electroporation, laser-based transfection, ultrasound transfection, and magnetofection. Those methods can be useful but usually require a higher skill level and are labor-intensive.

### Gene Editing

In some embodiments, the engineering can be performed using gene editing. The gene editing methods can be non-targeted gene editing, such as Sleeping Beauty transposon or I-Scel meganuclease-mediated transposon, or targeted gene editing, such as using transcription activator-like effector nuclease (TALEN) or clustered regularly interspaced short palindromic repeats (CRISPR)/CRISPR-associated protein. Gene editing provides integration of the exogenous nucleic acid into the genome of the host cell (e.g., the parental cell as described herein).

The targeted gene editing methods induce DNA double-strand breaks (DSBs) at a targeted locus, which enable the opportunities to alter the function (inserting an exogenous gene or silencing an endogenous gene) of a gene of interest.

The TALEN approach entails using a chimeric molecule consisting of a transcription activator-like effector (TALE) domain and a Fokl nuclease catalytic domain. The TALE domain comprises a DNA-binding motif, which can be designed to target a specific locus of the host cell's genome. Normally, a pair of two chimeric molecules, each targeting the forward and reverse directions respectively, are required. Once the TALE domains bind the targeted locus in the forward and reverse directions, the Fokl nuclease catalytic domains of the pair of two chimeric molecules form a dimer, thereby activating the catalytic activity of the nuclease to cleave the DNA and generate a DSB.

The CRISPR (Clustered Regularly Interspaced Short Palindromic Repeat) system is an adaptable, naturally occurring immune mechanism, used by prokaryotic organisms such as bacteria and archaea to defend against foreign nucleic acids such as viruses. CRISPR is a family of DNA sequences derived from DNA fragments of bacteriophages that the prokaryotic organism previously exposed to. These sequences are used to detect and destroy DNA from similar bacteriophages during subsequent infections. A set of enzymes called Cas (CRISPR-associated proteins) are nucleases found in association with these CRISPR sequences. Cas nucleases use CRISPR sequences as a guide to recognize and open up specific strands of DNA that are complementary to the CRISPR sequence. This CRISPR-Cas system has been adapted into a powerful gene-engineering tool for site specific editing. To perform site specific editing, a Cas nuclease must form complex with a guide RNA (gRNA), which comprises a CRISPR RNA (crRNA; spacer) and a trans-activating crRNA (tracrRNA; gRNA scaffold). The crRNA has a complementary sequence to a specific locus of the host cell's genome. Normally, the crRNA must have a sequence that is unique and specific to the target locus to prevent off-target binding, and the target locus is normally followed by a protospacer adjacent motif (PAM) site, which is necessary for Cas protein recognition. Once the guide RNA/Cas protein complex binds to the target site, the Cas nuclease activity will be activated and cleave the DNA to generate a DSB. Accordingly, a two-vector system is commonly used for CRISPR gene editing-one encodes the Cas protein, and the other for the gRNA.

The DSBs generated can be repaired by three different repair mechanisms, including homology-directed repair (HDR), non-homologous end joining (NHEJ), and micro-homology-mediated end joining (MMEJ). In the process of the repair mechanism, deletion or frameshift can result in gene silence, or, by using a donor vector comprising microhomology sequences homologous to a targeted locus, the repair mechanism can efficiently induce a targeted integration of exogenous genes into the genome.

### Methods for Glycoengineering a Recombinant Glycoprotein

One aspect of the present disclosure provides a method for glycoengineering a recombinant glycoprotein. The method comprises delivering an expression vector into a cell according to an embodiment of the present disclosure, wherein the expression vector comprises a payload nucleic acid configured to encode a recombinant glycoprotein; and expressing the payload nucleic acid in the cell, thereby obtaining a plurality of recombinant glycoproteins, where at least one recombinant glycoprotein of the plurality is conjugated with a desired glycan.

The glycoprotein can be as those described herein. In certain embodiments, the glycoprotein is a therapeutic antibody or an antigen-binding fragment thereof. In the embodiments that the glycoprotein is an antibody or an antigen-binding fragment thereof, the glycan can be located on a heavy chain or an Fc region. In some embodiments, the glycan is located at a glycosylation site of the antibody. In certain embodiments, the glycan can be located on N46, N402, and/or N297 of a heavy chain.

As used herein, “glycoengineering” refers to producing the recombinant glycoprotein using the cell whereby the recombinant glycoprotein carries or is conjugated with a desired glycoform or whereby the population of the glycoengineered recombinant glycoproteins has a desired glycan profile. In some embodiments, at least 50%, 55%, 60%, 65%, 70%, 75%, 80%, 85%, 90%, 95%, 99%, or 100% of the glycoengineered recombinant glycoproteins carry the desired glycoform (e.g., a sialylated glycan or a GlcNAc glycan), or any number ranging between any of the above two numbers, or any range defined by the foregoing endpoints, such as 50 to 100%, 50 to 99%, 50 to 95%, 50 to 90%, 50 to 85%, 50 to 80%, 50 to 75%, 50 to 70%, 50 to 65%, 50 to 60%, 50 to 55%, 60 to 100%, 60 to 99%, 60 to 95%, 60 to 90%, 60 to 85%, 60 to 80%, 60 to 75%, 60 to 70%, 60 to 65%, 70 to 100%, 70 to 99%, 70 to 95%, 70 to 90%, 70 to 85%, 70 to 80%, 70 to 75%, 80 to 100%, 80 to 99%, 80 to 95%, 80 to 90%, 80 to 85%, 90 to 100%, 90 to 99%, or 90 to 95%. All numbers can be modified by “about,” which is defined herein.

In some embodiments, the plurality of recombinant glycoproteins is conjugated with the desired glycoform (e.g., a sialylated glycan or a GlcNAc glycan) at homogeneity of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, or 88%, or any range defined by the foregoing endpoints, such as 1% to 88%, 1% to 80%, 1% to 70%, 1% to 60%, 1% to 50%, 1% to 40%, 1% to 30%, 1% to 20%, 1% to 10%, 5% to 100%, 5% to 95%, 5% to 85%, 5% to 75%, 5% to 65%, 5% to 55%, 5% to 45%, 5% to 35%, 5% to 25%, 5% to 10%, 10% to 88%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 20% to 88%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 40% to 88%, 40% to 85%, 40% to 80%, 40% to 75%, 40% to 70%, 40% to 65%, 40% to 60%, 40% to 55%, 40% to 50%, 60% to 88%, 60% to 85%, 60% to 80%, 60% to 75%, 50% to 88%, 50% to 87%, 50% to 86%, 50% to 85%, 50% to 80%, 50% to 75%, 50% to 70%, 50% to 65%, 50% to 60%, 55% to 88%, 55% to 87%, 55% to 86%, 55% to 85%, 55% to 80%, 55% to 75%, 55% to 70%, 55% to 65%, 55% to 60%, 70% to 88%, 70% to 85%, 70% to 80%, 70% to 75%, 80% to 88%, or 80% to 85%, including or excluding any foregoing numbers. All numbers can be modified by “about,” which is defined herein.

In some embodiments, the cell is according to the cell of the present disclosure described above for expressing a sialylated glycoprotein. In some embodiments, the sialylated glycoprotein comprises a sialyl complex type (SCT) glycan. In some embodiments, the method of the present disclosure is configured to express an SCT-enriched protein, which is defined as described herein. In some other embodiments, the cell is according to the cell of the present disclosure above for expressing a GlcNAc glycoprotein, which is defined as described herein.

In some embodiments, the method further comprises harvesting the recombinant glycoproteins within 200, 180, 150, 120, 100, 80, 50, 30, 20, 10, 5, or 1 hour from the expression of the payload nucleic acid, or an integer ranging between any of the above two numbers, or any range defined by the foregoing endpoints, such as 1 to 200 hours, 1 to 180 hours, 1 to 150 hours, 1 to 120 hours, 1 to 100 hours, 1 to 80 hours, 1 to 50 hours, 1 to 30 hours, 1 to 20 hours, 1 to 10 hours, 1 to 5 hours, 5 to 200 hours, 5 to 180 hours, 5 to 150 hours, 5 to 120 hours, 5 to 100 hours, 5 to 80 hours, 5 to 50 hours, 5 to 30 hours, 5 to 20 hours, or 5 to 10 hours. In some embodiments, the recombinant glycoprotein is harvested within 10, 9, 8, 7, 6, 5, 4, 3, 2, or 1 day from the expression of the payload nucleic acid, or any number ranging between any of the above two numbers, or any range defined by the foregoing endpoints, such as 1 to 10 days, 1 to 9 days, 1 to 8 days, 1 to 7 days, 1 to 6 days, 1 to 5 days, 1 to 4 days, 1 to 3 days, or 1 to 2 days. As described herein, “from the expression of the payload nucleic acid” means starting from the time point that the expression vector is delivered into the cell, or starting from the time point that the expression vector is delivered into the cell and the promoter controlling the expression of the payload nucleic acid is activated. All numbers can be modified by “about,” which is defined herein.

In some embodiments, the expression of the payload nucleic acid is constitutive or controllable. In certain embodiments, the expression of the payload nucleic acid is controlled by a constitutive promoter or an activable promoter. The constitutive promoter can be but is not limited to a CMV promoter, a T7 promoter, an EFIA promoter, a CAG promoter, or an SV40 promoter. The activable promoter provides a controllable expression of the exogenous sialyltransferase and the exogenous galactosyltransferase. The activable promoter can be but is not limited to a Tetracycline-Inducible Promoter (activable by doxycycline) or a dihydrofolate reductase (DHFR) gene promoter (used to select and amplify gene expression).

In some embodiments, the expression vector can be delivered via gene engineering, which can be performed using transfection approaches or gene editing approaches, which can be either stable or transient. The transfection approaches and the gene editing approaches can be as those described herein for producing the cells of the present disclosure.

Another aspect of the present disclosure provides a plurality of recombinant glycoproteins, wherein at least 50% of the plurality of the recombinant glycoproteins is configured with a desired glycan, which can be a sialylated glycan or a GlcNAc glycan. The recombinant glycoprotein can be described herein. In certain embodiments, the plurality of recombinant glycoproteins is obtained by using the methods for glycoengineering a recombinant glycoprotein of the present disclosure.

In some embodiments, the plurality of recombinant glycoproteins is a plurality of enriched recombinant glycoprotein. The term “enriched” describes that the plurality of recombinant glycoproteins of the present disclosure have a higher or enriched desired glycan percentage compared to the same proteins expressed by a parental cell or a wild-type cell used to produce the cells of the present disclosure, or the plurality of recombinant glycoproteins of the present disclosure have a higher or enriched desired glycan percentage compared to the desired glycan percentage of a plurality of naturally-occurred proteins having the same or similar amino acid sequences. In some embodiments, the desired glycan percentage is enriched by at least 30%, 35%, 40%, 45%, 50%, 55%, 60%, 65%, 70%, 75%, 80%, 85%, 90%, 95%, 100%, 150%, 200%, 250%, 300%, 350%, 400%, 450%, or 500%, or any number ranging between any of the above two numbers, or any range defined by the foregoing endpoints, such as 10% to 500%, 10% to 400%, 10% to 300%, 10% to 200%, 10% to 100%, 10% to 90%, 10% to 80%, 10% to 70%, 10% to 60%, 10% to 50%, 10% to 40%, 10% to 30%, 10% to 20%, 30% to 500%, 30% to 400%, 30% to 300%, 30% to 200%, 30% to 100%, 30% to 90%, 30% to 80%, 30% to 70%, 30% to 60%, 30% to 50%, or 30% to 40%, including or excluding any foregoing numbers. All numbers can be modified by “about,” which is defined herein. In certain embodiments, the desired glycan percentage is enriched to 1.2, 1.5, 2, 2.5, 3, 3.5, 4, 4.5, 5, 5.5, 6, 6.5, 7, 7.5, 8, 8.5, 9, 9.5, or 10 times the SCT glycan percentage of the proteins expressed by a parental cell, or an integer ranging between any of the above two numbers, or any range defined by the foregoing endpoints, such as 1.2 to 10 times, 1.2 to 9 times, 1.2 to 8 times, 1.2 to 7 times, 1.2 to 6 times, 1.2 to 5 times, 1.2 to 4 times, 1.2 to 3 times, 1.2 to 2 times, 2 to 10 times, 2 to 9 times, 2 to 8 times, 2 to 7 times, 2 to 6 times, 2 to 5 times, 2 to 4 times, 2 to 3 times, 5 to 10 times, 5 to 9 times, 5 to 8 times, 5 to 7 times, or 5 to 6 times, including or excluding any foregoing numbers. All numbers can be modified by “about,” which is defined herein.

In some embodiments, the desired glycan percentage is enriched by at least 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95%, or any number ranging between any of the above two numbers, or any range defined by the foregoing endpoints, such as 51% to 95%, 51% to 94%, 51% to 93%, 51% to 92%, 51% to 91%, 51% to 90%, 51% to 85%, 51% to 80%, 51% to 75%, 51% to 70%, 51% to 65%, 51% to 60%, 55% to 95%, 55% to 90%, 55% to 85%, 55% to 80%, 55% to 75%, 55% to 70%, 55% to 65%, 55% to 60%, 60% to 95%, 60% to 90%, 60% to 85%, 60% to 80%, 60% to 75%, 60% to 70%, 60% to 65%, 70% to 95%, 70% to 90%, 70% to 85%, 70% to 80%, or 70% to 75%, including or excluding any foregoing numbers. All numbers can be modified by “about,” which is defined herein.

In some embodiments, at least 50%, 55%, 60%, 65%, 70%, 75%, 80%, 85%, 90%, 95%, 99%, or 100% of the plurality of the recombinant glycoprotein carry the desired glycoform (e.g., a sialylated glycan or a GlcNAc glycan), or any number ranging between any of the above two numbers, or any range defined by the foregoing endpoints, such as 50 to 100%, 50 to 99%, 50 to 95%, 50 to 90%, 50 to 85%, 50 to 80%, 50 to 75%, 50 to 70%, 50 to 65%, 50 to 60%, 50 to 55%, 60 to 100%, 60 to 99%, 60 to 95%, 60 to 90%, 60 to 85%, 60 to 80%, 60 to 75%, 60 to 70%, 60 to 65%, 70 to 100%, 70 to 99%, 70 to 95%, 70 to 90%, 70 to 85%, 70 to 80%, 70 to 75%, 80 to 100%, 80 to 99%, 80 to 95%, 80 to 90%, 80 to 85%, 90 to 100%, 90 to 99%, or 90 to 95%. All numbers can be modified by “about,” which is defined herein.

In some embodiments, the plurality of recombinant glycoproteins is conjugated with the sialylated glycan at homogeneity of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, or 88%, or any range defined by the foregoing endpoints, such as 1% to 88%, 1% to 80%, 1% to 70%, 1% to 60%, 1% to 50%, 1% to 40%, 1% to 30%, 1% to 20%, 1% to 10%, 5% to 88%, 5% to 85%, 5% to 75%, 5% to 65%, 5% to 55%, 5% to 45%, 5% to 35%, 5% to 25%, 5% to 10%, 10% to 88%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 20% to 88%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 40% to 88%, 40% to 85%, 40% to 80%, 40% to 75%, 40% to 70%, 40% to 65%, 40% to 60%, 40% to 55%, 40% to 50%, 60% to 88%, 60% to 85%, 60% to 80%, 60% to 75%, 50% to 88%, 50% to 87%, 50% to 86%, 50% to 85%, 50% to 80%, 50% to 75%, 50% to 70%, 50% to 65%, 50% to 60%, 55% to 88%, 55% to 87%, 55% to 86%, 55% to 85%, 55% to 80%, 55% to 75%, 55% to 70%, 55% to 65%, 55% to 60%, 70% to 88%, 70% to 85%, 70% to 80%, 70% to 75%, or 80% to 88%, including or excluding any foregoing numbers. All numbers can be modified by “about,” which is defined herein.

In the embodiments that the cell is according to the cell of the present disclosure above for expressing a GlcNAc glycoprotein, the method of the present disclosure can further comprise a transglycosylation step. The transglycosylation step can comprise collecting the plurality of recombinant glycoproteins expressed in the preceding steps of the method and incubating the plurality of recombinant glycoproteins with an endoglycosidase and a donor glycan, thereby conjugating the donor glycan onto at least one glycoprotein of the plurality of recombinant glycoproteins (i.e., a transglycosylation reaction).

In some embodiments, the donor glycan comprises an oxazoline moiety configured to react with the endoglycosidase thereby conjugating the donor glycan onto at least one glycoprotein of the plurality of recombinant glycoproteins. In certain embodiments, the donor glycan is a sialyl complex type (SCT) glycan. In some embodiments, the endoglycosidase is an endoglycosidase H (Endo H), an endoglycosidase S2 (Endo S2), or a derivative thereof. In some embodiments, the derivative of the Endo S2 comprises an Endo S2 D184 mutant, including but not limited to, a D184M mutant or a D184Q mutant.

In some embodiments, the transglycosylation step generates a plurality of recombinant glycoprotein conjugated with the donor glycan at a homogeneity of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range defined by the foregoing endpoints, such as 1% to 100%, 1% to 90%, 1% to 80%, 1% to 70%, 1% to 60%, 1% to 50%, 1% to 40%, 1% to 30%, 1% to 20%, 1% to 10%, 5% to 100%, 5% to 95%, 5% to 85%, 5% to 75%, 5% to 65%, 5% to 55%, 5% to 45%, 5% to 35%, 5% to 25%, 5% to 10%, 10% to 100%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 20% to 100%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 40% to 100%, 40% to 99%, 40% to 95%, 40% to 90%, 40% to 85%, 40% to 80%, 40% to 75%, 40% to 70%, 40% to 65%, 40% to 60%, 40% to 55%, 40% to 50%, 60% to 100%, 60% to 99%, 60% to 95%, 60% to 90%, 60% to 85%, 60% to 80%, 60% to 75%, 50% to 100%, 50% to 99%, 50% to 95%, 50% to 90%, 50% to 85%, 50% to 80%, 50% to 75%, 50% to 70%, 50% to 65%, 50% to 60%, 55% to 100%, 55% to 99%, 55% to 98%, 55% to 97%, 55% to 96%, 55% to 95%, 55% to 94%, 55% to 93%, 55% to 92%, 55% to 91%, 55% to 90%, 55% to 85%, 55% to 80%, 55% to 75%, 55% to 70%, 55% to 65%, 55% to 60%, 70% to 100%, 70% to 99%, 70% to 95%, 70% to 90%, 70% to 85%, 70% to 80%, 70% to 75%, 80% to 100%, 80% to 99%, 80% to 95%, 80% to 90%, 80% to 85%, 90% to 100%, 90% to 99%, or 90% to 95%, including or excluding any foregoing numbers. All numbers can be modified by “about,” which is defined herein.

### Media

In some embodiments, the cell is cultured in a cell culture medium for producing a glycoprotein with 7-Fluorosialyl glycosides (e.g., having terminal 7-fluoro sialic acid). In those embodiments, the cell culture medium comprises (i) a basal medium or a complex medium, and (ii) peracetylated 4-fluoro N-Acetylmannosamine (peracetylated 4-F-ManNAc or O-acyl-modified 4-F-ManNAc). A “basal medium” and a “complex medium” are defined as it is defined in the art. Briefly, a basal medium typically provides a minimal nutrient solution with only the essentials required for growth, while a complex medium provides a wider range of nutrients. In some embodiments, the basal medium comprises a carbon source, amino acids, and minerals. Further in some embodiments, the cell culture medium might further comprise serum, vitamins, growth factors, a buffer system, a pH indicator, antibiotics, or a mixture thereof. In certain embodiments, the complex medium includes commercially-available products, but is not limited to, BalanCD CHO Growth A Medium, ExpiCHO™ Expression Medium, ExpiCHO™ Stable Production Medium, CD FortiCHO™ Medium, CD CHO AGT™ Medium, CD OptiCHO™ AGT™ Medium, CD CHO Medium, CHO-S-SFM II, High-Intensity Perfusion CHO Medium, Efficient-Pro™ Medium, Dulbecco's Modified Eagles Medium (DMEM), Roswell Park Memorial Institute medium (RPMI) 1640, DMEM F12, Modified Eagles Medium (MEM), or alpha-Minimum Essential Medium.

In some embodiments, the cell culture medium comprises about 10, 50, 100, 150, 200, 250, 300, 350, 400, 450, 500, 550, 600, 650, 700, 750, 800, 850, 900, 950, 1000, 1500, 2000, 2500, or 3000 uM (micromolar) of the peracetylated 4-F-ManNAc, or any range defined by the foregoing endpoints, such as 10 to 3000 uM, 10 to 2000 uM, 10 to 1000 uM, 10 to 900 uM, 10 to 800 uM, 10 to 700 uM, 10 to 600 uM, 10 to 500 uM, 10 to 400 uM, 10 to 300 uM, 10 to 200 uM, 10 to 100 uM, 100 to 3000 uM, 100 to 2500 uM, 100 to 2000 uM, 100 to 1500 uM, 100 to 1000 uM, 100 to 900 uM, 100 to 800 uM, 100 to 700 uM, 100 to 600 uM, 100 to 500 uM, 100 to 400 uM, 100 to 300 uM, 100 to 200 uM, 200 to 3000 uM, 200 to 2500 uM, 200 to 2000 uM, 200 to 1500 uM, 200 to 1000 uM, 200 to 900 uM, 200 to 800 uM, 200 to 700 uM, 200 to 600 uM, 200 to 500 uM, 200 to 400 uM, 200 to 350 uM, or 200 to 300 uM, including or excluding any foregoing numbers. All numbers can be modified by “about,” which is defined herein.

In some embodiments, the culture medium is devoid of sialyltransferase. As used herein, the phrase “devoid of sialyltransferase” means the culture medium, before being conditioned by the cell, is free of sialyltransferase or does not have detectable amount of sialyltransferase. That is to say, the phrase does not exclude the possibility that the cells cultured in the culture medium might express and release sialyltransferase into the cell culture medium.

In some embodiments, the carbon source comprises glucose, fructose, sucrose, sorbitol, mannitol, or a mixture thereof. In certain embodiments, the carbon source is at a working concentration of about 0.1 to 10 g/L. In some embodiments, the minerals comprise calcium, magnesium, potassium, sodium, phosphate, or a mixture thereof. In certain embodiments, the minerals are at a working concentration of about 0.1-5 mM. In some embodiments, the amino acids include, but are not limited to, glutamine, arginine, tyrosine, cystine, glycine, asparagine, or a mixture thereof. In certain embodiments, the amino acids are at a working concentration of about 0.1 to 100 mg/L. Nevertheless, the concentrations of the cell culture medium's components can vary depending on the type of cell being cultured and the desired outcome. Besides, as used herein, “working concentration” is defined as it is defined in the field, and the concentration defined herein mean to provide a reference concentration as the concentration of each component discussed here can be 2-time, 3-time, 5-time, 10-time higher for a stock medium.

### Methods of Increasing Binding Between a Fc Gamma Receptor (Fctr) and a Glycoprotein and Increasing Adcc, Cdc, Adcp, Vaccinal Effect, and Half-Live

One aspect of the present disclosure provides a method of increasing binding between a Fc gamma receptor (FcγR) and a glycoprotein. In another aspect, the present disclosure provides a method of increasing ADCC, CDC, ADCP, vaccinal effect, and half-live of a glycoprotein. The methods comprise engineering the glycoprotein to obtain an engineered glycoprotein population, wherein the engineered glycoprotein population is according to the engineered glycoprotein population of the present disclosure. In some embodiments, the glycoprotein comprises a first glycan and the engineering replaces the first glycan with a second glycan, and wherein the second glycan is a sialylated complex type glycan. The sialylated complex type glycan can be as described herein.

In some embodiments, the glycoprotein comprises a target-binding site and a glycosylation site where the first glycan or the second glycan conjugates. In some embodiments, the glycoprotein comprises a fragment crystallizable (Fc) region, which comprises the glycosylation site, and the first glycan and the second glycan is an Fc glycan. In certain embodiments, the glycoprotein is a therapeutic protein, as described herein. In some embodiments, the glycoprotein is an antibody or antigen-binding fragment thereof. In certain embodiments, the glycoprotein is Adalimumab (Humira®), Adalimumab-atto (Amjevita®), Bevacizumab (Avastin®), Alemtuzumab (Campath®), Ipilimumab (Yervoy®), Avelumab (Bavencio®), Durvalumab (IMFINZI®), Pembrolizumab (Keytruda®), Nivolumab (Opdivo®), Etanercept (Enbrel®), Trastuzumab (Herceptin®), Pertuzumab (Perjeta®), Cetuximab (Erbitux®), Rituximab (Rituxan®), Rituximab-atto (Truxima®), Obinutuzumab (Gazyva®), Infliximab (Remicade®), Ofatumumab (Arzerra®), Golimumab (Simponi®), Atezolizumab (Tecentriq®), Ocrelizumab (OCREVUS®), Gazyvaro Infliximab (Remicade®), Zanidatamab (Ziihera®). Daratumumab (Darzalex®), or Trastuzumab emtansinc (Kadcyla®).

In some embodiments, the engineering is performed by producing the glycoprotein by a cell according to the present disclosure. In certain embodiments, the cell constitutively and/or controllably expresses an exogenous sialyltransferase catalytic peptide and an exogenous galactosyltransferase catalytic peptide, wherein the exogenous sialyltransferase catalytic peptide and the exogenous galactosyltransferase catalytic peptide are translated in close proximity.

In certain embodiments, producing the glycoprotein by a cell according to the present disclosure is achieved by performing the method for glycoengineering a recombinant glycoprotein according to the present disclosure. The method, as described above, can, in some embodiments, comprise delivering an expression vector into a cell according to an embodiment of the present disclosure, wherein the expression vector comprises a payload nucleic acid configured to encode the glycoprotein; and expressing the payload nucleic acid in the cell, thereby obtaining an engineered glycoprotein population, where the engineered glycoprotein population comprises a heterogeneous glycan profile comprising at least 20%, 25%, 30%, 35%, 40%, 45%, 50%, 55%, 60%, 65%, 70%, 75%, or 80%, 85%, 86%, 88%, 90%, or 95% of heterogenous sialylated complex type (SCT) glycans, and the SCT glycans comprises two or more types of sialylated glycans. In some specific embodiments, the engineered glycoprotein population is the engineered glycoprotein population of the present disclosure.

### Definition

Unless specifically defined otherwise, all technical and scientific terms used herein have the same meaning as commonly understood by one of ordinary skill in the art to which this disclosure belongs. Unless mentioned otherwise, the techniques employed or contemplated herein are standard methodologies well known to one of ordinary skill in the art. The practice of the present disclosure will employ, unless otherwise indicated, conventional techniques of microbiology, tissue culture, molecular biology, chemistry, biochemistry, and recombinant DNA technology, which are within the skill of the art. The materials, methods, and examples are illustrative only and not limiting. The following is presented by way of illustration and is not intended to limit the scope of the disclosure.

Numbers expressing quantities of ingredients, properties such as homogeneity, enrich level, molecular weight, reaction conditions and results, and so forth, used to describe and claim certain embodiments of the present disclosure are to be understood as being modified in some instances by the term “about.” A skilled artisan in the field would understand the meaning of the term “about” in the context of the value that it qualifies. The numerical values presented in some embodiments of the present disclosure may contain certain errors resulting from the standard deviation in their respective testing measurements. For example, the term “about,” as used herein, refers to a measurable value such as an amount, a temporal duration, and the like and is meant to encompass variations of ±5%, ±1%, or ±0.1% from the specified value, as such variations are appropriate.

As used herein, “substantially” means sufficient to work for the intended purpose. The term “substantially” thus allows for minor, insignificant variations from an absolute or perfect state, dimension, measurement, result, or the like, such as expected by a person of ordinary skill in the field, but that does not appreciably affect overall performance. When used with respect to numerical values or parameters or characteristics expressed as numerical values, “substantially” means within ten percent.

As used herein, “treat,” “treatment,” and “treating” refer to an approach for obtaining beneficial or desired results, for example, clinical results. For this disclosure, beneficial or desired results may include inhibiting or suppressing the initiation or progression of an infection or a disease, ameliorating or reducing the development of symptoms of an infection or disease, or a combination thereof.

As used herein, “preventing” and “prevention” are used interchangeably with “prophylaxis” and can mean complete prevention of infection or prevention of the development of symptoms of that infection, a delay in the onset of a disease or its symptoms, or a decrease in the severity of a subsequently developed infection or its symptoms.

As used herein, “configured to” means the object is designed, produced, modified, or engineered to fulfill a purpose.

As used herein, “target,” when being used as a verb, means binding or specifically binding a molecule with a desired affinity. In some embodiments, the desired affinity can be defined as having a dissociation constant (K_{D}) less than or equal to about 10-3M, 104M, 105M, 10-6M, 10-7M, or 10-8M. In some embodiments, as used herein, the term “specifically binding” refers to the interaction between binding pairs (e.g., an antibody and an antigen). In various instances, specifically binding can be embodied by an affinity constant of about 10-6 moles/liter, about 10-7 moles/liter, or about 10-8 moles/liter, or less.

As used herein, “pharmaceutically effective amount” means an amount sufficient, at dosages and for periods of time needed, to achieve the desired clinical results or beneficial treatment, as described herein. A pharmaceutically effective amount can be determined based on several factors, including but not limited to the condition of the subjects (ages, gender, species, body weight, health status, etc.), the progress of the disease to be treated, the administration route, the administration frequency, the dosage and interval of the administration, and the nature of the disease. A pharmaceutically effective amount can be delivered in one or more administrations. If the administration is to a subject already known or confirmed to have a disease or disease state, the term “therapeutically effective amount” may be used in reference to treatment (A therapeutically effective amount is also one in which any toxic or detrimental effects of the substance/molecule are outweighed by the therapeutically beneficial effect), whereas “prophylactically effective amount” may be used to describe administering an effective amount to a subject that is susceptible to or at risk of developing a disease or disease-state (e.g., recurrence) as a beneficial and/or protective course of reducing (e.g., in a statistically significant manner relative to an untreated state) the likelihood of occurrence and/or severity of the disease or disease-state.

As used herein, “glycan” refers to a polysaccharide, oligosaccharide, or monosaccharide. Glycans can be monomers or polymers (homo or heteropolymers) of sugar residues and can be linear or branched. A glycan may include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′ sulfo N-acetylglucosamine, etc.). Glycan can also refer to the carbohydrate portion of a glycoconjugate, such as a glycoprotein, glycolipid, glycopeptide, glycoproteome, peptide-glycan, lipopolysaccharide, or a proteoglycan. Glycans referred to herein can be N-linked glycans or O-linked glycans. N-linked glycans are usually found attached to the R-group nitrogen (N) of asparagine in the sequon, consisting solely of an Asn-X-Ser or Asn-X-Thr sequence, where X can be any amino acid except for proline.

As used herein, the terms “fucose”, “core fucose” and “core fucose residue” are used interchangeably and refer to a fucose in a1,6-position linked to the N-acetylglucosamine.

As used herein, the terms “N-glycan”, “N-linked glycan”, “N-linked glycosylation”, “Fc glycan” and “Fc glycosylation” are used interchangeably and refer to an N-linked oligosaccharide attached by an N-acetylglucosamine (GlcNAc) linked to the amide nitrogen of an asparagine residue in a Fc-containing polypeptide. The term “Fc-containing polypeptide” refers to a polypeptide, such as an antibody, which comprises an Fc region.

As used herein, “Fc receptor” or “FcR” refers to a receptor to which the Fc region of an antibody binds. For example, a FcR can be a receptor to which an IgG antibody binds, which is also known as a gamma receptor (FcγR), including FcγRI (CD64), FcγRII (CD32), and FcγRIII (CD16) subclasses. FcγRII receptors further include FcγRIIA (“activating”) and FcγRIIB (“inhibiting”) receptors. FcγRIIA plays an important role in maturation and the upregulation of costimulatory molecules on the cell surface, strengthening the DC's ability to present antigen to T cells. FcγRIIA is also linked to autoimmunity. FcγRIII receptors also exist in two different forms: FcγRIIIA and FcγRIIIB. FcγRIIIA is known to be expressed on mast cells, macrophages, and natural killer cells, while FcγRIIIB is almost only expressed on neutrophils. FcγRIII receptors are required for inducing antibody-dependent cellular cytotoxicity (ADCC) in NK cells.

A basic antibody unit is a heterotetrameric glycoprotein composed of two identical light (L) chains and two identical heavy (H) chains. Each L chain is linked to an H chain by at least one (and typically one) covalent disulfide bond, while the two H chains are linked to each other by one or more disulfide bonds depending on the H chain isotype. Each H and L chain also has regularly spaced intrachain disulfide bridges. Each H chain has at the N-terminus a variable domain (V_{H}) followed by three constant domains (C_{H}) for each of the α and γ chains and four C_{H} domains for u and & isotypes. Each L chain has at the N-terminus, a variable domain (V_{L}) followed by a constant domain (CL) at its other end. The V_{L} is aligned with the V_{H}. and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site.

The L chain from any vertebrate species can be assigned to one of two clearly distinct types, called kappa (κ) and lambda (λ), based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha (α), delta (δ), epsilon (¿), gamma (γ), and mu (μ), respectively. The γ and α classes are further divided into subclasses on the basis of relatively minor differences in C_{H} sequence and function, e.g., humans express the following subclasses: IgG1, IgG2, IgG3, IgG4, IgA1, and IgA2. It will be appreciated that mammals encoding multiple Ig isotypes will be able to undergo isotype class switching.

An “antibody fragment” or “antigen-binding fragment of an antibody” is a polypeptide comprising or consisting of a portion of an intact antibody, preferably the antigen binding or variable region of the intact antibody. Examples of antibody fragments include an F(ab′)₂ fragment, an Fv fragment, a single-chain Fv (ScFv) antibody, a diabody, minibody, nanobody (VHH), and a linear antibody (see U.S. Pat. No. 5,641,870; Zapata et al., Protein Eng. 8 (10): 1057-1062 [1995]); single-chain antibody molecules; and multispecific antibodies formed from antibody fragments.

Papain digestion of antibodies produces two identical antigen-binding fragments, called “Fab” fragments, and a residual “Fe” fragment, a designation reflecting the ability to crystallize readily. The Fab fragment consists of an entire L chain along with the variable region domain of the H chain (V_{H}) and the first constant domain of one heavy chain (CHI). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of cross-linking antigen. Both the Fab and F(ab′)₂ are examples of “antigen-binding fragments.” Fab′ fragments differ from Fab fragments by having an additional few residues at the carboxy terminus of the C_{H}1 domain, including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

The “Fc” fragment comprises the carboxy-terminal portions (i.e., the C_{H2} and C_{H3} domains of IgG) of both H chains held together by disulfides. The effector functions of antibodies are determined by sequences in the Fc region. The Fc domain is the portion of the antibody recognized by cell receptors, such as the FcR, and to which the complement-activating protein, Clq, binds. As discussed herein, modifications (e.g., amino acid substitutions) may be made to an Fc domain in order to modify (e.g., improve, reduce, or ablate) one or more functionality of an Fc-containing polypeptide (e.g., an antibody of the present disclosure).

“Fv” is the minimum antibody fragment that contains a complete antigen-recognition and antigen-binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the V_{H} and V_{L} antibody domains connected into a single polypeptide chain. Preferably, the sFv polypeptide further comprises a polypeptide linker between the V_{H} and V_{L} domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L}, domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993). Other antibody fragments and molecules comprising the same include, for example, linear antibodies, tandem scFv, scFv-Fc, tandem scFv-Fc, scFv dimer, scFv-zipper, diabody-Fc, diabody-C_{H3}, scDiabodies, scDiabody-Fc, scDiabody-C_{H3}, nanobodies, TandAbs, minibodies, miniantibodies, triabodies, tetrabodies, scFab, Fab-scFv, Fab-scFv-Fc, scFv-C_{H}-C_{L}-scFv, and F(ab′)₂-scFv2, all of which are also contemplated herein.

As used herein, “glycoantibody” was coined by the inventor, Dr. Chi-Hucy Wong, to refer to an antibody (preferably, therapeutic monoclonal antibodies) having a glycoform bound to the Fc region, which can be monoclonal (i.e., binds to the same epitope) or polyclonal.

As used herein, the term “effector function” as used herein refers to a biochemical event that results from the interaction of an antibody Fc region with an Fc receptor or ligand. Exemplary “effector functions” include Clq binding; complement dependent cytotoxicity; Fc receptor binding; antibody-dependent cell-mediated cytotoxicity (ADCC); phagocytosis; down regulation of cell surface receptors (e.g. B cell receptor; BCR), etc. Such effector functions can be assessed using various assays known in the art.

As used herein, the term “Antibody-dependent cell-mediated cytotoxicity” or “ADCC” is used for its definition in the art and can refer to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or U.S. Pat. No. 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

As used herein, the term “Complement dependent cytotoxicity” or “CDC” is used for its definition in the art and can refer to the lysis of a target cell in the presence of complement. Activation of the classical complement pathway is initiated by the binding of the first component of the complement system (Clq) to antibodies (of the appropriate subclass), which are bound to their cognate antigen. To assess complement activation, a CDC assay, e.g. as described in Gazzano-Santoro et al., J. Immunol. Methods 202:163 (1996), may be performed.

As used herein, “Antibody-dependent cellular phagocytosis” or “ADCP” is used for its definition in the art and can refer to a highly regulated process in which an antibody eliminates binding target and initiates phagocytosis by linking its Fc domain to a specific Fcγ receptor on the phagocytic cell. Unlike ADCC, ADCP can be mediated by monocytes, macrophages, neutrophils and dendritic cells via FcγRIIa (CD32a), FcγRI (CD64), and FcγRIIIa (CD16), where FcγRIIa (CD32a) on macrophages represents the major pathway.

As used herein, “vaccinal effect” is used for its definition in the art and can refer to the protective immune response that can be triggered by a vaccine or by antibodies.

### EXAMPLE

### Example 1: Manufacturing Exemplary CHO Cells of The Present Disclosure for Expressing a Sialylated Glycoprotein

This experiment manufactured an exemplary cell according to an embodiment of the present disclosure from a parental CHO cell. ExpiCHO cells (Thermo Fisher Scientific) were purchased and maintained ExpiCHO™ Expression Medium according to the product manual. Then, the cells were transferred with a SWG-006 plasmid (i.e. an expression vector) using an ExpiFectamine™ CHO Transfection Kit according to the manufacturer's instructions. The SWG-006 plasmid (SEQ ID NO: 145) was made of pcDNA™3.1 plasmid and was constructed with a CMV enhancer, a CMV promoter, a ST6GAL1 gene (SEQ ID NO: 1352), a P2A sequence (SEQ ID NO: 141), and a B4GALTI gene (SEQ ID NO: 136). Alternatively, cells were co-transfected with a ST6GAL1 plasmid (SEQ ID NO: 147) and a B4GALT1 plasmid (SEQ ID NO: 148), each was constructed to express the ST6GAL1 gene and the B4GALTI gene separately under a CMV promoter.

Then, the cells were transfected with an Adalimumab plasmid (pcDNA2 TADA, SEQ ID NO: 149) using the ExpiFectamine™ CHO Transfection Kit to express Adalimumab (Humira). Three days after the transfection, the transfected cells (a transient clone) were washed with staining buffer (EDTA 1 mM, NaN₃ 0.02%, BSA 1%, in PBS) and stained with biotinylated SNA (VectorLabs), which is designed to bind a sialic acid linked to a N-acetylgalactosamine or galactose. After being washed, the cells were stained with Streptoavidin-Alexa647 (BioLegend) and analyzed using flow cytometry (BD LSRFortessa).

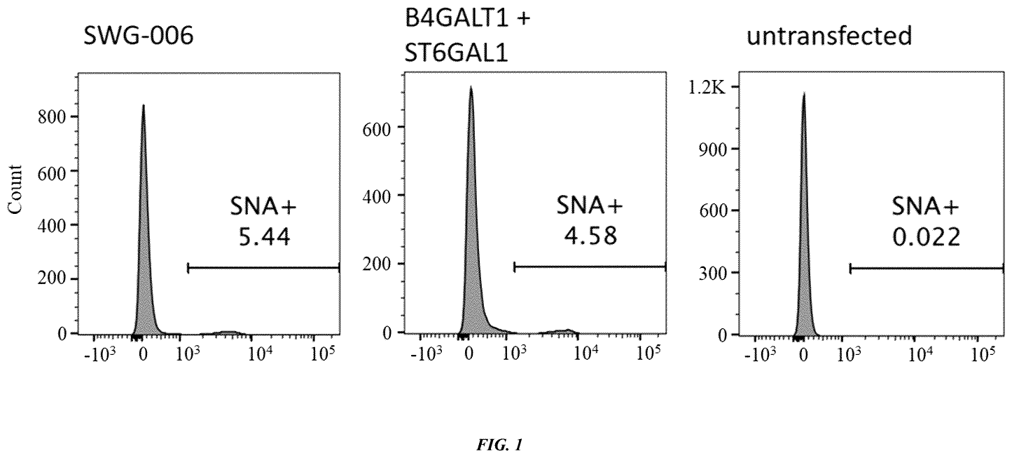

The results (FIG. 1) showed transfection with the SWG-006 plasmid or co-transfected with the ST6GAL1 plasmid and the B4GALTI plasmid were able to promote a2,6-sialylation. In comparison, the parent CHO cell (ExpiCHO cells) does not have an endogenous a2,6-sialyltransferase, so no signal was detected from untransfected cells. Most importantly, the results also showed that transfection with the SWG-006 plasmid provided higher a2,6-sialylation than co-transfection with the ST6GAL1 plasmid and the B4GALTI plasmid.

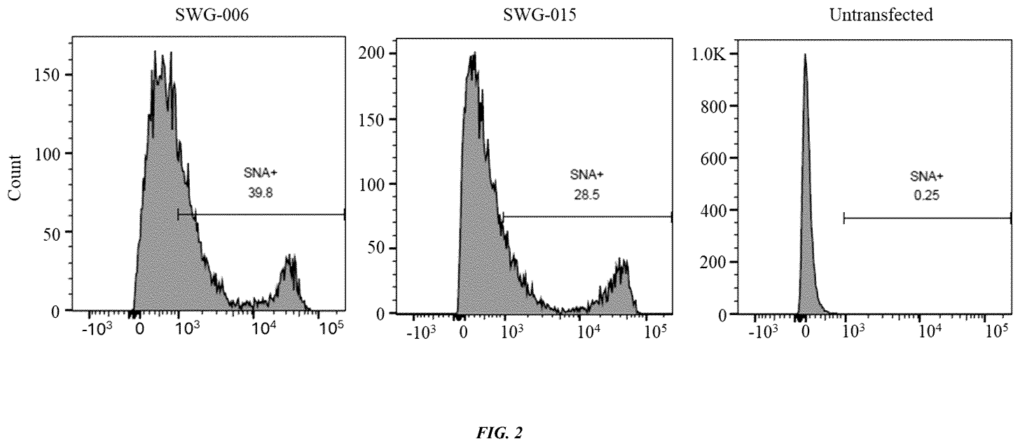

Similarly, another exemplary cell according to an embodiment of the present disclosure was made by transfecting an ExpiCHO cell (Thermo Fisher Scientific) using the ExpiFectamine™ CHO Transfection Kit with a SWG-015 plasmid (SEQ ID NO: 146). The SWG-015 vector wad made of pcDNA™3.1 plasmid and was constructed with a CMV enhancer, a CMV promoter, a modified PspST gene (SEQ ID NO: 1354), a P2A sequence (SEQ ID NO: 141), and a B4GALTI gene (SEQ ID NO: 136). The cell was also transfected with the Adalimumab plasmid (pcDNA2 TADA, SEQ ID NO: 149) to express Adalimumab (Humira). Then, the sialylation of Adalimumab produced by the cell, which is also a transient clone, was observed using SNA staining as described above. The cell transfected with the SWG-006 vector was also examined to compare with the cell transfected with the SWG-015 vector. The results showed that the cell transfected with the SWG-015 provided a similar level of sialylation as that of the cell transfected with the SWG-006 vector (FIG. 2).

### Example 2: Stabilization of the Exemplary Cells of the Present Disclosure

Plasmid SWG-006 was linearized by digestion with Pvul and then purification. The linearized SWG-006 was transfected into ExpiCHO cells by using ExpiFectamine™ CHO Transfection Kit (Thermo Fisher Scientific) according to the manufacturer's instructions. On day 2 post-transfection, G418 was added at 400 μg/ml for drug selection. After recovering to 90% survival, the cells were stained with biotinylated SNA and then streptavidin-BV421 wherein the SNA bound endogenous membrane proteins produced by the cells. The cells were then sorted for high SNA binding by using WOLF G2 Cell Sorter (Nanocellect Biomedical, Inc.) into single cells cultured in 96 well plates. After a period of time of culture, the single clone cells were stained with biotinylated SNA and then streptavidin-APC and analyzed by using flow cytometry to select clones with highly surface alpha2,6-sialylation. The selected cells (>99% SNA positive) were then transfected with Adalimumab expression plasmid (SEQ ID NO: 149) to overexpress Adalimumab. The expressed Adalimumab was purified by using protein A beads and analyzed by using an SNA hybridization blot to select clones that were able to express highly alpha2,6-sialylation Adalimumab. The glycoforms of Adalimumab were further analyzed by LC/MS-MS.

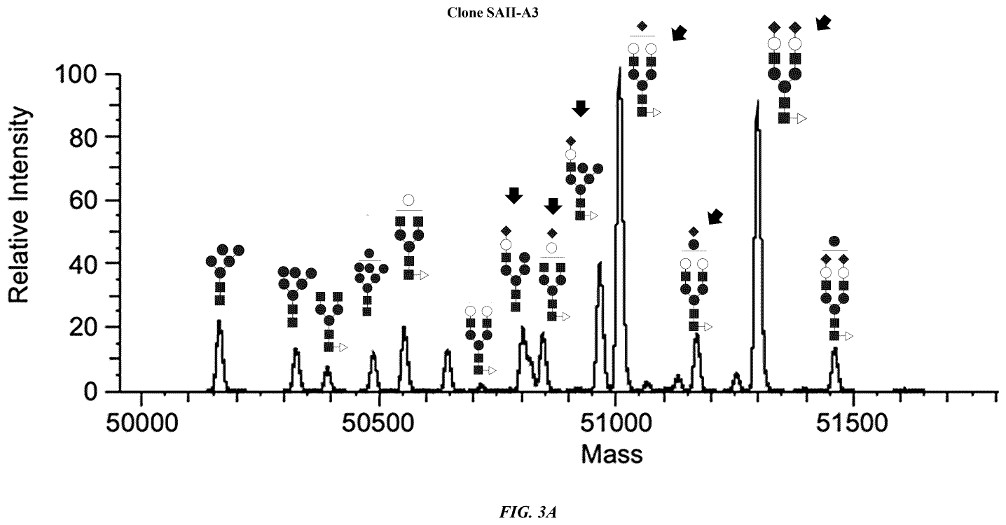

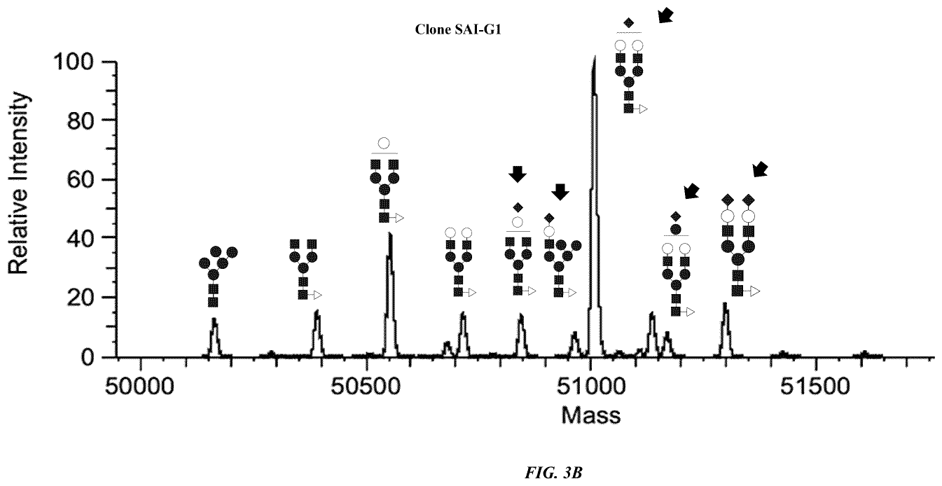

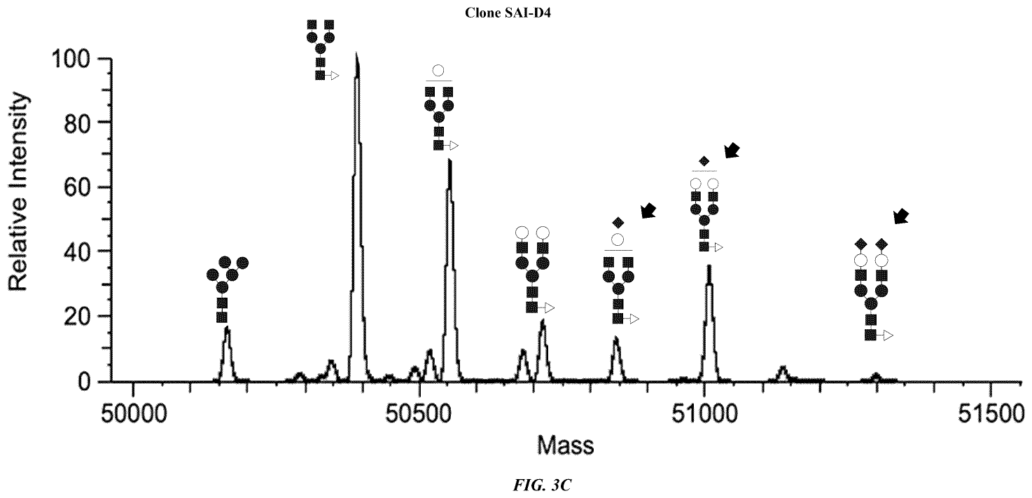

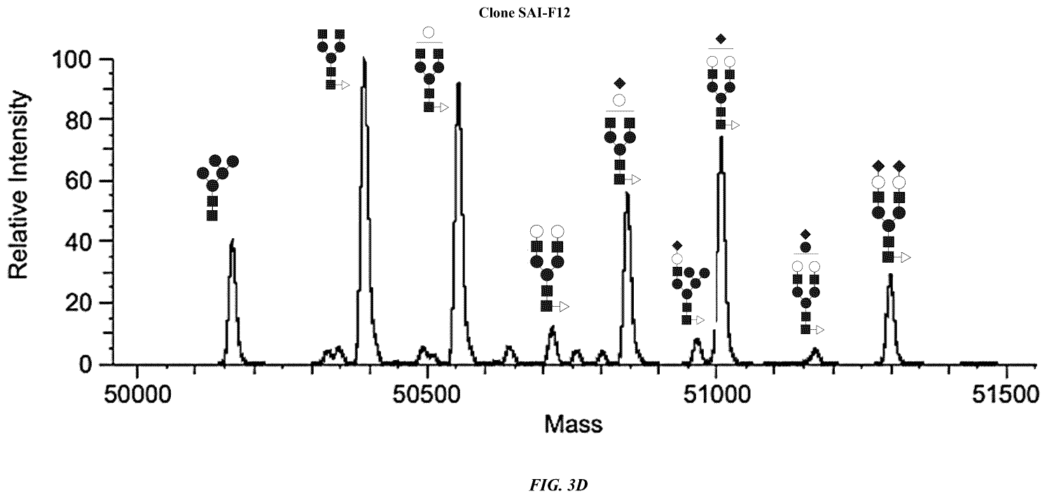

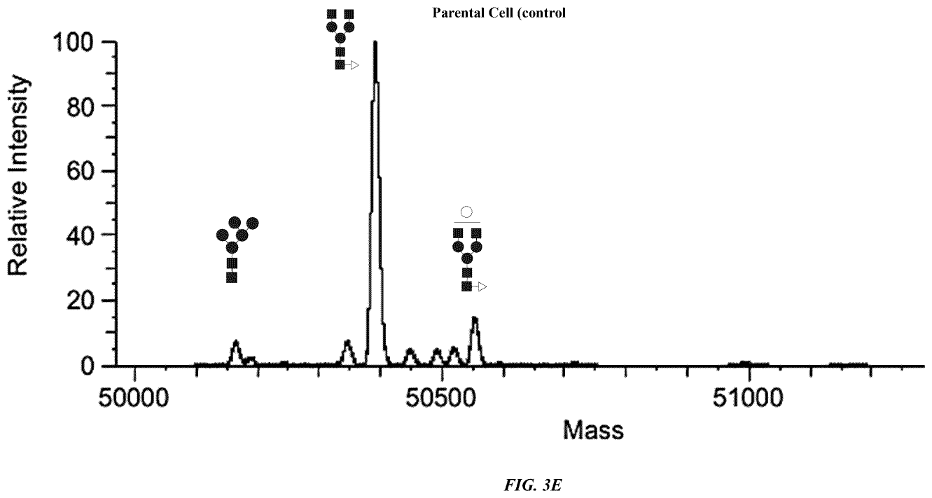

The LC/MS-MS data shows that the four stabilized clones tested in this example, SAII-A3, SAI-GI, SAI-D4, and SAI-F12, all expressed a high percentage of sialylation of Adalimumab. The stabilized Clone SAII-A3 showed 70.9% (about 70% to 72%) sialylation of the Adalimumab it expressed (FIG. 3A); the stabilized Clone SAI-GI showed 55.39% (about 55% to 57%) sialylation of the Adalimumab it expressed (FIG. 3B); the stabilized Clone SAI-D4 showed 16.8% (about 16% to 18%) sialylation of the Adalimumab it expressed (FIG. 3C); and the stabilized Clone SAI-F12 showed 36.14% (about 36% to 38%) sialylation of the Adalimumab it expressed (FIG. 3D). In comparison, the Adalimumab expressed by the parental cell did not show significant sialylation (FIG. 3E).

### Example 3: Glycoengineering Adalimumab Using an Exemplary Stabilized Cell According to the Present Disclosure

In this experiment, a stabilized clone (SAII-A3) of the SWG-006 cells obtained as described in Example 2 above was transfected with an Adalimumab expression vector (SEQ ID NO: 149) using an ExpiFectamine™ CHO Transfection Kit (Thermo Fisher Scientific) according to the manufacturer's instructions to express Adalimumab, and the glycoforms thereof was observed and compared with the same antibody expressed by a parent cell (ExpiCHO cell).

Briefly, cells were diluted to a final density of 510\hat{ }6 viable cells/mL with fresh 15 mL ExpiCHO™ Expression Medium in a flask, pre-warmed to 37° C. The flask was then swirled gently. The cells were then maintained in a 37° C. incubator with a humidified atmosphere of 8% CO₂ in the air on a 125 rpm orbital shaker before transfection. Plasmid DNA (i.e., the expression vector of SEQ ID NO: 149, 12 μg) was diluted with 0.6 mL of cold OptiPRO™ medium, and ExpiFectamine™ CHO Reagent (48 μL) was diluted with 552 μL of cold OptiPRO™ medium. The dilated DNA was then mixed with the diluted ExpiFectamine™ CHO Reagent to obtain ExpiFectamine™ CHO/plasmid DNA complexes. The ExpiFectamine™ CHO/plasmid DNA complexes were added to the cell culture in a 37° C. incubator with a humidified atmosphere of 8% CO₂ in the air on a 125 rpm orbital shaker. After transfection, 90 μl ExpiFectamine™ CHO Enhancer and 3.6 ml ExpiCHO™ Feed volumes were added to the cells. The cells in the flask were then maintained in the 37° C. incubator with a humidified atmosphere of 8% CO₂ with shaking. After 8-10 days post-transfection, the supernatant of the cell culture was harvested for protein purification.

Then, the antibodies produced by the SAII-A3 cells and the parental EXPICHO cells were isolated respectively using protein A beads (GE HealthCare). Briefly, 15 mL supernatant was passed through the 200 uL protein A column. The column was washed with 10-column volume (CV) PBS. The bound antibody was eluted with 15 CV 0.1 M glycine pH=3. The yielding antibodies were dialysis against 10 mM ammonium acetate. The antibodies were then reduced using 25 mM DTT and analyzed by LC/MS-MS. A commercial product of Adalimumab (Humira®) was purchased and analyzed using LC/MS-MS as a control.

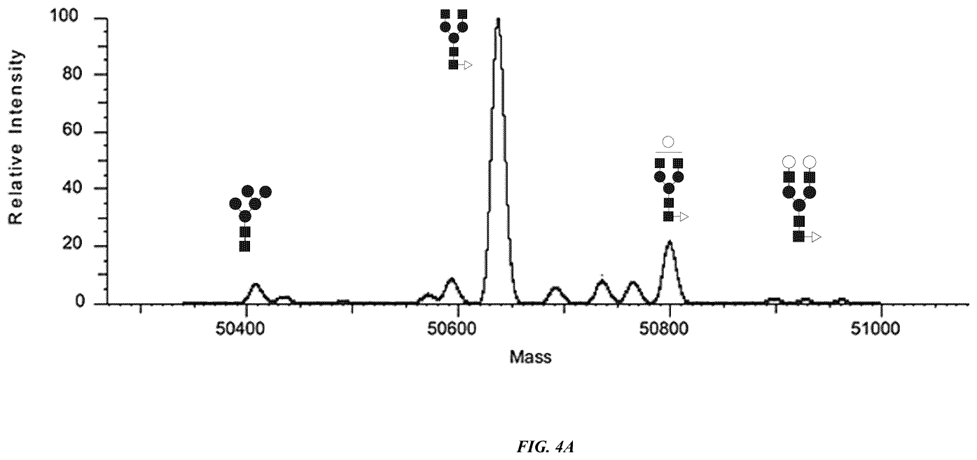

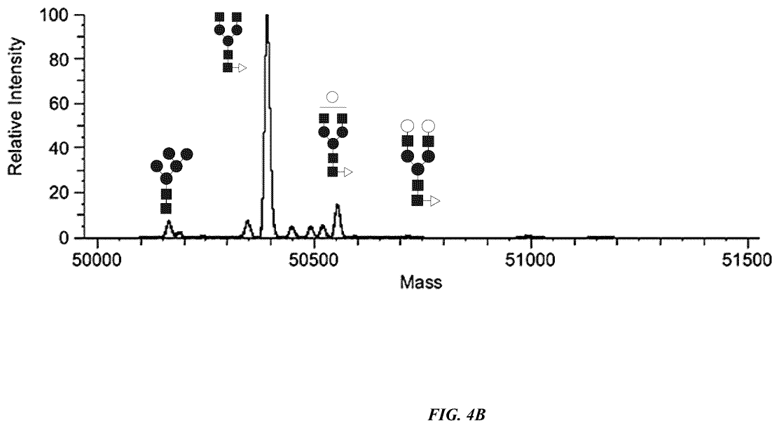

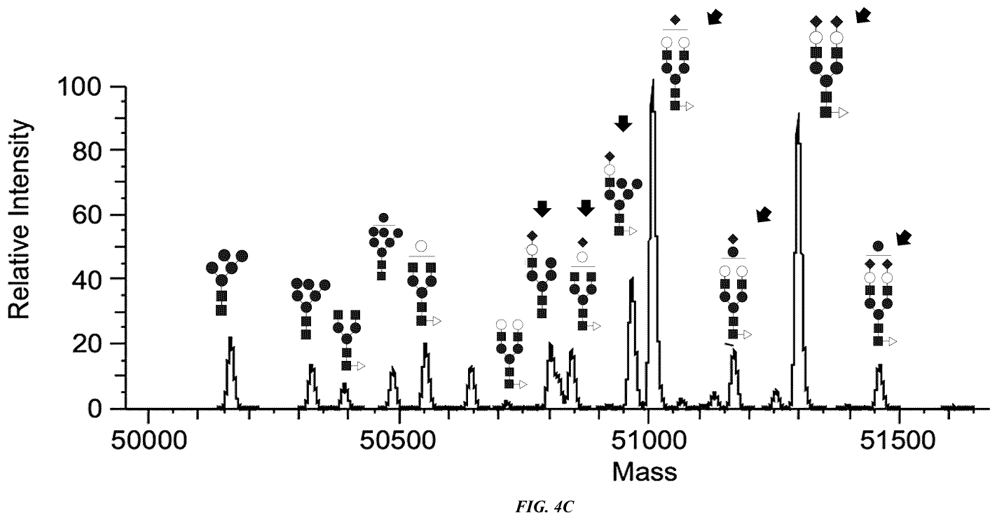

The LC/MS-MS data shows that the Adalimumab produced by the exemplary cells of the present disclosure had a variety of sialylated glycans (See FIG. 4C), the sialylated glycans are labeled with solid arrows), including G2S2F, G2SIF, GISIF, N3H6F1S1, N3H4S1, N3H5F1S1, N4H6F1S1, N4H6F1S2. The sialylation percentage of the total glycans was 70.9%, and the sialylation percentage of the bi-antennary complex type glycan was 79.8%. In comparison, although the commercial products (FIG. 4A) and the Adalimumab produced by parental ExpiCHO cells (FIG. 4B) also carried glycans, no sialylation was observed.

### Example 4: Manufacturing Exemplary CHO Cells of the Present Disclosure with Fut8 Knockout

Following the procedures described in Example 2, this experiment used CHO-S cells as the parent cells to obtain exemplary cells stably transfected with the SWG-006 vectors (the SAII-A3 cell). Furthermore, this Example further modified the cells to knock out the Fut8 gene thereof via Clustered Regularly Interspaced Short Palindromic Repeats (CRISPR) technology.

CRISPR transfection for FUT8 knock out. The plasmids (vector) for CRISPR/Cas9 expression were constructed as suggested by the kit manual (Thermo Fisher Scientific). Briefly, crRNAs of SEQ ID NO: 150, SEQ ID NO: 151, or SEQ ID NO: 152 were designed to target FUT8 (Fucosyltransferase 8) gene on different locations thereof. The crRNAs were respectively synthesized with tracrRNA of SEQ ID NO: 154 to form guide RNAs (gRNAs) (Integrated DNA Technologies) and cloned into a vector for gRNA expression. The vector was then co-transfected with another vector designed to express Cas9 into target cells (the SAII-A3 cell). On day 2 after transfection, Lens Culinaris Agglutinin (LCA, VectorLabs) was added to the culture at a final concentration of 50 μg/mL LCA in each culture. LCA is a lectin that binds alpha1-6 core fucose. Therefore, LCA positive signal represents a functioning FUT8 product; in contrast, a FUT8 knock-out cell will not express alpha1-6 fucose and will be LCA negative (low or intermediate signal). On day 6 after LCA treatment, cultured cells were sorted to identify cells of LCA negative, which suggested FUT8 knockout.

FUT8 KO Cell sorting. On day 6 after LCA treatment, cell cultures were centrifuged to collect pellets. The collected pellets were then washed with sorting buffer (EDTA 1 mM, BSA 1%, in 1×PBS, sterilized with 0.2 μm membrane filtration), and the cells of the pellets were stained with 5 μg/mL biotinylated LCA and stored on ice for 15 minutes. After that, the cells were washed and stained again with 400×diluted Streptoavidin-Alexa647 on ice. After 15 minutes, the cells were washed and re-suspended with 1000×diluted propidium iodide (PI) in sorting buffer. Cells were then sorted using BD FACSAria or comparable equipment to collect PI negative cells that were among the bottom 2% of APC intensity. The FUT8 knockout of the collected cells can be verified by another round of LCA staining.

PCR Verification. The FUT8 knockout can be verified by PCR. Briefly, cell cultures were centrifuged to collect pellets. The collected pellets were re-suspended with 50 uL cell lysis buffer A (25 mM NaOH/0.2 mM EDTA) and incubated at 95 degree Celsius for 10 minutes. Then, cell lysis buffer B (0.2M Tris-HCl pH 5.5) was added to the culture, and two microliter of the mixture was collected for PCR. The PCR was performed by adding following reagent into a PCR tube: 8.5 uL ddH20, 12.5 uL 2×SuperRed MasterMix (Biotools), 2 uL cell lysate, 1 uL 10 uM primer F(CCTGATACTGGGTAGCTAATTGT), 1 uL 10 uM primer R (AATTACCCACATTCCAGATGCAC). The PCR program was set to be 98 degree Celsius for 3 minutes, 98 degree Celsius for 15 seconds, 60 degree Celsius for 15 seconds, 72 degree Celsius for 1 minute for 35 cycle, and be finalized at 72 degree Celsius for 5 minutes.

LCA Staining Verification. FACS sorted cells were seeded into 96-well plate (3×10⁵ cells per well), and the plate was centrifuged at 600×g for 5 minutes to get rid of the supernatant. The cells were then washed with FACS-BSA buffer (1 mM EDTA, 0.02% NaN3, 1% BSA in PBS). After removing the buffer, each well was added with 50 μL of LCA-Biotin (B-1045-5, Vector laboratories) in FACS-BSA buffer (10 μg/mL), and the cells were incubated on ice for 15 minutes. After that, the cells were washed with FACS-BSA buffer and added with 0.5 μg/mL of streptavidin-APC reagent and 50 μL of diluted Ab. The cells were incubated on ice for another 10 minutes. Then, after washing the cells, 1000×diluted propidium iodide in FACS-BSA buffer was added, and the mixture was analyzed using BD FACSAria.

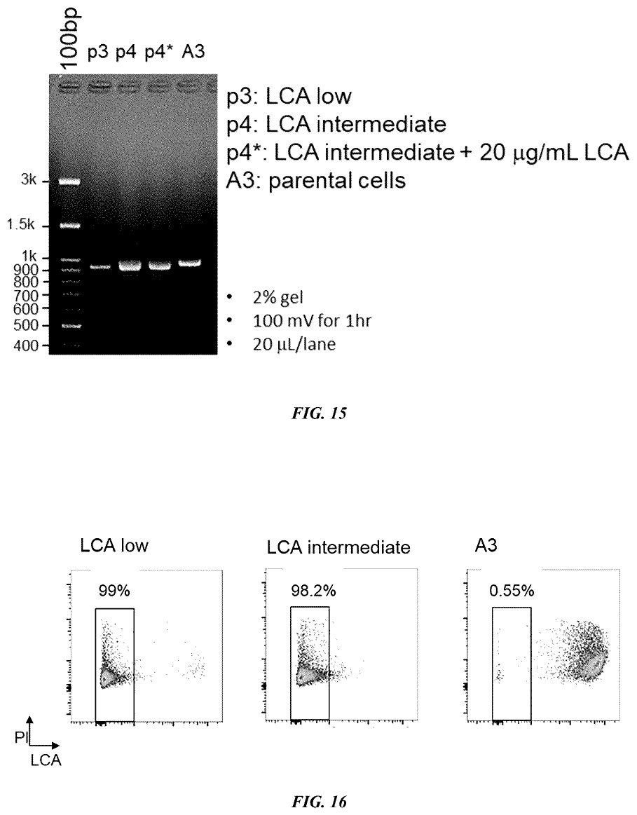

Results. The size of the PCR product was about 971 bp for a parental cell (without knockout). After deletion, the anticipated PCR product was between 825 to 906 bp. FIG. 15 shows four lanes of samples. The first three lanes, p3, p4, and p4*, were samples from cells showing low or intermediate LCA signals, and the PCR products were smaller than that from parental cells (the fourth lane). This data verified that the cells showing low or intermediate LCA signals were confirmed with smaller PCR products, indicating FUT8 knockout. Furthermore, the FACS spectrum verified that cells of low or intermediate LCA signals did not have functioning alpha1-6 core fucose (FIG. 16). Accordingly, this Example prepared FUT8 KO cells for further experiments.

### Example 5: Glycoengineering Using an Exemplary Stabilized Cell According to the Present Disclosure

The exemplary SAII-A3 cells and Fuc8 knockout cells (SAII-A3 Fuc KO cells) obtained in Example 4 were used to express FDA-approved therapeutic proteins thereby engineering them with a desired glycan profile. The process for expressing the therapeutic protein was substantially the same as the procedure described in Example 3 using the ExpiFectamine™ CHO Transfection Kit (Thermo Fisher Scientific) except that respective expression vectors were synthesized for each therapeutic proteins to be produced and glycoengineered in this Example The glycoforms (i.e., the glycan profiles) of the expressed samples were observed and compared with that of the commercially available counterpart therapeutic proteins and the same therapeutic proteins expressed by parent cells (CHO-S cells).

The percentage of the desired glycans (sialylated complex type glycans) was also determined in this Example. Briefly, purified biologics or commercial products were dialysis against 10 mM ammonia acetate by using 30 kDa cutoff Ultra Centrifugal Filter (UFC5030, Merck) and centrifugation at 14000 g. Dialysis was performed for 5 times buffer change. The testing samples (i.e., the expressed biologics or the commercial products) were concentrated to around 1 mg/mL. One hour before LC/MASS analysis, the samples were reduced by adding DTT to final concentration at 25 mM and incubated at 37° C. Intact protein LC/MASS was used to indicate the molecule weight of sample. The molecule weight of N-glycan on the heavy chain can be obtained by subtracting the molecular weight of the deglycosylated antibody heavy chain treated with PNGase F. Then, the molecular weight of the N-glycan was used to estimate the glycoforms. The relative abundance of a specific glycoform was estimated by calculating the area under the curve (AUC) of the individual glycoform as a proportion of the total AUC. The percentage of sialylation was determined by dividing the sum of the relative abundances of sialylated glycoforms by the total AUC.

The LC/MASS analysis showed that the therapeutic proteins produced according to the present disclosure exhibited a variety of sialylated complex type glycans. As shown in LC/MASS spectrum, each peak represents a kind of glycan, which was identified based on the molecular weight thereof (as described above). The sialic acid moiety was presented as diamond shapes in the glycan structures annotated in the spectrum, and the sialylated complex type glycans identified were labeled as indicated in Table 1 below. The results verified that the glycoproteins produced according to the present disclosure in this Example have enriched heterogenous sialylated complex type glycans to 50 to 86%.

TABLE 1
Identified sialylated complex type glycans on the therapeutic

| Peak No. | Sialylated complex type glycans |
|---|---|
| 1 | Sia₂(α2,6)Gal₂GlcNAc₂Man₃GlcNAc₂ or |
| | Sia₂(α2,6)Gal₂GlcNAc₂Man₃GlcNAc₂Fuc |
| 2 | Sia(α2,6)HexGal₂GlcNAc₂Man₃GlcNAc₂ or |
| | Sia(α2,6)HexGal₂GlcNAc₂Man₃GlcNAc₂Fuc |
| 3 | Sia(α2,6)Gal₂GlcNAc₂Man₃GlcNAc₂ or |
| | Sia(α2,6)Gal₂GlcNAc₂Man₃GlcNAc₂Fuc |
| 4 | Sia(α2,6)GalGlcNAcMan₅GlcNAc₂ or |
| | Sia(α2,6)GalGlcNAcMan₅GlcNAc₂Fuc |
| 5 | Sia(α2,6)GalGlcNAc₂Man₃GlcNAc₂ or |
| | Sia(α2,6)GalGlcNAc₂Man₃GlcNAc₂Fuc |
| 6 | Sia(α2,6)GalGlcNAcMan₄GlcNAc₂ or |
| | Sia(α2,6)GalGlcNAcMan₄GlcNAc₂Fuc |
| 7 | Sia(α2,6)GalGlcNAcMan₃GlcNAc₂ or |
| | Sia(α2,6)GalGlcNAcMan₃GlcNAc₂Fuc |

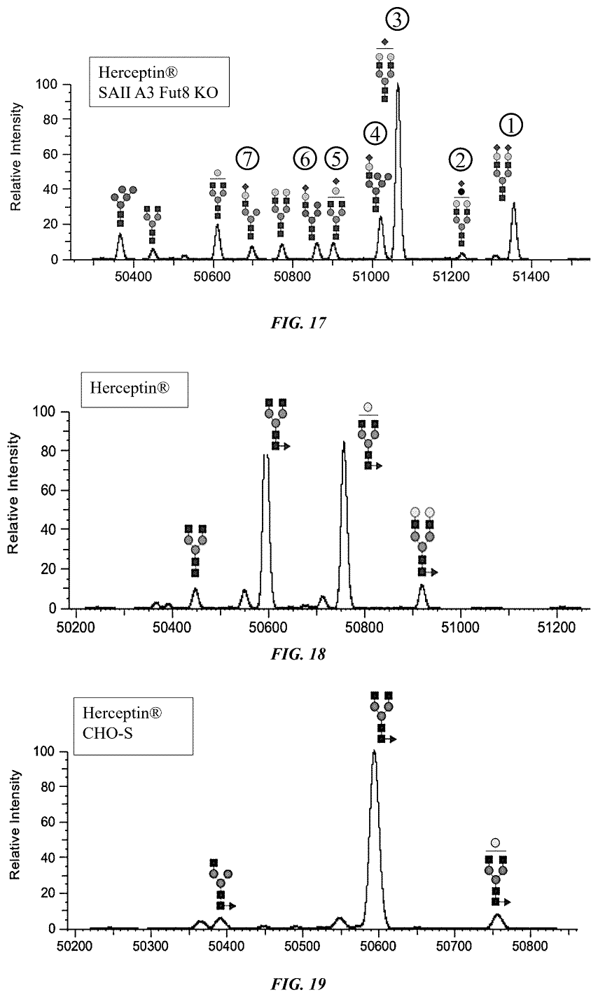

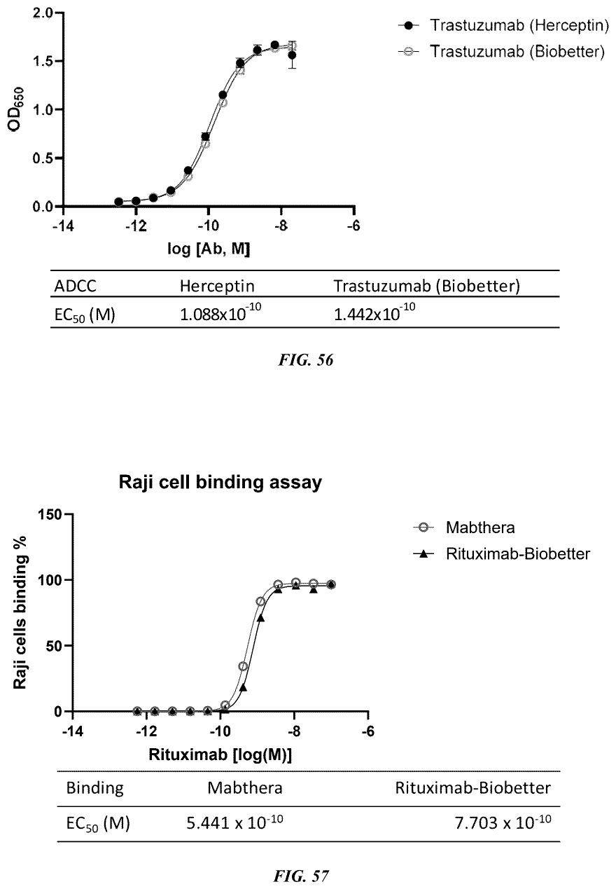

The Trastuzumab (Herceptin®) population produced by the SAII A3 Fut8 KO cells according to the present disclosure exhibited all the seven SCT glycans as indicated in Table 1. Those SCT glycans comprised around 75.1% of the glycans of the population (See Table 2 below for the percentage of each identified SCT glycans), and, among them, SiaGal2GlcNAc2Man3GlcNAc2 and Sia2Gal2GlcNAc2Man3GlcNAc2 were the two most abundant ones (FIG. 17). In comparison, the glycan profile of the commercial Herceptin® product tested here did not have sialylated glycans, nor did the Herceptin® population produced by parent CHO-S cells (FIG. 18 and FIG. 19). It was also noted that the commercial product and the CHO-S cell product both had glycans with core fucose, while the glycoprotein population of the present disclosure did not due to the Fut8 knock-out. It was also verified that the engineering did not affect the binding affinity to the antigen (FIG. 56).

TABLE 2

| Peak No. | Sialylated complex type glycans | Percentage |
|---|---|---|
| 1 | Sia2(α2,6)Gal2GlcNAc2Man3GlcNAc2 | 13.01 |
| 2 | Sia(α2,6)HexGal2GlcNAc2Man3GlcNAc2 | 1.44 |
| 3 | Sia(α2,6)Gal2GlcNAc2Man3GlcNAc2 | 40.19 |
| 4 | Sia(α2,6)GalGlcNAcMan5GlcNAc2 | 9.83 |
| 5 | Sia(α2,6)GalGlcNAc2Man3GlcNAc2 | 3.75 |
| 6 | Sia(α2,6)GalGlcNAcMan4GlcNAc2 | 3.83 |
| 7 | Sia(α2,6)GalGlcNAcMan3GlcNAc2 | 3.06 |

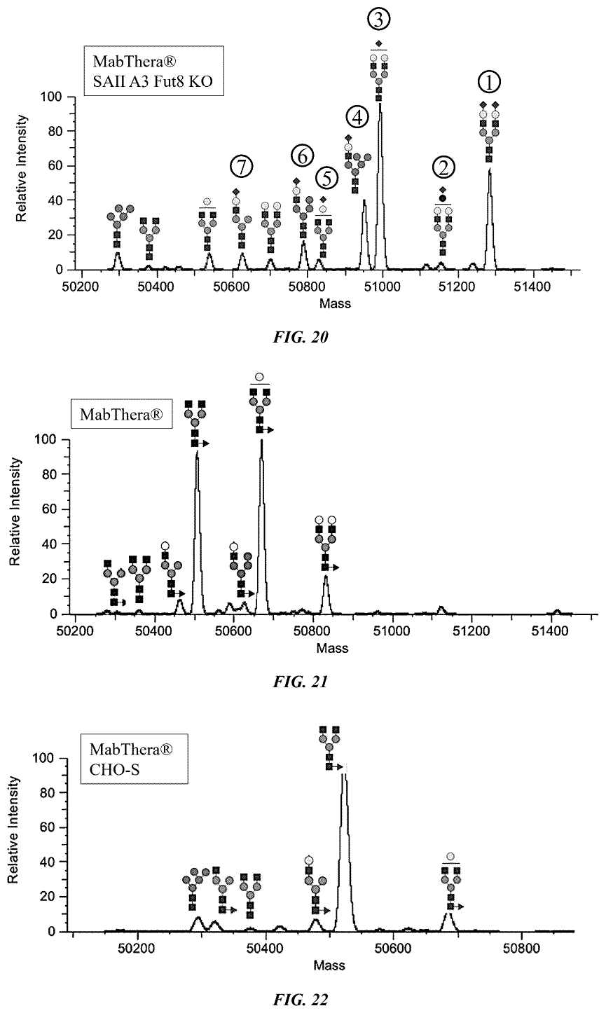

The Rituximab (Mab Thera® or Rituxan®) population produced by the SAII A3 Fut8 KO cells according to the present disclosure exhibited all the seven SCT glycans as indicated in Table 1. Those SCT glycans comprised around 83.4% of the glycans of the population (See Table 3 below for the percentage of each identified SCT glycans), and, among them, SiaGal2GlcNAc2Man3GlcNAc2 and Sia2Gal2GlcNAc2Man3GlcNAc2 were the two most abundant ones (FIG. 20). In comparison, the glycan profile of the commercial MabThera® product tested here did not have sialylated glycans, nor did the MabThera® population produced by parent CHO-S cells (FIG. 21 and FIG. 22). It was also noted that the commercial product and the CHO-S cell product both had glycans with core fucose, while the glycoprotein population of the present disclosure did not due to the Fut8 knock-out. It was also verified that the engineering did not affect the binding affinity to the antigen (FIG. 57).

TABLE 3

| Peak No. | Sialylated complex type glycans | Percentage |
|---|---|---|
| 1 | Sia2(α2,6)Gal2GlcNAc2Man3GlcNAc2 | 20.6 |
| 2 | Sia(α2,6)HexGal2GlcNAc2Man3GlcNAc2 | 1.49 |
| 3 | Sia(α2,6)Gal2GlcNAc2Man3GlcNAc2 | 35.64 |
| 4 | Sia(α2,6)GalGlcNAcMan5GlcNAc2 | 14.36 |
| 5 | Sia(α2,6)GalGlcNAc2Man3GlcNAc2 | 2.18 |
| 6 | Sia(α2,6)GalGlcNAcMan4GlcNAc2 | 5.86 |
| 7 | Sia(α2,6)GalGlcNAcMan3GlcNAc2 | 3.32 |

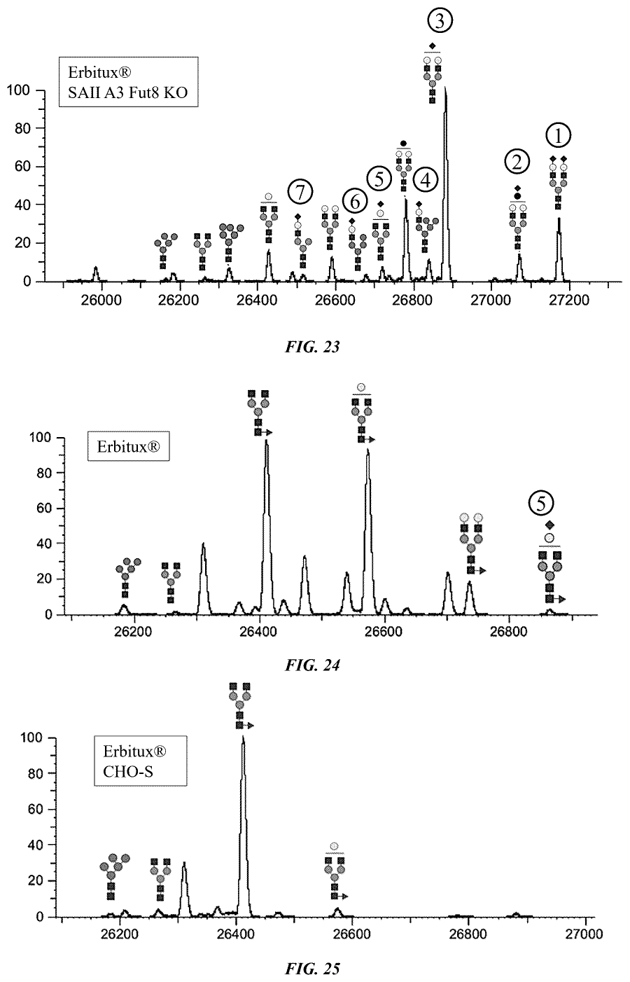

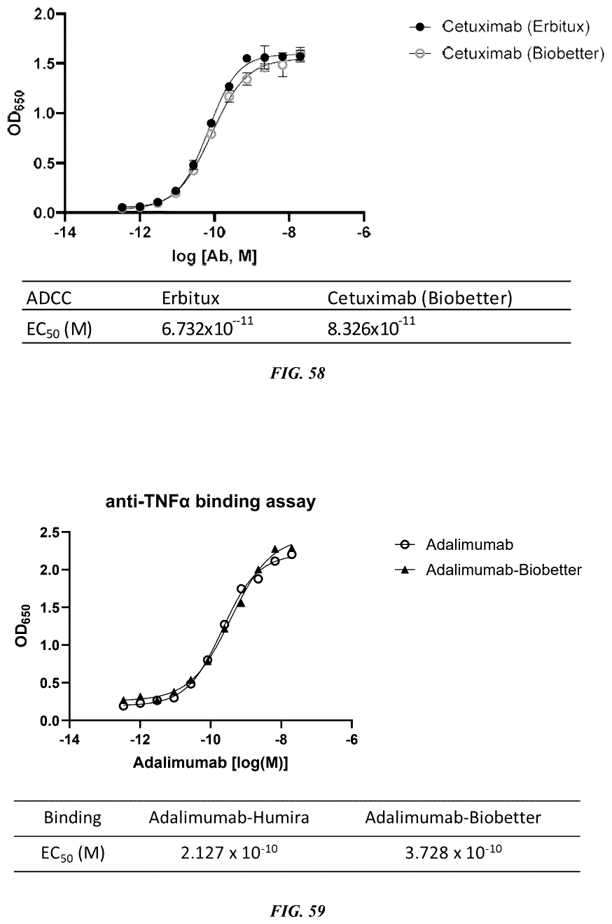

The Cetuximab (Erbitux®) population produced by the SAII A3 Fut8 KO cells according to the present disclosure exhibited all the seven SCT glycans as indicated in the Table. Those SCT glycans comprised around 58.1% of the glycans of the population (See Table 4 below for the percentage of each identified SCT glycans), and, among them, SiaGal2GlcNAc2Man3GlcNAc2 and Sia2Gal2GlcNAc2Man3GlcNAc2 were the two most abundant ones (FIG. 23). In comparison, the glycan profile of the commercial Erbitux® product tested here exhibited only around 0.74% sialylated glycans (FIG. 24), and the Erbitux® population produced by parent CHO-S cells (FIG. 25). It was also noted that the commercial product and the CHO-S cell product both had glycans with core fucose, while the glycoprotein population of the present disclosure did not due to the Fut8 knock-out. It was also verified that the engineering did not affect the binding affinity to the antigen (FIG. 58).

TABLE 4

| Peak No. | Sialylated complex type glycans | Percentage |
|---|---|---|
| 1 | Sia2(α2,6)Gal2GlcNAc2Man3GlcNAc2 | 11.18 |
| 2 | Sia(α2,6)HexGal2GlcNAc2Man3GlcNAc2 | 0.43 |
| 3 | Sia(α2,6)Gal2GlcNAc2Man3GlcNAc2 | 33.6 |
| 4 | Sia(α2,6)GalGlcNAcMan5GlcNAc2 | 3.79 |
| 5 | Sia(α2,6)GalGlcNAc2Man3GlcNAc2 | 2.48 |
| 6 | Sia(α2,6)GalGlcNAcMan4GlcNAc2 | 1.13 |
| 7 | Sia(α2,6)GalGlcNAcMan3GlcNAc2 | 1.19 |

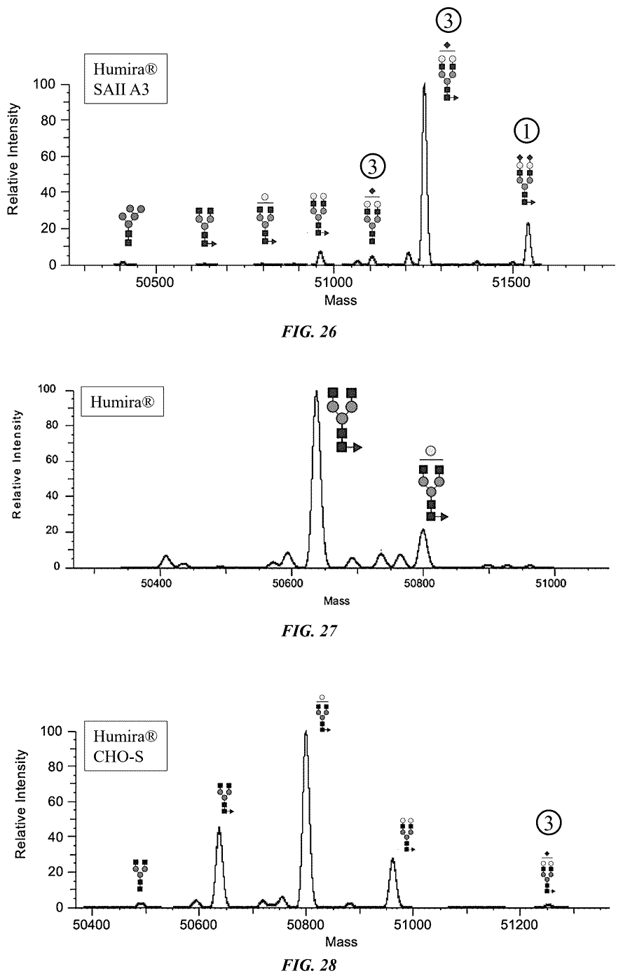

The Adalimumab (Humira®) population produced by the SAII A3 cells according to the present disclosure exhibited Sia2Gal2GlcNAc2Man3GlcNAc2 (not visible in FIG. 26), Sia2Gal2GlcNAc2Man3GlcNAc2Fuc, SiaGal2 HexGlcNAc2Man3GlcNAc2Fuc (not visible in FIG. 26), SiaGal2GlcNAc2Man3GlcNAc2, SiaGal2GlcNAc2Man3GlcNAc2Fuc, SiaGalGlcNAcMan5GlcNAc2Fuc (not visible in FIG. 26), SiaGalGlcNAcMan4GlcNAc2Fuc (not visible in FIG. 26), and SiaGalGlcNAcMan3GlcNAc2Fuc (not visible in FIG. 26). Although less kinds of SCT glycans were observed in this example, the population still exhibited diverse and heterogeneous sialylated glycans with a percentage of around 88.1% (FIG. 26) (See Table 5 below for the percentage of each identified SCT glycans). No sialylated glycans were observed in commercial Humira® product (FIG. 27), and the CHO-S cell product showed only SiaGal2GlcNAc2Man3GlcNAc2Fuc at barely over 1% (FIG. 28). It was also verified that the engineering did not affect the binding affinity to the antigen (FIG. 59).

TABLE 5

| Peak No. | Sialylated complex type glycans | Percentage |
|---|---|---|
| 1 | Sia2(α2,6)Gal2GlcNAc2Man3GlcNAc2 | 0.33 |
| 1 - Fuc | Sia2(α2,6)Gal2GlcNAc2Man3GlcNAc2Fuc | 14.95 |
| 2 | Sia(α2,6)HexGal2GlcNAc2Man3GlcNAc2 | ND |
| 3 | Sia(α2,6)Gal2GlcNAc2Man3GlcNAc2 | 3.34 |
| 3 - Fuc | Sia(α2,6)Gal2GlcNAc2Man3GlcNAc2Fuc | 64.06 |
| 4 - Fuc | Sia(α2,6)GalGlcNAcMan5GlcNAc2Fuc | 4.42 |
| 5 | Sia(α2,6)GalGlcNAc2Man3GlcNAc2 | ND |
| 6 - Fuc | Sia(α2,6)GalGlcNAcMan4GlcNAc2Fuc | 0.31 |
| 7 - Fuc | Sia(α2,6)GalGlcNAcMan3GlcNAc2Fuc | 0.53 |

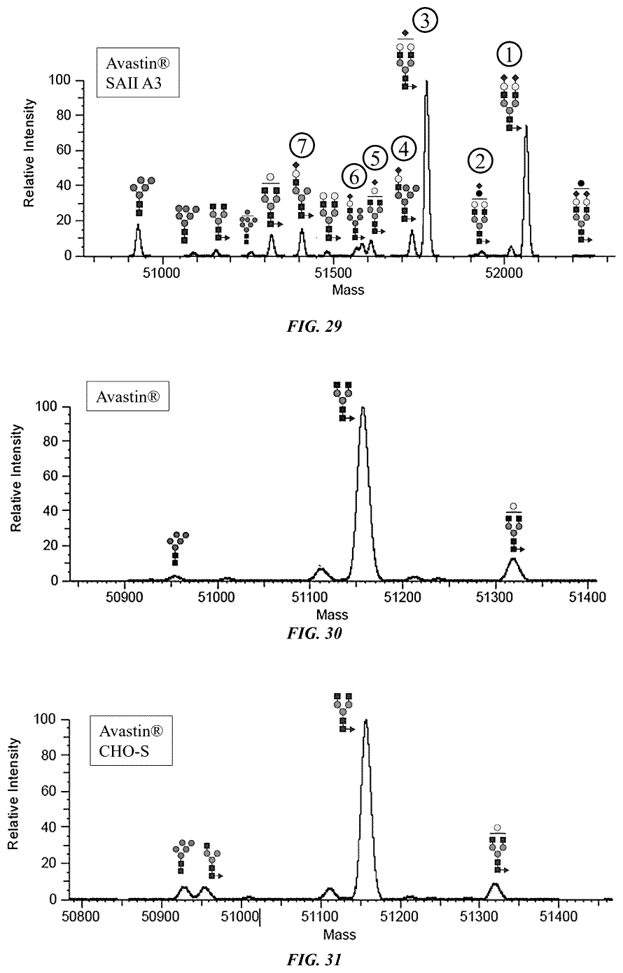

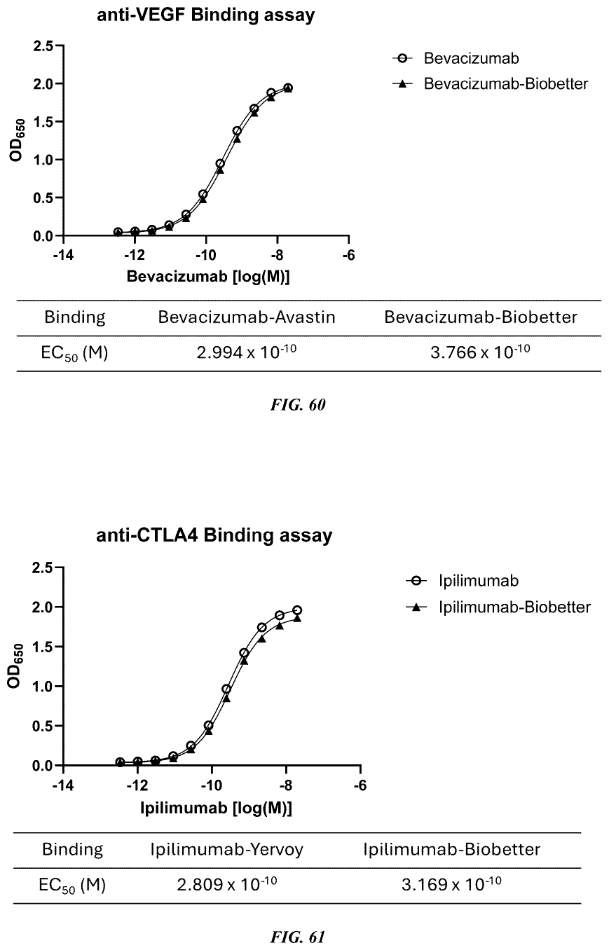

The Bevacizumab (Avastin®) population produced by the SAII A3 cells according to the present disclosure exhibited all the seven SCT glycans as indicated in Table 1. Those SCT glycans comprised around 82.6% of the glycans of the population (See Table 6 below for the percentage of each identified SCT glycans), and, among them, SiaGal2GlcNAc2Man3GlcNAc2Fuc and Sia2Gal2GlcNAc2Man3GlcNAc2Fuc were the two most abundant ones (FIG. 29). In comparison, the glycan profile of the commercial Avastin® product tested here did not have sialylated glycans, nor did the Avastin® population produced by parent CHO-S cells (FIG. 30 and FIG. 31). It was also verified that the engineering did not affect the binding affinity to the antigen (FIG. 60).

TABLE 6

| Peak No. | Sialylated complex type glycans | Percentage |
|---|---|---|
| 1 | Sia2(α2,6)Gal2GlcNAc2Man3GlcNAc2 | 0.3 |
| 1 - Fuc | Sia2(α2,6)Gal2GlcNAc2Man3GlcNAc2Fuc | 26.92 |
| 2 - Fuc | Sia(α2,6)HexGal2GlcNAc2Man3GlcNAc2Fuc | 0.94 |
| 3- Fuc | Sia(α2,6)Gal2GlcNAc2Man3GlcNAc2Fuc | 36.21 |
| 4 | Sia(α2,6)GalGlcNAcMan5GlcNAc2 | 2.43 |
| 4 - Fuc | Sia(α2,6)GalGlcNAcMan5GlcNAc2Fuc | 5.22 |
| 5 - Fuc | Sia(α2,6)GalGlcNAc2Man3GlcNAc2Fuc | 3.33 |
| 6 - Fuc | Sia(α2,6)GalGlcNAcMan4GlcNAc2Fuc | 1.7 |
| 7 - Fuc | Sia(α2,6)GalGlcNAcMan3GlcNAc2Fuc | 5.58 |

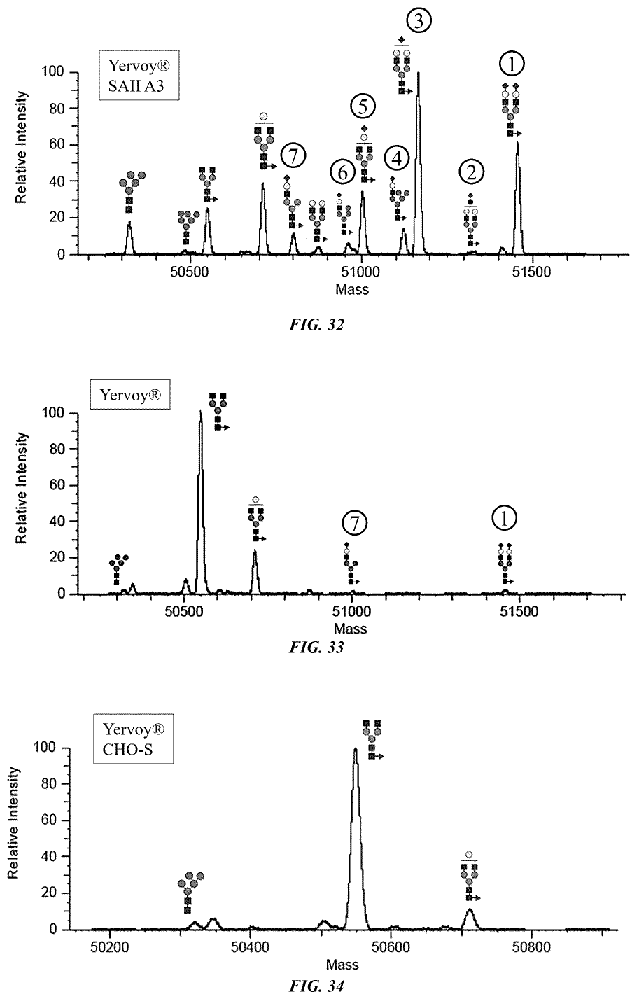

The Ipilimumab (Yervoy®) population produced by the SAII A3 cells according to the present disclosure exhibited all the seven SCT glycans as indicated in Table 1. Those SCT glycans comprised around 68.7% of the glycans of the population (See Table 7 below for the percentage of each identified SCT glycans), and, among them, SiaGal2GlcNAc2Man3GlcNAc2Fuc and Sia2Gal2GlcNAc2Man3GlcNAc2Fuc were the two most abundant ones (FIG. 32). The glycan profile of the commercial Avastin® product tested here comprised about 2.73% of sialylated glycans (FIG. 33). No sialylated glycans were observed in the CHO-S cell product (FIG. 34). It was also verified that the engineering did not affect the binding affinity to the antigen (FIG. 61).

TABLE 7

| Peak No. | Sialylated complex type glycans | Percentage |
|---|---|---|
| 1 | Sia2(α2,6)Gal2GlcNAc2Man3GlcNAc2 | 0.18 |
| 1 - Fuc | Sia2(α2,6)Gal2GlcNAc2Man3GlcNAc2Fuc | 17.9 |
| 2 - Fuc | Sia(α2,6)HexGal2GlcNAc2Man3GlcNAc2Fuc | 0.55 |
| 3 - Fuc | Sia(α2,6)Gal2GlcNAc2Man3GlcNAc2Fuc | 29.44 |
| 4 | Sia(α2,6)GalGlcNAcMan5GlcNAc2 | 0.82 |
| 4 - Fuc | Sia(α2,6)GalGlcNAcMan5GlcNAc2Fuc | 4.22 |
| 5 | Sia(α2,6)GalGlcNAc2Man3GlcNAc2 | 0.22 |
| 5 - Fuc | Sia(α2,6)GalGlcNAc2Man3GlcNAc2Fuc | 10.18 |
| 6 - Fuc | Sia(α2,6)GalGlcNAcMan4GlcNAc2Fuc | 1.9 |
| 7 - Fuc | Sia(α2,6)GalGlcNAcMan3GlcNAc2Fuc | 3.31 |

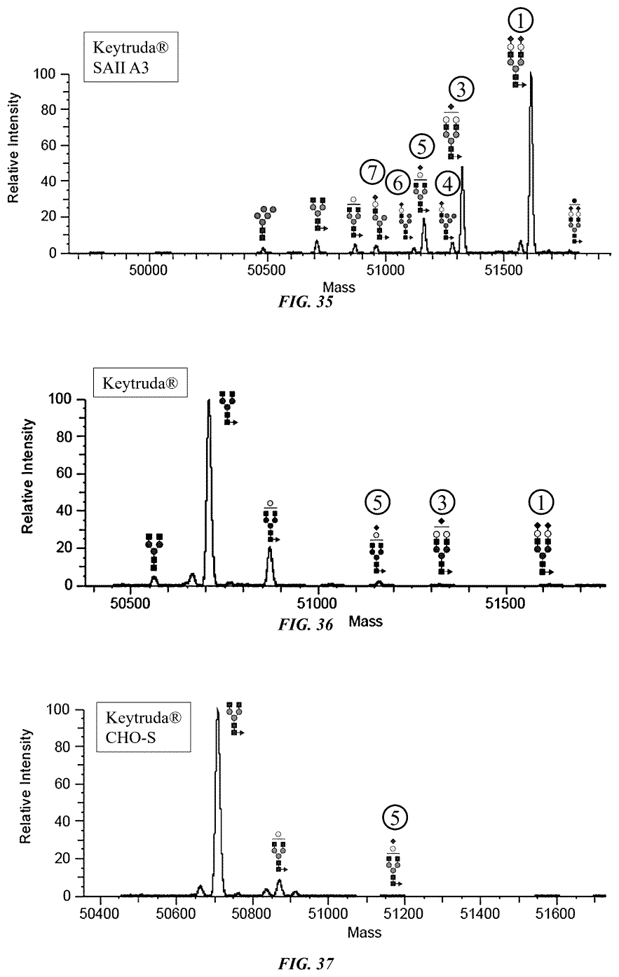

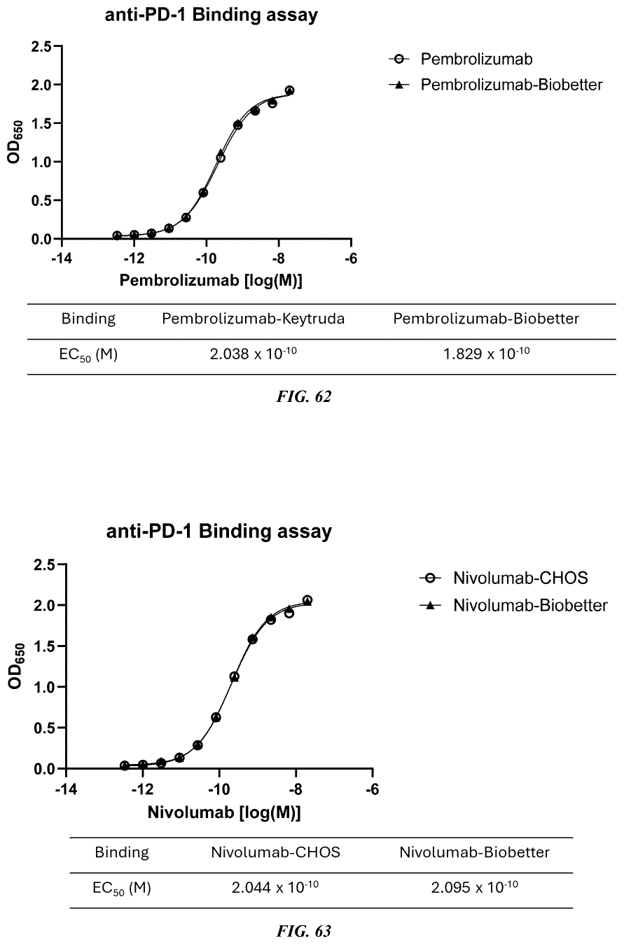

The Pembrolizumab (Keytruda®) population produced by the SAII A3 cells according to the present disclosure exhibited six kinds of SCT glycans as indicated in Table 1. Those SCT glycans comprised around 85.67% of the glycans of the population (See Table 8 below for the percentage of each identified SCT glycans), and, among them, SiaGal2GlcNAc2Man3GlcNAc2Fuc and Sia2Gal2GlcNAc2Man3GlcNAc2Fuc were the two most abundant ones (FIG. 35). The commercial Keytruda® product exhibited around 2.54% of sialylated glycans (FIG. 36), and the CHO-S cell product had them at around 0.11% (FIG. 37). Both were extremely low compared to the level that the present disclosure achieved. It was also verified that the engineering did not affect the binding affinity to the antigen (FIG. 62).

TABLE 8

| Peak No. | Sialylated complex type glycans | Percentage |
|---|---|---|
| 1 | Sia2(α2,6)Gal2GlcNAc2Man3GlcNAc2 | 0.45 |
| 1 - Fuc | Sia2(α2,6)Gal2GlcNAc2Man3GlcNAc2Fuc | 46.78 |
| 2 - Fuc | Sia(α2,6)HexGal2GlcNAc2Man3GlcNAc2Fuc | 0.45 |
| 3 - Fuc | Sia(α2,6)Gal2GlcNAc2Man3GlcNAc2Fuc | 22.5 |
| 4 | Sia(α2,6)GalGlcNAcMan5GlcNAc2 | 0.01 |
| 4 - Fuc | Sia(α2,6)GalGlcNAcMan5GlcNAc2Fuc | 2.73 |
| 5 | Sia(α2,6)GalGlcNAc2Man3GlcNAc2 | 0.01 |
| 5 - Fuc | Sia(α2,6)GalGlcNAc2Man3GlcNAc2Fuc | 9.16 |
| 6 - Fuc | Sia(α2,6)GalGlcNAcMan4GlcNAc2Fuc | 1.32 |
| 7 - Fuc | Sia(α2,6)GalGlcNAcMan3GlcNAc2Fuc | 2.09 |

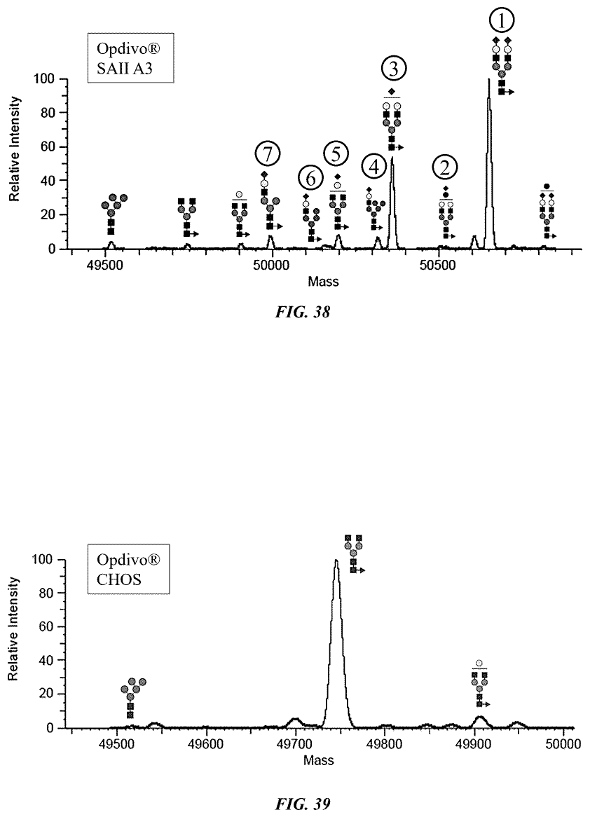

The Nivolumab (Opdivo®) population produced by the SAII A3 cells according to the present disclosure exhibited all the seven SCT glycans as indicated in Table 1. Those SCT glycans comprised around 85.79% of the glycans of the population (See Table 9 below for the percentage of each identified SCT glycans), and, among them, SiaGal2GlcNAc2Man3GlcNAc2Fuc and Sia2Gal2GlcNAc2Man3GlcNAc2Fuc were the two most abundant ones (FIG. 38). The CHO-S cell product, in contrast, had an ignorable 0.1% sialylated glycans (FIG. 39). It was also verified that the engineering did not affect the binding affinity to the antigen (FIG. 63).

TABLE 9

| Peak No. | Sialylated complex type glycans | Percentage |
|---|---|---|
| 1 | Sia2(α2,6)Gal2GlcNAc2Man3GlcNAc2 | 0.73 |
| 1 - Fuc | Sia2(α2,6)Gal2GlcNAc2Man3GlcNAc2Fuc | 46.83 |
| 2 - Fuc | Sia(α2,6)HexGal2GlcNAc2Man3GlcNAc2Fuc | 0.63 |
| 3 - Fuc | Sia(α2,6)Gal2GlcNAc2Man3GlcNAc2Fuc | 24.83 |
| 4 | Sia(α2,6)GalGlcNAcMan5GlcNAc2 | 0.77 |
| 4 - Fuc | Sia(α2,6)GalGlcNAcMan5GlcNAc2Fuc | 3.2 |
| 5 - Fuc | Sia(α2,6)GalGlcNAc2Man3GlcNAc2Fuc | 3.97 |
| 6 - Fuc | Sia(α2,6)GalGlcNAcMan4GlcNAc2Fuc | 1.16 |
| 7 - Fuc | Sia(α2,6)GalGlcNAcMan3GlcNAc2Fuc | 3.67 |

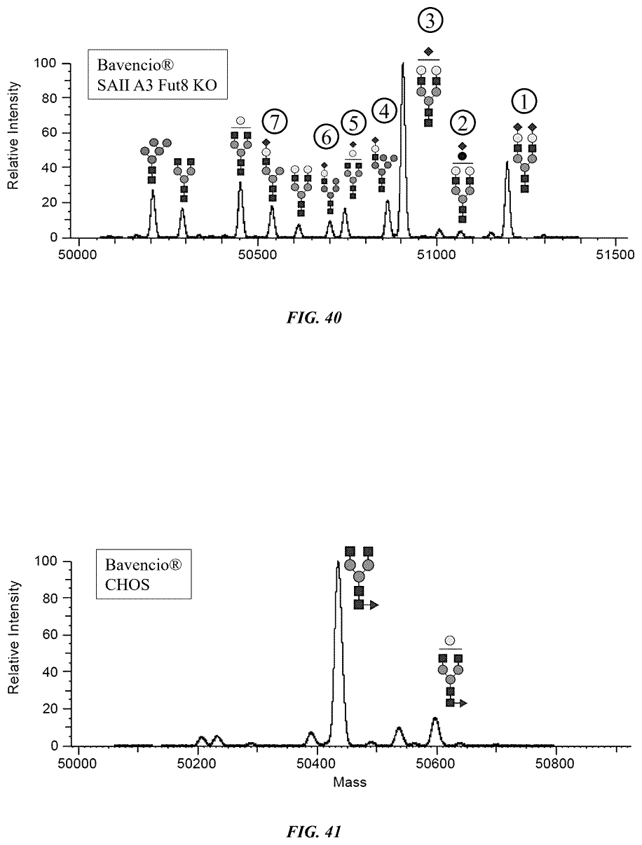

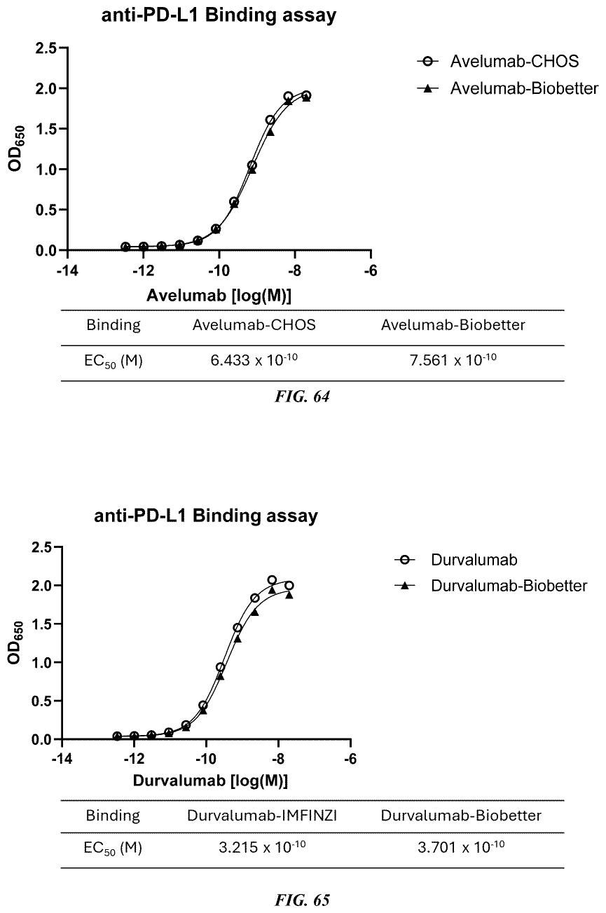

The Avelumab (Bavencio®) population produced by the SAII A3 Fut8 KO cells according to the present disclosure exhibited all the seven SCT glycans as indicated in the Table. Those SCT glycans comprised around 66.77% of the glycans of the population (See Table 10 below for the percentage of each identified SCT glycans), and, among them, SiaGal2GlcNAc2Man3GlcNAc2 and Sia2Gal2GlcNAc2Man3GlcNAc2 were the two most abundant ones (FIG. 40). No sialylated glycans were observed in the CHO-S cell product (FIG. 41). It was also verified that the engineering did not affect the binding affinity to the antigen (FIG. 64).

TABLE 10

| Peak No. | Sialylated complex type glycans | Percentage |
|---|---|---|
| 1 | Sia2(α2,6)Gal2GlcNAc2Man3GlcNAc2 | 13.58 |
| 2 | Sia(α2,6)HexGal2GlcNAc2Man3GlcNAc2 | 1.17 |
| 3 | Sia(α2,6)Gal2GlcNAc2Man3GlcNAc2 | 31.42 |
| 3 - Fuc | Sia(α2,6)Gal2GlcNAc2Man3GlcNAc2Fuc | 0.03 |
| 4 | Sia(α2,6)GalGlcNAcMan5GlcNAc2 | 6.73 |
| 5 | Sia(α2,6)GalGlcNAc2Man3GlcNAc2 | 5.21 |
| 6 | Sia(α2,6)GalGlcNAcMan4GlcNAc2 | 2.96 |
| 7 | Sia(α2,6)GalGlcNAcMan3GlcNAc2 | 5.67 |

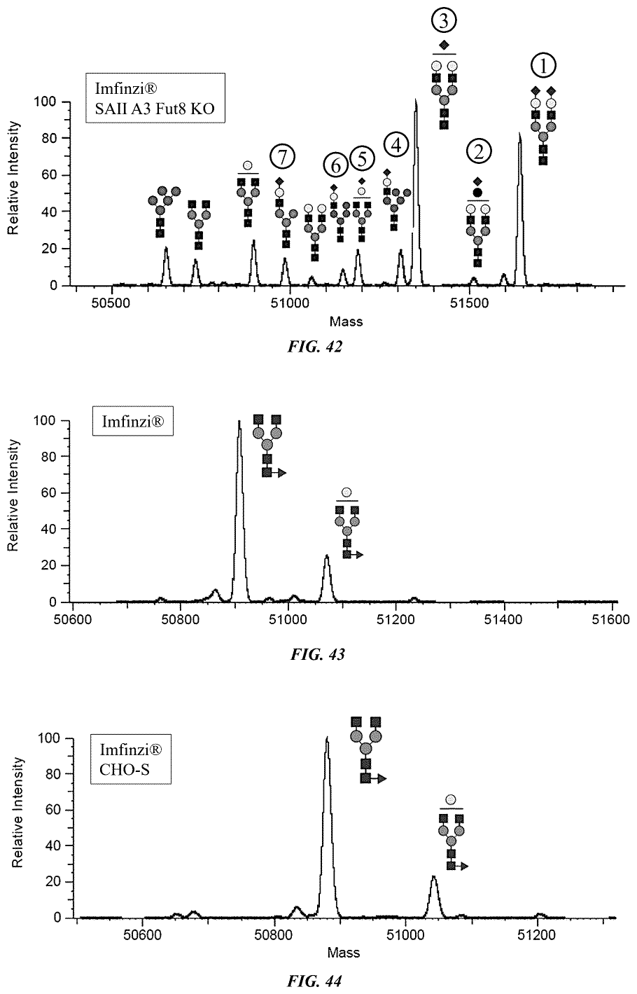

The Durvalumab (Imfinzi®) population produced by the SAII A3 Fut8 KO cells according to the present disclosure exhibited all the seven SCT glycans as indicated in Table 1. Those SCT glycans comprised around 74.36% of the glycans of the population (See Table 11 below for the percentage of each identified SCT glycans), and, among them, SiaGal2GlcNAc2Man3GlcNAc2 and Sia2Gal2GlcNAc2Man3GlcNAc2 were the two most abundant ones (FIG. 42). In comparison, the glycan profile of the commercial Imfinzi® product tested here exhibited no sialylated glycans (FIG. 43), nor did the CHO-S cell product show any (FIG. 44). It was also noted that the commercial product and the CHO-S cell product both had glycans without core fucose (despite a very small amount of SiaGal2GlcNAc2Man3GlcNAc2Fuc was detected, it was not visible in the figure, while the glycoprotein population of the present disclosure did not due to the Fut8 knock-out. It was also verified that the engineering did not affect the binding affinity to the antigen (FIG. 65).

TABLE 11

| Peak No. | Sialylated complex type glycans | Percentage |
|---|---|---|
| 1 | Sia2(α2,6)Gal2GlcNAc2Man3GlcNAc2 | 24.37 |
| 2 | Sia(α2,6)HexGal2GlcNAc2Man3GlcNAc2 | 1.21 |
| 3 | Sia(α2,6)Gal2GlcNAc2Man3GlcNAc2 | 29.96 |
| 3 - Fuc | Sia(α2,6)Gal2GlcNAc2Man3GlcNAc2Fuc | 0.19 |
| 4 | Sia(α2,6)GalGlcNAcMan5GlcNAc2 | 5.81 |
| 5 | Sia(α2,6)GalGlcNAc2Man3GlcNAc2 | 5.85 |
| 6 | Sia(α2,6)GalGlcNAcMan4GlcNAc2 | 2.59 |
| 7 | Sia(α2,6)GalGlcNAcMan3GlcNAc2 | 4.38 |

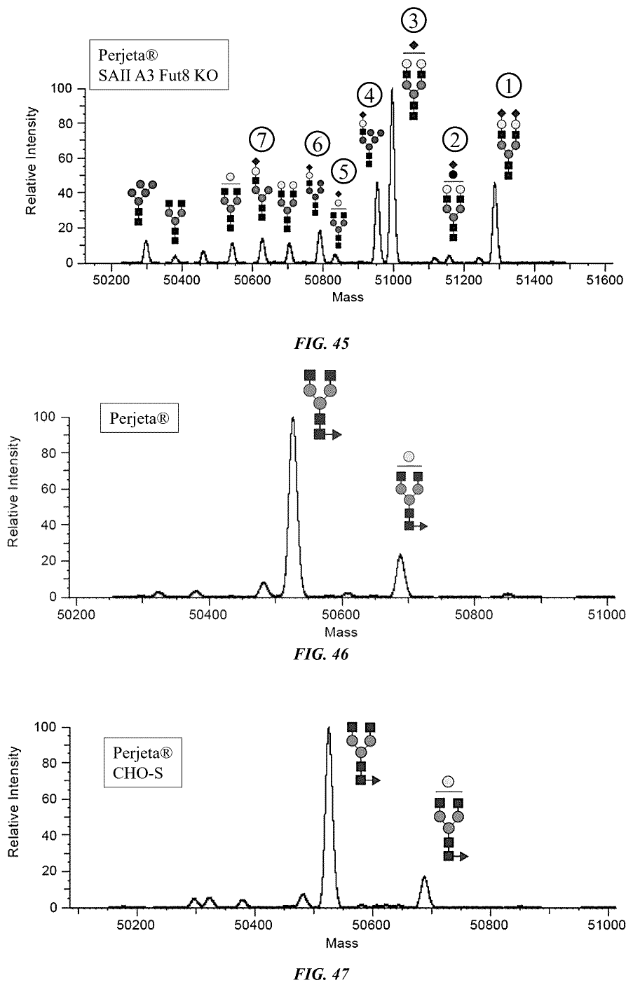

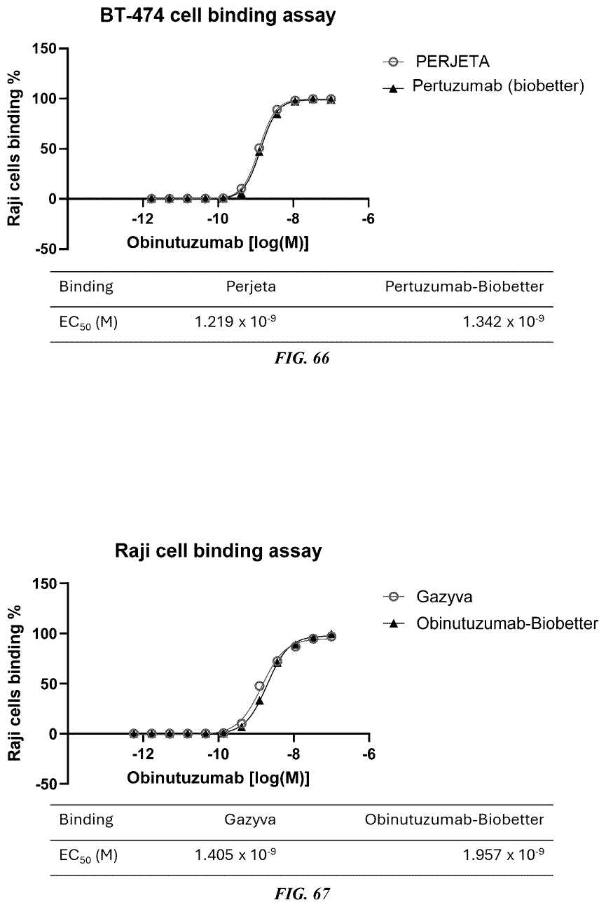

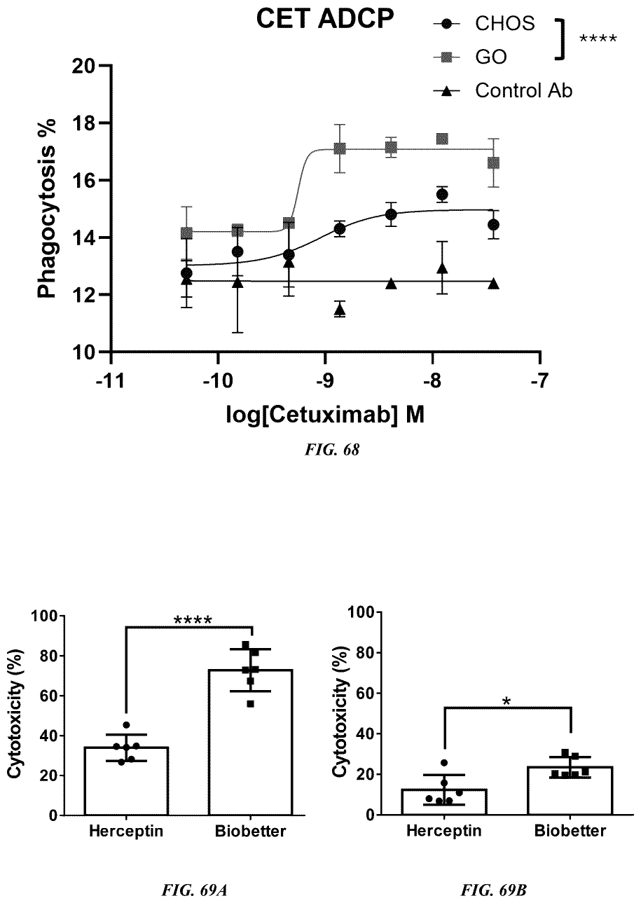

The Pertuzumab (Perjeta®) population produced by the SAII A3 Fut8 KO cells according to the present disclosure exhibited all the seven SCT glycans as indicated in Table 1. Those SCT glycans comprised around 79.19% of the glycans of the population (See Table 12 below for the percentage of each identified SCT glycans), and, among them, SiaGal2GlcNAc2Man3GlcNAc2 and Sia2Gal2GlcNAc2Man3GlcNAc2 were the two most abundant ones (FIG. 45). In comparison, the glycan profile of the commercial Perjeta® product tested here exhibited no sialylated glycans (FIG. 46), nor did the CHO-S cell product show any (FIG. 47). It was also noted that the commercial product and the CHO-S cell product both had glycans without core fucose, while the glycoprotein population of the present disclosure did not due to the Fut8 knock-out. It was also verified that the engineering did not affect the binding affinity to the antigen (FIG. 66).

TABLE 12

| Peak No. | Sialylated complex type glycans | Percentage |
|---|---|---|
| 1 | Sia2(α2,6)Gal2GlcNAc2Man3GlcNAc2 | 15.44 |
| 2 | Sia(α2,6)HexGal2GlcNAc2Man3GlcNAc2 | 1.44 |
| 3 | Sia(α2,6)Gal2GlcNAc2Man3GlcNAc2 | 33.79 |
| 4 | Sia(α2,6)GalGlcNAcMan5GlcNAc2 | 15.66 |
| 5 | Sia(α2,6)GalGlcNAc2Man3GlcNAc2 | 1.61 |
| 6 | Sia(α2,6)GalGlcNAcMan4GlcNAc2 | 6.4 |
| 7 | Sia(α2,6)GalGlcNAcMan3GlcNAc2 | 4.84 |

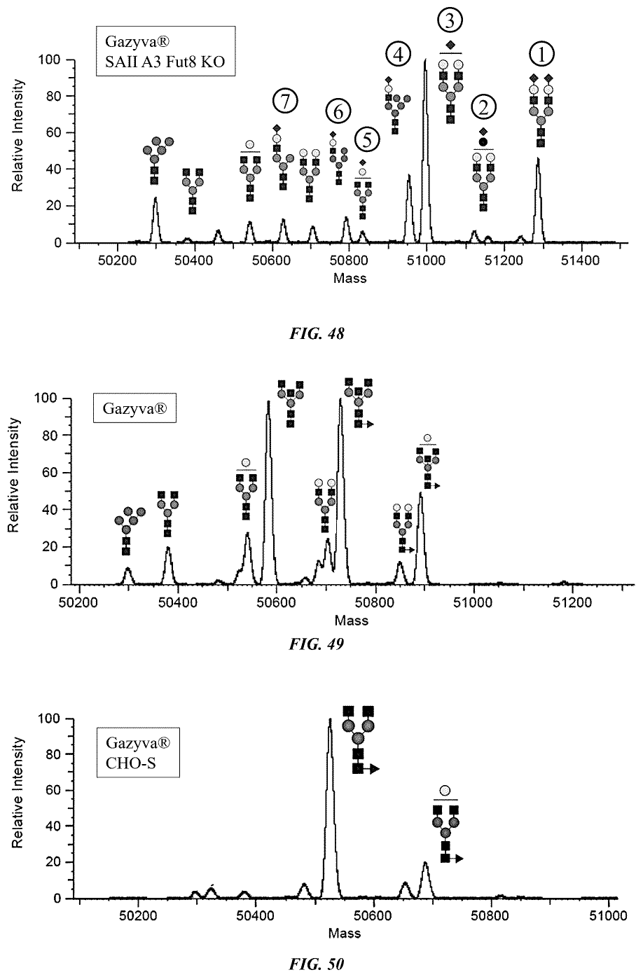

The Obinutuzumab (Gazyva®) population produced by the SAII A3 Fut8 KO cells according to the present disclosure exhibited all the seven SCT glycans as indicated in Table 1. Those SCT glycans comprised around 75.09% of the glycans of the population (See Table 13 below for the percentage of each identified SCT glycans), and, among them, SiaGal2GlcNAc2Man3GlcNAc2 and Sia2Gal2GlcNAc2Man3GlcNAc2 were the two most abundant ones (FIG. 48). In comparison, the glycan profile of the commercial Gazyva® product tested here exhibited no sialylated glycans (FIG. 49), nor did the CHO-S cell product show any (FIG. 50). It was also noted that the commercial product and the CHO-S cell product both had glycans with core fucose, while the glycoprotein population of the present disclosure did not due to the Fut8 knock-out. It was also verified that the engineering did not affect the binding affinity to the antigen (FIG. 67).

TABLE 13

| Peak No. | Sialylated complex type glycans | Percentage |
|---|---|---|
| 1 | Sia2(α2,6)Gal2GlcNAc2Man3GlcNAc2 | 15.73 |
| 2 | Sia(α2,6)HexGal2GlcNAc2Man3GlcNAc2 | 1.19 |
| 3 | Sia(α2,6)Gal2GlcNAc2Man3GlcNAc2 | 34.28 |
| 4 | Sia(α2,6)GalGlcNAcMan5GlcNAc2 | 12.6 |
| 5 | Sia(α2,6)GalGlcNAc2Man3GlcNAc2 | 2.05 |
| 6 | Sia(α2,6)GalGlcNAcMan4GlcNAc2 | 4.85 |
| 7 | Sia(α2,6)GalGlcNAcMan3GlcNAc2 | 4.38 |

In conclusion, the engineering method of the present disclosure provided enriched but heterogeneous glycan profile with more than one type of sialylated glycans for the glycoproteins. The engineering did not affect the target-binding affinities of the glycoproteins. In other words, the engineering method and the engineered glycoproteins population were able to obtain the desired glycan profile for better ADCC, CDC, ADCP, vaccinal effect, and half-live while maintaining the target-binding affinities (the data mentioned above are concluded in Table 14 below).

TABLE 14

| | | | | Binding affinity EC₅₀ (M) | |
|---|---|---|---|---|---|
| Glycoprotein | Brand Name | Sialylation | Purity | Biobetter | Commercial |
| Adalimumab | Humira ® | 81% | 95% | 3.728 × 10⁻¹⁰ | 2.127 × 10⁻¹⁰ |
| Bevacizumab | Avastin ® | 80% | 96*% | 3.766 × 10⁻¹⁰ | 2.994 × 10⁻¹⁰ |
| Ipilimumab | Yervoy ® | 68% | >99% | 3.169 × 10⁻¹⁰ | 2.809 × 10⁻¹⁰ |
| Avelumab | Bavencio ® | 67% | 98% | 7.561 × 10⁻¹⁰ | 6.433 × 10⁻¹⁰** |
| Durvalumab | IMFINZI ® | 74% | 97% | 3.701 × 10⁻¹⁰ | 3.215 × 10⁻¹⁰ |
| Pembrolizumab | Keytruda ® | 86% | >99*% | 1.829 × 10⁻¹⁰ | 2.038 × 10⁻¹⁰ |
| Nivolumab | Opdivo ® | 85% | >99*% | 2.095 × 10⁻¹⁰ | 2.044 × 10⁻¹⁰** |
| Trastuzumab | Herceptin ® | 75% | 99% | 1.442 × 10⁻¹⁰ | 1.088 × 10⁻¹⁰ |
| Pertuzumab | Perjeta ® | 79% | 97% | 1.342 × 10⁻⁹ | 1.219 × 10⁻⁹ |
| Cetuximab | Erbitux ® | 58% | >99% | 8.326 × 10⁻¹¹ | 6.732 × 10⁻¹¹ |
| Rituximab | Mabthera ® | 83% | >99% | 7.703 × 10⁻¹⁰ | 5.441 × 10⁻¹⁰ |
| Obinutuzumab | Gazyva ® | 75% | 96% | 1.957 × 10⁻⁹ | 1.405 × 10⁻⁹ |

### Example 6: Manufacturing Exemplary HEK293 Cells of the Present Disclosure for Expressing a Sialylated Glycoprotein

This experiment manufactured an exemplary cell according to an embodiment of the present disclosure from a parental HEK293T cell using CRISPR technologies. The results were very similar to the results of using the ExpiFectamine™ CHO Transfection Kit on CHO cells, as described in Example 1.

Cells, Plasmids, and Transfection. The parental HEK293T cells (ATCC) were cultured in the DMEM (Thermo Fisher Scientific) supplied with 10% FBS (Thermo Fisher Scientific) at 37° C. with 5% CO₂ in the cell incubator. The plasmids for CRISPR/Cas9 expression were constructed as suggested by the kit manual (Thermo Fisher Scientific). Briefly, the validated sgRNA sequence targeting glycosyltransferase was synthesized (Integrated DNA Technologies) and cloned into the vector for Cas9 and sgRNA expression. The synthetic codon-optimized gene insert (Integrated DNA Technologies), flanked by the homologous arm of the target gene at the sgRNA target site, was cloned into an empty vector as the donor plasmid. Transfection of 293T cells was mediated by TransIT-293 (Mirus Bio) following the reagent manual. A plasmid for expressing antibody chMC81370 (a humanized antibody targeting specific stage embryonic antigen 4 (SSEA4)) was transfected into cells followed by incubation. At the time of harvest, the culture medium was collected and then subjected to protein A sepharose beads (GE HealthCare) column to purify the antibodies.

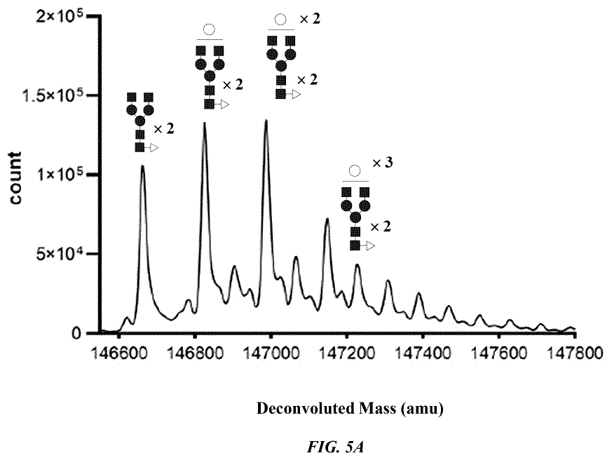

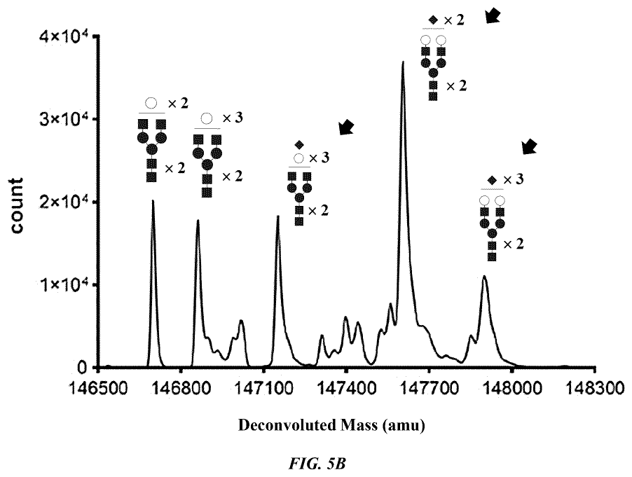

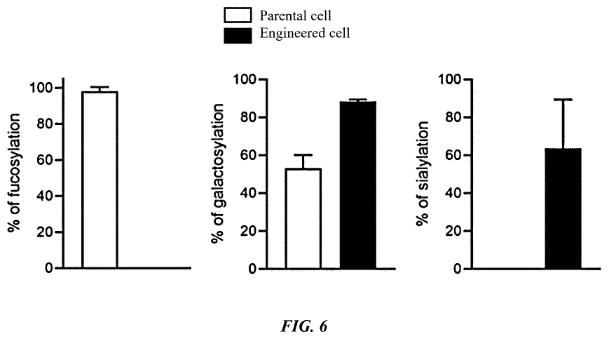

Analysis. The purified antibodies were analyzed using intact protein mass (IPM) analysis. Briefly, the samples were diluted with LCMS grade water at a concentration between 5-10 uM and analyzed by 6230 TOF LC/MS with a Dual AJS ESI ion source (Agilent Technologies) with PLRP-S 1000 Å 5 μm column (Agilent Technologies). Solvent A was 0.1% Formic Acid in H₂O and Solvent B was 0.1% Formic Acid in CAN. The results show a significant increase in galactosylated and sialylated antibody glycoforms (FIG. 5B) compared to the antibodies generated by the parental HEK293T cells (FIG. 5A). The results of the IPM Glycoform analysis were presented in bar charts, which revealed a significant increase in antibody galactosylation and sialylation, with a lack of core-fucosylation (FIG. 6).

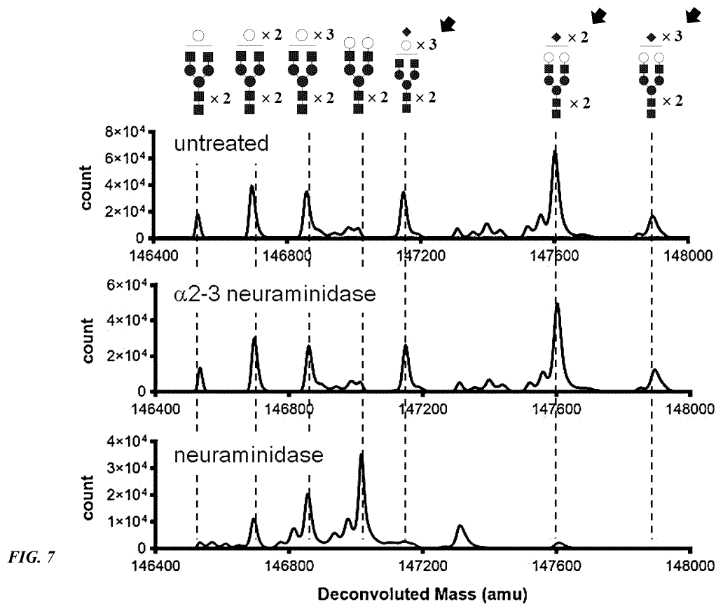

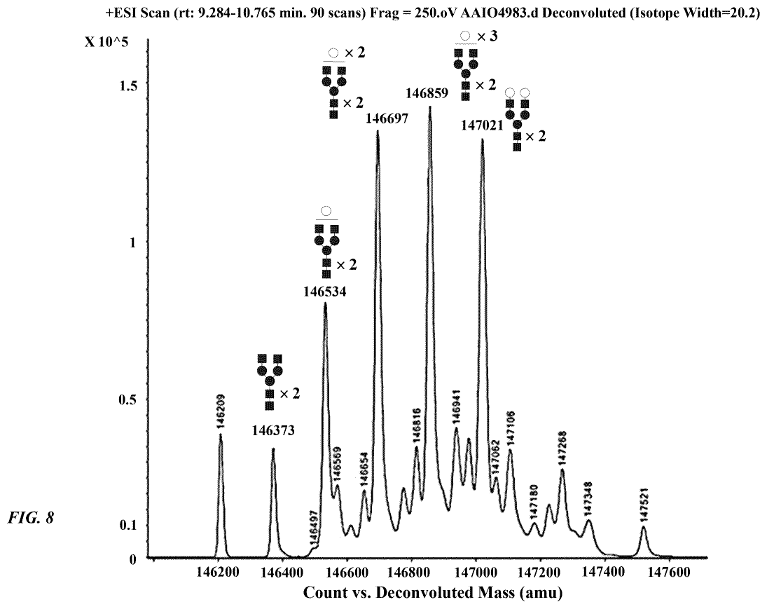

Characterizing the sialylation. To confirm the sialylation on the antibody was mediated by hST6Gall, the antibody was treated with two different sialidases, Streptococcus pneumonia a2-3 neuraminidase and Clostridium perfringens neuraminidase at 37° C. overnight respectively followed by purification or gel electrophoresis. The intact protein analysis did not show any obvious difference in the distribution of asialylated and sialylated antibodies between the untreated and a2-3 neuraminidase-treated group (FIG. 7 upper and middle; sialylated glycoforms are labeled with the solid arrows), indicating that the sialylation on antibody was not due to the endogenous a2-3 sialyltransferases. On the other hand, treatment with Clostridium perfringens neuraminidase, which could remove all linkages of sialylation, largely eliminated the terminal sialic acids (FIG. 7 bottom). In addition, the antibodies with increased galactosylation generated from cells with knock-in of hB4GalT1 only did not show terminal sialylation (FIG. 8). These results confirmed that sialylation on the antibody was introduced by hST6Gall and the increase of hB4Gall expression indeed elevated the galactosylation of antibody (FIG. 5).

The sialyltransferase hST6Gall is known as the only human glycosyltransferase that mediates a2-6 sialylation and preferentially uses the al-3 branch arm of the biantennary glycan as substrates. Some in vitro studies show glycoengineering on antibody glycans by hST6Gall takes a long time and is difficult to yield fully sialylated antibody glycans. Furthermore, this also could explain why only the antibody with at least one fully galactosylated glycan was sialylated. If the galactose on G1 glycan was not on the al-3 branch arm, the hST6Gall may not sialylate it. Therefore, the expression level of glycosyltransferases may not be the bottleneck in this situation. Rather, substrate preference and enzyme activity are the major factors. Using a2-6 sialylatranferases from other organisms may have similar or better activity than hST6Gall, which is evident by the other examples described herein that used a PspST gene.

### Example 7: Time Factor in Glycoengineering Using an Exemplary Cell of the Present Disclosure

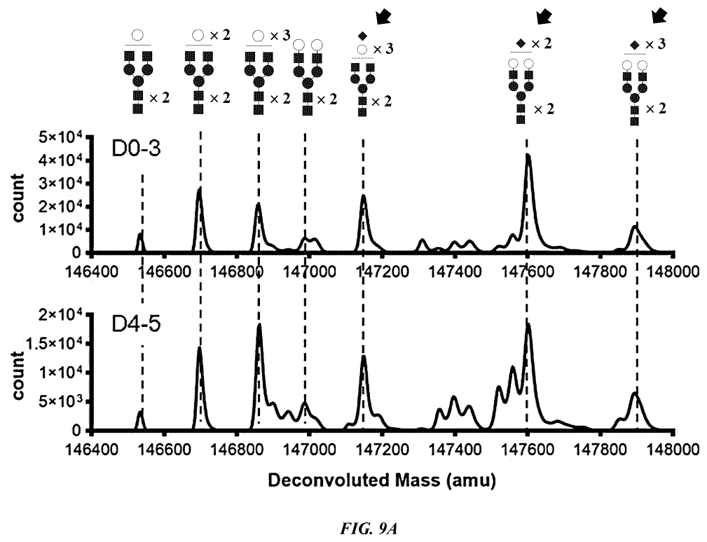

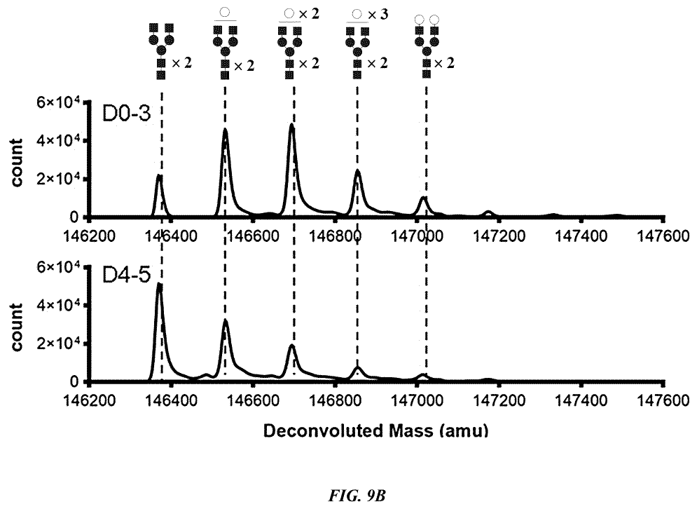

This experiment tested whether the amount and the time of antibody expression using an exemplary cell of the present disclosure affect glycosylation. The culture time was extended to 5 days, and the medium of the culture was collected on Day 3 and Day 5, respectively. The produced antibodies were examined using intact protein mass (IPM) analysis. The results show a relatively strong sialylation in the first collection (first three days) (FIG. 9A, DO-3), and then sialylation was decreased in the second collection (last two days), accompanied by an increase of terminal galatosylation (FIG. 9A, D4-5), suggesting that the proteins involved in glycosylation may not be sufficient to effectively produce Fc-SCT antibody at a later stage.

Because the hST6Gall and hB4GalT1 involved in the FC-SCT antibody production were almost artificially and constitutively expressed in the cells, we also examined if endogenous glycosyltransferases faced the same problem of reduced glycosylation. By expressing antibodies in FUT8 KO cells, the antibody was almost terminally glycosylated with at least one or more galactoses in the first collection (FIG. 9B, DO-3), but the proportion of galactosylated antibody decreased, and even the antibody glycans without any galactoses became the dominant glycoforms in the last two days (FIG. 9B, D4-5). The result showed that the dynamic glycosylation level after the antibody expression can be observed on endogenous glycosylation products as well.

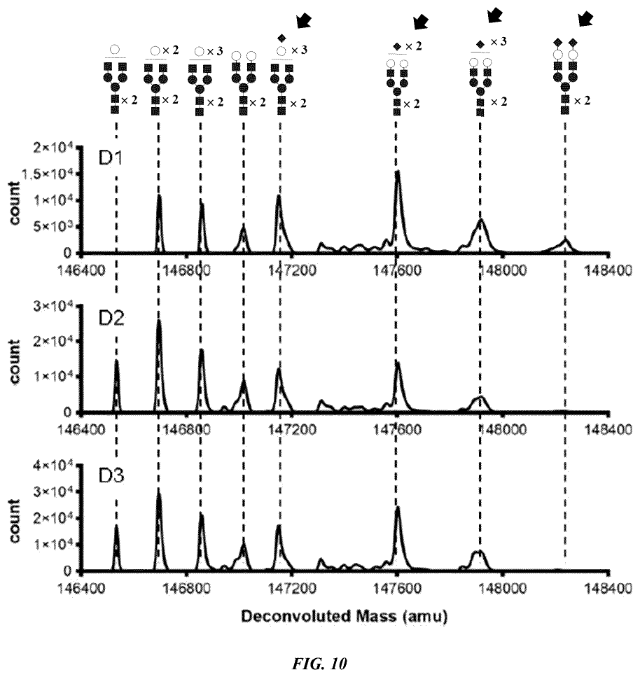

The aforementioned finding suggests that antibody production at different time points may impact glycosylation differently. Therefore, in the next experiment, antibodies were purified every day after plasmid transfection and analyzed the dynamic change of antibody glycans from multiple engineered cells. The antibody with full sialylation could be detected by intact protein mass analysis on the first day and then gradually decreased over time (FIG. 10). Regarding galactosylation, the antibody glycans were capped by at least two galactoses (2˜4 galactoses in total) on the first day, but started to decline on the second day, as evidenced by the appearance of one galactose on antibody glycans (FIG. 10). These observations suggest that antibody glycosylation was almost complete at the beginning of the protein expression and gradually decreased over time.

### Example 8: Binding Between Sialylated Antibodies and Fcγ Receptors

FcγRIIA (Fc gamma RIIA) and FcγRIIB (Fc gamma RIIB) are usually expressed simultaneously by the antigen-presenting cells, such as dendritic cells and macrophages, and these two receptors work together to modulate immune responses. FcγRIIIA is critical for NK cells to mediate ADCC3. This experiment tested whether the Fc-SCT-enriched antibodies produced using the exemplary cells of the present disclosure show better binding affinity than wide-type antibodies (i.e., antibodies produced by parental cells). The Fc-SCT-enriched antibodies were produced and purified as described in Example 4. The binding affinity was examined using an ELISA assay.

ELISA. The recombinant soluble FcγIIA, FcγIIB, and FcγRIIIA (Fc gamma RIIIA, R&D) was coated with 50 ng/well in bicarbonate/carbonate coating buffer (50 mM, pH 10) at 4° C. overnight before blocking the well with 5% BSA in TPBS (0.05% Tween 20 in PBS) at 4° C. overnight. The antibody was added into wells with final concentration started at 100 μg/ml with 5×series dilution and incubated at room temperature for 1 hour followed by incubation with HRP-conjugated goat anti-human IgG antibody (Jackson ImmunoResearch) for another 1 hour at room temperature. Finally, the TMB substrate (Bethyl Laboratories) was added to react with HRP at RT prior to stopping the reaction by H₂SO₄. The absorbance at 450 nm was detected by SpectraMax M5 spectrum reader (Molecular Device). The wells were washed by TPBS 3-5 times between each step.

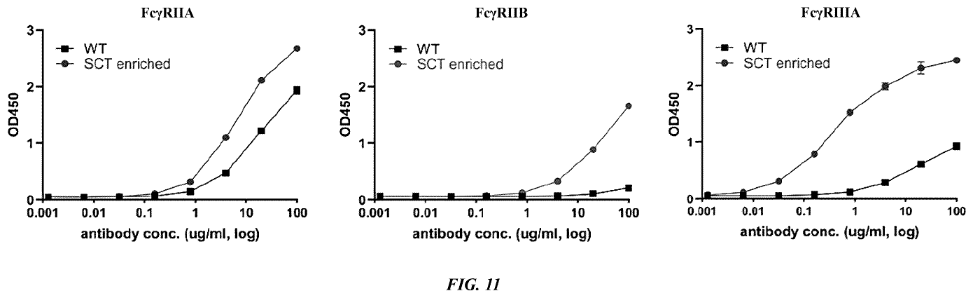

The results show that the binding of Fc-SCT-enriched antibodies to FcRs was increased in three tested Fc receptors (FIG. 11). Among these receptors, the increased binding of FcγRIIA and FcγRIIIA was consistent with previous studies. Interestingly, the WT antibody showed very weak binding to the inhibitory receptor FcγRIIB. Despite the improvement being less significant than that of FcγRIIA and FcγRIIIA, the glycoengineering by the cells of the present disclosure was able to improve the antibody's binding to receptor FcγRIIB. Although the increased binding to the two opposing functional receptors, FcγRIIA and FcγRIIB, which are typically expressed on the antigen-presenting cells (APCs) simultaneously, appears to be conflicting, the eventual immune response was determined by the expression level and signaling strength of FcγRIIA and FcγRIIB on effector cells. The Fc-SCT-enriched antibody also exhibited stronger FcγRIIIA binding ability than the antibody produced from wild-type cells (FIG. 11). Without wishing to be bound by theories, this enhancement was also contributed by the removal of core-fucosell and the addition of galactose.

### Example 9: Manufacturing Exemplary HEK293 Cells of the Present Disclosure for Expressing GlcNAc Glycoproteins

This experiment manufactured an exemplary cell according to an embodiment of the present disclosure from a parental GnT1 KO ExpiHEK293F cell using CRISPR technologies. The parental cells were prepared as described in Example 4. The N-glycan expressed by this cell line was a high mannose type which was preferentially cleaved by Endo H to generate Fc-GlcNAc antibody. Therefore, this experiment aims to knock in Endo H (UniProtKB/Swiss-Prot: P04067.1, Sec SEQ ID NO: 137 and SEQ ID NO: 139) in this cell line by CRISPR-Cas9. The plasmid for the CRISPR technology was prepared as described below.

Plasmids and Transfection. The plasmids for CRISPR/Cas9 expression were constructed as suggested by the kit manual (Thermo Fisher Scientific). Briefly, the validated sgRNA sequence targeting glycosyltransferase was synthesized (Integrated DNA Technologies) and cloned into the vector for Cas9 and sgRNA expression. The synthetic codon-optimized gene insert (Integrated DNA Technologies), flanked by the homologous arm of the target gene at the sgRNA target site, was cloned into an empty vector as the donor plasmid. Transfection of 293T cells was mediated by using an Expi293Fectamine™ Transfection Kit (Thermo Fisher Scientific) following the reagent manual. A plasmid for expressing antibody chMC81370 was transfected into cells followed by incubation. At the time of harvest, the culture medium was collected and then subjected to protein A sepharose beads (GE HealthCare) column to purify the antibody.

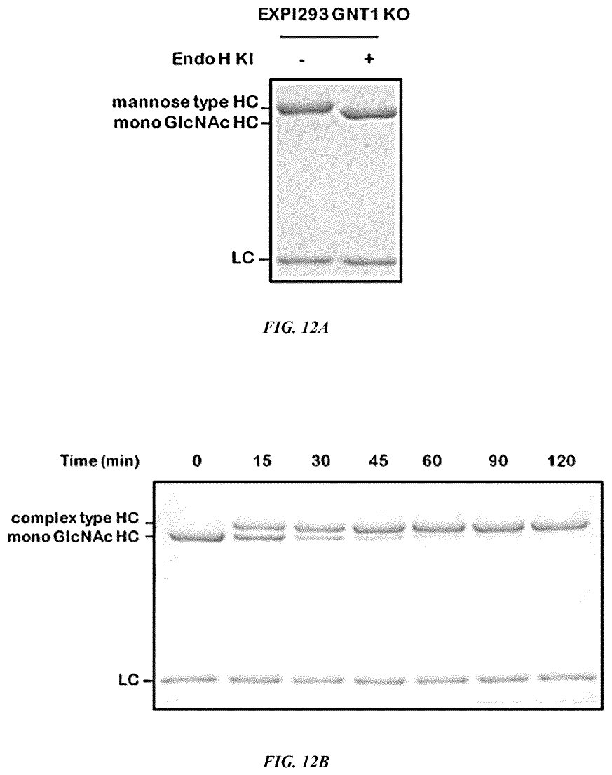

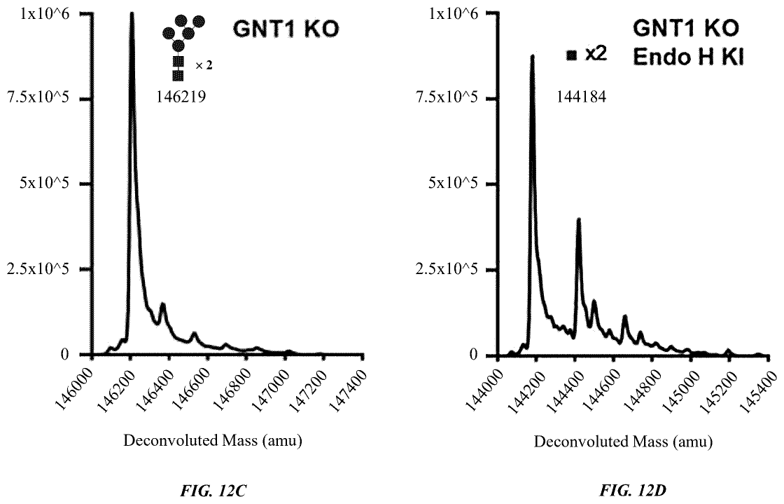

Analysis. The antibodies expressed by the cells were analyzed by electrophoresis. The protein was heated at 95° C. for 5 mins in the LDS sample buffer supplied with 2-Mercaptoethanol and then subjected to gel electrophoresis with 12% SDS-PAGE. The protein on the gel was stained by Coomassie blue buffer (ApexBio). The result showed that the antibody heavy chain (AbHC) expressed by these cells had faster mobility than AbHC from its parental cells (GnT1 KO), and most AbHCs were downshifted (FIG. 12A).

This antibody was also subjected to a transglycosylation assay. The antibody was incubated with SCT glycan-oxazoline and Endo S2 mutant in Tris buffer at 37° C. for the time indicated before harvest for purification or gel electrophoresis. The transglycosylation generated antibodies with homogenous glycan (in this experiment, homogenous SCT glycan). The efficiency of the transglycosylation was pretty good (FIG. 12B). The glycans on these antibodies were then by intact protein mass (IPM) analysis. The results showed that most antibodies (over 95%) contain only GlcNAc on each HC (FIG. 12C, FIG. 12D, and FIG. 12E).

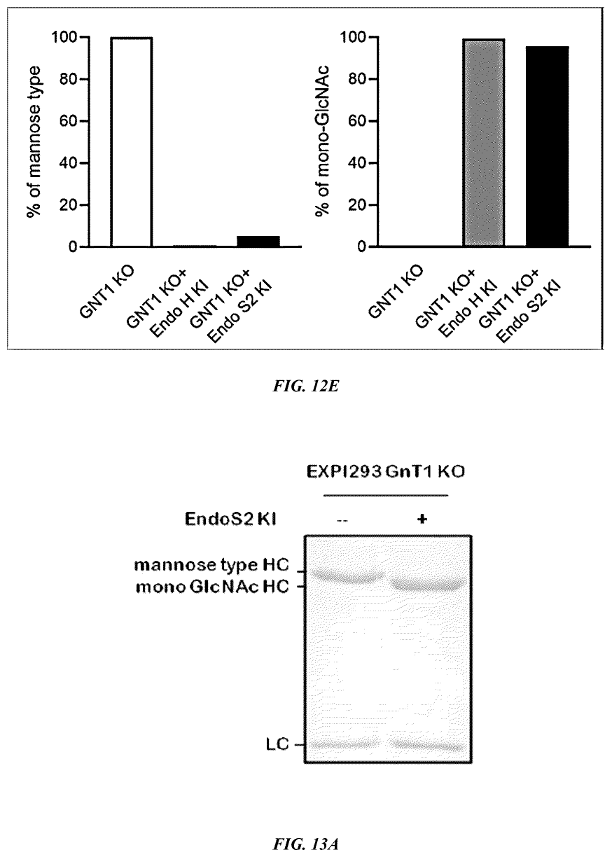

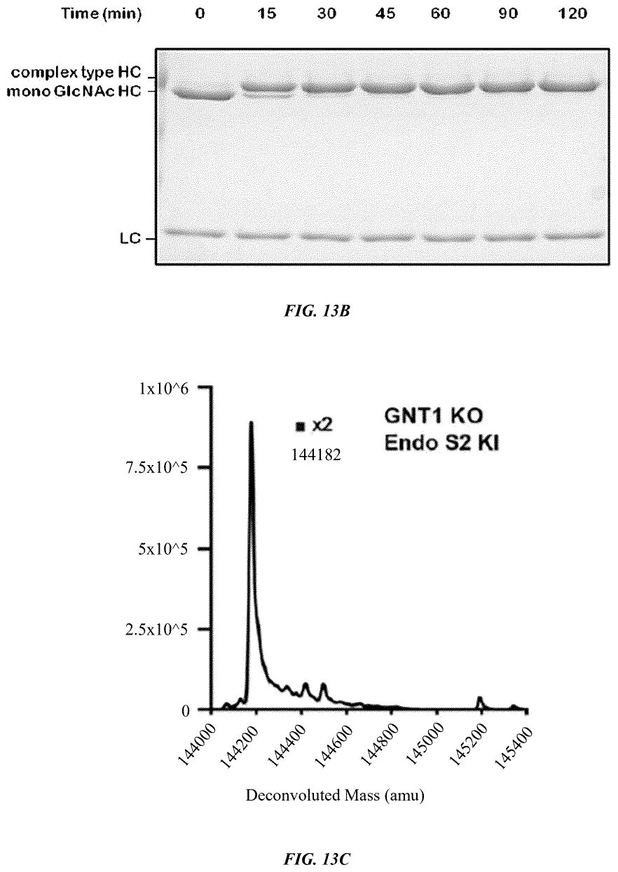

Alternatively, instead of knocking in Endo H, endoglycosidase S2 (Endo S2; UniProtKB/Swiss-Prot: T1WGN1.1, See SEQ ID NO: 138 and SEQ ID NO: 140) from Streptococcus pyogenes was knocked in because this enzyme can hydrolyze both high mannose and complex type N-glycans to Fc-GlcNAc. The donor vector and CRISPR-Cas9 plasmids were delivered to GnT1 KO cells. Then the antibody glycans generated by these cells were examined as previously described. The protein gel showed an obvious downshifted band of AbHC compared with its parental cells (GNT1 KO) (FIG. 13A). Besides, the glycans also can be added onto AbHC through enzymatic transglycosylation (FIG. 13B) and intact protein mass analysis showed a clear signal of Fc-GlcNAc antibody (FIG. 13C), most antibodies (over 90%) contain only GlcNAc on each HC (FIG. 12E).

Based on these results, it is concluded that both Endo H and Endo S2 can be introduced to cells by CRISPR-Cas9 to process the N-glycans to Fc-GlcNAc anti-body for in vitro transglycosylation. In addition, this cell-based method can be used for other glycoproteins, such as influenza hemagglutinin and SARS-COV-2 spike protein to generate the mono-GlcNAc decorated glycoforms as vaccines to elicit broadly protective immune responses

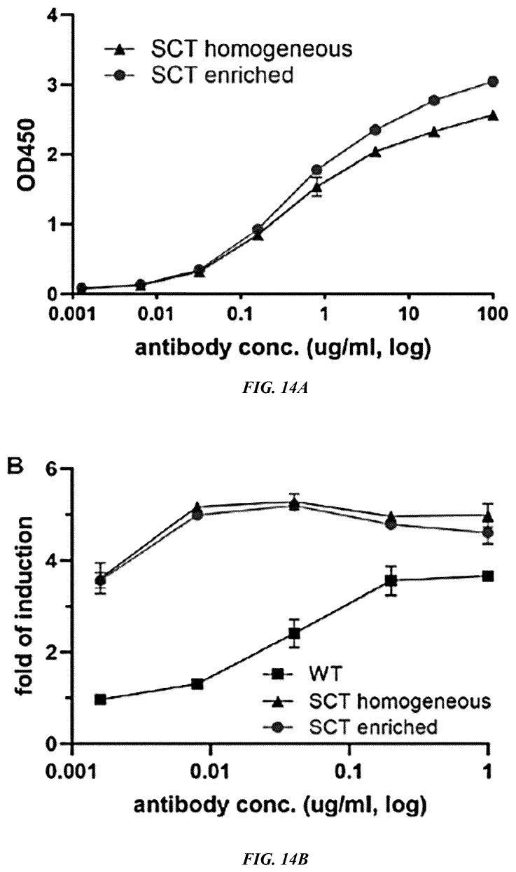

Next, since the binding of Fc-SCT enriched antibodies (i.e., the antibody obtained in Example 4) to FcγIIIA receptor was elevated, the homogeneous Fc-SCT antibodies obtained above through transglycosylation were compared with the Fc-SCT enriched antibodies for their binding affinity to FcγIIIA receptors. The result showed that Fc-SCT enriched antibodies exhibited similar binding avidity to homogenous SCT antibodies (FIG. 14A).

Furthermore, an ADCC reporter assay was conducted. Briefly, the assay was performed according to the kit manual. The SSEA4 expressing target cells SK-OV3 were seeded into a 96-well plate, followed by the addition of an antibody with the final concentration started at 1 g/ml with 5×series dilution. Then, the FcγRIIIA expressing effector cells (effector: target cell ratio, 6:1) were added and incubated with target cells in the incubator at 37° C. for 6 hrs. The plate was placed at room temperature for 15 min prior to adding the luciferase substrate. After 5 min incubation, the luminescence was measured by SpectraMax M5 spectrum reader. The induction fold was calculated by RLU (induced-background)/RLU (no antibody control-background). It was observed that at the lowest antibody concentration used for induction, WT antibody did not induce any obvious cell activation, but the homogenous Fc-SCT and Fc-SCT enriched antibodies can trigger cell activation and rapidly reached a maximum at next concentration (FIG. 14B). Even at the higher concentration, the WT antibody can reach a maximum induction, but the activity was still much lower than that of homogenous Fc-SCT or Fc-SCT enriched antibody (FIG. 14B).

### Example 10: Generation of 7-Fluorosialyl Glycoengineered Adalimumab

Adalimumab overexpression SWO-006 CHO cells were cultured at 5×10⁵/mL in 3 mL BalanCD CHO Growth A (FUJIFILM) supplement with 8 mM GlutaMAX (ThermoFisher Scientific) in a well of a 6-well plate. On day 0, peracetylated 4-F-ManNAc was added into the culture to give 300 μM. The same amount of peracetylated 4-F-ManNAc was added on day 3 to boost. The cells were incubated on a shaking platform at 37 degrees Celsius, 8% CO₂. On day 7, the supernatant was harvested for antibody purification using protein A beads. The collected antibodies were analyzed using glycopeptide LC/MS-MS to demonstrate their glycoforms and to confirm the modification of the terminal sialic acids.

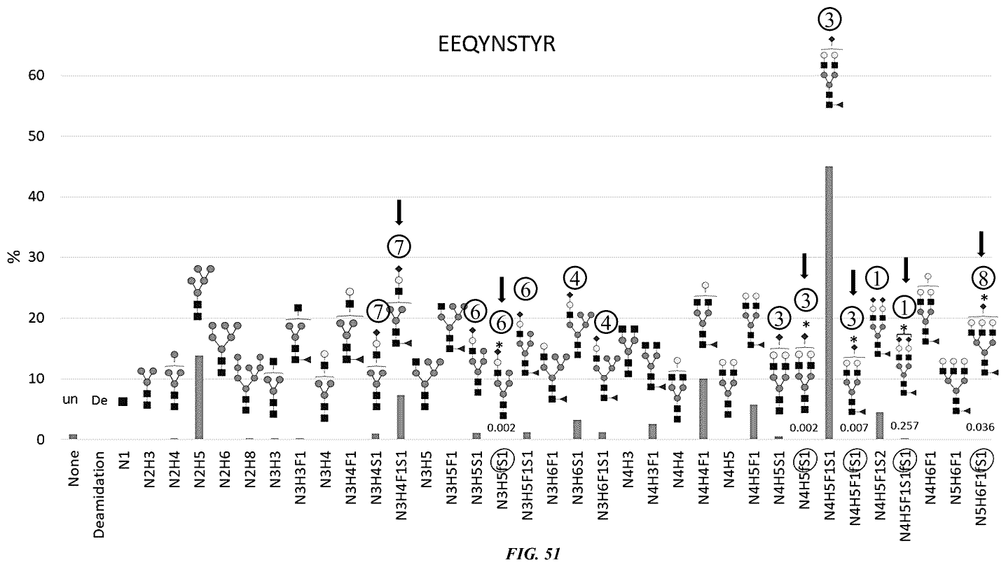

The LC/MS-MS spectrum (FIG. 51) shows that the Adalimumab produced in this experiment exhibited SCT glycans, including the Peak 1 glycan, Peak 3 glycan, Peak 4 glycan, Peak 6 glycan, and Peak 7 glycan (See Table 1 above). The above-identified SCT glycans observed here included both modified terminal sialic acids (7-fluoro sialic acid; which are indicated by solid arrows in the figure) and non-modified terminal sialic acids. The above-identified SCT glycans observed here also included both the form with and without fucose (fucose is shown as a triangle in the figure). Furthermore, the SCT glycans observed in this experiment also included a SiaGal3GlcNAc3Man3GlcNAc2Fuc glycan (which is marked as Peak 8). The Peak 8 glycan also exhibited modified terminal sialic acids. The glycans with modified terminal sialic acids were verified using LS/MS-MS (data not shown). They comprised around 8.63% of the sialylation. This data confirms that the concept of adding peracetylated 4-F-ManNAc can indeed generate 7-fluoro sialic acid-terminated glycans for the glycoproteins produced according to the present disclosure.

### Example 11: ADCC Evaluation

This experiment tested the exemplary glycoprotein population of the present disclosure's performance in respect to the ADCC effect incurred by the glycoprotein population. The Trastuzumab (Herceptin®) population, the Pertuzumab (Perjeta®) population, the Rituximab (MabThera® or Rituxan®) population, and the Cetuximab (Erbitux®) population prepared in Example 5 above were examined in this example in comparison with their commercially available counterpart. The ADCC evaluation was performed using ADCC Reporter Bioassay (Promega, GA1155) by following the manual. Briefly, the antibodies or glycoproteins to be examined were serial diluted with diluent (4% low IgG FBS in RPMI-1640) to 0.15 ng/mL. Target cells (Raji cells, BT-474, or Caco-2) were prepared into a suspension at a concentration of 5×10⁵ cells/mL in the same diluent. The target cell suspensions were then added to a 96-well U-bottom plate (25 microliters/well). The cells were then mixed with 25 uL serial diluted antibodies (or glycoproteins) and incubated at room temperature for 30 minutes to allow opsonization. For Rituximab, effector cells were added into the opsonized target cells at 7.5×10⁴/well. Alternatively, for Trastuzumab, Pertuzumab and Cetuximab, the opsonized target cells were added into wells containing adherent effector cells at 7.5×10⁴/well. The culture plates were incubated at 37 degree Celsius in a 5% CO₂ incubator for 16 hours.

After incubation, the culture plate was equilibrated to ambient temperature (22 to 25° C.) for 15 minutes. 75 μl of Bio-Glo™ Luciferase Assay Reagent was added to each well. The plate was incubated at ambient temperature for 15 minutes. The mixture was transferred to a white 96-well plate, and the reaction was detected by a plate reader (SpectraMax Mini Multi-Mode Microplate Reader).

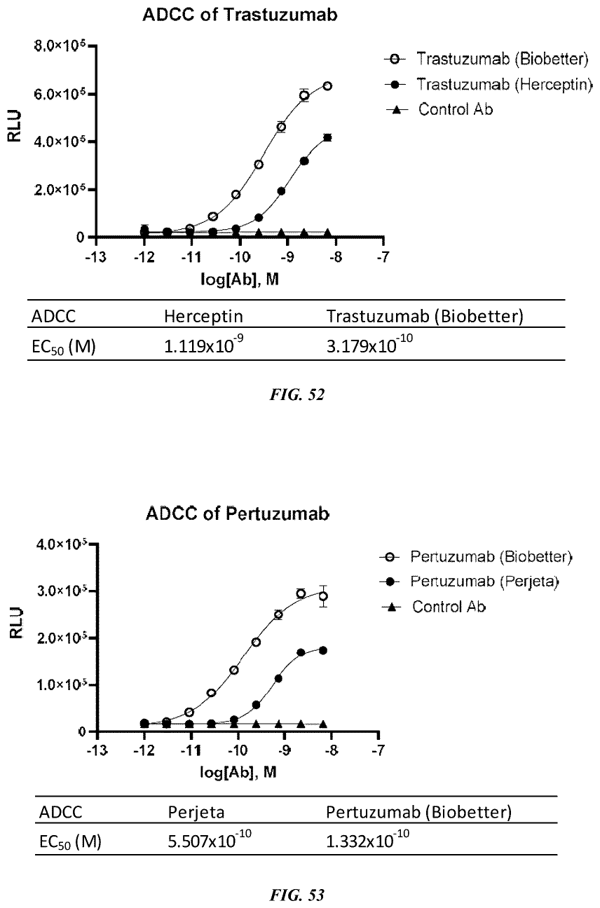

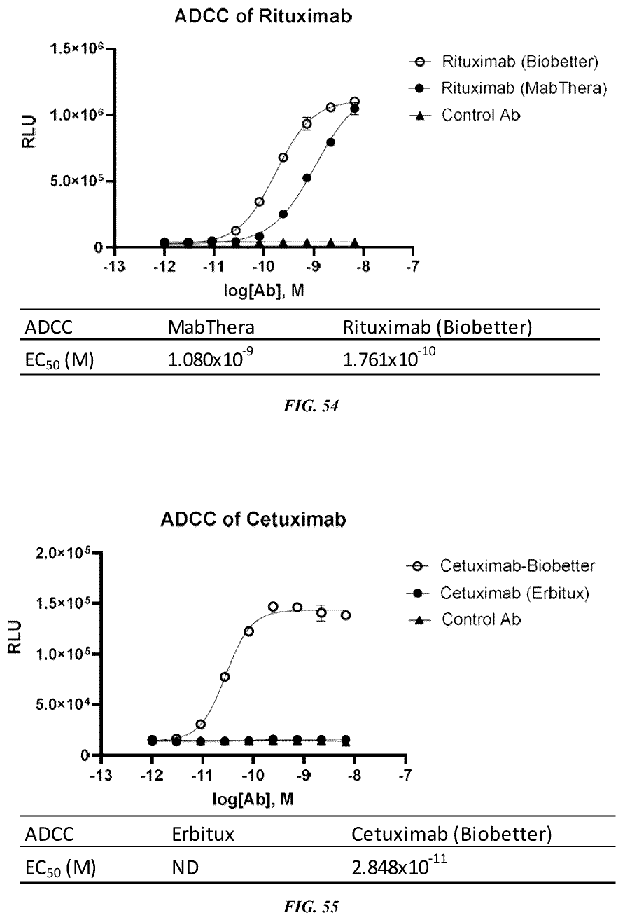

The result shows that all exemplary glycoprotein populations according to the present disclosure exhibited better ADCC effects compared to their commercially available counterparts. Specifically, the exemplary Trastuzumab antibody population of the present disclosure exhibited an EC₅₀ at around 3.179×10⁻¹⁰, which was lower than the commercial product Herceptin® (EC₅₀ 1.119×10⁻⁹) by almost 10 times (FIG. 52). The exemplary Rituximab antibody population also exhibited a 10-times incremental (1.080×10⁻⁹ vs. 1.76×10⁻¹⁰; FIG. 54). The improved ADCC effects of the Pertuzumab antibody population of the present disclosure was also noticeable with an EC₅₀ at around 1.332×10⁻¹⁰, compared to the commercial product Perjeta® at around 5.507×10⁻¹⁰ (FIG. 53). Lastly, the exemplary Cetuximab population's ADCC effect was way better than its commercial product Erbitux® as the two curves in FIG. 55 separated significantly. The data (concluded in Table 15 below) confirmed that the engineered glycoprotein population of the present disclosure can increase the ADCC effects of the glycoprotein through the desired glycan profiles.

TABLE 15

ADCC EC₅₀ (M)

| Glycoprotein | Brand Name | Biobetter | Commercial |
|---|---|---|---|
| Trastuzumab | Herceptin ® | 3.179 × 10⁻¹⁰ | 1.119 × 10⁻⁹ |
| Pertuzumab | Perjeta ® | 1.332 × 10⁻¹⁰ | 5.507 × 10⁻¹⁰ |
| Cetuximab | Erbitux ® | 2.848 × 10⁻¹¹ | ND |
| Rituximab | Mabthera ® | 1.761 × 10⁻¹⁰ | 1.08 × 10⁻⁹ |

Alternatively, the ADCC effect was evaluated and demonstrated using a scaffold-free single cell-derived 3D organoid culture platform (R³CE™, Acrocyte Therapeutics Inc.). Briefly, two organoids were respectively prepared using two breast cancer patient-derived cells as target cells. The two breast cancer patient-derived cells express different levels of HER2 (high HER2 expression is labeled as “HER2-3+”, and low HER2 expression is labeled as “HER2-1+”, the expression level is determined according to Sajjadi E, et al., Front Mol Biosci. 2023 Apr. 3). The Trastuzumab (Herceptin®) sample, according to an example of the present disclosure, was prepared as described in Example 5 (labeled as “BioBetter” in the data), and the commercially available Herceptin® product was obtained for comparison. The effector cells used in this experiment were NK-92 cells, and K562 cells were used as control target cells. The viability of the target organoids (patient-derived organoid, “PDO”) and effector cells was confirmed, and the cell numbers were determined before the experiment.

After cell counting, the target PDOs and effector immune cells were co-cultured in a round bottom plate at selected effector-to-target ratios (E: T=1:1, 3:1, or 0:1). Then, the BioBetter Herceptin® sample of the present disclosure and the commercial Herceptin® product were added to the culture respectively to determine the ADCC effect at indicated concentrations (0.08, 0.4, 2, or 10 μg/mL). After incubation for 18 to 20 hours, the cell viability of the cultures was assessed using the CellTiter-Glo® 3D Cell Viability Assay kit (Promega), and signals were detected using an ELISA plate reader. Based on the detected signals, the cytotoxicity of the BioBetter Herceptin® sample of the present disclosure and the commercial Herceptin® product was determined using the following formula: % Cytotoxicity=1−(((Target cell+ Effector cell)− Effector cell only)/(Target cell only))×100.

The results showed that the BioBetter Herceptin® sample and the commercial Herceptin® product exhibited similar binding abilities to the two organoids disregard the difference in HER2 expression level (Data not shown). This observation proves that both the BioBetter Herceptin® sample and the commercial Herceptin® product were able to recognize HER2, and the desired glycan profile according to the present disclosure did not substantially affect the antigen recognition ability of the antibodies.

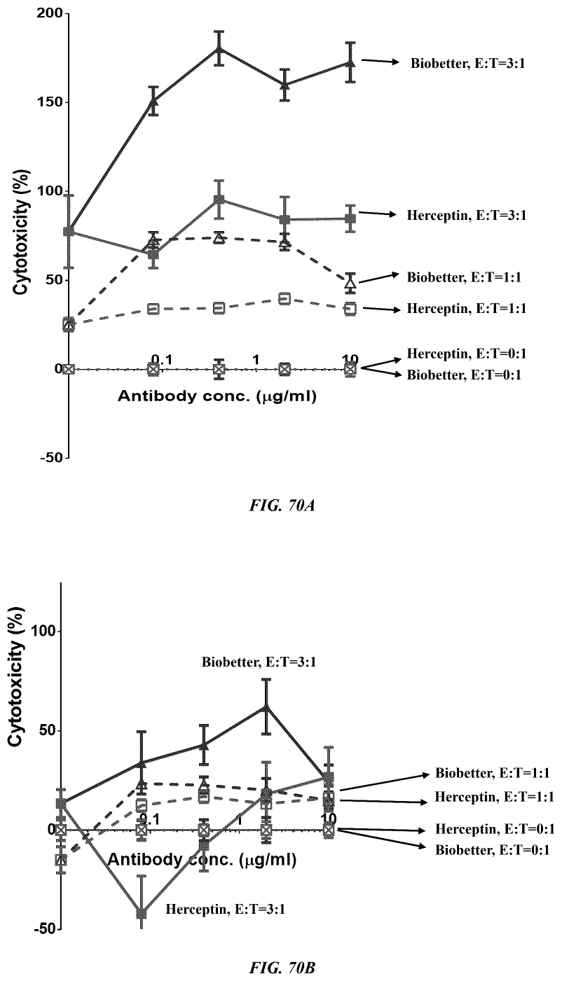

FIG. 69A and FIG. 69B (see also Table 16 below) showed the ADCC of the BioBetter Herceptin® sample and the commercial Herceptin® product against the HER3+ organoid and the HER 1+ organoid at a concentration of 0.08 μg/mL and a ET ratio of 1:1. The results demonstrate that the BioBetter exhibited significantly better ADCC than the commercial product in HER3+ organoid, suggesting the unique glycan profile of the present disclosure increased the ADCC effect (FIG. 69A). Moreover, the increasement was also noticeable and statistically significant in low HER expression organoid (FIG. 69B). Furthermore, FIG. 70A showed that the ADCC effects of the BioBetter Herceptin® sample were superior to that of the commercial Herceptin® product in all tested concentrations and E: T ratio. Besides, the observation in FIG. 70B demonstrated that the commercial Herceptin® product's ADCC effect against HER1+ organoid was less noticeable at low concentrations, while the BioBetter Herceptin® sample continued to show improved ADCC effect.

TABLE 16

| | | Ab Conc. | | | | | | | | |
|---|---|---|---|---|---|---|---|---|---|---|
| PDO | Antibody | (μg/ml) | E:T ratio | Cytotoxicity (%) | | | | | | Average |
| HER2-3+ | Herceptin | 0.08 | 1:1 | 34.5 | 34.8 | 34.1 | 26.7 | 45.3 | 28.2 | 33.9 |
| | Biobetter | | | 73.1 | 67.3 | 81.6 | 72.9 | 85.7 | 55.9 | 72.7 |
| HER2-1+ | Herceptin | | | 15.7 | 6.8 | 25.7 | 8.0 | 6.9 | 10.9 | 12.3 |
| | Biobetter | | | 20.3 | 30.8 | 19.7 | 19.5 | 21.2 | 28.9 | 23.4 |

### Example 12: Antibody-Dependent Cellular Phagocytosis Analysis

The glycoprotein populations of the present disclosure were evaluated for their efficacy in inducing antibody-dependent cellular phagocytosis (ADCP).

Cells. THP-1 cells were seeded in 10-cm Petri dishes and cultured in RPMI containing Phorbol 12-myristate 13-acetate (PMA; 50 ng/mL) and supplemented with 10% FBS for 72 hours to allow differentiation. Differentiated cells were then incubated in EDTA/PBS at 37 degrees Celsius for 15 minutes. The cultured cells were then transferred, centrifuged, and resuspended for staining with 1 uM Cell tracker DeepRed (ThermoFisher) in 10 mL 5 mM EDTA/PBS at 37 degrees Celsius for 15 minutes. After staining, cells were washed with PBS and collected using centrifugation. The collected cells were then resuspended in ADCP diluent (1% IgG-depleted FBS in RPMI-1647). Caco-2 cell line was used as the target cells for this experiment. The cells were maintained according to the manual and then collected using trypsin. The trypsin reaction was terminated in a cell culture medium with serum. The collected cells were then washed and resuspended in EDTA/PBS (10 mL, 5 mM) and then stained with 0.5 uM Calcein-AM at 37 degrees Celsius for 20 minutes. The stained cells were then collected using centrifugation and resuspended in EDTA/PBS (2 mL, 5 mM).

ADCP assay. Testing glycoprotein populations were prepared as described in Example 5. Particularly and as an example in this experiment, Cetuximab (Erbitux®) glycoproteins were produced by CHO-S cells or according to the present disclosure to obtain the desired heterogeneous glycan profile and were tested in comparison with a control antibody. The control antibody was an IgG1 antibody (PGT145), targeting HIV, which was used in this experiment as a negative control to establish a benchmark signal for phagocytosis determination. Serially diluted Cetuximab solutions were added to the Caco-2 cells (20 μL/well), and the incubation continued at 37 degrees Celsius for 1 hour. The antibodies (Cetuximab) were then removed by centrifugation, and the Caco-2 cells were resuspended with 100 μL ADCP diluent. After that, Caco-2 cells were co-cultured with the differentiated THP-1 cells at a ratio of 1:1 at 37 degrees Celsius for 2.5 hours. All cells were then washed with FACS buffer and collected by centrifugation for phagocytosis analysis.

The phagocytosis analysis was performed using flow cytometry where cells were stained with both FITC and Alexa-647. FITC was used to stain the target cells (Caco-2 cells), and Alexa-647 was for THP-1 cells. Therefore, Caco-2 cells that were engulfed by THP-1 cells (phagocytosis) would show positive signals on both FITC and Alexa-647. Based on the signals detected, ADCP efficacy was calculated by first determining the phagocytosis percentages respectively incurred by the control antibody PGT145, antibodies produced by CHO-S cells (labeled as “CHOS”), and antibodies produced according to the present disclosure (labeled as “GO”). The percentage of PGT145 was used as a benchmark to normalize the CHOS and GO's percentages to obtain respective ADCP values. Nonlinear regression of the ADCP values was then performed using a log (agonist) against a response model with a variable slope (four parameters). The span of the nonlinear fit was defined as the ADCP efficacy.

Alternatively, exemplary glycoprotein populations of Adalimumab (Humira®). Adalimumab-atto (Amjevita®), Bevacizumab (Avastin®), Alemtuzumab (Campath®), Ipilimumab (Yervoy®), Avelumab (Bavencio®), Durvalumab (IMFINZI®), Pembrolizumab (Keytruda®), Nivolumab (Opdivo®), Etanercept (Enbrel®), Trastuzumab (Herceptin®), Pertuzumab (Perjeta®), Cetuximab (Erbitux®), Rituximab (Rituxan®), Rituximab-atto (Truxima®), Obinutuzumab (Gazyva®), Infliximab (Remicade®), Ofatumumab (Arzerra®), Golimumab (Simponi®), Atezolizumab (Tecentriq®), Ocrelizumab (OCREVUS®), Gazyvaro Infliximab (Remicade®), Zanidatamab (Ziihera®), Daratumumab (Darzalex®), or Trastuzumab emtansine (Kadcyla®) were prepared as described in Example 5. The exemplary glycoprotein populations are serially diluted to different concentrations for the evaluation. Serially diluted glycoprotein populations are added respectively to Caco-2 cell cultures (20 μL/well), and the Caco-2 cells are then co-cultured with differentiated THP-1 cells as described above. The cells in the co-culture are then collected and evaluated using flow cytometry as described above.

Results. The results show that both the Cetuximab glycoproteins of the present disclosure (labeled as “GO”) and the Cetuximab glycoproteins produced by CHO-S cells (labeled as “CHOS”) exhibited phagocytosis in a dose-dependent manner while the control antibody's phagocytosis percentage was steady at around 12% throughout the serially diluted concentrations tested in the experiment. Moreover, GO glycoproteins showed a significantly higher engulfing percentage than CHOS glycoproteins with an ADCP efficacy of 3.1% compared to 3.1% of CHOS glycoprotein's ADCP efficacy. The difference was even more noticeable in higher concentrations of glycoproteins. Based on this data, the EC50 of GO glycoproteins was about 5.56-10M, which was much better than that of the CHOS glycoprotein, which was about 9.06-10M. Together, it was proved that the glycoprotein population of the present disclosure, which was modified to carry a desired heterogeneous glycan profile, exhibited superior ADCP efficacy.

### Exemplary Embodiments

Embodiment 1: An engineered glycoprotein population with a heterogeneous glycan profile, comprising a plurality of glycoproteins, wherein the heterogenous glycan profile comprises at least 20%, 25%, 30%, 35%, 40%, 45%, 50%, 55%, 60%, 65%, 70%, 75%, 80%, 85%, 86%, 88%, 90%, or 95% of sialylated complex type (SCT) glycans, and the SCT glycans comprises two or more types of sialylated glycans.

Embodiment 2: The engineered glycoprotein population of embodiment 1, wherein the heterogeneous glycan profile comprises no more than about 88% of SCT glycans.

Embodiment 3: The engineered glycoprotein population of embodiment 1 or embodiment 2, wherein the heterogenous glycan profile comprises about 20% to 95%, 20% to 90%, 20% to 88%, 20% to 86%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 30% to 95%, 30% to 90%, 30% to 88%, 30% to 86%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 40% to 95%, 40% to 90%, 40% to 88%, 40% to 86%, 40% to 85%, 40% to 80%, 40% to 75%, 40% to 70%, 40% to 65%, 40% to 60%, 40% to 55%, 40% to 50%, 40% to 45%, 50% to 95%, 50% to 90%, 50% to 88%, 50% to 86%, 50% to 85%, 50% to 80%, 50% to 75%, 50% to 70%, 50% to 65%, 50% to 60%, 50% to 55%, 60% to 95%, 60% to 90%, 60% to 88%, 60% to 86%, 60% to 85%, 60% to 80%, 60% to 75%, 60% to 70%, or 60% to 65% of SCT glycans.

Embodiment 4: The engineered glycoprotein population of any one of embodiments 1 to 3, wherein the sialylated glycans comprises a terminal sialic acid, optionally a 7-fluoro sialic acid.

Embodiment 5: The engineered glycoprotein population of embodiment 4, wherein the terminal sialic acid is connected to a preceding sugar residue of the glycans via a a2,6 linkage or a2,3 linkage; optionally, wherein the SCT glycans of the glycan profile can be monoantennary, biantennary, or triantennary, further optionally, wherein the SCT glycans comprise biantennary glycans, which comprise at least 5%, 10%, 15%, 20%, 25%, 30%, 35%, 40%, 45%, 50%, 55%, 60%, 65%, 70%, 75%, 80%, 85%, 90%, 95%, or 100% of the glycans of the engineered glycoprotein population's glycan profile, or any range defined by the foregoing endpoints, such as 5 to 100%, 5 to 95%, 5 to 90%, 5 to 85%, 5 to 80%, 5 to 70%, 5 to 60%, 5 to 50%, 5 to 40%, 5 to 30%, 5 to 20%, 5 to 10%, 10 to 100%, 10 to 95%, 10 to 90%, 10 to 85%, 10 to 80%, 10 to 70%, 10 to 60%, 10 to 50%, 10 to 40%, 10 to 30%, 10 to 20%, 20 to 100%, 20 to 90%, 20 to 80%, 20 to 70%, 20 to 60%, 20 to 50%, 20 to 40%, 20 to 30%, 50 to 100%, 50 to 95%, 50 to 90%, 50 to 85%, 50 to 80%, 50 to 70%, or 50 to 60%, including or excluding any foregoing numbers; or the SCT glycans comprise triantennary glycans, which comprise at least 0.1%, 0.5%, 1%, 2%, 3%, 4%, 5%, 6%, 7%, 8%, 9%, 10%, 11%, 12%, 13%, 14%, or 15% of the glycans of the engineered glycoprotein population's glycan profile, or any range defined by the foregoing endpoints, such as 0.1 to 15%, 0.1 to 14%, 0.1 to 12%, 0.1 to 10%, 0.1 to 8%, 0.1 to 5%, 0.1 to 3%, 0.1 to 1%, 1 to 15%, 1 to 14%, 1 to 12%, 1 to 10%, 1 to 8%, 1 to 5%, 1 to 3%, 3 to 15%, 3 to 14%, 3 to 12%, 3 to 10%, 3 to 8%, 3 to 5%, 5 to 15%, 5 to 14%, 5 to 12%, 5 to 10%, 5 to 8%, including or excluding any foregoing numbers.

Embodiment 6: The engineered glycoprotein population of any one of embodiments 1 to 5, wherein the heterogenous glycan profile comprises (a) SiaGal2GlcNAc2Man3GlcNAc2 or SiaGal2GlcNAc2Man3GlcNAc2Fuc and (b) Sia2Gal2GlcNAc2Man3GlcNAc2 or Sia2Gal2GlcNAc2Man3GlcNAc2Fuc.

Embodiment 7: The engineered glycoprotein population of embodiment 6, wherein the heterogenous glycan profile further comprises (c) SiaGalGlcNAcMan5GlcNAc2 or SiaGalGlcNAcMan5GlcNAc2Fuc, (d) SiaGalGlcNAc2Man3GlcNAc2 or SiaGalGlcNAc2Man3GlcNAc2Fuc, (e) SiaGalGlcNAcMan4GlcNAc2 or SiaGalGlcNAcMan4GlcNAc2Fuc, (f) SiaHexGal2GlcNAc2Man3GlcNAc2 or SiaHexGal2GlcNAc2Man3GlcNAc2Fuc, (g) SiaGalGlcNAcMan3GlcNAc2 or SiaGalGlcNAcMan3GlcNAc2Fuc, (h) SiaGal3GlcNAc3Man3GlcNAc2 or SiaGal3GlcNAc3Man3GlcNAc2Fuc, or a mixture thereof.

Embodiment 8: The engineered glycoprotein population of embodiment 6, wherein the heterogenous glycan profile comprises (c) SiaGalGlcNAcMan5GlcNAc2 or SiaGalGlcNAcMan5GlcNAc2Fuc, (d) SiaGalGlcNAc2Man3GlcNAc2 or SiaGalGlcNAc2Man3GlcNAc2Fuc, (e) SiaGalGlcNAcMan4GlcNAc2 or SiaGalGlcNAcMan4GlcNAc2Fuc, (f) SiaHexGal2GlcNAc2Man3GlcNAc2 or SiaHexGal2GlcNAc2Man3GlcNAc2Fuc, (g) SiaGalGlcNAcMan3GlcNAc2 or SiaGalGlcNAcMan3GlcNAc2Fuc, and (h) SiaGal3GlcNAc3Man3GlcNAc2 or SiaGal3GlcNAc3Man3GlcNAc2Fuc.

Embodiment 9: The engineered glycoprotein population of any one of embodiments 1 to 8, wherein the SCT glycans comprise a first glycan and a second glycan, and each or any of the first SCT glycan and the second SCT glycan comprises no more than 50%, 45%, 40%, 35%, 30%, 25%, 20%, 15%, 10%, 9%, 8%, 7%, 6%, 5%, 4%, 3.5%, 3%, 2.5%, 2%, 1.5%, or 1% of the heterogenous glycan profile.

Embodiment 10: The engineered glycoprotein population of any one of embodiments 1 to 9, wherein the SCT glycans do not have a core fucose.

Embodiment 11: The engineered glycoprotein population of any one of embodiments 1 to 10, wherein the glycoprotein comprises a target-binding site and a glycosylation site.

Embodiment 12: The engineered glycoprotein population of any one of embodiments 1 to 11, wherein the glycoprotein comprises a fragment crystallizable (Fc) region, which comprises the glycosylation site.

Embodiment 13: The engineered glycoprotein population of embodiment 11 or embodiment 12, wherein the target-binding site is an antigen-binding site, and wherein the glycoprotein comprises a first peptide and a second peptide, and the first peptide and the second peptide form the antigen-binding site.

Embodiment 14: The engineered glycoprotein population of embodiment 13, wherein the glycoprotein is an antibody or antigen-binding fragment thereof.

Embodiment 15: The engineered glycoprotein population of embodiment 14, wherein the antibody is a monoclonal antibody.

Embodiment 16: The engineered glycoprotein population of embodiment 14 or embodiment 15, wherein the antibody is a bi-specific antibody, a tri-specific antibody, or a multi-specific antibody.

Embodiment 17: The engineered glycoprotein population of any one of embodiments 14 to 16, wherein the sialylated glycan is conjugated to the antibody at a N297 site thereof.

Embodiment 18: The engineered glycoprotein population of any one of embodiments 11 to 17, wherein the target is tumor necrosis factor-alpha (TNFα), Vascular Endothelial Growth Factor (VEGF), Cytotoxic T-Lymphocyte Associated protein 4 (CTLA-4), PD-L1, PD-1, Human Epidermal Growth Factor Receptor 2 (HER2), Epidermal Growth Factor Receptor (EGFR), CD38, CD52, or CD20.

Embodiment 19: The engineered glycoprotein population of any one of embodiments 1 to 18, wherein the glycoprotein is a therapeutic protein.

Embodiment 20: The engineered glycoprotein population of any one of embodiments 1 to 19, wherein the glycoprotein is Adalimumab (Humira®), Adalimumab-atto (Amjevita®), Bevacizumab (Avastin®), Alemtuzumab (Campath®), Ipilimumab (Yervoy®), Avelumab (Bavencio®), Durvalumab (IMFINZI®), Pembrolizumab (Keytruda®), Nivolumab (Opdivo®), Etanercept (Enbrel®), Trastuzumab (Herceptin®), Pertuzumab (Perjeta®), Cetuximab (Erbitux®), Rituximab (Rituxan®), Rituximab-atto (Truxima®), Obinutuzumab (Gazyva®), Infliximab (Remicade®), Ofatumumab (Arzerra®), Golimumab (Simponi®), Atezolizumab (Tecentriq®), Ocrelizumab (OCREVUS®), Gazyvaro Infliximab (Remicade®), Zanidatamab (Ziihera®), Daratumumab (Darzalex®), or Trastuzumab emtansine (Kadcyla®); optionally, provided that when the glycoprotein is Adalimumab (Humira®), Bevacizumab (Avastin®), Ipilimumab (Yervoy®), Avelumab (Bavencio®), Durvalumab (IMFINZI®), Pembrolizumab (Keytruda®), Nivolumab (Opdivo®), or Etanercept (Enbrel®), the heterogenous glycan profile comprises core fucose; and/or when the glycoprotein is Trastuzumab (Herceptin®), Pertuzumab (Perjeta®), Cetuximab (Erbitux®), Rituximab (Rituxan®), or Obinutuzumab (Gazyva®), the heterogenous glycan profile comprises no core fucose.

Embodiment 21: The engineered glycoprotein population of any one of embodiments 1 to 20, wherein the glycoprotein comprises a heavy chain variable complementarity-determining region 1 (VHCDR1) comprising an amino acid sequence as set forth in SEQ ID NO: 01, a VHCDR2 comprising an amino acid sequence as set forth in SEQ ID NO: 02, a VHCDR3 comprising an amino acid sequence as set forth in SEQ ID NO: 03, a light chain variable complementarity-determining region 1 (VLCDR1) comprising an amino acid sequence as set forth in SEQ ID NO: 06, a VLCDR2 comprising an amino acid sequence as set forth in SEQ ID NO: 07, and a VLCDR3 comprising an amino acid sequence as set forth in SEQ ID NO: 08.

Embodiment 22: The engineered glycoprotein population of any one of embodiments 1 to 21, wherein the glycoprotein comprises a heavy chain variable region comprising an amino acid sequence having at least 70%, 80%, 90%, 95% or 100% identity as the amino acid sequence set forth in SEQ ID NO: 04.

Embodiment 23: The engineered glycoprotein population of any one of embodiments 1 to 22, wherein the glycoprotein comprises a light chain variable region comprising an amino acid sequence having at least 70%, 80%, 90%, 95% or 100% identity as the amino acid sequence set forth in SEQ ID NO: 09.

Embodiment 24: The engineered glycoprotein population of any one of embodiments 1 to 23, wherein the glycoprotein comprises a heavy chain comprising an amino acid sequence having at least 70%, 80%, 90%, 95% or 100% identity as the amino acid sequence set forth in SEQ ID NO: 05.

Embodiment 25: The engineered glycoprotein population of any one of embodiments 1 to 24, wherein the glycoprotein comprises a light chain comprising an amino acid sequence having at least 70%, 80%, 90%, 95% or 100% identity as the amino acid sequence set forth in SEQ ID NO: 10.

Embodiment 26: The engineered glycoprotein population of any one of embodiments 1 to 20, wherein the glycoprotein comprises a heavy chain variable complementarity-determining region 1 (VHCDR1) comprising an amino acid sequence as set forth in SEQ ID NO: 11, a VHCDR2 comprising an amino acid sequence as set forth in SEQ ID NO: 12, a VHCDR3 comprising an amino acid sequence as set forth in SEQ ID NO: 13, a light chain variable complementarity-determining region 1 (VLCDR1) comprising an amino acid sequence as set forth in SEQ ID NO: 16, a VLCDR2 comprising an amino acid sequence as set forth in SEQ ID NO: 17, and a VLCDR3 comprising an amino acid sequence as set forth in SEQ ID NO: 18.

Embodiment 27: The engineered glycoprotein population of any one of embodiments 1 to 20 and 26, wherein the glycoprotein comprises a heavy chain variable region comprising an amino acid sequence having at least 70%, 80%, 90%, 95% or 100% identity as the amino acid sequence set forth in SEQ ID NO: 14.

Embodiment 28: The engineered glycoprotein population of any one of embodiments 1 to 20, 26, and 27, wherein the glycoprotein comprises a light chain variable region comprising an amino acid sequence having at least 70%, 80%, 90%, 95% or 100% identity as the amino acid sequence set forth in SEQ ID NO: 19.

Embodiment 29: The engineered glycoprotein population of any one of embodiments 1 to 20 and 26 to 28, wherein the glycoprotein comprises a heavy chain comprising an amino acid sequence having at least 70%, 80%, 90%, 95% or 100% identity as the amino acid sequence set forth in SEQ ID NO: 15.

Embodiment 30: The engineered glycoprotein population of any one of embodiments 1 to 20 and 26 to 29, wherein the glycoprotein comprises a light chain comprising an amino acid sequence having at least 70%, 80%, 90%, 95% or 100% identity as the amino acid sequence set forth in SEQ ID NO: 20.

Embodiment 31: The engineered glycoprotein population of any one of embodiments 1 to 20, wherein the glycoprotein comprises a heavy chain variable complementarity-determining region 1 (VHCDR1) comprising an amino acid sequence as set forth in SEQ ID NO: 21, a VHCDR2 comprising an amino acid sequence as set forth in SEQ ID NO: 22, a VHCDR3 comprising an amino acid sequence as set forth in SEQ ID NO: 23, a light chain variable complementarity-determining region 1 (VLCDR1) comprising an amino acid sequence as set forth in SEQ ID NO: 26, a VLCDR2 comprising an amino acid sequence as set forth in SEQ ID NO: 27, and a VLCDR3 comprising an amino acid sequence as set forth in SEQ ID NO: 28.

Embodiment 32: The engineered glycoprotein population of any one of embodiments 1 to 20 and 31, wherein the glycoprotein comprises a heavy chain variable region comprising an amino acid sequence having at least 70%, 80%, 90%, 95% or 100% identity as the amino acid sequence set forth in SEQ ID NO: 24.

Embodiment 33: The engineered glycoprotein population of any one of embodiments 1 to 20, 31, and 32, wherein the glycoprotein comprises a light chain variable region comprising an amino acid sequence having at least 70%, 80%, 90%, 95% or 100% identity as the amino acid sequence set forth in SEQ ID NO: 29.

Embodiment 34: The engineered glycoprotein population of any one of embodiments 1 to 20 and 31 to 33, wherein the glycoprotein comprises a heavy chain comprising an amino acid sequence having at least 70%, 80%, 90%, 95% or 100% identity as the amino acid sequence set forth in SEQ ID NO: 25.

Embodiment 35: The engineered glycoprotein population of any one of embodiments 1 to 20 and 31 to 34, wherein the glycoprotein comprises a light chain comprising an amino acid sequence having at least 70%, 80%, 90%, 95% or 100% identity as the amino acid sequence set forth in SEQ ID NO: 30.

Embodiment 36: The engineered glycoprotein population of any one of embodiments 1 to 20, wherein the glycoprotein comprises a heavy chain variable complementarity-determining region 1 (VHCDR1) comprising an amino acid sequence as set forth in SEQ ID NO: 31, a VHCDR2 comprising an amino acid sequence as set forth in SEQ ID NO: 32, a VHCDR3 comprising an amino acid sequence as set forth in SEQ ID NO: 33, a light chain variable complementarity-determining region 1 (VLCDR1) comprising an amino acid sequence as set forth in SEQ ID NO: 36, a VLCDR2 comprising an amino acid sequence as set forth in SEQ ID NO: 37, and a VLCDR3 comprising an amino acid sequence as set forth in SEQ ID NO: 38.

Embodiment 37: The engineered glycoprotein population of any one of embodiments 1 to 20 and 36, wherein the glycoprotein comprises a heavy chain variable region comprising an amino acid sequence having at least 70%, 80%, 90%, 95% or 100% identity as the amino acid sequence set forth in SEQ ID NO: 34.

Embodiment 38: The engineered glycoprotein population of any one of embodiments 1 to 20, 36, and 37, wherein the glycoprotein comprises a light chain variable region comprising an amino acid sequence having at least 70%, 80%, 90%, 95% or 100% identity as the amino acid sequence set forth in SEQ ID NO: 39.

Embodiment 39: The engineered glycoprotein population of any one of embodiments 1 to 20 and 36 to 38, wherein the glycoprotein comprises a heavy chain comprising an amino acid sequence having at least 70%, 80%, 90%, 95% or 100% identity as the amino acid sequence set forth in SEQ ID NO: 35.

Embodiment 40: The engineered glycoprotein population of any one of embodiments 1 to 20 and 36 to 39, wherein the glycoprotein comprises a light chain comprising an amino acid sequence having at least 70%, 80%, 90%, 95% or 100% identity as the amino acid sequence set forth in SEQ ID NO: 40.

Embodiment 41: The engineered glycoprotein population of any one of embodiments 1 to 20, wherein the glycoprotein comprises a heavy chain variable complementarity-determining region 1 (VHCDR1) comprising an amino acid sequence as set forth in SEQ ID NO: 41, a VHCDR2 comprising an amino acid sequence as set forth in SEQ ID NO: 42, a VHCDR3 comprising an amino acid sequence as set forth in SEQ ID NO: 43, a light chain variable complementarity-determining region 1 (VLCDR1) comprising an amino acid sequence as set forth in SEQ ID NO: 46, a VLCDR2 comprising an amino acid sequence as set forth in SEQ ID NO: 47, and a VLCDR3 comprising an amino acid sequence as set forth in SEQ ID NO: 48.

Embodiment 42: The engineered glycoprotein population of any one of embodiments 1 to 20 and 41, wherein the glycoprotein comprises a heavy chain variable region comprising an amino acid sequence having at least 70%, 80%, 90%, 95% or 100% identity as the amino acid sequence set forth in SEQ ID NO: 44.

Embodiment 43: The engineered glycoprotein population of any one of embodiments 1 to 20, 41, and 42, wherein the glycoprotein comprises a light chain variable region comprising an amino acid sequence having at least 70%, 80%, 90%, 95% or 100% identity as the amino acid sequence set forth in SEQ ID NO: 49.

Embodiment 44: The engineered glycoprotein population of any one of embodiments 1 to 20 and 41 to 43, wherein the glycoprotein comprises a heavy chain comprising an amino acid sequence having at least 70%, 80%, 90%, 95% or 100% identity as the amino acid sequence set forth in SEQ ID NO: 45.

Embodiment 45: The engineered glycoprotein population of any one of embodiments 1 to 20 and 41 to 44, wherein the glycoprotein comprises a light chain comprising an amino acid sequence having at least 70%, 80%, 90%, 95% or 100% identity as the amino acid sequence set forth in SEQ ID NO: 50.

Embodiment 46: The engineered glycoprotein population of any one of embodiments 1 to 20, wherein the glycoprotein comprises a heavy chain variable complementarity-determining region 1 (VHCDR1) comprising an amino acid sequence as set forth in SEQ ID NO: 51, a VHCDR2 comprising an amino acid sequence as set forth in SEQ ID NO: 52, a VHCDR3 comprising an amino acid sequence as set forth in SEQ ID NO: 53, a light chain variable complementarity-determining region 1 (VLCDR1) comprising an amino acid sequence as set forth in SEQ ID NO: 56, a VLCDR2 comprising an amino acid sequence as set forth in SEQ ID NO: 57, and a VLCDR3 comprising an amino acid sequence as set forth in SEQ ID NO: 58.

Embodiment 47: The engineered glycoprotein population of any one of embodiments 1 to 20 and 46, wherein the glycoprotein comprises a heavy chain variable region comprising an amino acid sequence having at least 70%, 80%, 90%, 95% or 100% identity as the amino acid sequence set forth in SEQ ID NO: 54.

Embodiment 48: The engineered glycoprotein population of any one of embodiments 1 to 20, 46, and 47, wherein the glycoprotein comprises a light chain variable region comprising an amino acid sequence having at least 70%, 80%, 90%, 95% or 100% identity as the amino acid sequence set forth in SEQ ID NO: 59.

Embodiment 49: The engineered glycoprotein population of any one of embodiments 1 to 20 and 46 to 48, wherein the glycoprotein comprises a heavy chain comprising an amino acid sequence having at least 70%, 80%, 90%, 95% or 100% identity as the amino acid sequence set forth in SEQ ID NO: 55.

Embodiment 50: The engineered glycoprotein population of any one of embodiments 1 to 20 and 46 to 49, wherein the glycoprotein comprises a light chain comprising an amino acid sequence having at least 70%, 80%, 90%, 95% or 100% identity as the amino acid sequence set forth in SEQ ID NO: 60.

Embodiment 51: The engineered glycoprotein population of any one of embodiments 1 to 20, wherein the glycoprotein comprises a heavy chain variable complementarity-determining region 1 (VHCDR1) comprising an amino acid sequence as set forth in SEQ ID NO: 61, a VHCDR2 comprising an amino acid sequence as set forth in SEQ ID NO: 62, a VHCDR3 comprising an amino acid sequence as set forth in SEQ ID NO: 63, a light chain variable complementarity-determining region 1 (VLCDR1) comprising an amino acid sequence as set forth in SEQ ID NO: 66, a VLCDR2 comprising an amino acid sequence as set forth in SEQ ID NO: 67, and a VLCDR3 comprising an amino acid sequence as set forth in SEQ ID NO: 68.

Embodiment 52: The engineered glycoprotein population of any one of embodiments 1 to 20 and 51, wherein the glycoprotein comprises a heavy chain variable region comprising an amino acid sequence having at least 70%, 80%, 90%, 95% or 100% identity as the amino acid sequence set forth in SEQ ID NO: 64.

Embodiment 53: The engineered glycoprotein population of any one of embodiments 1 to 20, 51, and 52, wherein the glycoprotein comprises a light chain variable region comprising an amino acid sequence having at least 70%, 80%, 90%, 95% or 100% identity as the amino acid sequence set forth in SEQ ID NO: 69.

Embodiment 54: The engineered glycoprotein population of any one of embodiments 1 to 20 and 51 to 53, wherein the glycoprotein comprises a heavy chain comprising an amino acid sequence having at least 70%, 80%, 90%, 95% or 100% identity as the amino acid sequence set forth in SEQ ID NO: 65.

Embodiment 55: The engineered glycoprotein population of any one of embodiments 1 to 20 and 51 to 54, wherein the glycoprotein comprises a light chain comprising an amino acid sequence having at least 70%, 80%, 90%, 95% or 100% identity as the amino acid sequence set forth in SEQ ID NO: 70.

Embodiment 56: The engineered glycoprotein population of any one of embodiments 1 to 20, wherein the glycoprotein comprises an amino acid sequence having at least 70%, 80%, 90%, 95% or 100% identity as the amino acid sequence set forth in SEQ ID NO: 71 or SEQ ID NO: 72.

Embodiment 57: The engineered glycoprotein population of any one of embodiments 1 to 20, wherein the glycoprotein comprises a heavy chain variable complementarity-determining region 1 (VHCDR1) comprising an amino acid sequence as set forth in SEQ ID NO: 73, a VHCDR2 comprising an amino acid sequence as set forth in SEQ ID NO: 74, a VHCDR3 comprising an amino acid sequence as set forth in SEQ ID NO: 75, a light chain variable complementarity-determining region 1 (VLCDR1) comprising an amino acid sequence as set forth in SEQ ID NO: 78, a VLCDR2 comprising an amino acid sequence as set forth in SEQ ID NO: 79, and a VLCDR3 comprising an amino acid sequence as set forth in SEQ ID NO: 80.

Embodiment 58: The engineered glycoprotein population of any one of embodiments 1 to 20 and 57, wherein the glycoprotein comprises a heavy chain variable region comprising an amino acid sequence having at least 70%, 80%, 90%, 95% or 100% identity as the amino acid sequence set forth in SEQ ID NO: 76.

Embodiment 59: The engineered glycoprotein population of any one of embodiments 1 to 20, 57, and 58, wherein the glycoprotein comprises a light chain variable region comprising an amino acid sequence having at least 70%, 80%, 90%, 95% or 100% identity as the amino acid sequence set forth in SEQ ID NO: 81.

Embodiment 60: The engineered glycoprotein population of any one of embodiments 1 to 20 and 57 to 59, wherein the glycoprotein comprises a heavy chain comprising an amino acid sequence having at least 70%, 80%, 90%, 95% or 100% identity as the amino acid sequence set forth in SEQ ID NO: 77.

Embodiment 61: The engineered glycoprotein population of any one of embodiments 1 to 20 and 57 to 60, wherein the glycoprotein comprises a light chain comprising an amino acid sequence having at least 70%, 80%, 90%, 95% or 100% identity as the amino acid sequence set forth in SEQ ID NO: 82.

Embodiment 62: The engineered glycoprotein population of any one of embodiments 1 to 20, wherein the glycoprotein comprises a heavy chain variable complementarity-determining region 1 (VHCDR1) comprising an amino acid sequence as set forth in SEQ ID NO: 83, a VHCDR2 comprising an amino acid sequence as set forth in SEQ ID NO: 84, a VHCDR3 comprising an amino acid sequence as set forth in SEQ ID NO: 85, a light chain variable complementarity-determining region 1 (VLCDR1) comprising an amino acid sequence as set forth in SEQ ID NO: 88, a VLCDR2 comprising an amino acid sequence as set forth in SEQ ID NO: 89, and a VLCDR3 comprising an amino acid sequence as set forth in SEQ ID NO: 90.

Embodiment 63: The engineered glycoprotein population of any one of embodiments 1 to 20 and 62, wherein the glycoprotein comprises a heavy chain variable region comprising an amino acid sequence having at least 70%, 80%, 90%, 95% or 100% identity as the amino acid sequence set forth in SEQ ID NO: 86.

Embodiment 64: The engineered glycoprotein population of any one of embodiments 1 to 20, 62, and 63, wherein the glycoprotein comprises a light chain variable region comprising an amino acid sequence having at least 70%, 80%, 90%, 95% or 100% identity as the amino acid sequence set forth in SEQ ID NO: 91.

Embodiment 65: The engineered glycoprotein population of any one of embodiments 1 to 20 and 62 to 64, wherein the glycoprotein comprises a heavy chain comprising an amino acid sequence having at least 70%, 80%, 90%, 95% or 100% identity as the amino acid sequence set forth in SEQ ID NO: 87.

Embodiment 66: The engineered glycoprotein population of any one of embodiments 1 to 20 and 62 to 65, wherein the glycoprotein comprises a light chain comprising an amino acid sequence having at least 70%, 80%, 90%, 95% or 100% identity as the amino acid sequence set forth in SEQ ID NO: 92.

Embodiment 67: The engineered glycoprotein population of any one of embodiments 1 to 20, wherein the glycoprotein comprises a heavy chain variable complementarity-determining region 1 (VHCDR1) comprising an amino acid sequence as set forth in SEQ ID NO: 93, a VHCDR2 comprising an amino acid sequence as set forth in SEQ ID NO: 94, a VHCDR3 comprising an amino acid sequence as set forth in SEQ ID NO: 95, a light chain variable complementarity-determining region 1 (VLCDR1) comprising an amino acid sequence as set forth in SEQ ID NO: 98, a VLCDR2 comprising an amino acid sequence as set forth in SEQ ID NO: 99, and a VLCDR3 comprising an amino acid sequence as set forth in SEQ ID NO: 100.

Embodiment 68: The engineered glycoprotein population of any one of embodiments 1 to 20 and 67, wherein the glycoprotein comprises a heavy chain variable region comprising an amino acid sequence having at least 70%, 80%, 90%, 95% or 100% identity as the amino acid sequence set forth in SEQ ID NO: 96.

Embodiment 69: The engineered glycoprotein population of any one of embodiments 1 to 20, 67, and 68, wherein the glycoprotein comprises a light chain variable region comprising an amino acid sequence having at least 70%, 80%, 90%, 95% or 100% identity as the amino acid sequence set forth in SEQ ID NO: 101.

Embodiment 70: The engineered glycoprotein population of any one of embodiments 1 to 20 and 67 to 69, wherein the glycoprotein comprises a heavy chain comprising an amino acid sequence having at least 70%, 80%, 90%, 95% or 100% identity as the amino acid sequence set forth in SEQ ID NO: 97.

Embodiment 71: The engineered glycoprotein population of any one of embodiments 1 to 20 and 67 to 70, wherein the glycoprotein comprises a light chain comprising an amino acid sequence having at least 70%, 80%, 90%, 95% or 100% identity as the amino acid sequence set forth in SEQ ID NO: 102.

Embodiment 72: The engineered glycoprotein population of any one of embodiments 1 to 20, wherein the glycoprotein comprises a heavy chain variable complementarity-determining region 1 (VHCDR1) comprising an amino acid sequence as set forth in SEQ ID NO: 103, a VHCDR2 comprising an amino acid sequence as set forth in SEQ ID NO: 104, a VHCDR3 comprising an amino acid sequence as set forth in SEQ ID NO: 105, a light chain variable complementarity-determining region 1 (VLCDR1) comprising an amino acid sequence as set forth in SEQ ID NO: 108, a VLCDR2 comprising an amino acid sequence as set forth in SEQ ID NO: 109, and a VLCDR3 comprising an amino acid sequence as set forth in SEQ ID NO: 110.

Embodiment 73: The engineered glycoprotein population of any one of embodiments 1 to 20 and 72, wherein the glycoprotein comprises a heavy chain variable region comprising an amino acid sequence having at least 70%, 80%, 90%, 95% or 100% identity as the amino acid sequence set forth in SEQ ID NO: 106.

Embodiment 74: The engineered glycoprotein population of any one of embodiments 1 to 20, 72, and 73, wherein the glycoprotein comprises a light chain variable region comprising an amino acid sequence having at least 70%, 80%, 90%, 95% or 100% identity as the amino acid sequence set forth in SEQ ID NO: 111.

Embodiment 75: The engineered glycoprotein population of any one of embodiments 1 to 20 and 72 to 74, wherein the glycoprotein comprises a heavy chain comprising an amino acid sequence having at least 70%, 80%, 90%, 95% or 100% identity as the amino acid sequence set forth in SEQ ID NO: 107.

Embodiment 76: The engineered glycoprotein population of any one of embodiments 1 to 20 and 72 to 75, wherein the glycoprotein comprises a light chain comprising an amino acid sequence having at least 70%, 80%, 90%, 95% or 100% identity as the amino acid sequence set forth in SEQ ID NO: 112.

Embodiment 77: The engineered glycoprotein population of any one of embodiments 1 to 20, wherein the glycoprotein comprises a heavy chain variable complementarity-determining region 1 (VHCDR1) comprising an amino acid sequence as set forth in SEQ ID NO: 13, a VHCDR2 comprising an amino acid sequence as set forth in SEQ ID NO: 114, a VHCDR3 comprising an amino acid sequence as set forth in SEQ ID NO: 115, a light chain variable complementarity-determining region 1 (VLCDR1) comprising an amino acid sequence as set forth in SEQ ID NO: 118, a VLCDR2 comprising an amino acid sequence as set forth in SEQ ID NO: 119, and a VLCDR3 comprising an amino acid sequence as set forth in SEQ ID NO: 120.

Embodiment 78: The engineered glycoprotein population of any one of embodiments 1 to 20 and 77, wherein the glycoprotein comprises a heavy chain variable region comprising an amino acid sequence having at least 70%, 80%, 90%, 95% or 100% identity as the amino acid sequence set forth in SEQ ID NO: 116.

Embodiment 79: The engineered glycoprotein population of any one of embodiments 1 to 20, 77, and 78, wherein the glycoprotein comprises a light chain variable region comprising an amino acid sequence having at least 70%, 80%, 90%, 95% or 100% identity as the amino acid sequence set forth in SEQ ID NO: 121.

Embodiment 80: The engineered glycoprotein population of any one of embodiments 1 to 20 and 77 to 79, wherein the glycoprotein comprises a heavy chain comprising an amino acid sequence having at least 70%, 80%, 90%, 95% or 100% identity as the amino acid sequence set forth in SEQ ID NO: 117.

Embodiment 81: The engineered glycoprotein population of any one of embodiments 1 to 20 and 72 to 75, wherein the glycoprotein comprises a light chain comprising an amino acid sequence having at least 70%, 80%, 90%, 95% or 100% identity as the amino acid sequence set forth in SEQ ID NO: 122.

Embodiment 82: The engineered glycoprotein population of any one of embodiments 1 to 81, being produced by a cell, wherein the cell constitutively and/or controllably expresses an exogenous sialyltransferase catalytic peptide and an exogenous galactosyltransferase catalytic peptide, wherein the exogenous sialyltransferase catalytic peptide and the exogenous galactosyltransferase catalytic peptide are translated in close proximity.

Embodiment 83: The engineered glycoprotein population of embodiment 82, wherein the engineered glycoprotein population is produced by the cell in the presence of a culture medium comprising O-acyl-modified (peracetylated) 4-fluoro N-Acetylmannosamine (4-F-ManNAc).

Embodiment 84: The engineered glycoprotein population of embodiment 83, wherein the culture medium is devoid of sialyltransferase before being conditioned by the cell.

Embodiment 85: The engineered glycoprotein population of any one of embodiments 82 to 84, wherein the exogenous sialyltransferase catalytic peptide and the exogenous galactosyltransferase catalytic peptide are expressed in a single transcript.

Embodiment 86: The engineered glycoprotein population of any one of embodiments 82 to 85, wherein the cell comprises a first nucleic acid configured to express the exogenous sialyltransferase catalytic peptide and a second nucleic acid configured to express the exogenous galactosyltransferase catalytic peptide, wherein the first nucleic and the second nucleic acid are transcriptionally controlled by the same promoter.

Embodiment 87: The engineered glycoprotein population of embodiment 86, wherein the first nucleic acid and the second nucleic acid are connected to each other via a connecting nucleic acid, which is configured to encode a ribosomal shifting peptide.

Embodiment 88: The engineered glycoprotein population of embodiment 87, wherein the ribosomal shifting peptide comprises an amino acid sequence of DxExNPGP, wherein x denotes any amino acid, D denotes aspartic acid, E denotes glutamic acid, N denotes asparagine, P denotes proline, and G denotes glycine.

Embodiment 89: The engineered glycoprotein population of embodiment 87 or embodiment 88, wherein the ribosomal shifting peptide comprises an amino acid sequence as set forth in SEQ ID NO: 128, SEQ ID NO: 129, SEQ ID NO: 130, or SEQ ID NO: 131.

Embodiment 90: The engineered glycoprotein population of embodiment 86, wherein the exogenous sialyltransferase catalytic peptide and the exogenous galactosyltransferase catalytic peptide are configured to be expressed as a fusion protein.

Embodiment 91: The engineered glycoprotein population of embodiment 90, wherein the fusion protein comprises a first portion having the sialyltransferase catalytic peptide domain and a second portion having the galactosyltransferase catalytic peptide domain, wherein the first portion and the second portion are connected to each other via a cleavable linker, further wherein the cleavable linker is configured to be cleavable post-translation of the fusion protein, thereby upon cleavage releasing the sialyltransferase catalytic peptide and the galactosyltransferase catalytic peptide as separate proteins.

Embodiment 92: The engineered glycoprotein population of any one of embodiments 86 to 91, wherein the first nucleic acid is derived from an ST6Gall gene or a PspST gene.

Embodiment 93: The engineered glycoprotein population of any one of embodiments 86 to 92, wherein the first nucleic acid comprises a nucleotide sequence as set forth in SEQ ID NO: 132 or SEQ ID NO: 133.

Embodiment 94: The engineered glycoprotein population of embodiment 93, wherein the first nucleic acid comprises a nucleotide sequence as set forth in SEQ ID NO: 134 or SEQ ID NO: 135.

Embodiment 95: The engineered glycoprotein population of any one of embodiments 86 to 94, wherein the second nucleic acid is derived from an B4GALT1 gene.

Embodiment 96: The engineered glycoprotein population of any one of embodiments 86 to 95, wherein the second nucleic acid comprises a nucleotide sequence as set forth in SEQ ID NO: 136.

Embodiment 97: The engineered glycoprotein population of any one of embodiments 86 to 96, wherein the promoter is a constitutive promoter or an activable promoter.

Embodiment 98: The engineered glycoprotein population of embodiment 97, wherein the constitutive promoter is a CMV promoter, T7 promoter, a Human Elongation Factor 1 Alpha (EFla) promoter, a Chicken β-Actin (CAG) promoter, or an SV40 promoter.

Embodiment 99: The engineered glycoprotein population of embodiment 97, wherein the activable promoter is a Tetracycline-Inducible Promoter (activable by doxycycline) or a dihydrofolate reductase (DHFR) gene promoter.

Embodiment 100: The engineered glycoprotein population of any one of embodiments 84 to 99, wherein the sialyltransferase catalytic peptide is an alpha-2,6-sialyltransferase.

Embodiment 101: The engineered glycoprotein population of embodiment 100, wherein the sialyltransferase is a beta-galactoside alpha-2,6-sialyltransferase 1.

Embodiment 102: The engineered glycoprotein population of any one of embodiments 84 to 101, wherein the sialyltransferase catalytic peptide comprises an amino acid sequence as set forth in SEQ ID NO: 123 or SEQ ID NO: 124.

Embodiment 103: The engineered glycoprotein population of embodiment 102, wherein the sialyltransferase catalytic peptide comprises an amino acid sequence as set forth in SEQ ID NO: 125 or SEQ ID NO: 126.

Embodiment 104: The engineered glycoprotein population of any one of embodiments 84 to 103, wherein the galactosyltransferase catalytic peptide is a beta-1,4-galactosyltransferase 1.

Embodiment 105: The engineered glycoprotein population of any one of embodiments 84 to 104, wherein the galactosyltransferase catalytic peptide comprises an amino acid sequence as set forth in SEQ ID NO: 127.

Embodiment 106: The engineered glycoprotein population of any one of embodiments 84 to 105, wherein the cell is deficient in fucosyltransferase activity.

Embodiment 107: The engineered glycoprotein population of embodiment 106, wherein the cell is deficient in fucosyltransferase 8 activity.

Embodiment 108: The engineered glycoprotein population of embodiment 106 or embodiment 107, wherein the cell is deficient in a FUT8 gene encoding a product of the fucosyltransferase activity.

Embodiment 109: The engineered glycoprotein population of embodiment 84, wherein the cell comprises a vector comprising: a nucleic acid encoding a Clustered Regularly Interspaced Short Palindromic Repeat (CRISPR) crRNA comprising a nucleotide sequence as set forth in SEQ ID NO: 150, SEQ ID NO: 151, or SEQ ID NO: 152; a nucleic acid encoding a tracrRNA; and a nucleic acid encoding a CRISPR-associated (Cas) protein.

Embodiment 110: The engineered glycoprotein population of embodiment 109, wherein the tracrRNA.

Embodiment 111: The engineered glycoprotein population of embodiment 109 or embodiment 110, wherein the Cas protein is Cas9, Cas12a, Cas13, Cpf1, Cas8, or Cas5 nuclease.

Embodiment 112: The engineered glycoprotein population of any one of embodiments 109 to 111, wherein the nucleic acid encoding the Cas protein comprises a nucleotide sequence having at least 70%, 80%, 90%, 95% or 100% identity as the nucleotide sequence set forth in SEQ ID NO: 153.

Embodiment 113: A composition comprising an engineered glycoprotein population of any one of embodiments 1 to 112 and a pharmaceutically acceptable excipient.

Embodiment 114: The composition of embodiment 113, wherein the pharmaceutically acceptable excipient comprises a solvent, dispersion media, diluent, dispersion, suspension aid, surface active agent, isotonic agent, thickening or emulsifying agent, preservative, polymer, peptide, protein, cell, hyaluronidase, or mixtures thereof.

Embodiment 115: The composition of embodiment 113 or embodiment 114, wherein the composition is formulated in a form of tablets (including chewable tabelts), capsules, strips, syrups, powders, pastilles, sachets, effervescent compositions, pills, coated bead systems, granules, microspheres, dragées, films, orally administrable films, liquid, solutions, solids, suspensions, or emulsions.

Embodiment 116: The composition of any one of embodiments 113 to 115, comprising at least 5, 10, 20, 25, 30, 40,50, 60, 70,80, 90, 100, 120, 150, 175, 200, 220, 250, 275, 300, 350, 450, 500, 550, 600, 650, 700, 750, 800, 850, 900, 950, or 1000 mg/mL of the engineered glycoprotein population of any one of embodiments 1 to 112.

Embodiment 117: The composition of any one of embodiments 113 to 116, comprising 5 to 1000 mg/mL, 5 to 900 mg/mL, 5 to 800 mg/mL, 5 to 700 mg/mL, 5 to 600 mg/mL, 5 to 500 mg/mL, 5 to 400 mg/mL, 5 to 300 mg/mL, 5 to 200 mg/mL, 5 to 150 mg/mL, 5 to 100 mg/mL, 5 to 50 mg/mL, 25 to 1000 mg/mL, 25 to 900 mg/mL, 25 to 800 mg/mL, 25 to 700 mg/mL, 25 to 600 mg/mL, 25 to 500 mg/mL, 25 to 400 mg/mL, 25 to 300 mg/mL, 25 to 200 mg/mL, 25 to 150 mg/mL, 25 to 100 mg/mL, 25 to 50 mg/mL, 50 to 1000 mg/mL, 50 to 900 mg/mL, 50 to 800 mg/mL, 50 to 700 mg/mL, 50 to 600 mg/mL, 50 to 500 mg/mL, 50 to 400 mg/mL, 50 to 300 mg/mL, 50 to 200 mg/mL, 50 to 150 mg/mL, 50 to 100 mg/mL, 100 to 1000 mg/mL, 100 to 900 mg/mL, 100 to 800 mg/mL, 100 to 700 mg/mL, 100 to 600 mg/mL, 100 to 500 mg/mL, 100 to 400 mg/mL, 100 to 300 mg/mL, 100 to 200 mg/mL, or 100 to 150 mg/mL of the engineered glycoprotein population of any one of embodiments 1 to 112.

Embodiment 118: The composition of any one of embodiments 113 to 117, being used for treating a disease.

Embodiment 119: The composition of embodiment 118, wherein the disease comprises an autoimmune disease or cancer.

Embodiment 120: A method of increasing binding between a Fc gamma receptor (FcγR) and a glycoprotein, comprising: engineering the glycoprotein to obtain an engineered glycoprotein population, wherein the engineered glycoprotein population is according to the engineered glycoprotein population of any one of embodiments 1 to 112.

Embodiment 121: The method of embodiment 120, wherein the glycoprotein comprises a first glycan and the engineering modifies the first glycan with a second glycan, and wherein the second glycan is a sialylated complex type glycan.

Embodiment 122: The method of embodiment 120 or embodiment 121, wherein the engineering is performed by producing the glycoprotein by a cell, wherein the cell constitutively and/or controllably expresses an exogenous sialyltransferase catalytic peptide and an exogenous galactosyltransferase catalytic peptide, wherein the exogenous sialyltransferase catalytic peptide and the exogenous galactosyltransferase catalytic peptide are translated in close proximity.

Embodiment 123: The method of embodiment 122, wherein the glycoprotein is produced by the cell in the presence of a culture medium comprising O-acyl-modified (peracetylated) 4-fluoro N-Acetylmannosamine (4-F-ManNAc).

Embodiment 124: The method of embodiment 123, wherein the culture medium is devoid of sialyltransferase before being conditioned by the cell.

Embodiment 125: The method of any one of embodiments 122 to 124, wherein the exogenous sialyltransferase catalytic peptide and the exogenous galactosyltransferase catalytic peptide are expressed in a single transcript.

Embodiment 126: The method of any one of embodiments 122 to 125, comprising a first nucleic acid configured to express the exogenous sialyltransferase catalytic peptide and a second nucleic acid configured to express the exogenous galactosyltransferase catalytic peptide, wherein the first nucleic and the second nucleic acid are transcriptionally controlled by the same promoter.

Embodiment 127: The method of embodiment 126, wherein the first nucleic acid and the second nucleic acid are connected to each other via a connecting nucleic acid, which is configured to encode a ribosomal shifting peptide.

Embodiment 128: The method of embodiment 127, wherein the ribosomal shifting peptide comprises an amino acid sequence of DxExNPGP, wherein x denotes any amino acid, D denotes aspartic acid, E denotes glutamic acid, N denotes asparagine, P denotes proline, and G denotes glycine.

Embodiment 129: The method of embodiment 127 or embodiment 128, wherein the ribosomal shifting peptide comprises an amino acid sequence as set forth in SEQ ID NO: 06, SEQ ID NO: 07, SEQ ID NO: 08, or SEQ ID NO: 09.

Embodiment 130: The method of embodiment 126, wherein the exogenous sialyltransferase catalytic peptide and the exogenous galactosyltransferase catalytic peptide are co-configured to be expressed as a fusion protein.

Embodiment 131: The method of embodiment 130, wherein the fusion protein comprises a first portion having the sialyltransferase catalytic peptide domain and a second portion having the galactosyltransferase catalytic peptide domain, wherein the first portion and the second portion are connected to each other via a cleavable linker, further wherein the cleavable linker is configured to be cleavable post-translation of the fusion protein, thereby upon cleavage releasing the sialyltransferase catalytic peptide and the galactosyltransferase catalytic peptide as separate proteins.

Embodiment 132: The method of any one of embodiments 126 to 131, wherein the first nucleic acid is derived from an ST6Gall gene or a PspST gene.

Embodiment 133: The method any one of embodiments 126 to 132, wherein the first nucleic acid comprises a nucleotide sequence as set forth in SEQ ID NO: 132 or SEQ ID NO: 133.

Embodiment 134: The method of embodiment 133, wherein the first nucleic acid comprises a nucleotide sequence as set forth in SEQ ID NO: 134 or SEQ ID NO: 135.

Embodiment 135: The method of any one of embodiments 126 to 134, wherein the second nucleic acid is derived from an B4GALT1 gene.

Embodiment 136: The method of any one of embodiments 126 to 135, wherein the second nucleic acid comprises a nucleotide sequence as set forth in SEQ ID NO: 136.

Embodiment 137: The method of any one of embodiments 126 to 136, wherein the promoter is a constitutive promoter or an activable promoter.

Embodiment 138: The method of embodiment 137, wherein the constitutive promoter is a CMV promoter, T7 promoter, a Human Elongation Factor 1 Alpha (EFla) promoter, a Chicken β-Actin (CAG) promoter, or an SV40 promoter.

Embodiment 139: The method of embodiment 137, wherein the activable promoter is a Tetracycline-Inducible Promoter (activable by doxycycline) or a dihydrofolate reductase (DHFR) gene promoter.

Embodiment 140: The method of any one of embodiments 122 to 139, wherein the sialyltransferase catalytic peptide is an alpha-2,6-sialyltransferase.

Embodiment 141: The method of embodiment 140, wherein the sialyltransferase is a beta-galactoside alpha-2,6-sialyltransferase 1.

Embodiment 142: The method of any one of embodiments 122 to 141, wherein the sialyltransferase catalytic peptide comprises an amino acid sequence as set forth in SEQ ID NO: 123 or SEQ ID NO: 124.

Embodiment 143: The method of embodiment 142, wherein the sialyltransferase catalytic peptide comprises an amino acid sequence as set forth in SEQ ID NO: 125 or SEQ ID NO: 126.

Embodiment 144: The method of any one of embodiments 122 to 143, wherein the galactosyltransferase catalytic peptide is a beta-1,4-galactosyltransferase 1.

Embodiment 145: The method of any one of embodiments 122 to 144, wherein the galactosyltransferase catalytic peptide comprises an amino acid sequence as set forth in SEQ ID NO: 127.

Embodiment 146: The method of any one of embodiments 122 to 145, wherein the cell is deficient in fucosyltransferase activity.

Embodiment 147: The method of embodiment 146, wherein the cell is deficient in fucosyltransferase 8 activity.

Embodiment 148: The method of embodiment 146 or embodiment 147, wherein the cell is deficient in a FUT8 gene encoding a product of the fucosyltransferase activity.

Embodiment 149: The method of any one of embodiments 120 to 148, wherein the FcγR is FcγRIIA or FcγRIIIA.

Embodiment 150: The method of any one of embodiments 120 to 149 wherein the glycoprotein is a therapeutic protein, comprising a target-binding site and a glycosylation site.

Embodiment 151: The method of any one of embodiments 120 to 150 wherein the glycoprotein is an antibody or antigen-binding fragment thereof.

Embodiment 152: The method of embodiment 120 to 151, wherein the glycoprotein is Adalimumab (Humira®), Adalimumab (Humira®), Adalimumab-atto (Amjevita®), Bevacizumab (Avastin®), Alemtuzumab (Campath®), Ipilimumab (Yervoy®), Avelumab (Bavencio®), Durvalumab (IMFINZI®), Pembrolizumab (Keytruda®), Nivolumab (Opdivo®), Etanercept (Enbrel®), Trastuzumab (Herceptin®), Pertuzumab (Perjeta®), Cetuximab (Erbitux®), Rituximab (Rituxan®), Rituximab-atto (Truxima®), Obinutuzumab (Gazyva®), Infliximab (Remicade®), Ofatumumab (Arzerra®), Golimumab (Simponi®), Atezolizumab (Tecentriq®), Ocrelizumab (OCREVUS®), Gazyvaro Infliximab (Remicade®), Zanidatamab (Ziihera®), Daratumumab (Darzalex®), or Trastuzumab emtansine (Kadcyla®).

Embodiment 153: A method of treating a disease caused by a dysfunctional cell, comprising administering a subject in need the engineered glycoprotein population of any one of embodiments 1 to 112 at a pharmaceutically effective amount, wherein the glycoprotein is configured to target the dysfunctional cell.

Embodiment 154: The method of embodiment 153, wherein the dysfunctional cell is an immune cell or a cancer cell.

Embodiment 155: The method of embodiment 153 or embodiment 154, wherein the glycoprotein binds tumor necrosis factor-alpha (TNFα), Vascular Endothelial Growth Factor (VEGF), Cytotoxic T-Lymphocyte Associated protein 4 (CTLA-4), PD-L1, PD-1, Human Epidermal Growth Factor Receptor 2 (HER2), Epidermal Growth Factor Receptor (EGFR), CD38, CD52, or CD20.

Embodiment 156: The method of any one of embodiments 153 to 155, wherein the disease is an autoimmune disease or cancer.

Embodiment 157: The method of any one of embodiments 153 to 156, wherein the disease is arthritis (including, rheumatoid arthritis, juvenile rheumatoid arthritis, juvenile idiopathic arthritis, psoriatic arthritis, polyarthritis, oligoarthritis, enthesitis-related arthritis, axial spondyloarthritis), ankylosing spondylitis, Crohn's disease, ulcerative colitis, plaque psoriasis, hidradenitis suppurativa, uveitis, ankylosing spondylitis, granulomatosis with polyangiitis (GPA), microscopic polyangiitis (MPA), pemphigus vulgaris, idiopathic thrombocytopeni purpura, myasthenia gravis, Epstein-Barr virus-positive mucocutaneous ulcers, macular degeneration, breast cancer, colorectal cancer, lung cancer (e.g., non-small cell lung cancer), kidney cancer, brain cancer, bladder cancer, ovarian cancer, cervical cancer, melanoma, prostate cancer, urothelial cancer, skin cancer (e.g., Merkel-cell carcinoma), head and neck cancer, Hodgkin lymphoma, Non-Hodgkin's lymphoma (NHL; e.g., follicular lymphoma), chronic lymphocytic leukemia (CLL), and mature B-cell acute leukemia (B-AL), gastric cancer, malignant pleural mesothelioma, urothelial carcinoma, colon cancer, esophageal squamous cell carcinoma, liver cancer, gastric cancer, esophageal cancer, or gastroesophageal junction cancer.

Embodiment 158: The method of any one of embodiments 153 to 157, wherein the glycoprotein is Adalimumab (Humira®), Adalimumab-atto (Amjevita®), Bevacizumab (Avastin®), Alemtuzumab (Campath®), Ipilimumab (Yervoy®), Avelumab (Bavencio®), Durvalumab (IMFINZI®), Pembrolizumab (Keytruda®), Nivolumab (Opdivo®), Etanercept (Enbrel®), Trastuzumab (Herceptin®), Pertuzumab (Perjeta®), Cetuximab (Erbitux®), Rituximab (Rituxan®), Rituximab-atto (Truxima®), Obinutuzumab (Gazyva®), Infliximab (Remicade®), Ofatumumab (Arzerra®), Golimumab (Simponi®), Atezolizumab (Tecentriq®), Ocrelizumab (OCREVUS®), Gazyvaro Infliximab (Remicade®), Zanidatamab (Ziihera®), Daratumumab (Darzalex®), or Trastuzumab emtansine (Kadcyla®).

Embodiment 159: The method of any one of embodiments 153 to 158, wherein the glycoprotein is Adalimumab (Humira®), and the disease is rheumatoid arthritis (including, rheumatoid arthritis, juvenile rheumatoid arthritis, juvenile idiopathic arthritis, psoriatic arthritis, polyarthritis, oligoarthritis, enthesitis-related arthritis, axial spondyloarthritis), ankylosing spondylitis, Crohn's disease, ulcerative colitis, plaque psoriasis, hidradenitis suppurativa, uveitis, ankylosing spondylitis, granulomatosis with polyangiitis (GPA), microscopic polyangiitis (MPA), pemphigus vulgaris, idiopathic thrombocytopenia purpura, myasthenia gravis, or Epstein-Barr virus-positive mucocutaneous ulcers.

Embodiment 160: The method of any one of embodiments 153 to 159, wherein the glycoprotein is Bevacizumab (Avastin®), and the disease is breast cancer, colorectal cancer, kidney cancer, lung cancer, brain cancer, ovarian cancer, cervical cancer, or macular degeneration.

Embodiment 161: The method of any one of embodiments 153 to 159, wherein the glycoprotein is Ipilimumab (Yervoy®), and the disease is melanoma, prostate cancer, lung cancer, or urothelial cancer.

Embodiment 162: The method of any one of embodiments 153 to 159, wherein the glycoprotein is Avelumab (Bavencio®), and the disease is bladder cancer, kidney cancer, or skin cancer.

Embodiment 163: The method of any one of embodiments 153 to 159, wherein the glycoprotein is Durvalumab (IMFINZI®), and the disease is non-small cell lung cancer.

Embodiment 164: The method of any one of embodiments 153 to 159, wherein the glycoprotein is Pembrolizumab (Keytruda®), and the disease is Non-Hodgkin's lymphoma (NHL) or chronic lymphocytic leukemia (CLL).

Embodiment 165: The method of any one of embodiments 153 to 159, wherein the glycoprotein is Nivolumab (Opdivo®), and the disease is melanoma, non-small cell lung cancer, malignant pleural mesothelioma, kidney cancer, Hodgkin lymphoma, head and neck cancer, urothelial cancer, colorectal cancer, liver cancer, esophageal cancer, or gastric cancer.

Embodiment 166: The method of any one of embodiments 153 to 159, wherein the glycoprotein is Etanercept (Enbrel®), and the disease is arthritis (including, rheumatoid arthritis, juvenile rheumatoid arthritis, juvenile idiopathic arthritis, psoriatic arthritis, polyarthritis, oligoarthritis, enthesitis-related arthritis, axial spondyloarthritis), ankylosing spondylitis, Crohn's disease, ulcerative colitis, plaque psoriasis, hidradenitis suppurativa, uveitis, ankylosing spondylitis, granulomatosis with polyangiitis (GPA), microscopic polyangiitis (MPA), pemphigus vulgaris, idiopathic thrombocytopeniarpura, myasthenia gravis, or Epstein-Barr virus-positive mucocutaneous ulcers.

Embodiment 167: The method of any one of embodiments 153 to 159, wherein the glycoprotein is Etanercept (Enbrel®), and the disease is breast cancer or gastric cancer.

Embodiment 168: The method of any one of embodiments 153 to 159, wherein the glycoprotein is Pertuzumab (Perjeta®), and the disease is breast cancer.

Embodiment 169: The method of any one of embodiments 153 to 159, wherein the glycoprotein is Cetuximab (Erbitux®), and the disease is colorectal cancer or head and neck cancer.

Embodiment 170: The method of any one of embodiments 153 to 159, wherein the glycoprotein is Rituximab (Rituxan®), and the disease is non-Hodgkin lymphoma (NHL), chronic lymphocytic leukemia (CLL), rheumatoid arthritis, granulomatosis with polyangiitis, idiopathic thrombocytopenia purpura, pemphigus vulgaris, myasthenia gravis, or Epstein-Barr virus-positive mucocutaneous ulcers.

Embodiment 171: The method of any one of embodiments 153 to 159, wherein the glycoprotein is Obinutuzumab (Gazyva®), and the disease is chronic lymphocytic leukemia (CLL) or follicular lymphoma.

Embodiment 172: The method of any one of embodiments 153 to 159, wherein the engineered glycoprotein population is administered together with a pharmaceutically acceptable excipient.

Embodiment 173: The method of embodiment 172, wherein the pharmaceutically acceptable excipient comprises a solvent, dispersion media, diluent, dispersion, suspension aid, surface active agent, isotonic agent, thickening or emulsifying agent, preservative, polymer, peptide, protein, cell, hyaluronidase, or mixtures thereof.

Embodiment 174: The method of any one of embodiments 153 to 173, wherein the administering is made or via orally, intravenously (IV) intramuscularly (IM), intrathecally, subcutaneously, sublingually, buccally, rectally, vaginally, ocular route, otic route, nasally, inhalation, nebulization, cutaneously (topical or systemic), or transdermally.

Embodiment 175: The method of any one of embodiments 153 to 174, wherein the engineered glycoprotein population is formulated in a form of tablets (including chewable tabelts), capsules, strips, syrups, powders, pastilles, sachets, effervescent compositions, pills, coated bead systems, granules, microspheres, dragées, films, orally administrable films, liquid, solutions, solids, suspensions, or emulsions.

Embodiment 176: The method of any one of embodiments 153 to 175, wherein the pharmaceutically effective amount is at least 5, 10, 20, 25, 30, 40,50, 60, 70,80, 90, 100, 120, 150, 175, 200, 220, 250, 275, 300, 350, 450, 500, 550, 600, 650, 700, 750, 800, 850, 900, 950, or 1000 mg per day.

Embodiment 177: The method of any one of embodiments 153 to 176, wherein the effective amount is 5 to 1000 mg per day, 5 to 900 mg per day, 5 to 800 mg per day, 5 to 700 mg per day, 5 to 600 mg per day, 5 to 500 mg per day, 5 to 400 mg per day, 5 to 300 mg per day, 5 to 200 mg per day, 5 to 150 mg per day, 5 to 100 mg per day, 5 to 50 mg per day, 25 to 1000 mg per day, 25 to 900 mg per day, 25 to 800 mg per day, 25 to 700 mg per day, 25 to 600 mg per day, 25 to 500 mg per day, 25 to 400 mg per day, 25 to 300 mg per day, 25 to 200 mg per day, 25 to 150 mg per day, 25 to 100 mg per day, 25 to 50 mg per day, 50 to 1000 mg per day, 50 to 900 mg per day, 50 to 800 mg per day, 50 to 700 mg per day, 50 to 600 mg per day, 50 to 500 mg per day, 50 to 400 mg per day, 50 to 300 mg per day, 50 to 200 mg per day, 50 to 150 mg per day, 50 to 100 mg per day, 100 to 1000 mg per day, 100 to 900 mg per day, 100 to 800 mg per day, 100 to 700 mg per day, 100 to 600 mg per day, 100 to 500 mg per day, 100 to 400 mg per day, 100 to 300 mg per day, 100 to 200 mg per day, or 100 to 150 mg per day.

Embodiment 178: The method of embodiment 176 or embodiment 177, wherein the pharmaceutically effective amount is achieved by multiple administrations.

Embodiment 179: The method of embodiment 178, wherein the multiple administrations is performed at an interval of at least 0.5, 1, 2, 3, 4, 5, 6, 7, 8, 9, 10, 11, 12, 13, 14, 15, 16, 17, 18, 19, 20, 21, 22, 23, or 24 hours.

Embodiment 180: The method of embodiment 178 or embodiment 179, wherein the multiple administrations is performed at an interval of 0.5 to 24 hours, 0.5 to 20 hours, 0.5 to 16 hours, 0.5 to 12 hours, 0.5 to 8 hours, 0.5 to 4 hours, 0.5 to 2 hours, 2 to 24 hours, 2 to 20 hours, 2 to 16 hours, 2 to 12 hours, 2 to 8 hours, 2 to 4 hours, 4 to 24 hours, 4 to 20 hours, 4 to 16 hours, 4 to 12 hours, 4 to 8 hours, 12 to 24 hours, 12 to 20 hours, or 12 to 16 hours.

Embodiment 181: The method of any one of embodiments 153 to 180, wherein the subject in need is a human.

Embodiment 182: A cell for expressing a sialylated glycoprotein, constitutively and/or controllably expressing an exogenous sialyltransferase catalytic peptide and an exogenous galactosyltransferase catalytic peptide, wherein the exogenous sialyltransferase catalytic peptide and the exogenous galactosyltransferase catalytic peptide are translated in close proximity; and wherein the cell is deficient in endogenous fucosyltransferase activity, and deficiency in endogenous fucosyltransferase activity is made by introducing a vector into a parent cell of the cell, wherein the vector comprises: a nucleic acid encoding a Clustered Regularly Interspaced Short Palindromic Repeat (CRISPR) crRNA comprising a nucleotide sequence as set forth in SEQ ID NO: 150, SEQ ID NO: 151, or SEQ ID NO: 152; and a nucleic acid encoding a tracrRNA.

Embodiment 183: The cell of embodiment 182, wherein the exogenous sialyltransferase catalytic peptide and the exogenous galactosyltransferase catalytic peptide are expressed in a single transcript.

Embodiment 184: The cell of embodiment 182 or embodiment 183, comprising a first nucleic acid configured to express the exogenous sialyltransferase catalytic peptide and a second nucleic acid configured to express the exogenous galactosyltransferase catalytic peptide, wherein the first nucleic and the second nucleic acid are transcriptionally controlled by the same promoter.

Embodiment 185: The cell of embodiment 184, wherein the first nucleic acid and the second nucleic acid are connected to each other via a connecting nucleic acid, which is configured to encode a ribosomal shifting peptide.

Embodiment 186: The cell of embodiment 185, wherein the ribosomal shifting peptide comprises an amino acid sequence of DxExNPGP, wherein x denotes any amino acid, D denotes aspartic acid, E denotes glutamic acid, N denotes asparagine, P denotes proline, and G denotes glycine.

Embodiment 187: The cell of embodiment 185 or embodiment 186, wherein the ribosomal shifting peptide comprises an amino acid sequence as set forth in SEQ ID NO: 06, SEQ ID NO: 07, SEQ ID NO: 08, or SEQ ID NO: 09.

Embodiment 188: The cell of embodiment 184 wherein the exogenous sialyltransferase catalytic peptide and the exogenous galactosyltransferase catalytic peptide are co-configured to be expressed as a fusion protein.

Embodiment 189: The cell of embodiment 188, wherein the fusion protein comprises a first portion having the sialyltransferase catalytic peptide domain and a second portion having the galactosyltransferase catalytic peptide domain, wherein the first portion and the second portion are connected to each other via a cleavable linker, further wherein the cleavable linker is configured to be cleavable post-translation of the fusion protein, thereby upon cleavage releasing the sialyltransferase catalytic peptide and the galactosyltransferase catalytic peptide as separate proteins.

Embodiment 190: The cell of any one of embodiments 184 to 189, wherein the first nucleic acid is derived from an ST6Gall gene or a PspST gene.

Embodiment 191: The cell of any one of embodiments 184 to 190, wherein the first nucleic acid comprises a nucleotide sequence as set forth in SEQ ID NO: 10 or SEQ ID NO: 11.

Embodiment 192: The cell of any one of embodiment 191, wherein the first nucleic acid comprises a nucleotide sequence as set forth in SEQ ID NO: 12 or SEQ ID NO: 13.

Embodiment 193: The cell of any one of embodiments 184 to 192, wherein the second nucleic acid is derived from an B4GALT1 gene.

Embodiment 194: The cell of any one of embodiments 184 to 193, wherein the second nucleic acid comprises a nucleotide sequence as set forth in SEQ ID NO: 14.

Embodiment 195: The cell of any one of embodiments 184 to 194, wherein the promoter is a constitutive promoter or an activable promoter.

Embodiment 196: The cell of embodiment 195, wherein the constitutive promoter is a CMV promoter, T7 promoter, a Human Elongation Factor 1 Alpha (EFla) promoter, a Chicken β-Actin (CAG) promoter, or an SV40 promoter.

Embodiment 197: The cell of embodiment 195, wherein the activable promoter is a Tetracycline-Inducible Promoter (activable by doxycycline) or a dihydrofolate reductase (DHFR) gene promoter.

Embodiment 198: The cell of any one of embodiments 182 to 196, wherein the sialyltransferase catalytic peptide is an alpha-2,6-sialyltransferase.

Embodiment 199: The cell of embodiment 198, wherein the sialyltransferase is a beta-galactoside alpha-2,6-sialyltransferase 1.

Embodiment 200: The cell of any one of embodiments 182 to 199, wherein the sialyltransferase catalytic peptide comprises an amino acid sequence as set forth in SEQ ID NO: 01 or SEQ ID NO: 02.

Embodiment 201: The cell of embodiment 200, wherein the sialyltransferase catalytic peptide comprises an amino acid sequence as set forth in SEQ ID NO: 03 or SEQ ID NO: 04.

Embodiment 202: The cell of any one of embodiments 182 to 201, wherein the galactosyltransferase catalytic peptide is a beta-1,4-galactosyltransferase 1.

Embodiment 203: The cell of embodiment 202, wherein the galactosyltransferase catalytic peptide comprises an amino acid sequence as set forth in SEQ ID NO: 05.

Embodiment 204: The cell of any one of embodiments 182 to 203, wherein the tracerRNA comprises a nucleotide sequence having at least 70%, 80%, 90%, 95% or 100% identity as the nucleotide sequence set forth in SEQ ID NO: 154.

Embodiment 205: The cell of any one of embodiments 182 to 204, wherein the vector further comprises a nucleic acid encoding a CRISPR-associated (Cas) protein, or the cell further comprises a vector comprising the nucleic acid encoding the Cas protein.

Embodiment 206: The cell of embodiment 205, wherein the Cas protein is a Cas9, Cas12a, Cas13, Cpf1, Cas8, or Cas5nuclease,

Embodiment 207: The cell of embodiment 205 or embodiment 206, wherein the nucleic acid encoding the Cas protein comprises a nucleotide sequence having at least 70%, 80%, 90%, 95% or 100% identity as the nucleotide sequence set forth in SEQ ID NO: 153.

Embodiment 208: The cell of any one of embodiments 182 to 207, wherein the cell is derived from a Chinese hamster ovary cell or a HEK293 cell.

Embodiment 209: The cell of any one of embodiments 182 to 208, wherein the cell further comprises a payload nucleic acid configured to encode a recombinant glycoprotein, and the expression of the payload nucleic acid is transcriptionally controlled by a constitutive or an activable promoter.

Embodiment 210: The cell of embodiment 209, wherein the recombinant glycoprotein comprises a target-binding domain, wherein the target is tumor necrosis factor-alpha (TNFα), Vascular Endothelial Growth Factor (VEGF), Cytotoxic T-Lymphocyte Associated protein 4 (CTLA-4), PD-L1, PD-1, Human Epidermal Growth Factor Receptor 2 (HER2), Epidermal Growth Factor Receptor (EGFR), CD38, CD52, or CD20.

Embodiment 211: The cell of embodiment 209 or embodiment 210, wherein the recombinant glycoprotein is an antibody or an antigen-binding fragment thereof.

Embodiment 212: The cell of any one of embodiments 209 to 211, wherein the recombinant glycoprotein is Adalimumab (Humira®), Adalimumab-atto (Amjevita®), Bevacizumab (Avastin®), Alemtuzumab (Campath®), Ipilimumab (Yervoy®), Avelumab (Bavencio®), Durvalumab (IMFINZI®), Pembrolizumab (Keytruda®), Nivolumab (Opdivo®), Etanercept (Enbrel®), Trastuzumab (Herceptin®), Pertuzumab (Perjeta®), Cetuximab (Erbitux®), Rituximab (Rituxan®), Rituximab-atto (Truxima®), Obinutuzumab (Gazyva®), Infliximab (Remicade®), Ofatumumab (Arzerra®), Golimumab (Simponi®), Atezolizumab (Tecentriq®), Ocrelizumab (OCREVUS®), Gazyvaro Infliximab (Remicade®), Zanidatamab (Ziihera®), Daratumumab (Darzalex®), or Trastuzumab emtansine (Kadcyla®).

Embodiment 213: A cell for expressing a sialylated glycoprotein, comprising: a first nucleic acid, encoding an exogenous sialyltransferase catalytic peptide; a second nucleic acid, encoding an exogenous galactosyltransferase catalytic peptide; and a vector comprising: a nucleic acid encoding a Clustered Regularly Interspaced Short Palindromic Repeat (CRISPR) crRNA comprising a nucleotide sequence as set forth in SEQ ID NO: 150, SEQ ID NO: 151, or SEQ ID NO: 152; and a nucleic acid encoding a tracrRNA.

Embodiment 214: The cell of embodiment 213, wherein the exogenous sialyltransferase catalytic peptide and the exogenous galactosyltransferase catalytic peptide are expressed in a single transcript.

Embodiment 215: The cell of embodiment 213 or embodiment 214, comprising a first nucleic acid configured to express the exogenous sialyltransferase catalytic peptide and a second nucleic acid configured to express the exogenous galactosyltransferase catalytic peptide, wherein the first nucleic and the second nucleic acid are transcriptionally controlled by the same promoter.

Embodiment 216: The cell of embodiment 215, wherein the first nucleic acid and the second nucleic acid are connected to each other via a connecting nucleic acid, which is configured to encode a ribosomal shifting peptide.

Embodiment 217: The cell of embodiment 216, wherein the ribosomal shifting peptide comprises an amino acid sequence of DxExNPGP, wherein x denotes any amino acid, D denotes aspartic acid, E denotes glutamic acid, N denotes asparagine, P denotes proline, and G denotes glycine.

Embodiment 218: The cell of embodiment 216 or embodiment 217, wherein the ribosomal shifting peptide comprises an amino acid sequence as set forth in SEQ ID NO: 128, SEQ ID NO: 129, SEQ ID NO: 130, or SEQ ID NO: 131.

Embodiment 219: The cell of embodiment 215 wherein the exogenous sialyltransferase catalytic peptide and the exogenous galactosyltransferase catalytic peptide are configured to be expressed as a fusion protein.

Embodiment 220: The cell of embodiment 219, wherein the fusion protein comprises a first portion having the sialyltransferase catalytic peptide domain and a second portion having the galactosyltransferase catalytic peptide domain, wherein the first portion and the second portion are connected to each other via a cleavable linker, further wherein the cleavable linker is configured to be cleavable post-translation of the fusion protein, thereby upon cleavage releasing the sialyltransferase catalytic peptide and the galactosyltransferase catalytic peptide as separate proteins.

Embodiment 221: The cell of any one of embodiments 215 to 220, wherein the first nucleic acid is derived from an ST6Gall gene or a PspST gene.

Embodiment 222: The cell of any one of embodiments 215 to 221, wherein the first nucleic acid comprises a nucleotide sequence as set forth in SEQ ID NO: 132 or SEQ ID NO: 133.

Embodiment 223: The cell of any one of embodiment 222, wherein the first nucleic acid comprises a nucleotide sequence as set forth in SEQ ID NO: 134 or SEQ ID NO: 135.

Embodiment 224: The cell of any one of embodiments 215 to 223, wherein the second nucleic acid is derived from an B4GALT1 gene.

Embodiment 225: The cell of any one of embodiments 215 to 224, wherein the second nucleic acid comprises a nucleotide sequence as set forth in SEQ ID NO: 136.

Embodiment 226: The cell of any one of embodiments 215 to 225, wherein the promoter is a constitutive promoter or an activable promoter.

Embodiment 227: The cell of embodiment 226, wherein the constitutive promoter is a CMV promoter, T7 promoter, a Human Elongation Factor 1 Alpha (EFla) promoter, a Chicken β-Actin (CAG) promoter, or an SV40 promoter.

Embodiment 228: The cell of embodiment 226, wherein the activable promoter is a Tetracycline-Inducible Promoter (activable by doxycycline) or a dihydrofolate reductase (DHFR) gene promoter.

Embodiment 229: The cell of any one of embodiments 213 to 228, wherein the sialyltransferase catalytic peptide is an alpha-2,6-sialyltransferase.

Embodiment 230: The cell of embodiment 229, wherein the sialyltransferase is a beta-galactoside alpha-2,6-sialyltransferase 1.

Embodiment 231: The cell of any one of embodiments 213 to 230, wherein the sialyltransferase catalytic peptide comprises an amino acid sequence as set forth in SEQ ID NO: 01 or SEQ ID NO: 02.

Embodiment 232: The cell of embodiment 231, wherein the sialyltransferase catalytic peptide comprises an amino acid sequence as set forth in SEQ ID NO: 03 or SEQ ID NO: 04.

Embodiment 233: The cell of any one of embodiments 213 to 232, wherein the galactosyltransferase catalytic peptide is a beta-1,4-galactosyltransferase 1.

Embodiment 234: The cell of embodiment 233, wherein the galactosyltransferase catalytic peptide comprises an amino acid sequence as set forth in SEQ ID NO: 05.

Embodiment 235: The cell of any one of embodiments 213 to 234, wherein the tracerRNA comprises a nucleotide sequence having at least 70%, 80%, 90%, 95% or 100% identity as the nucleotide sequence set forth in SEQ ID NO: 154.

Embodiment 236: The cell of any one of embodiments 213 to 235, wherein the vector further comprises a nucleic acid encoding a CRISPR-associated (Cas) protein, or the cell further comprises a vector comprising the nucleic acid encoding the Cas protein.

Embodiment 237: The cell of embodiment 236, wherein the Cas protein is a Cas9, Cas12a, Cas13, Cpf1, Cas8, or Cas5 nuclease.

Embodiment 238: The cell of embodiment 236 or embodiment 237, wherein the nucleic acid encoding the Cas protein comprises a nucleotide sequence having at least 70%, 80%, 90%, 95% or 100% identity as the nucleotide sequence set forth in SEQ ID NO: 153.

Embodiment 239: The cell of any one of embodiments 213 to 238, wherein the cell is derived from a Chinese hamster ovary cell or a HEK293 cell.

Embodiment 240: The cell of any one of embodiments 213 to 239, wherein the cell further comprises a payload nucleic acid configured to encode a recombinant glycoprotein, and the expression of the payload nucleic acid is transcriptionally controlled by a constitutive or an activable promoter.

Embodiment 241: The cell of embodiment 240, wherein the recombinant glycoprotein comprises a target-binding domain, wherein the target is tumor necrosis factor-alpha (TNFα), Vascular Endothelial Growth Factor (VEGF), Cytotoxic T-Lymphocyte Associated protein 4 (CTLA-4), PD-L1, PD-1, Human Epidermal Growth Factor Receptor 2 (HER2), Epidermal Growth Factor Receptor (EGFR), CD38, CD52, or CD20.

Embodiment 242: The cell of embodiment 240 or embodiment 241, wherein the recombinant glycoprotein is an antibody or an antigen-binding fragment thereof.

Embodiment 243: The cell of any one of embodiments 240 to 242, wherein the recombinant glycoprotein is Adalimumab (Humira®), Adalimumab-atto (Amjevita®), Bevacizumab (Avastin®), Alemtuzumab (Campath®), Ipilimumab (Yervoy®), Avelumab (Bavencio®), Durvalumab (IMFINZI®), Pembrolizumab (Keytruda®), Nivolumab (Opdivo®), Etanercept (Enbrel®), Trastuzumab (Herceptin®), Pertuzumab (Perjeta®), Cetuximab (Erbitux®), Rituximab (Rituxan®), Rituximab-atto (Truxima®), Obinutuzumab (Gazyva®), Infliximab (Remicade®), Ofatumumab (Arzerra®), Golimumab (Simponi®), Atezolizumab (Tecentriq®), Ocrelizumab (OCREVUS®), Gazyvaro Infliximab (Remicade®), Zanidatamab (Ziihera®), Daratumumab (Darzalex®), or Trastuzumab emtansine (Kadcyla®).

Embodiment 244: A cell culture medium, comprising a basal medium and a peracetylated 4-fluoro N-Acetylmannosamine (4-F-ManNAc), wherein the basal medium comprises a carbon source, amino acids, and minerals, and the cell culture medium is devoid of sialyltransferase.

Embodiment 245: The cell culture medium of embodiment 244, comprising about 10, 50, 100, 150, 200, 250, 300, 350, 400, 450, 500, 550, 600, 650, 700, 750, 800, 850, 900, 950, 1000, 1500, 2000, 2500, or 3000 uM (micromolar) of the peracetylated 4-fluoro N-Acetylmannosamine (peracetylated 4-F-ManNAc).

Embodiment 246: The cell culture medium of embodiment 244 or embodiment 245, comprising 10 to 3000 uM, 10 to 2000 uM, 10 to 1000 uM, 10 to 900 uM, 10 to 800 uM, 10 to 700 uM, 10 to 600 uM, 10 to 500 uM, 10 to 400 uM, 10 to 300 uM, 10 to 200 uM, 10 to 100 uM, 100 to 3000 uM, 100 to 2500 uM, 100 to 2000 uM, 100 to 1500 uM, 100 to 1000 uM, 100 to 900 uM, 100 to 800 uM, 100 to 700 uM, 100 to 600 uM, 100 to 500 uM, 100 to 400 uM, 100 to 300 uM, 100 to 200 uM, 200 to 3000 uM, 200 to 2500 uM, 200 to 2000 uM, 200 to 1500 uM, 200 to 1000 uM, 200 to 900 uM, 200 to 800 uM, 200 to 700 uM, 200 to 600 uM, 200 to 500 uM, 200 to 400 uM, 200 to 350 uM, or 200 to 300 uM of the peracetylated 4-F-ManNAc.

Embodiment 247: The cell culture medium of any one of embodiments 244 to 246, wherein the carbon source comprises glucose, fructose, sucrose, sorbitol, mannitol, or a mixture thereof.

Embodiment 248: The cell culture medium of any one of embodiments 244 to 247, wherein the minerals comprise calcium, magnesium, potassium, sodium, phosphate, or a mixture thereof.

Embodiment 249: The cell culture medium of any one of embodiments 244 to 248, further comprising serum, vitamins, growth factors, a buffer system, a pH indicator, antibiotics, or a mixture thereof.

Embodiment 250: A cell culture medium, comprising a complex culture medium and a peracetylated 4-fluoro N-Acetylmannosamine (4-F-ManNAc), wherein the cell culture medium is devoid of sialyltransferase.

Embodiment 251: The cell culture medium of embodiment 250, comprising about 10, 50, 100, 150, 200, 250, 300, 350, 400, 450, 500, 550, 600, 650, 700, 750, 800, 850, 900, 950, 1000, 1500, 2000, 2500, or 3000 uM (micromolar) of the peracetylated 4-fluoro N-Acetylmannosamine (4-F-ManNAc).

Embodiment 252: The cell culture medium of embodiment 250 or embodiment 251, comprising 10 to 3000 uM, 10 to 2000 uM, 10 to 1000 uM, 10 to 900 uM, 10 to 800 uM, 10 to 700 uM, 10 to 600 uM, 10 to 500 uM, 10 to 400 uM, 10 to 300 uM, 10 to 200 uM, 10 to 100 uM, 100 to 3000 uM, 100 to 2500 uM, 100 to 2000 uM, 100 to 1500 uM, 100 to 1000 uM, 100 to 900 uM, 100 to 800 uM, 100 to 700 uM, 100 to 600 uM, 100 to 500 uM, 100 to 400 uM, 100 to 300 uM, 100 to 200 uM, 200 to 3000 uM, 200 to 2500 uM, 200 to 2000 uM, 200 to 1500 uM, 200 to 1000 uM, 200 to 900 uM, 200 to 800 uM, 200 to 700 uM, 200 to 600 uM, 200 to 500 uM, 200 to 400 uM, 200 to 350 uM, or 200 to 300 uM of the peracetylated 4-F-ManNAc.

Embodiment 253: The cell culture medium of any one of embodiments 250 to 252, wherein the complex medium is BalanCD CHO Growth A Medium, ExpiCHO™ Expression Medium, ExpiCHO™ Stable Production Medium, CD FortiCHO™ Medium, CD CHO AGT™ Medium, CD OptiCHO™ AGT™ Medium, CD CHO Medium, CHO-S-SFM II, High-Intensity Perfusion CHO Medium, Efficient-Pro™ Medium, Dulbecco's Modified Eagles Medium (DMEM), Roswell Park Memorial Institute medium (RPMI) 1640, DMEM F12, Modified Eagles Medium (MEM), or alpha-Minimum Essential Medium.

Embodiment 254: A method of increasing ADCC, CDC, ADCP, vaccinal effect, and half-live of a glycoprotein, comprising: engineering the glycoprotein to obtain an engineered glycoprotein population, wherein the engineered glycoprotein population is according to the engineered glycoprotein population of any one of embodiments 1 to 112.

Embodiment 255: The method of embodiment 254, wherein the glycoprotein comprises a first glycan and the engineering modifies the first glycan with a second glycan, and wherein the second glycan is a sialylated complex type glycan.

Embodiment 256: The method of embodiment 254 or embodiment 255, wherein the engineering is performed by producing the glycoprotein by a cell, wherein the cell constitutively and/or controllably expresses an exogenous sialyltransferase catalytic peptide and an exogenous galactosyltransferase catalytic peptide, wherein the exogenous sialyltransferase catalytic peptide and the exogenous galactosyltransferase catalytic peptide are translated in close proximity.

Embodiment 257: The method of embodiment 256, wherein the glycoprotein is produced by the cell in the presence of a culture medium comprising O-acyl-modified (peracetylated) 4-fluoro N-Acetylmannosamine (4-F-ManNAc).

Embodiment 258: The method of embodiment 257, wherein the culture medium is devoid of sialyltransferase before being conditioned by the cell.

Embodiment 259: The method of any one of embodiments 256 to 258, wherein the exogenous sialyltransferase catalytic peptide and the exogenous galactosyltransferase catalytic peptide are expressed in a single transcript.

Embodiment 260: The method of any one of embodiments 256 to 259, comprising a first nucleic acid configured to express the exogenous sialyltransferase catalytic peptide and a second nucleic acid configured to express the exogenous galactosyltransferase catalytic peptide, wherein the first nucleic and the second nucleic acid are transcriptionally controlled by the same promoter.

Embodiment 261: The method of embodiment 260, wherein the first nucleic acid and the second nucleic acid are connected to each other via a connecting nucleic acid, which is configured to encode a ribosomal shifting peptide.

Embodiment 262: The method of embodiment 261, wherein the ribosomal shifting peptide comprises an amino acid sequence of DxExNPGP, wherein x denotes any amino acid, D denotes aspartic acid, E denotes glutamic acid, N denotes asparagine, P denotes proline, and G denotes glycine.

Embodiment 263: The method of embodiment 261 or embodiment 262, wherein the ribosomal shifting peptide comprises an amino acid sequence as set forth in SEQ ID NO: 06, SEQ ID NO: 07, SEQ ID NO: 08, or SEQ ID NO: 09.

Embodiment 264: The method of embodiment 260, wherein the exogenous sialyltransferase catalytic peptide and the exogenous galactosyltransferase catalytic peptide are co-configured to be expressed as a fusion protein.

Embodiment 265: The method of embodiment 264, wherein the fusion protein comprises a first portion having the sialyltransferase catalytic peptide domain and a second portion having the galactosyltransferase catalytic peptide domain, wherein the first portion and the second portion are connected to each other via a cleavable linker, further wherein the cleavable linker is configured to be cleavable post-translation of the fusion protein, thereby upon cleavage releasing the sialyltransferase catalytic peptide and the galactosyltransferase catalytic peptide as separate proteins.

Embodiment 266: The method of any one of embodiments 260 to 265, wherein the first nucleic acid is derived from an ST6Gall gene or a PspST gene.

Embodiment 267: The method any one of embodiments 260 to 266, wherein the first nucleic acid comprises a nucleotide sequence as set forth in SEQ ID NO: 132 or SEQ ID NO: 133.

Embodiment 268: The method of embodiment 267, wherein the first nucleic acid comprises a nucleotide sequence as set forth in SEQ ID NO: 134 or SEQ ID NO: 135.

Embodiment 269: The method of any one of embodiments 260 to 268, wherein the second nucleic acid is derived from an B4GALT1 gene.

Embodiment 270: The method of any one of embodiments 260 to 269, wherein the second nucleic acid comprises a nucleotide sequence as set forth in SEQ ID NO: 136.

Embodiment 271: The method of any one of embodiments 260 to 270, wherein the promoter is a constitutive promoter or an activable promoter.

Embodiment 272: The method of embodiment 271, wherein the constitutive promoter is a CMV promoter, T7 promoter, a Human Elongation Factor 1 Alpha (EFla) promoter, a Chicken β-Actin (CAG) promoter, or an SV40 promoter.

Embodiment 273: The method of embodiment 271, wherein the activable promoter is a Tetracycline-Inducible Promoter (activable by doxycycline) or a dihydrofolate reductase (DHFR) gene promoter.

Embodiment 274: The method of any one of embodiments 256 to 273, wherein the sialyltransferase catalytic peptide is an alpha-2,6-sialyltransferase.

Embodiment 275: The method of embodiment 274, wherein the sialyltransferase is a beta-galactoside alpha-2,6-sialyltransferase 1.

Embodiment 276: The method of any one of embodiments 256 to 275, wherein the sialyltransferase catalytic peptide comprises an amino acid sequence as set forth in SEQ ID NO: 123 or SEQ ID NO: 124.

Embodiment 277: The method of embodiment 276, wherein the sialyltransferase catalytic peptide comprises an amino acid sequence as set forth in SEQ ID NO: 125 or SEQ ID NO: 126.

Embodiment 278: The method of any one of embodiments 256 to 277, wherein the galactosyltransferase catalytic peptide is a beta-1,4-galactosyltransferase 1.

Embodiment 279: The method of any one of embodiments 256 to 278, wherein the galactosyltransferase catalytic peptide comprises an amino acid sequence as set forth in SEQ ID NO: 127.

Embodiment 280: The method of any one of embodiments 256 to 279, wherein the cell is deficient in fucosyltransferase activity.

Embodiment 281: The method of embodiment 280, wherein the cell is deficient in fucosyltransferase 8 activity.

Embodiment 282: The method of embodiment 280 or embodiment 281, wherein the cell is deficient in a FUT8 gene encoding a product of the fucosyltransferase activity.

Embodiment 283: The method of any one of embodiments 254 to 282 wherein the glycoprotein is a therapeutic protein, comprising a target-binding site and and a glycosylation site.

Embodiment 284: The method of any one of embodiments 254 to 283 wherein the glycoprotein is an antibody or antigen-binding fragment thereof.

Embodiment 284: The method of embodiment 254 to 284, wherein the glycoprotein is Adalimumab (Humira®), Adalimumab (Humira®), Adalimumab-atto (Amjevita®), Bevacizumab (Avastin®), Alemtuzumab (Campath®), Ipilimumab (Yervoy®), Avelumab (Bavencio®), Durvalumab (IMFINZI®), Pembrolizumab (Keytruda®), Nivolumab (Opdivo®), Etanercept (Enbrel®), Trastuzumab (Herceptin®), Pertuzumab (Perjeta®), Cetuximab (Erbitux®), Rituximab (Rituxan®), Rituximab-atto (Truxima®), Obinutuzumab (Gazyva®), Infliximab (Remicade®), Ofatumumab (Arzerra®), Golimumab (Simponi®), Atezolizumab (Tecentriq®), Ocrelizumab (OCREVUS®), Gazyvaro Infliximab (Remicade®), Zanidatamab (Ziihera®), Daratumumab (Darzalex®), or Trastuzumab emtansine (Kadcyla®).

SEQUENCES

| | |
|---|---|
| SEQ ID NO: 01 | DYAMH |
| Adalimumab | |
| (Humira®) | |
| VHCDR1 | |
| amino acid | |
| SEQ ID NO: 02 | AITWNSGHID YADSVEG |
| Adalimumab | |
| (Humira®) | |
| VHCDR2 | |
| amino acid | |
| SEQ ID NO: 03 | VSYLSTASSL DY |
| Adalimumab | |
| (Humira®) | |
| VHCDR3 | |
| amino acid | |
| SEQ ID NO: 04 | EVQLVESGGG LVQPGRSLRL SCAASGETED DYAMHWVRQA PGKGLEWVSA |
| Adalimumab | ITWNSGHIDY ADSVEGRETI SRDNAKNSLY LQMNSLRAED TAVYYCAKVS |
| (Humira®) | YLSTASSLDY WGQGTLVTVS S |
| VH | |
| amino acid | |
| SEQ ID NO: 05 | EVQLVESGGG LVQPGRSLRL SCAASGETED DYAMHWVRQA PGKGLEWVSA |
| Adalimumab | ITWNSGHIDY ADSVEGRETI SRDNAKNSLY LQMNSLRAED TAVYYCAKVS |
| (Humira®) | YLSTASSLDY WGQGTLVTVS SASTKGPSVF PLAPSSKSTS GGTAALGCLV |
| Heavy chain | KDYFPEPVTV SWNSGALTSG VHTFPAVLQS SGLYSLSSVV TVPSSSIGTQ |
| amino acid | TYICNVNHKP SNTKVDKKVE PKSCDKTHTC PPCPAPELLG GPSVELFPPK |
| | PKDTLMISRT PEVTCVVVDV SHEDPEVKEN WYVDGVEVHN AKTKPREEQY |
| | NSTYRVVSVL TVLHQDWING KEYKCKVSNK ALPAPIEKTI SKAKGQPREP |
| | QVYTLPPSRD ELTKNQVSLT CLVKGFYPSD LAVEWESNGQ PENNYKTTPP |
| | VLDSDGSFEL YSKLTVDKSR WQQGNVESCS VMHEALHNHY TQKSLSLSPG K |
| | RASQGIRNYL A |
| SEQ ID NO: 06 | |
| Adalimumab | |
| (Humira®) | |
| VLCDR1 | |
| amino acid | |
| SEQ ID NO: 07 | AASTLQS |
| Adalimumab | |
| (Humira®) | |
| VLCDR2 | |
| amino acid | |
| SEQ ID NO: 08 | QRYNRAPYT |
| Adalimumab | |
| (Humira®) | |
| VLCDR3 | |
| amino acid | |
| SEQ ID NO: 09 | DIQMTQSPSS LSASVGDRVT ITCRASQGIR NYLAWYQQKP GKAPKLLIYA |
| Adalimumab | ASTLQSGVPS RESGSGSGTD FTLTISSLQP EDVATYYCQR YNRAPYTEGQ |
| (Humira®) | GTKVEIK |
| VL | |
| amino acid | |
| SEQ ID NO: 10 | DIQMIQSPSS LSASVGDRVT ITCRASQGIR NYLAWYQQKP GKAPKLLIYA |
| Adalimumab | ASTLQSGVPS RESGSGSGTD FILTISSLQP EDVATYYCQR YNRAPYTEGQ |
| (Humira®) | GTKVEIKRTV AAPSVFIFPP SDEQLKSGTA SVVCLLNNEY PREAKVQWKV |
| Light chain | DNALQSGNSQ ESVTEQDSKD STYSLSSTLT LSKADYEKHK VYACEVTHQG |
| amino acid | LSSPVTKSEN RGEC |
| SEQ ID NO: 11 | GYTFTNYG |
| Bevacizumab | |
| (Avastin®) | |
| VHCDR1 | |
| amino acid | |
| SEQ ID NO: 12 | INTYTGEP |
| Bevacizumab | |
| (Avastin®) | |
| VHCDR2 | |
| amino acid | |
| SEQ ID NO: 13 | AKYPHYYGSS HWYFDV |
| Bevacizumab | |
| (Avastin®) | |
| VHCDR3 | |
| amino acid | |
| SEQ ID NO: 14 | EVQLVESGGG LVQPGGSLRL SCAASGYTET NYGMNWVRQA PGKGLEWVGW |
| Bevacizumab | INTYTGEPTY AADEKRRETF SLDTSKSTAY LQMNSLRAED TAVYYCAKYP |
| (Avastin®) | HYYGSSHWYF DVWGQGTLVT VSS |
| VH | |
| amino acid | |
| SEQ ID NO: 15 | EVQLVESGGG LVQPGGSLRL SCAASGYTET NYGMNWVRQA PGKGLEWVGW |
| Bevacizumab | INTYTGEPTY AADEKRRETF SLDTSKSTAY LQMNSLRAED TAVYYCAKYP |
| (Avastin®) | HYYGSSHWYF DVWGQGTLVT VSSASTKGPS VFPLAPSSKS TSGGTAALGC |
| Heavy chain | LVKDYFPEPV TVSWNSGALT SGVHTFPAVL QSSGLYSLSS VVTVPSSSLG |
| amino acid | TQTYICNVNH KPSNTKVDKK VEPKSCDKTH TCPPCPAPEL LGGPSVELEP |
| | PKPKDTLMIS RTPEVTCVVV DVSHEDPEVK ENWYVDGVEV HNAKTKPREE |
| | QYNSTYRVVS VLIVLHQDWL NGKEYKCKVS NKALPAPIEK TISKAKGQPR |
| | EPQVYTLPPS REEMTKNQVS LTCLVKGFYP SDIAVEWESN GQPENNYKTT |
| | PPVLDSDGSF FLYSKLTVDK SRWQQGNVES CSVMHEALHN HYTQKSLSLS PGK |
| SEQ ID NO: 16 | QDISNY |
| Bevacizumab | |
| (Avastin®) | |
| VLCDR1 | |
| amino acid | |
| SEQ ID NO: 17 | FTS |
| Bevacizumab | |
| (Avastin®) | |
| VLCDR2 | |
| amino acid | |
| SEQ ID NO: 18 | QQYSTVPWT |
| Bevacizumab | |
| (Avastin®) | |
| VLCDR3 | |
| amino acid | |
| SEQ ID NO: 19 | DIQAATMIQS PSSLSASVGD RVTITCSASQ DISNYLNWYQ QKPGKAPKVL |
| Bevacizumab | IYFTSSLHSG VPSRESGSGS GTDETLTISS LQPEDFATYY CQQYSTVPWT |
| (Avastin®) | FGQGTKVEIK |
| VL | |
| amino acid | |
| SEQ ID NO: 20 | DIQAATMTQS PSSLSASVGD RVTITCSASQ DISNYLNWYQ QKPGKAPKVL |
| Bevacizumab | IYFTSSLHSG VPSRESGSGS GTDETLTISS LQPEDEATYY CQQYSTVPWT |
| (Avastin®) | FGQGTKVEIK RTVAAPSVEI FPPSDEQLKS GTASVVCLIN NEYPREAKVQ |
| Light chain | WKVDNALQSG NSQESVTEQD SKDSTYSLSS TLTLSKADYE KHKVYACEVT |
| amino acid | HQGLSSPVTK SENRGEC |
| SEQ ID NO: 21 | GETFSSYT |
| Ipilimumab | |
| (Yervoy®) | |
| VHCDR1 | |
| amino acid | |
| SEQ ID NO: 22 | TFISYDGNNK Y |
| Ipilimumab | |
| (Yervoy®) | |
| VHCDR2 | |
| amino acid | |
| SEQ ID NO: 23 | ARTGWLGPED Y |
| Ipilimumab | |
| (Yervoy®) | |
| VHCDR3 | |
| amino acid | |
| SEQ ID NO: 24 | QVQLVESGGG VVQPGRSLRL SCAASGETES SYTMHWVRQA PGKGLEWVTF |
| Ipilimumab | ISYDGNNKYY ADSVKGRETI SRDNSKNTLY LQMNSLRAED TATYYCARTG |
| (Yervoy®) | WLGPEDYWGQ GTLVTVSS |
| VH | |
| amino acid | |
| SEQ ID NO: 25 | QVQLVESGGG VVQPGRSLRL SCAASGETES SYTMHWVRQA PGKGLEWVTF |
| Ipilimumab | ISYDGNNKYY ADSVKGRETI SRDNSKNTLY LQMNSLRAED TAIYYCARTG |
| (Yervoy®) | WLGPEDYWGQ GTLVTVSSAS TKGPSVEPLA PSSKSTSGGT AALGCLVKDY |
| Heavy chain | FPEPVTVSWN SGALTSGVHT FPAVLQSSGL YSLSSVVTVP SSSLGIQTYI |
| amino acid | CNVNHKPSNT KVDKRVEPKS CDKTHTCPPC PAPELLGGPS VELFPPKPKD |
| | TIMISRTPEV TCVVVDVSHE DPEVKENWYV DGVEVHNAKT KPREEQYNST |
| | YRVVSVLTVL HQDWLNGKEY KCKVSNKALP APIEKTISKA KGQPREPQVY |
| | TLPPSRDELT KNQVSLICLV KGFYPSDIAV EWESNGQPEN NYKTTPPVLD |
| | SDGSFFLYSK LTVDKSRWQQ GNVESCSVMH EALHNHYTQK SLSLSPGK |
| SEQ ID NO: 26 | QSVGSSY |
| Ipilimumab | |
| (Yervoy®) | |
| VLCDR1 | |
| amino acid | |
| SEQ ID NO: 27 | GAF |
| Ipilimumab | |
| (Yervoy®) | |
| VLCDR2 | |
| amino acid | |
| SEQ ID NO: 28 | QQYGSSPWT |
| Ipilimumab | |
| (Yervoy®) | |
| VLCDR3 | |
| amino acid | |
| SEQ ID NO: 29 | EIVLTQSPGT LSLSPGERAT LSCRASQSVG SSYLAWYQQK PGQAPRILIY |
| Ipilimumab | GAFSRATGIP DRESGSGSGT DETLTISRLE PEDFAVYYCQ QYGSSPWTFG |
| (Yervoy®) | QGTKVEIK |
| VL | |
| amino acid | |
| SEQ ID NO: 30 | EIVLTQSPGT LSLSPGERAT LSCRASQSVG SSYLAWYQQK PGQAPRLLIY |
| Ipilimumab | GAFSRATGIP DRESGSGSGT DETLTISRLE PEDFAVYYCQ QYGSSPWTEG |
| (Yervoy®) | QGTKVEIKRT VAAPSVFIFP PSDEQLKSGT ASVVCLINNF YPREAKVQWK |
| Light chain | VDNALQSGNS QESVTEQDSK DSTYSLSSTL TLSKADYEKH KVYACEVTHQ |
| amino acid | GLSSPVTKSF NRGEC |
| SEQ ID NO: 31 | SYIMM |
| Avelumab | |
| (Bavencio®) | |
| VHCDR1 | |
| amino acid | |
| SEQ ID NO: 32 | SIYPSGGITF |
| Avelumab | |
| (Bavencio®) | |
| VHCDR2 | |
| amino acid | |
| SEQ ID NO: 33 | IKLGTVTTV |
| Avelumab | |
| (Bavencio®) | |
| VHCDR3 | |
| amino acid | |
| SEQ ID NO: 34 | EVQLLESGGG LVQPGGSLRL SCAASGETES SYIMMWVRQA PGKGLEWVSS |
| Avelumab | IYPSGGITFY ADTVKGRETI SRDNSKNTLY LQMNSLRAED TAVYYCARIK |
| (Bavencio®) | LGTVTTVDYW GQGTLVIVSS |
| VH | |
| amino acid | |
| SEQ ID NO: 35 | EVQLLESGGG LVQPGGSLRL SCAASGETES SYIMMWVRQA PGKGLEWVSS |
| Avelumab | IYPSGGITFY ADTVKGRETI SRDNSKNTLY LQMNSLRAED TAVYYCARIK |
| (Bavencio®) | LGTVTTVDYW GQGTLVTVSS ASTKGPSVEP LAPSSKSTSG GTAALGCLVK |
| Heavy chain | DYFPEPVTVS WNSGALTSGV HTFPAVLQSS GLYSLSSVVT VPSSSLGTQT |
| amino acid | YICNVNHKPS NTKVDKKVEP KSCDKTHTCP PCPAPELLGG PSVELFPPKP |
| | KDTLMISRTP EVTCVVVDVS HEDPEVKENW YVDGVEVHNA KTKPREEQYN |
| | STYRVVSVLT VLHQDWLNGK EYKCKVSNKA LPAPIEKTIS KAKGQPREPQ |
| | VYTLPPSRDE LTKNQVSLTC LVKGFYPSDI AVEWESNGQP ENNYKTTPPV |
| | LDSDGSFFLY SKLTVDKSRW QQGNVESCSV MHEALHNHYT QKSLSLSPGK |
| | LDSDGSFFLY |
| SEQ ID NO: 36 | VGGYNYVS |
| Avelumab | |
| (Bavencio®) | |
| VLCDR1 | |
| amino acid | |
| SEQ ID NO: 37 | DVSNRP |
| Avelumab | |
| (Bavencio®) | |
| VLCDR2 | |
| amino acid | |
| SEQ ID NO: 38 | SSYTSSSTRV |
| Avelumab | |
| (Bavencio®) | |
| VLCDR3 | |
| amino acid | |
| SEQ ID NO: 39 | QSALTQPASV SGSPGQSITI SCTGTSSDVG GYNYVSWYQQ HPGKAPKLMI |
| Avelumab | YDVSNRPSGV SNRESGSKSG NTASLTISGL QAEDEADYYC SSYTSSSTRV |
| (Bavencio®) | FGTGTKVTVL |
| VL | |
| amino acid | |
| SEQ ID NO: 40 | QSALTQPASV SGSPGQSITI SCTGTSSDVG GYNYVSWYQQ HPGKAPKLMI |
| Avelumab | YDVSNRPSGV SNRFSGSKSG NTASLTISGL QAEDEADYYC SSYTSSSTRV |
| (Bavencio®) | FGTGTKVTVL GQPKANPTVT LFPPSSEELQ ANKATLVCLI SDEYPGAVTV |
| Light chain | AWKADGSPVK AGVETTKPSK QSNNKYAASS YLSLTPEQWK SHRSYSCQVT |
| amino acid | HEGSTVEKTV APTECS |
| SEQ ID NO: 41 | RYWMS |
| Durvalumab | |
| (IMFINZI®) | |
| VHCDR1 | |
| amino acid | |
| SEQ ID NO: 42 | NIKQDGSEKY |
| Durvalumab | |
| (IMFINZI®) | |
| VHCDR2 | |
| amino acid | |
| SEQ ID NO: 43 | EGGWEGELAF |
| Durvalumab | |
| (IMFINZI®) | |
| VHCDR3 | |
| amino acid | |
| SEQ ID NO: 44 | EVQLVESGGG LVQPGGSLRL SCAASGETFS RYWMSWVRQA PGKGLEWVAN |
| Durvalumab | IKQDGSEKYY VDSVKGRETI SRDNAKNSLY LQMNSLRAED TAVYYCAREG |
| (IMFINZI®) | GWFGET AFDY WGQGTTNVTVS S |
| VH | |
| amino acid | |
| SEQ ID NO: 45 | EVQLVESGGG LVQPGGSLRL SCAASGETFS RYWMSWVRQA PGKGLEWVAN |
| Durvalumab | IKQDCSEKYY VDSVKCRFTI SRDNAKNSLY LQMNSLRAED TAVYYCAREG |
| (IMFINZI®) | GWFGELAFDY WGQGTLVTVS SASTKGPSVF PLAPSSKSTS GGTAALGCLV |
| Heavy chain | KDYFPEPVTV SWNSGALTSG VHTFPAVLQS SGLYSLSSVV TVPSSSIGTQ |
| amino acid | TYICNVNHKP SNTKVDKRVE PKSCDKTHTC PPCPAPEFEG GPSVFLFPPK |
| | PKDTIMISRT PEVTCVVVDV SHEDPEVKEN WYVDGVEVHN AKTKPREEQY |
| | NSTYRVVSVL TVLHQDWING KEYKCKVSNK ALPASIEKTI SKAKGQPREP |
| | QVYTLPPSRE FMTKNQVSLT CLVKGFYPSD IAVEWFSNGQ PENNYKTTPP |
| | VLDSDGSFFL YSKLTVDKSR WQQGNVESCS VMHEALHNHY TQKSLSLSPG |
| | K |
| SEQ ID NO: 46 | RVSSSYLA |
| Durvalumab | |
| (IMFINZI®) | |
| VLCDR1 | |
| amino acid | |
| SEQ ID NO: 47 | DASSRA |
| Durvalumab | |
| (IMFINZI®) | |
| VLCDR2 | |
| amino acid | |
| SEQ ID NO: 48 | QQYGSLPWT |
| Durvalumab | |
| (IMFINZI®) | |
| VLCDR3 | |
| amino acid | |
| SEQ ID NO: 49 | EIVLTQSPGT LSLSPGERAT LSCRASQRVS SSYLAWYQQK PGQAPRLLIY |
| Durvalumab | DASSRATGIP DRESGSGSGT DETLTISRLE PEDFAVYYCQ QYGSLPWTFG |
| (IMFINZI®) | QGTKVEIK |
| VL | |
| amino acid | |
| SEQ ID NO: 50 | EIVLTQSPGT LSLSPGERAT LSCRASQRVS SSYLAWYQQK PGQAPRLLIY |
| Durvalumab | DASSRATGIP DRESGSGSGT DETLTISRLE PEDFAVYYCQ QYGSLPWTFG |
| (IMFINZI®) | QGTKVEIKRT VAAPSVEIFP PSDEQLKSGT ASVVCLINNF YPREAKVQWK |
| Light chain | VDNALQSGNS QESVTEQDSK DSTYSLSSTL TLSKADYEKH KVYACEVTHQ |
| amino acid | GLSSPVTKSF NRGEC |
| SEQ ID NO: 51 | KASGYTFINY YMY |
| Pembrolizumab | |
| (Keytruda®) | |
| VHCDR1 | |
| amino acid | |
| SEQ ID NO: 52 | GINPSNGGTN |
| Pembrolizumab | |
| (Keytruda®) | |
| VHCDR2 | |
| amino acid | |
| SEQ ID NO: 53 | ARRDYREDMG FDY |
| Pembrolizumab | |
| (Keytruda®) | |
| VHCDR3 | |
| amino acid | |
| SEQ ID NO: 54 | QVQLVQSGVE VKKPGASVKV SCKASGYTFT NYYMYWVRQA PGQGLEWMGG |
| Pembrolizumab | INPSNGGTNF NEKFKNRVTL TTDSSTTTAY MELKSLQEDD TAVYYCARRD |
| (Keytruda®) | YRFDMGEDYW GQGTTVTVSS |
| VH | |
| amino acid | |
| SEQ ID NO: 55 | QVQLVQSGVE VKKPGASVKV SCKASGYTFT NYYMYWVRQA PGQGLEWMGG |
| Pembrolizumab | NPSNGGTNEN EKFKNRVTLT TDSSTTTAYM ELKSLQEDDT AVYYCARRDY |
| (Keytruda®) | REDMGEDYWG QGTTVTVSSA STKGPSVEPL APCSRSTSES TAALGCLVKD |
| Heavy chain | YFPEPVTVSW NSGALTSGVH TEPAVLQSSG LYSLSSVVTV PSSSLGTKTY |
| amino acid | TCNVDHKPSN TKVDKRVESK YGPPCPPCPA PEFLGGPSVF LFPPKPKDTL |
| | MISRTPEVTC VVVDVSQEDP EVQENWYVDG VEVHNAKTKP REEQENSTYR |
| | VVSVLTVLHQ DWLNGKEYKC KVSNKGLPSS IEKTISKAKG QPREPQVYTL |
| | PPSQEEMTKN QVSLTCLVKG FYPSDIAVEW ESNGQPENNY KTTPPVLDSD |
| | GSFFLYSRLT VDKSRWQEGN VESCSVMHEA LHNHYTQKSL SLSLGK |
| SEQ ID NO: 56 | RASKGVSTSG YSYLH |
| Pembrolizumab | |
| (Keytruda®) | |
| VLCDR1 | |
| amino acid | |
| SEQ ID NO: 57 | YLASYLES |
| Pembrolizumab | |
| (Keytruda®) | |
| VLCDR2 | |
| amino acid | |
| SEQ ID NO: 58 | QHSRDLPLT |
| Pembrolizumab | |
| (Keytruda®) | |
| VLCDR3 | |
| amino acid | |
| SEQ ID NO: 59 | EIVLTQSPAT LSLSPGERAT LSCRASKGVS TSGYSYLHWY QQKPGQAPRL |
| Pembrolizumab | LIYLASYLES GVPARFSGSG SGTDETLTIS SLEPEDFAVY YCQHSRDLPL |
| (Keytruda®) | TFGGGTKVEI K |
| VL | |
| amino acid | |
| SEQ ID NO: 60 | EIVLTQSPAT LSLSPGERAT LSCRASKGVS TSGYSYLHWY QQKPGQAPRL |
| Pembrolizumab | LIYLASYLES GVPARESGSG SGTDETLTIS SLEPEDFAVY YCQHSRDLPL |
| (Keytruda®) | TFGGGTKVEI KRTVAAPSVE IFPPSDEQLK SGTASVVCLL NNEYPREAKV |
| Light chain | QWKVDNALQS GNSQESVTEQ DSKDSTYSLS STLTLSKADY EKHKVYACEV |
| amino acid | THQGLSSPVT KSENRGEC |
| SEQ ID NO: 61 | ITESNSG |
| Nivolumab | |
| (Opdivo®) | |
| VHCDR1 | |
| amino acid | |
| SEQ ID NO: 62 | WYDGSKRY |
| Nivolumab | |
| (Opdivo®) | |
| VHCDR2 | |
| amino acid | |
| SEQ ID NO: 63 | TND |
| Nivolumab | |
| (Opdivo®) | |
| VHCDR3 | |
| amino acid | |
| SEQ ID NO: 64 | QVQLVESGGG VVQPGRSLRL DCKASGITES NSGMHWVRQA PGKGLEWVAV |
| Nivolumab | IWYDGSKRYY ADSVKGRETI SRDNSKNTLF LQMNSLRAED TAVYYCATND |
| (Opdivo®) | DYWGQGTLVT VSS |
| VH | |
| amino acid | |
| SEQ ID NO: 65 | QVQLVESGGG VVQPGRSLRL DCKASGITES NSGMHWVRQA PGKGLEWVAV |
| Nivolumab | IWYDGSKRYY ADSVKGRETI SRDNSKNTLF LQMNSLRAED TAVYYCATND |
| (Opdivo®) | DYWGQGTLVT VSSASTKGPS VEPLAPCSRS TSESTAALGC LVKDYFPEPV |
| Heavy chain | VLHQDWLNGK SGVHTFPAVL QSSGLYSLSS VVTVPSSSLG TKTYTCNVDH |
| amino acid | TVSWNSGALT VESKYGPPCP PCPAPEELGG PSVELFPPKP KDTLMISRTP |
| | KPSNTKVDKR QEDPEVQENW YVDGVEVHNA KTKPREEQEN STYRVVSVLT |
| | EVTCVVVDVS EYKCKVSNKG LPSSIEKTIS KAKGQPREPQV YTLPPSQEEM |
| | TKNQVSLTCL VKGFYPSDIA VEWESNGQPE NNYKTTPPVL DSDGSFFLYS |
| | RLTVDKSRWQ EGNVESCSVM HEALHNHYTQ KSLSLSLGK |
| SEQ ID NO: 66 | QSVSSY |
| Nivolumab | |
| (Opdivo®) | |
| VLCDR1 | |
| amino acid | |
| SEQ ID NO: 67 | DASNRAT |
| Nivolumab | |
| (Opdivo®) | |
| VLCDR2 | |
| amino acid | |
| SEQ ID NO: 68 | SSNWPR |
| Nivolumab | |
| (Opdivo®) | |
| VLCDR3 | |
| amino acid | |
| SEQ ID NO: 69 | EIVLTQSPAT LSLSPGERAT LSCRASQSVS SYLAWYQQKP GQAPRLLIYD |
| Nivolumab | ASNRATGIPA RESGSGSGTD FTLTISSLEP EDFAVYYCQQ SSNWPRTFGQ |
| (Opdivo®) | GTKVEIK |
| VL | |
| amino acid | |
| SEQ ID NO: 70 | EIVLTQSPAT LSLSPGERAT LSCRASQSVS SYLAWYQQKP GQAPRLLIYD |
| Nivolumab | ASNRATGIPA RFSGSGSGTD FTLTISSLEP EDFAVYYCQQ SSNWPRTFGQ |
| (Opdivo®) | GTKVEIKRTV AAPSVFIFPP SDEQLKSGTA SVVCLINNFY PREAKVQWKV |
| Light chain | DNALQSGNSQ ESVTEQDSKD STYSLSSTLT LSKADYEKHK VYACEVTHQG |
| amino acid | LSSPVTKSEN RGEC |
| SEQ ID NO: 71 | LPAQVAFTPY APEPGSTCRL REYYDQTAQM CCSKCSPGQH AKVECTKTSD |
| Etanercept | TVCDSCEDST YTQLWNWVPE CLSCGSRCSS DQVETQACTR EQNRICTCRP |
| (Enbrel®) | GWYCALSKQE GCRLCAPLRK CRPGFGVARP GTETSDVVCK PCAPGTESNT |
| TNFα | TSSTDICRPH QICNVVAIPG NASMDAVCTS TSPTRSMAPG AVHLPQPVST |
| receptor | RSQHTQPTPE PSTAPSTSEL LPMGPSPPAE GSTGD |
| domain | |
| amino acid | |
| SEQ ID NO: 72 | LPAQVAFTPY APEPGSTCRL REYYDQTAQM CCSKCSPGQH AKVECTKTSD |
| Etanercept | TVCDSCEDST YTQLWNWVPE CLSCGSRCSS DQVETQACTR EQNRICTCRP |
| (Enbrel®) | GWYCALSKQE GCRLCAPLRK CRPGFGVARP GTETSDVVCK PCAPGTESNT |
| TNFα | TSSTDICRPH QICNVVAIPG NASMDAVCTS TSPTRSMAPG AVHLPQPVST |
| receptor | RSQHTQPTPE PSTAPSTSEL LPMGPSPPAE GSTGDEPKSC DKTHTCPPCP |
| domain-Fc | APELLGGPSV FLFPPKPKDT LMISRTPEVT CVVVDVSHED PEVKENWYVD |
| Domain | GVEVHNAKTK PREEQYNSTY RVVSVLTVLH QDWLNGKEYK CKVSNKALPA |
| amino acid | PIEKTISKAK GQPREPQVYT LPPSREEMTK NQVSLTCLVK GFYPSDIAVE |
| | WESNGQPENN YKTTPPVLDS DGSFFLYSKL TVDKSRWQQG NVESCSVMHE |
| | ALHNHYTQKS LSLSPGK |
| SEQ ID NO: 73 | GENIKDTYIH |
| Trastuzumab | |
| (Herceptin®) | |
| VHCDR1 | |
| amino acid | |
| SEQ ID NO: 74 | RIYPINGYTRYADSVKG |
| Trastuzumab | |
| (Herceptin®) | |
| VHCDR2 | |
| amino acid | |
| SEQ ID NO: 75 | WGGDGFYAMDY |
| Trastuzumab | |
| (Herceptin®) | |
| VHCDR3 | |
| amino acid | |
| SEQ ID NO: 76 | EVQLVESGGGLVQPGGSLRLSCAASGENIKDTYIHWVRQAPGKGLEWVARIYPINGYTRYA |
| Trastuzumab | DSVKGRETISADTSKNTAYLQMNSLRAEDTAVYYCSRWGGDGFYAMDYWGQGTLVTVSS |
| (Herceptin®) | |
| VH | |
| amino acid | |
| SEQ ID NO: 77 | EVQLVESGGGLVQPGGSLRLSCAASGENIKDTYIHWVRQAPGKGLEWVARIYPINGYTRYA |
| Trastuzumab | DSVKGRETISADTSKNTAYLQMNSLRAEDTAVYYCSRWGGDGFYAMDYWGQGTLVTVSSAS |
| (Herceptin®) | TKGPSVFPLAPSSKSTSGGTAALGCLVKDYFPEPVTVSWNSGALTSGVHTFPAVLQSSGLY |
| Heavy chain | SLSSVVTVPSSSLGTQTYICNVNHKPSNTKVDKKVEPKSCDKTHTCPPCPAPELLGGPSVE |
| amino acid | LFPPKPKDTLMISRTPEVTCVVVDVSHEDPEVKENWYVDGVEVHNAKTKPREEQYNSTYRV |
| | VSVLTVLHQDWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSRWQQGNVES |
| | CSVMHEALHNHYTQKSLSLSPGK |
| SEQ ID NO: 78 | RASQDVNTAVA |
| Trastuzumab | |
| (Herceptin®) | |
| VLCDR1 | |
| amino acid | |
| SEQ ID NO: 79 | SASELYS |
| Trastuzumab | |
| (Herceptin®) | |
| VLCDR2 | |
| amino acid | |
| SEQ ID NO: 80 | QQHYTTPPT |
| Trastuzumab | |
| (Herceptin®) | |
| VLCDR3 | |
| amino acid | |
| SEQ ID NO: 81 | DIQMTQSPSSLSASVGDRVTITCRASQDVNTAVAWYQQKPGKAPKLLIYSASFLYSGVPSR |
| Trastuzumab | FSGSRSGTDETLTISSLQPEDFATYYCQQHYTTPPTEGQGTKVEIK |
| (Herceptin®) | |
| VL | |
| amino acid | |
| SEQ ID NO: 82 | DIQMTQSPSSLSASVGDRVTITCRASQDVNTAVAWYQQKPGKAPKLLIYSASFLYSGVPSR |
| Trastuzumab | FSGSRSGTDETLTISSLQPEDFATYYCQQHYTTPPTFGQGTKVEIKRTVAAPSVFIFPPSD |
| (Herceptin®) | EQLKSGTASVVCLLNNEYPREAKVQWKVDNALQSGNSQESVTEQDSKDSTYSLSSTLTLSK |
| Light chain | ADYEKHKVYACEVTHQGLSSPVTKSENRGEC |
| amino acid | |
| SEQ ID NO: 83 | GETETDYT |
| Pertuzumab | |
| (Perjeta®) | |
| VHCDR1 | |
| amino acid | |
| SEQ ID NO: 84 | VNPNSGGS |
| Pertuzumab | |
| (Perjeta®) | |
| VHCDR2 | |
| amino acid | |
| SEQ ID NO: 85 | ARNLGPSFYEDY |
| Pertuzumab | |
| (Perjeta®) | |
| VHCDR3 | |
| amino acid | |
| SEQ ID NO: 86 | EVQLVESGGGLVQPGGSLRLSCAASGETETDYTMDWVRQAPGKGLEWVADVNPNSGGSIYN |
| Pertuzumab | QRFKGRETLSVDRSKNTLYLQMNSLRAEDTAVYYCARNLGPSFYEDYWGQGTLVTVSS |
| (Perjeta®) | |
| VH | |
| amino acid | |
| SEQ ID NO: 87 | EVQLVESGGGLVQPGGSLRLSCAASGETFTDYTMDWVRQAPGKGLEWVADVNPNSGGSIYN |
| Pertuzumab | QRFKGRETLSVDRSKNTLYLQMNSLRAEDTAVYYCARNLGPSFYFDYWGQGTLVTVSSAST |
| (Perjeta®) | KGPSVFPLAPSSKSTSGGTAALGCLVKDYFPEPVTVSWNSGALTSGVHTFPAVLQSSGLYS |
| Heavy chain | LSSVVTVPSSSIGTQTYICNVNHKPSNTKVDKKVEPKSCDKTHTCPPCPAPELLGGPSVEL |
| amino acid | FPPKPKDTLMISRTPEVTCVVVDVSHEDPEVKENWYVDGVEVHNAKTKPREEQYNSTYRVV |
| | SVLTVLHQDWLNGKEYKCKVSNKALPAPIEKTISKAKGQPREPQVYTLPPSREEMTKNQVS |
| | LTCLVKGFYPSDIAVEWESNGQPENNYKTTPPVLDSDGSFFLYSKLTVDKSRWQQGNVESC |
| | SVMHEALHNHYTQKSLSLSPG |
| SEQ ID NO: 88 | QDVSIG |
| Pertuzumab | |
| (Perjeta®) | |
| VLCDR1 | |
| amino acid | |
| SEQ ID NO: 89 | SAS |
| Pertuzumab | |
| (Perjeta®) | |
| VLCDR2 | |
| amino acid | |
| SEQ ID NO: 90 | QQYYIYPYT |
| Pertuzumab | |
| (Perjeta®) | |
| VLCDR3 | |
| amino acid | |
| SEQ ID NO: 91 | DIQMTQSPSSLSASVGDRVTITCKASQDVSIGVAWYQQKPGKAPKLLIYSASYRYTGVPSR |
| Pertuzumab | FSGSGSGTDETLTISSLQPEDFATYYCQQYYTYPYTFGQGTKVEIK |
| (Perjeta®) | |
| VL | |
| amino acid | |
| SEQ ID NO: 92 | DIQMTQSPSSLSASVGDRVTITCKASQDVSIGVAWYQQKPGKAPKLLIYSASYRYTGVPSR |
| Pertuzumab | FSGSGSGTDETLTISSLQPEDFATYYCQQYYIYPYTFGQGTKVEIKRTVAAPSVFIFPPSD |
| (Perjeta®) | EQLKSGTASVVCLLNNEYPREAKVQWKVDNALQSGNSQESVTEQDSKDSTYSLSSTLTLSK |
| Light chain | ADYEKHKVYACEVTHQGLSSPVTKSENRGEC |
| amino acid | |
| SEQ ID NO: 93 | GESLINYGVH |
| Cetuximab | |
| (Erbitux®) | |
| VHCDR1 | |
| amino acid | |
| SEQ ID NO: 94 | VIWSGGNTDY |
| Cetuximab | |
| (Erbitux®) | |
| VHCDR2 | |
| amino acid | |
| SEQ ID NO: 95 | ARALTYYDYEFAY |
| Cetuximab | |
| (Erbitux®) | |
| VHCDR3 | |
| amino acid | |
| SEQ ID NO: 96 | QVQLKQSGPGLVQPSQSLSITCTVSGESLTNYGVHWVRQSPGKGLEWLGVIWSGGNTDYNT |
| Cetuximab | PFTSRLSINKDNSKSQVFFKMNSLQSNDTAIYYCARALTYYDYEFAYWGQGTLVTVSA |
| (Erbitux®) | |
| VH | |
| amino acid | |
| SEQ ID NO: 97 | QVQLKQSGPGLVQPSQSLSITCTVSGESLINYGVHWVRQSPGKGLEWLGVIWSGGNTDYNT |
| Cetuximab | PETSRLSINKDNSKSQVFFKMNSLQSNDTAIYYCARALTYYDYEFAYWGQGTLVTVSAAST |
| (Erbitux®) | KGPSVFPLAPSSKSTSGGTAALGCLVKDYFPEPVTVSWNSGALTSGVHTFPAVLQSSGLYS |
| Heavy chain | LSSVVTVPSSSLGTQTYICNVNHKPSNTKVDKRVEPKSCDKTHTCPPCPAPELLGGPSVEL |
| amino acid | FPPKPKDTLMISRTPEVTCVVVDVSHEDPEVKENWYVDGVEVHNAKTKPREEQYNSTYRVV |
| | SVLTVLHQDWLNGKEYKCKVSNKALPAPIEKTISKAKGQPREPQVYTLPPSREEMTKNQVS |
| | LTCLVKGFYPSDIAVEWESNGQPENNYKTTPPVLDSDGSFFLYSKLTVDKSRWQQGNVESC |
| | SVMHEALHNHYTQKSLSLSPGK |
| SEQ ID NO: 98 | RASQSIGTNIH |
| Cetuximab | |
| (Erbitux®) | |
| VLCDR1 | |
| amino acid | |
| SEQ ID NO: 99 | YASESIS |
| Cetuximab | |
| (Erbitux®) | |
| VLCDR2 | |
| amino acid | |
| SEQ ID NO: 100 | QQNNNWPTTE |
| Cetuximab | |
| (Erbitux®) | |
| VLCDR3 | |
| amino acid | |
| SEQ ID NO: 101 | DILLTQSPVILSVSPGERVSESCRASQSIGTNIHWYQQRTNGSPRLLIKYASESISGIPSR |
| Cetuximab | FSGSGSGTDFTLSINSVESEDIADYYCQQNNNWPTTEGAGTKLELK |
| (Erbitux®) | |
| VL | |
| amino acid | |
| SEQ ID NO: 102 | DILLTQSPVILSVSPGERVSESCRASQSIGTNIHWYQQRINGSPRLLIKYASESISGIPSR |
| Cetuximab | FSGSGSGTDETLSINSVESEDIADYYCQQNNNWPTTEGAGTKLELKRTVAAPSVEIFPPSD |
| (Erbitux®) | EQLKSGTASVVCLLNNEYPREAKVQWKVDNALQSGNSQESVTEQDSKDSTYSLSSTLTLSK |
| Light chain | ADYEKHKVYACEVTHQGLSSPVTKSENRGEC |
| amino acid | |
| SEQ ID NO: 103 | GYTFTSYNMH |
| Rituximab | |
| (Rituxan®) | |
| VHCDR1 | |
| amino acid | |
| SEQ ID NO: 104 | AIYPGNGDTSYNQKFKG |
| Rituximab | |
| (Rituxan®) | |
| VHCDR2 | |
| amino acid | |
| SEQ ID NO: 105 | ARSTYYGGDWYENV |
| Rituximab | |
| (Rituxan®) | |
| VHCDR3 | |
| amino acid | |
| SEQ ID NO: 106 | QVQLQQPGAELVKPGASVKMSCKASGYTFTSYNMHWVKQTPGRGLEWIGATYPGNGDTSYN |
| Rituximab | QKFKGKATLTADKSSSTAYMQLSSLTSEDSAVYYCARSTYYGGDWYENVWGAGTTVTVSA |
| (Rituxan®) | |
| VH | |
| amino acid | |
| SEQ ID NO: 107 | QVQLQQPGAELVKPGASVKMSCKASGYTFTSYNMHWVKQTPGRGLEWIGATYPGNGDTSYN |
| Rituximab | QKFKGKATLTADKSSSTAYMQLSSLTSEDSAVYYCARSTYYGGDWYENVWGAGTTVTVSAA |
| (Rituxan®) | STKGPSVFPLAPSSKSTSGGTAALGCLVKDYFPEPVTVSWNSGALTSGVHTFPAVLQSSGL |
| Heavy chain | YSLSSVVTVPSSSLGTQTYICNVNHKPSNTKVDKKAEPKSCDKTHTCPPCPAPELLGGPSV |
| amino acid | FLFPPKPKDTLMISRTPEVTCVVVDVSHEDPEVKENWYVDGVEVHNAKTKPREEQYNSTYR |
| | VVSVLTVLHQDWLNGKEYKCKVSNKALPAPIEKTISKAKGQPREPQVYTLPPSRDELTKNQ |
| | VSLTCLVKGFYPSDIAVEWESNGQPENNYKTTPPVLDSDGSFFLYSKLTVDKSRWQQGNVE |
| | SCSVMHEALHNHYTQKSLSLSPGK |
| SEQ ID NO: 108 | RASSSVSYIH |
| Rituximab | |
| (Rituxan®) | |
| VLCDR1 | |
| amino acid | |
| SEQ ID NO: 109 | ATSNLASGV |
| Rituximab | |
| (Rituxan®) | |
| VLCDR2 | |
| amino acid | |
| SEQ ID NO: 110 | QQWTSNPPT |
| Rituximab | |
| (Rituxan®) | |
| VLCDR3 | |
| amino acid | |
| SEQ ID NO: 111 | QIVLSQSPAILSASPGEKVTMTCRASSSVSYIHWFQQKPGSSPKPWIYATSNLASGVPVRE |
| Rituximab | SGSGSGTSYSLTISRVEAEDAATYYCQQWTSNPPTFGGGTKLEIK |
| (Rituxan®) | |
| VL | |
| amino acid | |
| SEQ ID NO: 112 | QIVLSQSPAILSASPGEKVTMICRASSSVSYIHWFQQKPGSSPKPWIYATSNLASGVPVRF |
| Rituximab | SGSGSGTSYSLTISRVEAEDAATYYCQQWTSNPPTFGGGTKLEIKRTVAAPSVFIFPPSDE |
| (Rituxan®) | QLKSGTASVVCLLNNEYPREAKVQWKVDNALQSGNSQESVTEQDSKDSTYSLSSTLTLSKA |
| Light chain | DYEKHKVYACEVTHQGLSSPVTKSENRGEC |
| amino acid | |
| SEQ ID NO: 113 | GYAFSYSWIN |
| Obinutuzumab | |
| (Gazyva®) | |
| VHCDR1 | |
| amino acid | |
| SEQ ID NO: 114 | RIFPGDGDTDYNGKFKG |
| Obinutuzumab | |
| (Gazyva®) | |
| VHCDR2 | |
| amino acid | |
| SEQ ID NO: 115 | ARNVEDGYWLVY |
| Obinutuzumab | |
| (Gazyva®) | |
| VHCDR3 | |
| amino acid | |
| SEQ ID NO: 116 | QVQLVQSGAEVKKPGSSVKVSCKASGYAFSYSWINWVRQAPGQGLEWMGRIFPGDGDTDYN |
| Obinutuzumab | GKFKGRVTITADKSTSTAYMELSSLRSEDTAVYYCARNVEDGYWLVYWGQGTLVTVSS |
| (Gazyva®) | |
| VH | |
| amino acid | |
| SEQ ID NO: 117 | QVQLVQSGAEVKKPGSSVKVSCKASGYAFSYSWINWVRQAPGQGLEWMGRIFPGDGDTDYN |
| Obinutuzumab | GKFKGRVTITADKSTSTAYMELSSLRSEDTAVYYCARNVEDGYWLVYWGQGTLVTVSSAST |
| (Gazyva®) | KGPSVFPLAPSSKSTSGGTAALGCLVKDYFPEPVTVSWNSGALTSGVHTEPAVLQSSGLYS |
| Heavy chain | LSSVVTVPSSSLGTQTYICNVNHKPSNTKVDKKVEPKSCDKTHTCPPCPAPELLGGPSVEL |
| amino acid | FPPKPKDTLMISRTPEVTCVVVDVSHEDPEVKENWYVDGVEVHNAKTKPREEQYNSTYRVV |
| | SVLTVLHQDWLNGKEYKCKVSNKALPAPIEKTISKAKGQPREPQVYTLPPSRDELTKNQVS |
| | LTCLVKGFYPSDTAVEWESNGQPENNYKTTPPVLDSDGSFFLYSKLIVDKSRWQQGNVFSC |
| | SVMHEALHNHYTQKSLSLSPGK |
| SEQ ID NO: 118 | RSSKSLLHSNGITYLY |
| Obinutuzumab | |
| (Gazyva®) | |
| VLCDR1 | |
| amino acid | |
| SEQ ID NO: 119 | QMSNLVSGV |
| Obinutuzumab | |
| (Gazyva®) | |
| VLCDR2 | |
| amino acid | |
| SEQ ID NO: 120 | AQNLELPYT |
| Obinutuzumab | |
| (Gazyva®) | |
| VLCDR3 | |
| amino acid | |
| SEQ ID NO: 121 | DIVMTQTPLSLPVTPGEPASISCRSSKSLLHSNGITYLYWYLQKPGQSPQLLIYQMSNLVS |
| Obinutuzumab | GVPDRESGSGSGTDETLKISRVEAEDVGVYYCAQNLELPYTEGGGTKVEIK |
| (Gazyva®) | |
| VL | |
| amino acid | |
| SEQ ID NO: 122 | DIVMTQTPLSLPVTPGEPASISCRSSKSLLHSNGITYLYWYLQKPGQSPQLLIYQMSNLVS |
| Obinutuzumab | GVPDRFSGSGSGTDFTLKISRVEAEDVGVYYCAQNLELPYTFGGGTKVEIKRTVAAPSVFI |
| (Gazyva®) | FPPSDEQLKSGTASVVCLLNNEYPREAKVQWKVDNALQSGNSQESVTEQDSKDSTYSLSST |
| Light chain | LTLSKADYEKHKVYACEVTHQGLSSPVTKSENRGEC |
| amino acid | |
| SEQ ID NO: 123 | MIHTNLKKKF SCCVLVELLF AVICVWKEKK KGSYYDSFKL |
| ST6GAL1, aa | QTKEFQVLKS LGKLAMGSDS QSVSSSSTQD PHRGRQTLGS |
| | LRGLAKAKPE ASFQVWNKDS SSKNLIPRLQ KIWKNYLSMN |
| | KYKVSYKGPG PGIKFSAEAL RCHLRDHVNV SMVEVTDEPE |
| | NTSEWEGYLP KESIRTKAGP WGRCAVVSSA GSLKSSQLGR |
| | EIDDHDAVLR FNGAPTANFQ QDVGTKTTIR LMNSQLVTTE |
| | KRFLKDSLYN EGILIVWDPS VYHSDIPKWY QNPDYNFENN |
| | YKTYRKLHPN QPFYILKPQM PWELWDILQE ISPEEIQPNP |
| | PSSGMLGIII MMTLCDQVDI YEFLPSKRKT DVCYYYQKFF |
| | DSACTMGAYH PLLYEKNLVK HLNQGTDEDI YLLGKATLPG FRTIHC |
| SEQ ID NO: 124 | NVVAPSLEVY VDHASLPTLQ QLMDIIKSEE ENPTAQRYIA |
| PspST only, aa | WGRIVPTDEQ MKELNITSFA LINNHTPADL VQEIVKQAQT |
| | KHRLNVKLSS NTAHSFDNLV PILKELNSEN NVTVTNIDLY |
| | DDGSMEYVNL YNWRDTLNKT DNLKIGKDYL EDVINGINED |
| | TSNTGTSSVY NWQKLYPANY HELRKDYLTL EPSLHELRDY |
| | IGDSLKQMQW DGFKKENSKQ QELFLSIVNF DKQKLQNEYN |
| | SSNLPNFVFT GTTVWGGNHE REYYAKQQIN VINNAINESS |
| | PHYLGNSYDL FFKGHPGGGI INTLIMQNYP SMVDIPSKIS |
| | FEVLMMTDML PDAVAGIASS LYFTIPAEKI KFIVETSTET |
| | ITDRETALRS PLVQVMIKLG IVKEENVLEW ADLPNCETGV C |
| SEQ ID NO: 125 | MIHTNLKKKF SCCVLVELLF AVICVWKEKK KGSYYDSFKL |
| Modified PspST | QTKEFQVLKS LGKLAMGSDS QSVSSSSTQD PHRGRQTLGS |
| (ST6GAL1 signal | LRGLAKAKPG GGGSGGGGSG GGGSNVVAPS LEVYVDHASL |
| peptide), aa | PTLQQLMDII KSEEENPTAQ RYIAWGRIVP TDEQMKELNI |
| | TSFALINNHT PADLVQEIVK QAQTKHRLNV KLSSNTAHSF |
| | DNLVPILKEL NSFNNVTVTN IDLYDDGSME YVNLYNWRDT |
| | LNKTDNLKIG KDYLEDVING INEDTSNTGT SSVYNWQKLY |
| | PANYHELRKD YLTLEPSLHE LRDYIGDSLK QMQWDGEKKE |
| | NSKQQELFLS IVNEDKQKLQ NEYNSSNLPN FVFTGTTVWG |
| | GNHEREYYAK QQINVINNAI NESSPHYLGN SYDLFFKGHP |
| | GGGIINTLIM QNYPSMVDIP SKISFEVLMM TDMLPDAVAG |
| | IASSLYFTIP AEKIKFIVFT STETITDRET ALRSPLVQVM |
| | IKLGIVKEEN VLFWADLPNC ETGVCGSGAT NESLLKQAGD |
| | VEENPGPMRL REPLLSGSAA MPGASLQRAC RLLVAVCALH |
| | LGVTLVYYLA GRDLSRLPQL VGVSTPLQGG SNSAAAIGQS |
| | SGELRTGGAR PPPPLGASSQ PRPGGDSSPV VDSGPGPASN |
| | LTSVPVPHTT ALSLPACPEE SPLLVGPMLI EENMPVDLEL |
| | VAKQNPNVKM GGRYAPRDCV SPHKVAIIIP FRNRQEHLKY |
| | WLYYLHPVLQ RQQLDYGIYV INQAGDTIEN RAKLLNVGFQ |
| | EALKDYDYTC FVFSDVDLIP MNDHNAYRCF SQPRHISVAM |
| | DKFGFSLPYV QYFGGVSALS KQQFLTINGE PNNYWGWGGE |
| | DDDIFNRLVF RGMSISRPNA VVGRCRMIRH SRDKKNEPNP |
| | QRFDRIAHTK ETMLSDGLNS LTYQVLDVQR YPLYTQITVD IGTPS |
| SEQ ID NO: 126 | MRLREPLLSG SAAMPGASLQ RACRLLVAVC ALHLGVTLVY |
| Modified PspST | YLAGRDLSRL PQLVGVSTPL QGGSNSAAAI GQSSGELRTG |
| (B4GALT1 signal | GARPPPPLGA SSQPRPGGGG GGSGGGGSGG GGSNVVAPSL |
| peptide), aa | EVYVDHASLP TLQQLMDIIK SEEENPTAQR YIAWGRIVPT |
| | DEQMKELNIT SFALINNHTP ADLVQEIVKQ AQTKHRLNVK |
| | LSSNTAHSED NLVPILKELN SENNVTVTNI DLYDDGSMEY |
| | VNLYNWRDTL NKTDNLKIGK DYLEDVINGI NEDTSNTGTS |
| | SVYNWQKLYP ANYHELRKDY LTLEPSLHEL RDYIGDSLKQ |
| | MQWDGEKKEN SKQQELFLSI VNEDKQKLQN EYNSSNLPNE |
| | VFTGTTVWGG NHEREYYAKQ QINVINNAIN ESSPHYLGNS |
| | YDLFFKGHPG GGIINTLIMQ NYPSMVDIPS KISFEVLMMT |
| | DMLPDAVAGI ASSLYFTIPA EKIKFIVETS TETITDRETA |
| | LRSPLVQVMI KLGIVKEENV LFWADLPNCE TGVCGSGATN |
| | FSLLKQAGDV EENPGPMRLR EPLLSGSAAM PGASLQRACR |
| | LLVAVCALHL GVTLVYYLAG RDLSRLPQLV GVSTPLQGGS |
| | NSAAAIGQSS GELRTGGARP PPPLGASSQP RPGGDSSPVV |
| | DSGPGPASNL TSVPVPHTTA LSLPACPEES PLLVGPMLIE |
| | FNMPVDLELV AKQNPNVKMG GRYAPRDCVS PHKVAIIIPF |
| | RNRQEHLKYW LYYLHPVLQR QQLDYGIYVI NQAGDTIENR |
| | AKLLNVGFQE ALKDYDYTCF VESDVDLIPM NDHNAYRCES |
| | QPRHISVAMD KFGESLPYVQ YFGGVSALSK QQFLTINGFP |
| | NNYWGWGGED DDIFNRLVER GMSISRPNAV VGRCRMIRHS |
| | RDKKNEPNPQ REDRIAHTKE TMLSDGLNSL TYQVLDVQRY |
| | PLYTQITVDI GTPS |
| SEQ ID NO: 127 | MRLREPLLSGSAAMPGASLQRACRLLVAVCALHLGVTLVYYLAGRDLSRLPQ |
| B4GALT1, aa | LVGVSTPLQGGSNSAAAIGQSSGELRTGGARPPPPLGASSQPRPGGDSSPVV |
| | DSGPGPASNLTSVPVPHTTALSLPACPEESPLLVGPMLIEFNMPVDLELVAK |
| | QNPNVKMGGRYAPRDCVSPHKVAIIIPFRNRQEHLKYWLYYLHPVLQRQQLD |
| | YGIYVINQAGDTIFNRAKLLNVGFQEALKDYDYTCFVESDVDLIPMNDHNAY |
| | RCFSQPRHISVAMDKFGFSLPYVQYFGGVSALSKQQFLTINGFPNNYWGWGG |
| | EDDDIFNRLVERGMSISRPNAVVGRCRMIRHSRDKKNEPNPQREDRIAHTKE |
| | TMLSDGLNSLTYQVLDVQRYPLYTQITVDIGTPS |
| SEQ ID NO: 128 | GSGATNFSLLKQAGDVEENPGP |
| Ribosomal | |
| Shifting | |
| peptide-P2A, | |
| aa | |
| SEQ ID NO: 129 | GSGEGRGSLLTCGDVEENPGP |
| Ribosomal | |
| Shifting | |
| peptide-T2A, | |
| aa | |
| SEQ ID NO: 130 | GSGQCTNYALLKLAGDVESNPGP |
| Ribosomal | |
| Shifting | |
| peptide-E2A, | |
| aa | |
| SEQ ID NO: 131 | GSGVKQTLNFDLLKLAGDVESNPGP |
| Ribosomal | |
| Shifting | |
| peptide-F2A, | |
| aa | |
| SEQ ID NO: 132 | ATGATCCACACCAATCTGAAGAAGAAGTTTAGCTGTTGCGTGCTGGTGTTCC |
| ST6GAL1, nt | TGCTGTTCGCTGTGATCTGCGTGTGGAAGGAGAAGAAGAAGGGCAGCTATTA |
| | TGACTCCTTTAAGCTGCAGACCAAGGAGTTCCAGGTGCTGAAGTCCCTGGGC |
| | AAGCTGGCCATGGGCAGCGACTCCCAGTCCGTGAGCTCCTCCAGCACCCAGG |
| | ACCCCCACCGGGGAAGGCAGACCCTGGGATCCCTGAGGGGCCTGGCTAAGGC |
| | CAAGCCTGAGGCTTCCTTCCAGGTGTGGAATAAGGATAGCTCCAGCAAGAAC |
| | CTGATCCCTAGGCTGCAGAAGATCTGGAAGAACTATCTGAGCATGAACAAGT |
| | ATAAGGTGAGCTATAAGGGCCCTGGCCCTGGCATCAAGTTCTCCGCCGAGGC |
| | CCTGAGGTGCCACCTGAGAGATCACGTGAATGTGAGCATGGTGGAGGTGACC |
| | GACTTCCCTTTTAACACCTCCGAGTGGGAGGGCTACCTGCCCAAGGAGAGCA |
| | TCCGGACCAAGGCCGGCCCTTGGGGCAGATGTGCCGTGGTGTCCAGCGCCGG |
| | CTCCCTGAAGAGCTCCCAGCTGGGCCGGGAGATCGATGACCACGATGCTGTG |
| | CTGAGGTTCAATGGCGCTCCTACCGCTAACTTTCAGCAGGATGTGGGCACCA |
| | AGACCACCATCCGGCTGATGAATAGCCAGCTGGTGACCACCGAGAAGCGGTT |
| | TCTGAAGGACTCCCTGTACAACGAGGGCATCCTGATCGTGTGGGACCCCTCC |
| | GTGTACCACAGCGACATCCCCAAGTGGTATCAGAACCCCGACTACAACTTCT |
| | TCAATAATTACAAGACCTACCGGAAGCTGCACCCCAACCAGCCCTTTTATAT |
| | CCTGAAGCCCCAGATGCCTTGGGAGCTGTGGGACATCCTGCAGGAGATCTCC |
| | CCCGAGGAGATCCAGCCTAACCCCCCTAGCAGCGGCATGCTGGGCATCATCA |
| | TCATGATGACCCTGTGCGATCAGGTGGATATCTACGAGTTCCTGCCCAGCAA |
| | GCGGAAGACCGACGTGTGTTATTATTATCAGAAGTTCTTCGATTCCGCCTGC |
| | ACCATGGGCGCCTATCACCCTCTGCTGTACGAGAAGAATCTGGTGAAGCACC |
| | TGAACCAGGGCACCGACGAGGACATCTATCTGCTGGGCAAGGCCACCCTGCC |
| | CGGCTTTAGGACCATCCACTGCTGA |
| SEQ ID NO: 133 | ATGAAAAACTTTTTATTATTAACTTTAATATTACTTACTGCTTGTAATAATT |
| PspST only, nt | CAGAAGAAAATACACAATCTATTATTAAAAATGATATTAATAAAACTATTAT |
| | TGATGAGGAGTATGTTAATTTAGAGCCAATTAATCAATCAAACATCTCTTTT |
| | ACAAAACACTCTTGGGTACAAACTTGTGGTACGCAACAACTATTAACAGAAC |
| | AAAATAAAGAGTCAATATCATTATCTGTAGTGGCGCCACGATTAGATGACGA |
| | TGAAAAGTACTGCTTTGATTTTAATGGTGTTAGTAATAAAGGTGAAAAATAT |
| | ATAACAAAAGTAACATTAAACGTAGTGGCTCCATCTTTAGAGGTTTATGTTG |
| | ATCATGCATCTCTTCCAACTCTTCAGCAGCTAATGGATATTATTAAATCGGA |
| | AGAAGAAAATCCTACAGCACAAAGATATATAGCTTGGGGGAGAATAGTTCCG |
| | ACTGATGAGCAAATGAAAGAGTTAAATATTACATCGTTTGCATTGATAAATA |
| | ACCATACACCAGCTGACTTAGTACAAGAAATTGTTAAGCAAGCACAAACAAA |
| | GCATAGATTGAATGTTAAACTTAGCTCTAACACTGCTCATTCATTTGATAAT |
| | TTAGTGCCAATACTAAAAGAATTAAATTCGTTTAATAACGTTACGGTAACAA |
| | ATATAGATTTATATGATGATGGTTCAGCAGAATATGTAAATTTATATAACTG |
| | GAGAGATACATTAAATAAAACAGATAATTTAAAAATTGGTAAAGATTATCTT |
| | GAGGATGTCATTAATGGTATCAATGAAGACACTTCAAATACAGGAACATCAT |
| | CTGTTTATAACTGGCAAAAACTATATCCAGCTAACTACCATTTTTTAAGAAA |
| | AGATTATTTAACTTTAGAACCATCATTACATGAGTTACGAGACTATATTGGT |
| | GATAGTTTAAAGCAAATGCAATGGGATGGTTTCAAAAAATTCAATAGCAAAC |
| | AACAAGAATTATTCTTATCGATTGTTAATTTTGACAAACAAAAATTACAAAA |
| | TGAATATAATTCATCTAATTTACCAAACTTTGTGTTTACAGGTACGACTGTA |
| | TGGGCTGGTAACCATGAAAGAGAGTATTATGCGAAACAACAAATTAATGTCA |
| | TTAATAATGCAATTAATGAATCGAGCCCACATTATTTAGGCAATAGTTATGA |
| | TTTGTTCTTCAAAGGTCACCCTGGTGGCGGTATCATTAATACATTAATAATG |
| | CAAAACTATCCTTCAATGGTTGATATTCCATCAAAAATATCATTTGAAGTTT |
| | TGATGATGACAGATATGCTTCCTGATGCAGTTGCTGGTATAGCGAGCTCTTT |
| | ATATTTCACGATACCAGCTGAAAAAATTAAATTTATAGTTTTTACATCGACA |
| | GAAACTATAACTGATCGTGAAACTGCTTTGAGAAGTCCTTTAGTTCAAGTAA |
| | TGATAAAACTAGGTATTGTAAAAGAAGAGAATGTACTTTTTTGGGCTGATCT |
| | GCCAAATTGTGAAACAGGTGTTTGTATTGCAGTCTAG |
| SEQ ID NO: 134 | ATGATCCACACCAACCTGAAGAAGAAGTTCTCCTGCTGTGTGCTGGTGTTTC |
| Modified PspST | TGCTGTTCGCTGTGATCTGCGTGTGGAAGGAGAAGAAGAAGGGCAGCTATTA |
| (ST6GAL1 signal | TGACAGCTTTAAGCTGCAGACCAAGGAGTTTCAGGTGCTGAAGTCCCTGGGC |
| peptide), nt | AAGCTGGCCATGGGCTCCGACTCCCAGTCCGTGTCCAGCTCCAGCACCCAGG |
| | ATCCTCACCGGGGCAGGCAGACCCTGGGCTCTCTGAGAGGCCTGGCTAAGGC |
| | TAAGCCCGGCGGCGGAGGCAGCGGAGGAGGAGGATCTGGCGGCGGAGGATCC |
| | AATGTGGTGGCCCCTTCCCTGGAGGTGTATGTGGACCACGCCTCCCTGCCCA |
| | CCCTGCAGCAGCTGATGGATATCATCAAGAGCGAGGAGGAGAATCCTACCGC |
| | CCAGCGGTATATCGCCTGGGGCCGGATCGTGCCTACCGACGAGCAGATGAAG |
| | GAGCTGAACATCACCTCCTTCGCTCTGATCAACAATCACACCCCCGCCGACC |
| | TGGTGCAGGAGATCGTGAAGCAGGCCCAGACCAAGCACCGGCTGAACGTGAA |
| | GCTGTCCTCCAACACCGCCCACTCCTTTGACAACCTGGTGCCTATCCTGAAG |
| | GAGCTGAATTCCTTTAATAACGTGACCGTGACCAACATCGACCTGTACGACG |
| | ACGGCTCCATGGAGTATGTGAACCTGTATAACTGGAGGGACACCCTGAATAA |
| | GACCGACAATCTGAAGATCGGCAAGGACTATCTGGAGGACGTGATCAATGGC |
| | ATCAATGAGGACACCTCCAACACCGGCACCAGCAGCGTGTATAACTGGCAGA |
| | AGCTGTACCCTGCTAATTACCACTTTCTGAGGAAGGATTATCTGACCCTGGA |
| | GCCTTCCCTGCACGAGCTGAGGGACTACATCGGCGATAGCCTGAAGCAGATG |
| | CAGTGGGATGGCTTTAAGAAGTTCAACAGCAAGCAGCAGGAGCTGTTTCTGA |
| | GCATCGTGAATTTTGACAAGCAGAAGCTGCAGAACGAGTATAACTCCTCCAA |
| | TCTGCCTAACTTTGTGTTCACCGGCACCACCGTGTGGGGCGGCAATCACGAG |
| | CGGGAGTACTATGCCAAGCAGCAGATCAACGTGATCAATAATGCCATCAATG |
| | AGAGCAGCCCCCACTACCTGGGCAATAGCTACGATCTGTTCTTCAAGGGCCA |
| | CCCTGGCGGCGGCATCATCAACACCCTGATCATGCAGAACTATCCTTCCATG |
| | GTGGATATCCCTAGCAAGATCTCCTTCGAGGTGCTGATGATGACCGACATGC |
| | TGCCCGACGCCGTGGCTGGCATCGCCTCTAGCCTGTATTTTACCATCCCCGC |
| | TGAGAAGATCAAGTTCATCGTGTTTACCAGCACCGAGACCATCACCGACCGG |
| | GAGACCGCCCTGAGGTCCCCACTGGTGCAGGTGATGATCAAGCTGGGCATCG |
| | TGAAGGAGGAGAACGTGCTGTTTTGGGCCGACCTGCCCAACTGTGAGACCGG |
| | CGTGTGTGGCTCCGGCGCTACCAACTTCTCCCTGCTGAAGCAGGCTGGCGAT |
| | GTGGAGGAGAATCCCGGCCCTATGCGGCTGCGGGAGCCCCTGTTGTCCGGCT |
| | CTGCCGCTATGCCCGGCGCTTCCCTGCAGAGAGCCTGTCGGCTGCTGGTGGC |
| | TGTGTGCGCTCTGCACCTGGGCGTGACCCTGGTGTACTATCTGGCTGGCCGG |
| | GATCTGAGCCGGCTGCCTCAGCTGGTGGGCGTGAGCACCCCCCTGCAGGGAG |
| | GATCCAACTCCGCCGCTGCCATCGGCCAGTCCTCCGGAGAGCTGCGGACCGG |
| | AGGCGCTAGGCCTCCACCACCACTGGGCGCTTCTTCCCAGCCTCGGCCCGGA |
| | GGAGATAGCAGCCCCGTGGTGGACTCCGGCCCTGGACCTGCTTCCAACCTGA |
| | CCAGCGTGCCCGTGCCTCACACCACCGCTCTGTCCCTGCCCGCCTGTCCCGA |
| | GGAGTCCCCTCTGCTGGTGGGCCCTATGCTGATCGAGTTTAATATGCCTGTG |
| | GACCTGGAGCTGGTGGCTAAGCAGAATCCTAACGTGAAGATGGGCGGCAGGT |
| | ACGCTCCCAGGGACTGCGTGAGCCCTCACAAGGTGGCCATCATCATCCCTTT |
| | CCGGAACCGGCAGGAGCACCTGAAGTACTGGCTGTACTACCTGCACCCTGTG |
| | CTGCAGAGGCAGCAGCTGGACTATGGCATCTACGTGATCAACCAGGCCGGCG |
| | ACACCATCTTTAACCGGGCCAAGCTGCTGAATGTGGGCTTTCAGGAGGCCCT |
| | GAAGGATTATGACTACACCTGCTTTGTGTTTAGCGATGTGGATCTGATCCCT |
| | ATGAACGATCACAACGCCTACCGGTGCTTCAGCCAGCCTCGGCACATCAGCG |
| | TGGCCATGGACAAGTTCGGCTTCTCCCTGCCTTACGTGCAGTACTTCGGCGG |
| | CGTGTCCGCCCTGTCCAAGCAGCAGTTCCTGACCATCAACGGCTTTCCTAAT |
| | AACTACTGGGGCTGGGGCGGCGAGGATGATGACATCTTTAATCGGCTGGTGT |
| | TTAGGGGCATGAGCATCAGCCGGCCTAACGCCGTGGTGGGCAGGTGCAGGAT |
| | GATCAGGCACTCCCGGGACAAGAAGAACGAGCCTAATCCTCAGAGGTTTGAC |
| | AGGATCGCTCACACCAAGGAGACCATGCTGAGCGACGGCCTGAATAGCCTGA |
| | CCTACCAGGTGCTGGACGTGCAGAGGTACCCTCTGTATACCCAGATCACCGT |
| | GGATATCGGCACCCCTAGCTGA |
| SEQ ID NO: 135 | ATGAGGCTGCGGGAGCCATTGCTGAGCGGCTCCGCTGCCATGCCTGGCGCTT |
| Modified PspST | CTCTGCAGAGGGCTTGTAGGCTGCTGGTGGCCGTGTGCGCCCTGCACCTGGG |
| (B4GALT1 signal | AGTGACCCTGGTGTACTACCTGGCTGGCCGGGACCTGTCCCGGCTGCCTCAG |
| peptide), nt | TTGGTGGGCGTGTCCACCCCTCTGCAGGGCGGCGGAGGCAGCGGAGGAGGAG |
| | GATCTGGCGGCGGAGGATCCAATGTGGTGGCCCCTTCCCTGGAGGTGTATGT |
| | GGACCACGCCTCCCTGCCCACCCTGCAGCAGCTGATGGATATCATCAAGAGC |
| | GAGGAGGAGAATCCTACCGCCCAGCGGTATATCGCCTGGGGCCGGATCGTGC |
| | CTACCGACGAGCAGATGAAGGAGCTGAACATCACCTCCTTCGCTCTGATCAA |
| | CAATCACACCCCCGCCGACCTGGTGCAGGAGATCGTGAAGCAGGCCCAGACC |
| | AAGCACCGGCTGAACGTGAAGCTGTCCTCCAACACCGCCCACTCCTTTGACA |
| | ACCTGGTGCCTATCCTGAAGGAGCTGAATTCCTTTAATAACGTGACCGTGAC |
| | CAACATCGACCTGTACGACGACGGCTCCATGGAGTATGTGAACCTGTATAAC |
| | TGGAGGGACACCCTGAATAAGACCGACAATCTGAAGATCGGCAAGGACTATC |
| | TGGAGGACGTGATCAATGGCATCAATGAGGACACCTCCAACACCGGCACCAG |
| | CAGCGTGTATAACTGGCAGAAGCTGTACCCTGCTAATTACCACTTTCTGAGG |
| | AAGGATTATCTGACCCTGGAGCCTTCCCTGCACGAGCTGAGGGACTACATCG |
| | GCGATAGCCTGAAGCAGATGCAGTGGGATGGCTTTAAGAAGTTCAACAGCAA |
| | GCAGCAGGAGCTGTTTCTGAGCATCGTGAATTTTGACAAGCAGAAGCTGCAG |
| | AACGAGTATAACTCCTCCAATCTGCCTAACTTTGTGTTCACCGGCACCACCG |
| | TGTGGGGCGGCAATCACGAGCGGGAGTACTATGCCAAGCAGCAGATCAACGT |
| | GATCAATAATGCCATCAATGAGAGCAGCCCCCACTACCTGGGCAATAGCTAC |
| | GATCTGTTCTTCAAGGGCCACCCTGGCGGCGGCATCATCAACACCCTGATCA |
| | TGCAGAACTATCCTTCCATGGTGGATATCCCTAGCAAGATCTCCTTCGAGGT |
| | GCTGATGATGACCGACATGCTGCCCGACGCCGTGGCTGGCATCGCCTCTAGC |
| | CTGTATTTTACCATCCCCGCTGAGAAGATCAAGTTCATCGTGTTTACCAGCA |
| | CCGAGACCATCACCGACCGGGAGACCGCCCTGAGGTCCCCACTGGTGCAGGT |
| | GATGATCAAGCTGGGCATCGTGAAGGAGGAGAACGTGCTGTTTTGGGCCGAC |
| | CTGCCCAACTGTGAGACCGGCGTGTGT |
| SEQ ID NO: 136 | ATGAGGCTGCGGGAGCCATTGCTGAGCGGCTCCGCTGCCATGCCTGGCGCTT |
| B4GALT1, nt | CTCTGCAGAGGGCTTGTAGGCTGCTGGTGGCCGTGTGCGCCCTGCACCTGGG |
| | AGTGACCCTGGTGTACTACCTGGCTGGCCGGGACCTGTCCCGGCTGCCTCAG |
| | TTGGTGGGCGTGTCCACCCCTCTGCAGGGCGGAAGCAATAGCGCCGCTGCCA |
| | TCGGCCAGTCCTCCGGAGAGCTGCGGACCGGAGGCGCTAGGCCACCTCCACC |
| | TCTGGGCGCTTCCTCCCAGCCTCGGCCTGGAGGAGATAGCAGCCCCGTGGTG |
| | GATAGCGGCCCTGGCCCAGCTTCTAACCTGACCAGCGTGCCTGTGCCCCACA |
| | CCACCGCCCTGAGCCTGCCTGCTTGCCCCGAGGAGTCCCCCCTGCTGGTGGG |
| | ACCAATGCTGATCGAGTTCAATATGCCTGTGGATCTGGAGCTGGTGGCCAAG |
| | CAGAATCCCAATGTGAAGATGGGCGGCCGGTACGCTCCCCGGGATTGTGTGT |
| | CCCCTCACAAGGTGGCTATCATCATCCCTTTCCGGAACCGGCAGGAGCACCT |
| | GAAGTACTGGCTGTATTACCTGCACCCCGTGCTGCAGAGGCAGCAGCTGGAC |
| | TATGGCATCTACGTGATCAATCAGGCTGGCGACACCATCTTCAATCGGGCTA |
| | AGCTGCTGAACGTGGGCTTTCAGGAGGCTCTGAAGGACTACGACTACACCTG |
| | CTTTGTGTTCAGCGATGTGGACCTGATCCCCATGAACGACCACAACGCTTAT |
| | AGGTGCTTCTCCCAGCCTAGGCACATCTCCGTGGCTATGGACAAGTTTGGCT |
| | TCTCCCTGCCCTACGTGCAGTATTTTGGCGGCGTGTCCGCCCTGAGCAAGCA |
| | GCAGTTTCTGACCATCAATGGCTTTCCTAACAATTACTGGGGCTGGGGCGGC |
| | GAGGATGATGACATCTTCAACCGGCTGGTGTTCAGGGGCATGTCCATCAGCA |
| | GGCCTAACGCTGTGGTGGGCCGGTGTAGGATGATCAGGCACTCCCGGGACAA |
| | GAAGAATGAGCCCAATCCTCAGCGGTTTGACCGGATCGCCCACACCAAGGAG |
| | ACCATGCTGTCCGACGGCCTGAATTCCCTGACCTACCAGGTGCTGGACGTGC |
| | AGAGGTATCCTCTGTACACCCAGATCACCGTGGATATCGGCACCCCTTCCTG |
| | A |
| SEQ ID NO: 137 | MFTPVRRRVRTAALALSAAAALVLGSTAASGASATPSPAPAPAPAPVKQGPT |
| Endo H, aa | SVAYVEVNNNSMLNVGKYTLADGGGNAFDVAVIFAANINYDTGTKTAYLHEN |
| | ENVQRVLDNAVTQIRPLQQQGIKVLLSVLGNHQGAGFANFPSQQAASAFAKQ |
| | LSDAVAKYGLDGVDEDDEYAEYGNNGTAQPNDSSFVHLVTALRANMPDKIIS |
| | LYNIGPAASRLSYGGVDVSDKFDYAWNPYYGTWQVPGIALPKAQLSPAAVEI |
| | GRTSRSTVADLARRTVDEGYGVYLTYNLDGGDRTADVSAFTRELYGSEAVRT |
| | P |
| SEQ ID NO: 138 | MDKHLLVKRTLGCVCAATLMGAALATHHDSLNTVKAEEKTVQTGKTDQQVGA |
| Endo S2, aa | KLVQEIREGKRGPLYAGYFRTWHDRASTGIDGKQQHPENTMAEVPKEVDILE |
| | VFHDHTASDSPFWSELKDSYVHKLHQQGTALVQTIGVNELNGRTGLSKDYPD |
| | TPEGNKALAAAIVKAFVTDRGVDGLDIDIEHEFTNKRTPEEDARALNVEKEI |
| | AQLIGKNGSDKSKLLIMDTTLSVENNPIFKGIAEDLDYLLRQYYGSQGGEAE |
| | VDTINSDWNQYQNYIDASQFMIGESFFEESASKGNLWEDVNEYDPNNPEKGK |
| | DIEGTRAKKYAEWQPSTGGLKAGIFSYAIDRDGVAHVPSTYKNRTSTNLQRH |
| | EVDNISHTDYTVSRKLKTLMTEDKRYDVIDQKDIPDPALREQIIQQVGQYKG |
| | DLERYNKTLVLTGDKIQNLKGLEKLSKLQKLELRQLSNVKEITPELLPESMK |
| | KDAELVMVGMTGLEKLNLSGLNRQTLDGIDVNSITHLTSEDISHNSLDLSEK |
| | SEDRKLLMTLMEQVSNHQKITVKNTAFENQKPKGYYPQTYDTKEGHYDVDNA |
| | EHDILTDFVFGTVTKRNTFIGDEEAFAIYKEGAVDGRQYVSKDYTYEAFRKD |
| | YKGYKVHLTASNLGETVTSKVTATTDETYLVDVSDGEKVVHHMKLNIGSGAI |
| | MMENLAKGAKVIGTSGDFEQAKKIFDGEKSDRFFTWGQTNWIAFDLGEINLA |
| | KEWRLFNAETNTEIKTDSSLNVAKGRLQILKDTTIDLEKMDIKNRKEYLSND |
| | ENWTDVAQMDDAKAIFNSKLSNVLSRYWRFCVDGGASSYYPQYTELQILGQR |
| | LSNDVANTLKD |
| SEQ ID NO: 139 | GACTCGAAGCACTCGCTCGGCGCTTCCGACTGGCGGGGCGCCGACCATCCCG |
| Endo H, nt | TTGGGAGACCATCAGCCATGTTCACTCCGGTTCGCAGAAGGGTGCGGACGGC |
| | TGCGCTCGCGCTCTCGGCCGCCGCGGCCCTCGTCCTCGGTTCCACCGCCGCG |
| | AGCGGCGCGTCAGCGACCCCCTCACCCGCTCCGGCCCCGGCCCCGGCCCCGG |
| | TGAAGCAGGGGCCGACCTCGGTGGCCTACGTCGAGGTGAACAACAACAGCAT |
| | GCTCAACGTCGGCAAGTACACCCTGGCGGACGGAGGCGGCAACGCCTTCGAC |
| | GTAGCCGTGATCTTCGCGGCGAACATCAACTACGACACCGGCACGAAGACGG |
| | CCTACCTGCACTTCAACGAGAACGTGCAGCGCGTCCTTGACAACGCTGTCAC |
| | GCAGATACGGCCGTTGCAGCAACAGGGCATCAAGGTCCTCCTCTCGGTGCTC |
| | GGCAACCACCAGGGCGCCGGGTTCGCGAACTTCCCCTCACAGCAGGCGGCTT |
| | CGGCGTTCGCGAAGCAGCTCTCGGACGCCGTGGCGAAGTACGGCCTCGACGG |
| | CGTCGACTTCGACGACGAATACGCCGAGTACGGCAACAACGGCACCGCGCAG |
| | CCCAACGACAGTTCGTTCGTGCACCTGGTGACGGCACTGCGCGCGAACATGC |
| | CCGACAAGATCATCAGCCTCTACAACATCGGCCCGGCCGCGTCCCGCCTGTC |
| | GTACGGCGGTGTCGACGTCTCCGACAAGTTCGACTACGCCTGGAATCCCTAC |
| | TACGGCACCTGGCAGGTCCCCGGCATCGCACTGCCCAAGGCGCAGCTGTCGC |
| | CGGCGGCCGTCGAGATCGGCCGGACCTCACGGAGCACCGTCGCCGACCTCGC |
| | CCGTCGCACCGTCGACGAGGGGTACGGCGTCTATCTGACGTACAACCTCGAC |
| | GGCGGCGATCGCACCGCCGACGTCTCCGCGTTCACCAGGGAGCTGTACGGCA |
| | GCGAGGCGGTCCGGACGCCGTAGGGGCGTCGGGGCCTGCCGTCAGTCCAGTA |
| | CGAAGGTGCCGCCGGCGGTGGTCGCCTGGCCGTGCCCGAAAGCGGCCGCCGG |
| | CGTCCAGGATCC |
| SEQ ID NO: 140 | GGCGAACTATAGGAATGCGCTTACATAGATGGTATATCAGATGGGAAGCCAT |
| Endo S2, nt | GACTTAGTACCAAAAATAAGGAGTGTCCAAATGGATAAACATTTGTTGGTAA |
| | AAAGAACACTAGGGTGTGTTTGTGCTGCAACGTTGATGGGAGCTGCCTTAGC |
| | GACCCACCATGATTCACTCAATACTGTAAAAGCGGAGGAGAAGACTGTTCAA |
| | ACAGGAAAGACAGATCAGCAGGTTGGTGCTAAATTGGTACAGGAAATCCGTG |
| | AAGGAAAACGCGGACCACTATATGCTGGTTATTTTAGGACATGGCATGATCG |
| | TGCTTCAACAGGAATAGATGGTAAACAGCAACATCCAGAAAATACTATGGCT |
| | GAGGTCCCAAAAGAAGTTGATATCTTATTTGTTTTTCATGACCATACAGCTT |
| | CAGATAGTCCATTTTGGTCTGAATTAAAGGACAGTTATGTCCATAAATTACA |
| | TCAACAGGGAACGGCACTTGTTCAGACAATTGGTGTTAACGAATTAAATGGA |
| | CGTACAGGTTTATCTAAAGATTATCCTGATACTCCTGAGGGGAACAAAGCTT |
| | TAGCAGCAGCCATTGTCAAGGCATTTGTAACTGATCGTGGTGTCGATGGACT |
| | AGATATTGATATTGAGCACGAATTTACGAACAAAAGAACACCTGAAGAAGAT |
| | GCTCGTGCTCTAAATGTTTTTAAAGAGATTGCGCAGTTAATAGGTAAAAATG |
| | GTAGTGATAAATCTAAATTGCTCATCATGGACACTACCCTAAGTGTTGAAAA |
| | TAATCCAATATTTAAAGGGATAGCGGAAGATCTTGATTATCTTCTTAGACAA |
| | TATTATGGTTCACAAGGTGGAGAAGCTGAAGTGGATACTATAAACTCTGATT |
| | GGAACCAATATCAGAATTATATTGATGCTAGCCAGTTCATGATTGGATTCTC |
| | CTTTTTTGAAGAATCTGCGTCCAAAGGGAATTTATGGTTTGATGTTAACGAA |
| | TACGACCCTAACAATCCTGAAAAAGGGAAAGATATTGAAGGAACACGTGCTA |
| | AAAAATATGCAGAGTGGCAACCTAGTACAGGTGGTTTAAAAGCAGGTATATT |
| | CTCTTATGCTATTGATCGTGATGGAGTGGCTCATGTTCCTTCAACATATAAA |
| | AATAGGACTAGTACAAATTTACAACGGCATGAAGTCGATAATATCTCACATA |
| | CTGACTACACCGTATCTCGAAAATTAAAAACATTGATGACCGAAGACAAACG |
| | CTATGATGTCATTGATCAAAAAGACATTCCTGACCCAGCATTAAGAGAACAA |
| | ATCATTCAACAAGTTGGACAGTATAAAGGCGATTTGGAACGTTATAACAAGA |
| | CATTGGTGCTTACAGGAGATAAGATTCAAAATCTTAAAGGACTAGAAAAATT |
| | AAGCAAGTTACAAAAATTAGAGTTGCGCCAGCTATCTAACGTTAAAGAAATT |
| | ACTCCAGAACTTTTGCCGGAAAGCATGAAAAAAGATGCTGAGCTTGTTATGG |
| | TAGGCATGACTGGTTTAGAAAAACTAAACCTTAGTGGTCTAAATCGTCAAAC |
| | TTTAGACGGTATAGACGTGAATAGTATTACGCATTTGACATCATTTGATATT |
| | TCACATAATAGTTTGGACTTGTCGGAAAAGAGTGAAGACCGTAAACTATTAA |
| | TGACTTTGATGGAGCAGGTTTCAAATCATCAAAAAATAACGGTGAAAAATAC |
| | GGCTTTTGAAAATCAAAAACCGAAAGGTTATTATCCTCAGACGTATGATACC |
| | AAAGAAGGTCATTATGATGTTGATAATGCAGAACATGATATTTTAACTGATT |
| | TTGTTTTTGGAACTGTTACTAAACGTAATACCTTTATTGGAGACGAAGAAGC |
| | ATTTGCTATCTATAAAGAAGGAGCTGTCGATGGTCGACAATATGTGTCTAAA |
| | GACTATACTTATGAAGCTTTTCGTAAAGACTATAAAGGTTACAAGGTTCATT |
| | TAACTGCTTCTAACCTAGGAGAAACAGTTACTTCTAAGGTAACTGCTACTAC |
| | TGATGAAACTTACTTAGTAGATGTTTCTGATGGGGAAAAAGTTGTTCACCAC |
| | ATGAAACTCAATATAGGATCTGGTGCCATCATGATGGAAAATCTGGCAAAAG |
| | GGGCTAAAGTGATTGGTACATCTGGGGACTTTGAGCAAGCAAAGAAGATTTT |
| | CGATGGTGAAAAGTCAGATAGATTCTTCACTTGGGGACAAACTAACTGGATA |
| | GCTTTTGATCTAGGAGAAATTAATCTTGCGAAGGAATGGCGTTTATTTAATG |
| | CAGAGACAAATACTGAAATAAAGACAGATAGTAGCTTAAACGTGGCTAAAGG |
| | ACGTCTTCAGATTTTAAAAGATACAACTATTGATTTAGAAAAAATGGACATA |
| | AAAAATCGTAAAGAGTATCTGTCGAATGATGAAAATTGGACTGATGTTGCTC |
| | AGATGGATGATGCAAAAGCGATATTTAATAGTAAATTATCCAATGTTTTATC |
| | TCGGTATTGGCGGTTTTGTGTAGATGGTGGAGCTAGCTCTTATTACCCTCAA |
| | TATACCGAACTTCAAATCCTCGGACAACGTTTATCAAATGATGTCGCTAATA |
| | CGCTGAAGGATTGATATTAAAGTCCTACGATTACAAAGATAAACAGTTTCTC |
| | AAAGTTGATTTAAGCACTTTAAAACCTAACTAAAAATCTGAGATGAATAGTC |
| | CCAGATTTTTAGTCTTTTATAGGTTTTGATGACATAAAGCTAAATAATCGTT |
| | AGACTACCAGAAAGTGGCGC |
| SEQ ID NO: 141 | GGCTCCGGCGCTACCAACTTCTCCCTGCTGAAGCAGGCTGGCGATGTGGAGG |
| Ribosomal | AGAATCCCGGCCCT |
| Shifting | |
| peptide-P2A, | |
| nt | |
| SEQ ID NO: 142 | GGTTCAGGTGAGGGCCGCGGCAGTCTGCTGACTTGCGGCGACGTCGAGGAGA |
| Ribosomal | ACCCCGGTCCT |
| Shifting | |
| peptide-T2A, | |
| nt | |
| SEQ ID NO: 143 | GGGAGTGGGCAATGCACTAACTATGCATTGCTTAAGTTGGCTGGTGATGTAG |
| Ribosomal | AATCTAATCCTGGGCCC |
| Shifting | |
| peptide-E2A, | |
| nt | |
| SEQ ID NO: 144 | GGCAGTGGGGTGAAGCAGACCCTCAATTTTGACTTGTTGAAACTCGCAGGCG |
| Ribosomal | ACGTAGAATCTAACCCCGGACCT |
| Shifting | |
| peptide-F2A, | |
| nt | |
| SEQ ID NO: 145 | GACGGATCGGGAGATCTCCCGATCCCCTATGGTGCACTCTCAGTACAATCTG |
| SWG-006, nt | CTCTGATGCCGCATAGTTAAGCCAGTATCTGCTCCCTGCTTGTGTGTTGGAG |
| | GTCGCTGAGTAGTGCGCGAGCAAAATTTAAGCTACAACAAGGCAAGGCTTGA |
| | CCGACAATTGCATGAAGAATCTGCTTAGGGTTAGGCGTTTTGCGCTGCTTCG |
| | CGATGTACGGGCCAGATATACGCGTTGACATTGATTATTGACTAGTTATTAA |
| | TAGTAATCAATTACGGGGTCATTAGTTCATAGCCCATATATGGAGTTCCGCG |
| | TTACATAACTTACGGTAAATGGCCCGCCTGGCTGACCGCCCAACGACCCCCG |
| | CCCATTGACGTCAATAATGACGTATGTTCCCATAGTAACGCCAATAGGGACT |
| | TTCCATTGACGTCAATGGGTGGAGTATTTACGGTAAACTGCCCACTTGGCAG |
| | TACATCAAGTGTATCATATGCCAAGTACGCCCCCTATTGACGTCAATGACGG |
| | TAAATGGCCCGCCTGGCATTATGCCCAGTACATGACCTTATGGGACTTTCCT |
| | ACTTGGCAGTACATCTACGTATTAGTCATCGCTATTACCATGGTGATGCGGT |
| | TTTGGCAGTACATCAATGGGCGTGGATAGCGGTTTGACTCACGGGGATTTCC |
| | AAGTCTCCACCCCATTGACGTCAATGGGAGTTTGTTTTGGCACCAAAATCAA |
| | CGGGACTTTCCAAAATGTCGTAACAACTCCGCCCCATTGACGCAAATGGGCG |
| | GTAGGCGTGTACGGTGGGAGGTCTATATAAGCAGAGCTCTCTGGCTAACTAG |
| | AGAACCCACTGCTTACTGGCTTATCGAAATTAATACGACTCACTATAGGGAG |
| | ACCCAAGCTGGCTAGCGCCGCCACCATGATCCACACCAATCTGAAGAAGAAG |
| | TTTAGCTGTTGCGTGCTGGTGTTCCTGCTGTTCGCTGTGATCTGCGTGTGGA |
| | AGGAGAAGAAGAAGGGCAGCTATTATGACTCCTTTAAGCTGCAGACCAAGGA |
| | GTTCCAGGTGCTGAAGTCCCTGGGCAAGCTGGCCATGGGCAGCGACTCCCAG |
| | TCCGTGAGCTCCTCCAGCACCCAGGACCCCCACCGGGGAAGGCAGACCCTGG |
| | GATCCCTGAGGGGCCTGGCTAAGGCCAAGCCTGAGGCTTCCTTCCAGGTGTG |
| | GAATAAGGATAGCTCCAGCAAGAACCTGATCCCTAGGCTGCAGAAGATCTGG |
| | AAGAACTATCTGAGCATGAACAAGTATAAGGTGAGCTATAAGGGCCCTGGCC |
| | CTGGCATCAAGTTCTCCGCCGAGGCCCTGAGGTGCCACCTGAGAGATCACGT |
| | GAATGTGAGCATGGTGGAGGTGACCGACTTCCCTTTTAACACCTCCGAGTGG |
| | GAGGGCTACCTGCCCAAGGAGAGCATCCGGACCAAGGCCGGCCCTTGGGGCA |
| | GATGTGCCGTGGTGTCCAGCGCCGGCTCCCTGAAGAGCTCCCAGCTGGGCCG |
| | GGAGATCGATGACCACGATGCTGTGCTGAGGTTCAATGGCGCTCCTACCGCT |
| | AACTTTCAGCAGGATGTGGGCACCAAGACCACCATCCGGCTGATGAATAGCC |
| | AGCTGGTGACCACCGAGAAGCGGTTTCTGAAGGACTCCCTGTACAACGAGGG |
| | CATCCTGATCGTGTGGGACCCCTCCGTGTACCACAGCGACATCCCCAAGTGG |
| | TATCAGAACCCCGACTACAACTTCTTCAATAATTACAAGACCTACCGGAAGC |
| | TGCACCCCAACCAGCCCTTTTATATCCTGAAGCCCCAGATGCCTTGGGAGCT |
| | GTGGGACATCCTGCAGGAGATCTCCCCCGAGGAGATCCAGCCTAACCCCCCT |
| | AGCAGCGGCATGCTGGGCATCATCATCATGATGACCCTGTGCGATCAGGTGG |
| | ATATCTACGAGTTCCTGCCCAGCAAGCGGAAGACCGACGTGTGTTATTATTA |
| | TCAGAAGTTCTTCGATTCCGCCTGCACCATGGGCGCCTATCACCCTCTGCTG |
| | TACGAGAAGAATCTGGTGAAGCACCTGAACCAGGGCACCGACGAGGACATCT |
| | ATCTGCTGGGCAAGGCCACCCTGCCCGGCTTTAGGACCATCCACTGCGGCAG |
| | CGGCGCTACCAATTTCAGCCTGCTGAAGCAGGCTGGCGATGTGGAGGAGAAC |
| | CCCGGCCCCATGAGGCTGCGGGAGCCATTGCTGAGCGGCTCCGCTGCCATGC |
| | CTGGCGCTTCTCTGCAGAGGGCTTGTAGGCTGCTGGTGGCCGTGTGCGCCCT |
| | GCACCTGGGAGTGACCCTGGTGTACTACCTGGCTGGCCGGGACCTGTCCCGG |
| | CTGCCTCAGTTGGTGGGCGTGTCCACCCCTCTGCAGGGCGGAAGCAATAGCG |
| | CCGCTGCCATCGGCCAGTCCTCCGGAGAGCTGCGGACCGGAGGCGCTAGGCC |
| | ACCTCCACCTCTGGGCGCTTCCTCCCAGCCTCGGCCTGGAGGAGATAGCAGC |
| | CCCGTGGTGGATAGCGGCCCTGGCCCAGCTTCTAACCTGACCAGCGTGCCTG |
| | TGCCCCACACCACCGCCCTGAGCCTGCCTGCTTGCCCCGAGGAGTCCCCCCT |
| | GCTGGTGGGACCAATGCTGATCGAGTTCAATATGCCTGTGGATCTGGAGCTG |
| | GTGGCCAAGCAGAATCCCAATGTGAAGATGGGCGGCCGGTACGCTCCCCGGG |
| | ATTGTGTGTCCCCTCACAAGGTGGCTATCATCATCCCTTTCCGGAACCGGCA |
| | GGAGCACCTGAAGTACTGGCTGTATTACCTGCACCCCGTGCTGCAGAGGCAG |
| | CAGCTGGACTATGGCATCTACGTGATCAATCAGGCTGGCGACACCATCTTCA |
| | ATCGGGCTAAGCTGCTGAACGTGGGCTTTCAGGAGGCTCTGAAGGACTACGA |
| | CTACACCTGCTTTGTGTTCAGCGATGTGGACCTGATCCCCATGAACGACCAC |
| | AACGCTTATAGGTGCTTCTCCCAGCCTAGGCACATCTCCGTGGCTATGGACA |
| | AGTTTGGCTTCTCCCTGCCCTACGTGCAGTATTTTGGCGGCGTGTCCGCCCT |
| | GAGCAAGCAGCAGTTTCTGACCATCAATGGCTTTCCTAACAATTACTGGGGC |
| | TGGGGCGGCGAGGATGATGACATCTTCAACCGGCTGGTGTTCAGGGGCATGT |
| | CCATCAGCAGGCCTAACGCTGTGGTGGGCCGGTGTAGGATGATCAGGCACTC |
| | CCGGGACAAGAAGAATGAGCCCAATCCTCAGCGGTTTGACCGGATCGCCCAC |
| | ACCAAGGAGACCATGCTGTCCGACGGCCTGAATTCCCTGACCTACCAGGTGC |
| | TGGACGTGCAGAGGTATCCTCTGTACACCCAGATCACCGTGGATATCGGCAC |
| | CCCTTCCTGATGATGACTCGAGTCTAGAGGGCCCGTTTAAACCCGCTGATCA |
| | GCCTCGACTGTGCCTTCTAGTTGCCAGCCATCTGTTGTTTGCCCCTCCCCCG |
| | TGCCTTCCTTGACCCTGGAAGGTGCCACTCCCACTGTCCTTTCCTAATAAAA |
| | TGAGGAAATTGCATCGCATTGTCTGAGTAGGTGTCATTCTATTCTGGGGGGT |
| | GGGGTGGGGCAGGACAGCAAGGGGGAGGATTGGGAAGACAATAGCAGGCATG |
| | CTGGGGATGCGGTGGGCTCTATGGCTTCTGAGGCGGAAAGAACCAGCTGGGG |
| | CTCTAGGGGGTATCCCCACGCGCCCTGTAGCGGCGCATTAAGCGCGGCGGGT |
| | GTGGTGGTTACGCGCAGCGTGACCGCTACACTTGCCAGCGCCCTAGCGCCCG |
| | CTCCTTTCGCTTTCTTCCCTTCCTTTCTCGCCACGTTCGCCGGCTTTCCCCG |
| | TCAAGCTCTAAATCGGGGGCTCCCTTTAGGGTTCCGATTTAGTGCTTTACGG |
| | CACCTCGACCCCAAAAAACTTGATTAGGGTGATGGTTCACGTAGTGGGCCAT |
| | CGCCCTGATAGACGGTTTTTCGCCCTTTGACGTTGGAGTCCACGTTCTTTAA |
| | TAGTGGACTCTTGTTCCAAACTGGAACAACACTCAACCCTATCTCGGTCTAT |
| | TCTTTTGATTTATAAGGGATTTTGCCGATTTCGGCCTATTGGTTAAAAAATG |
| | AGCTGATTTAACAAAAATTTAACGCGAATTAATTCTGTGGAATGTGTGTCAG |
| | TTAGGGTGTGGAAAGTCCCCAGGCTCCCCAGCAGGCAGAAGTATGCAAAGCA |
| | TGCATCTCAATTAGTCAGCAACCAGGTGTGGAAAGTCCCCAGGCTCCCCAGC |
| | AGGCAGAAGTATGCAAAGCATGCATCTCAATTAGTCAGCAACCATAGTCCCG |
| | CCCCTAACTCCGCCCATCCCGCCCCTAACTCCGCCCAGTTCCGCCCATTCTC |
| | CGCCCCATGGCTGACTAATTTTTTTTATTTATGCAGAGGCCGAGGCCGCCTC |
| | TGCCTCTGAGCTATTCCAGAAGTAGTGAGGAGGCTTTTTTGGAGGCCTAGGC |
| | TTTTGCAAAAAGCTCCCGGGAGCTTGTATATCCATTTTCGGATCTGATCAAG |
| | AGACAGGATGAGGATCGTTTCGCATGATTGAACAAGATGGATTGCACGCAGG |
| | TTCTCCGGCCGCTTGGGTGGAGAGGCTATTCGGCTATGACTGGGCACAACAG |
| | ACAATCGGCTGCTCTGATGCCGCCGTGTTCCGGCTGTCAGCGCAGGGGCGCC |
| | CGGTTCTTTTTGTCAAGACCGACCTGTCCGGTGCCCTGAATGAACTGCAGGA |
| | CGAGGCAGCGCGGCTATCGTGGCTGGCCACGACGGGCGTTCCTTGCGCAGCT |
| | GTGCTCGACGTTGTCACTGAAGCGGGAAGGGACTGGCTGCTATTGGGCGAAG |
| | TGCCGGGGCAGGATCTCCTGTCATCTCACCTTGCTCCTGCCGAGAAAGTATC |
| | CATCATGGCTGATGCAATGCGGCGGCTGCATACGCTTGATCCGGCTACCTGC |
| | CCATTCGACCACCAAGCGAAACATCGCATCGAGCGAGCACGTACTCGGATGG |
| | AAGCCGGTCTTGTCGATCAGGATGATCTGGACGAAGAGCATCAGGGGCTCGC |
| | GCCAGCCGAACTGTTCGCCAGGCTCAAGGCGCGCATGCCCGACGGCGAGGAT |
| | CTCGTCGTGACCCATGGCGATGCCTGCTTGCCGAATATCATGGTGGAAAATG |
| | GCCGCTTTTCTGGATTCATCGACTGTGGCCGGCTGGGTGTGGCGGACCGCTA |
| | TCAGGACATAGCGTTGGCTACCCGTGATATTGCTGAAGAGCTTGGCGGCGAA |
| | TGGGCTGACCGCTTCCTCGTGCTTTACGGTATCGCCGCTCCCGATTCGCAGC |
| | GCATCGCCTTCTATCGCCTTCTTGACGAGTTCTTCTGAGCGGGACTCTGGGG |
| | TTCGAAATGACCGACCAAGCGACGCCCAACCTGCCATCACGAGATTTCGATT |
| | CCACCGCCGCCTTCTATGAAAGGTTGGGCTTCGGAATCGTTTTCCGGGACGC |
| | CGGCTGGATGATCCTCCAGCGCGGGGATCTCATGCTGGAGTTCTTCGCCCAC |
| | CCCAACTTGTTTATTGCAGCTTATAATGGTTACAAATAAAGCAATAGCATCA |
| | CAAATTTCACAAATAAAGCATTTTTTTCACTGCATTCTAGTTGTGGTTTGTC |
| | CAAACTCATCAATGTATCTTATCATGTCTGTATACCGTCGACCTCTAGCTAG |
| | AGCTTGGCGTAATCATGGTCATAGCTGTTTCCTGTGTGAAATTGTTATCCGC |
| | TCACAATTCCACACAACATACGAGCCGGAAGCATAAAGTGTAAAGCCTGGGG |
| | TGCCTAATGAGTGAGCTAACTCACATTAATTGCGTTGCGCTCACTGCCCGCT |
| | TTCCAGTCGGGAAACCTGTCGTGCCAGCTGCATTAATGAATCGGCCAACGCG |
| | CGGGGAGAGGCGGTTTGCGTATTGGGCGCTCTTCCGCTTCCTCGCTCACTGA |
| | CTCGCTGCGCTCGGTCGTTCGGCTGCGGCGAGCGGTATCAGCTCACTCAAAG |
| | GCGGTAATACGGTTATCCACAGAATCAGGGGATAACGCAGGAAAGAACATGT |
| | GAGCAAAAGGCCAGCAAAAGGCCAGGAACCGTAAAAAGGCCGCGTTGCTGGC |
| | GTTTTTCCATAGGCTCCGCCCCCCTGACGAGCATCACAAAAATCGACGCTCA |
| | AGTCAGAGGTGGCGAAACCCGACAGGACTATAAAGATACCAGGCGTTTCCCC |
| | CTGGAAGCTCCCTCGTGCGCTCTCCTGTTCCGACCCTGCCGCTTACCGGATA |
| | CCTGTCCGCCTTTCTCCCTTCGGGAAGCGTGGCGCTTTCTCATAGCTCACGC |
| | TGTAGGTATCTCAGTTCGGTGTAGGTCGTTCGCTCCAAGCTGGGCTGTGTGC |
| | ACGAACCCCCCGTTCAGCCCGACCGCTGCGCCTTATCCGGTAACTATCGTCT |
| | TGAGTCCAACCCGGTAAGACACGACTTATCGCCACTGGCAGCAGCCACTGGT |
| | AACAGGATTAGCAGAGCGAGGTATGTAGGCGGTGCTACAGAGTTCTTGAAGT |
| | GGTGGCCTAACTACGGCTACACTAGAAGAACAGTATTTGGTATCTGCGCTCT |
| | GCTGAAGCCAGTTACCTTCGGAAAAAGAGTTGGTAGCTCTTGATCCGGCAAA |
| | CAAACCACCGCTGGTAGCGGTTTTTTTGTTTGCAAGCAGCAGATTACGCGCA |
| | GAAAAAAAGGATCTCAAGAAGATCCTTTGATCTTTTCTACGGGGTCTGACGC |
| | TCAGTGGAACGAAAACTCACGTTAAGGGATTTTGGTCATGAGATTATCAAAA |
| | AGGATCTTCACCTAGATCCTTTTAAATTAAAAATGAAGTTTTAAATCAATCT |
| | AAAGTATATATGAGTAAACTTGGTCTGACAGTTACCAATGCTTAATCAGTGA |
| | GGCACCTATCTCAGCGATCTGTCTATTTCGTTCATCCATAGTTGCCTGACTC |
| | CCCGTCGTGTAGATAACTACGATACGGGAGGGCTTACCATCTGGCCCCAGTG |
| | CTGCAATGATACCGCGAGACCCACGCTCACCGGCTCCAGATTTATCAGCAAT |
| | AAACCAGCCAGCCGGAAGGGCCGAGCGCAGAAGTGGTCCTGCAACTTTATCC |
| | GCCTCCATCCAGTCTATTAATTGTTGCCGGGAAGCTAGAGTAAGTAGTTCGC |
| | CAGTTAATAGTTTGCGCAACGTTGTTGCCATTGCTACAGGCATCGTGGTGTC |
| | ACGCTCGTCGTTTGGTATGGCTTCATTCAGCTCCGGTTCCCAACGATCAAGG |
| | CGAGTTACATGATCCCCCATGTTGTGCAAAAAAGCGGTTAGCTCCTTCGGTC |
| | CTCCGATCGTTGTCAGAAGTAAGTTGGCCGCAGTGTTATCACTCATGGTTAT |
| | GGCAGCACTGCATAATTCTCTTACTGTCATGCCATCCGTAAGATGCTTTTCT |
| | GTGACTGGTGAGTACTCAACCAAGTCATTCTGAGAATAGTGTATGCGGCGAC |
| | CGAGTTGCTCTTGCCCGGCGTCAATACGGGATAATACCGCGCCACATAGCAG |
| | AACTTTAAAAGTGCTCATCATTGGAAAACGTTCTTCGGGGCGAAAACTCTCA |
| | AGGATCTTACCGCTGTTGAGATCCAGTTCGATGTAACCCACTCGTGCACCCA |
| | ACTGATCTTCAGCATCTTTTACTTTCACCAGCGTTTCTGGGTGAGCAAAAAC |
| | AGGAAGGCAAAATGCCGCAAAAAAGGGAATAAGGGCGACACGGAAATGTTGA |
| | ATACTCATACTCTTCCTTTTTCAATATTATTGAAGCATTTATCAGGGTTATT |
| | GTCTCATGAGCGGATACATATTTGAATGTATTTAGAAAAATAAACAAATAGG |
| | GGTTCCGCGCACATTTCCCCGAAAAGTGCCACCTGACGTC |
| SEQ ID NO: 146 | GACGGATCGGGAGATCTCCCGATCCCCTATGGTGCACTCTCAGTACAATCTG |
| SWG-015, nt | CTCTGATGCCGCATAGTTAAGCCAGTATCTGCTCCCTGCTTGTGTGTTGGAG |
| | GTCGCTGAGTAGTGCGCGAGCAAAATTTAAGCTACAACAAGGCAAGGCTTGA |
| | CCGACAATTGCATGAAGAATCTGCTTAGGGTTAGGCGTTTTGCGCTGCTTCG |
| | CGATGTACGGGCCAGATATACGCGTTGACATTGATTATTGACTAGTTATTAA |
| | TAGTAATCAATTACGGGGTCATTAGTTCATAGCCCATATATGGAGTTCCGCG |
| | TTACATAACTTACGGTAAATGGCCCGCCTGGCTGACCGCCCAACGACCCCCG |
| | CCCATTGACGTCAATAATGACGTATGTTCCCATAGTAACGCCAATAGGGACT |
| | TTCCATTGACGTCAATGGGTGGAGTATTTACGGTAAACTGCCCACTTGGCAG |
| | TACATCAAGTGTATCATATGCCAAGTACGCCCCCTATTGACGTCAATGACGG |
| | TAAATGGCCCGCCTGGCATTATGCCCAGTACATGACCTTATGGGACTTTCCT |
| | ACTTGGCAGTACATCTACGTATTAGTCATCGCTATTACCATGGTGATGCGGT |
| | TTTGGCAGTACATCAATGGGCGTGGATAGCGGTTTGACTCACGGGGATTTCC |
| | AAGTCTCCACCCCATTGACGTCAATGGGAGTTTGTTTTGGCACCAAAATCAA |
| | CGGGACTTTCCAAAATGTCGTAACAACTCCGCCCCATTGACGCAAATGGGCG |
| | GTAGGCGTGTACGGTGGGAGGTCTATATAAGCAGAGCTCTCTGGCTAACTAG |
| | AGAACCCACTGCTTACTGGCTTATCGAAATTAATACGACTCACTATAGGGAG |
| | ACCCAAGCTGGCTAGCGCCGCCACCATGATCCACACCAACCTGAAGAAGAAG |
| | TTCTCCTGCTGTGTGCTGGTGTTTCTGCTGTTCGCTGTGATCTGCGTGTGGA |
| | AGGAGAAGAAGAAGGGCAGCTATTATGACAGCTTTAAGCTGCAGACCAAGGA |
| | GTTTCAGGTGCTGAAGTCCCTGGGCAAGCTGGCCATGGGCTCCGACTCCCAG |
| | TCCGTGTCCAGCTCCAGCACCCAGGATCCTCACCGGGGCAGGCAGACCCTGG |
| | GCTCTCTGAGAGGCCTGGCTAAGGCTAAGCCCGGCGGCGGAGGCAGCGGAGG |
| | AGGAGGATCTGGCGGCGGAGGATCCAATGTGGTGGCCCCTTCCCTGGAGGTG |
| | TATGTGGACCACGCCTCCCTGCCCACCCTGCAGCAGCTGATGGATATCATCA |
| | AGAGCGAGGAGGAGAATCCTACCGCCCAGCGGTATATCGCCTGGGGCCGGAT |
| | CGTGCCTACCGACGAGCAGATGAAGGAGCTGAACATCACCTCCTTCGCTCTG |
| | ATCAACAATCACACCCCCGCCGACCTGGTGCAGGAGATCGTGAAGCAGGCCC |
| | AGACCAAGCACCGGCTGAACGTGAAGCTGTCCTCCAACACCGCCCACTCCTT |
| | TGACAACCTGGTGCCTATCCTGAAGGAGCTGAATTCCTTTAATAACGTGACC |
| | GTGACCAACATCGACCTGTACGACGACGGCTCCATGGAGTATGTGAACCTGT |
| | ATAACTGGAGGGACACCCTGAATAAGACCGACAATCTGAAGATCGGCAAGGA |
| | CTATCTGGAGGACGTGATCAATGGCATCAATGAGGACACCTCCAACACCGGC |
| | ACCAGCAGCGTGTATAACTGGCAGAAGCTGTACCCTGCTAATTACCACTTTC |
| | TGAGGAAGGATTATCTGACCCTGGAGCCTTCCCTGCACGAGCTGAGGGACTA |
| | CATCGGCGATAGCCTGAAGCAGATGCAGTGGGATGGCTTTAAGAAGTTCAAC |
| | AGCAAGCAGCAGGAGCTGTTTCTGAGCATCGTGAATTTTGACAAGCAGAAGC |
| | TGCAGAACGAGTATAACTCCTCCAATCTGCCTAACTTTGTGTTCACCGGCAC |
| | CACCGTGTGGGGCGGCAATCACGAGCGGGAGTACTATGCCAAGCAGCAGATC |
| | AACGTGATCAATAATGCCATCAATGAGAGCAGCCCCCACTACCTGGGCAATA |
| | GCTACGATCTGTTCTTCAAGGGCCACCCTGGCGGCGGCATCATCAACACCCT |
| | GATCATGCAGAACTATCCTTCCATGGTGGATATCCCTAGCAAGATCTCCTTC |
| | GAGGTGCTGATGATGACCGACATGCTGCCCGACGCCGTGGCTGGCATCGCCT |
| | CTAGCCTGTATTTTACCATCCCCGCTGAGAAGATCAAGTTCATCGTGTTTAC |
| | CAGCACCGAGACCATCACCGACCGGGAGACCGCCCTGAGGTCCCCACTGGTG |
| | CAGGTGATGATCAAGCTGGGCATCGTGAAGGAGGAGAACGTGCTGTTTTGGG |
| | CCGACCTGCCCAACTGTGAGACCGGCGTGTGTGGCTCCGGCGCTACCAACTT |
| | CTCCCTGCTGAAGCAGGCTGGCGATGTGGAGGAGAATCCCGGCCCTATGCGG |
| | CTGCGGGAGCCCCTGTTGTCCGGCTCTGCCGCTATGCCCGGCGCTTCCCTGC |
| | AGAGAGCCTGTCGGCTGCTGGTGGCTGTGTGCGCTCTGCACCTGGGCGTGAC |
| | CCTGGTGTACTATCTGGCTGGCCGGGATCTGAGCCGGCTGCCTCAGCTGGTG |
| | GGCGTGAGCACCCCCCTGCAGGGAGGATCCAACTCCGCCGCTGCCATCGGCC |
| | AGTCCTCCGGAGAGCTGCGGACCGGAGGCGCTAGGCCTCCACCACCACTGGG |
| | CGCTTCTTCCCAGCCTCGGCCCGGAGGAGATAGCAGCCCCGTGGTGGACTCC |
| | GGCCCTGGACCTGCTTCCAACCTGACCAGCGTGCCCGTGCCTCACACCACCG |
| | CTCTGTCCCTGCCCGCCTGTCCCGAGGAGTCCCCTCTGCTGGTGGGCCCTAT |
| | GCTGATCGAGTTTAATATGCCTGTGGACCTGGAGCTGGTGGCTAAGCAGAAT |
| | CCTAACGTGAAGATGGGCGGCAGGTACGCTCCCAGGGACTGCGTGAGCCCTC |
| | ACAAGGTGGCCATCATCATCCCTTTCCGGAACCGGCAGGAGCACCTGAAGTA |
| | CTGGCTGTACTACCTGCACCCTGTGCTGCAGAGGCAGCAGCTGGACTATGGC |
| | ATCTACGTGATCAACCAGGCCGGCGACACCATCTTTAACCGGGCCAAGCTGC |
| | TGAATGTGGGCTTTCAGGAGGCCCTGAAGGATTATGACTACACCTGCTTTGT |
| | GTTTAGCGATGTGGATCTGATCCCTATGAACGATCACAACGCCTACCGGTGC |
| | TTCAGCCAGCCTCGGCACATCAGCGTGGCCATGGACAAGTTCGGCTTCTCCC |
| | TGCCTTACGTGCAGTACTTCGGCGGCGTGTCCGCCCTGTCCAAGCAGCAGTT |
| | CCTGACCATCAACGGCTTTCCTAATAACTACTGGGGCTGGGGCGGCGAGGAT |
| | GATGACATCTTTAATCGGCTGGTGTTTAGGGGCATGAGCATCAGCCGGCCTA |
| | ACGCCGTGGTGGGCAGGTGCAGGATGATCAGGCACTCCCGGGACAAGAAGAA |
| | CGAGCCTAATCCTCAGAGGTTTGACAGGATCGCTCACACCAAGGAGACCATG |
| | CTGAGCGACGGCCTGAATAGCCTGACCTACCAGGTGCTGGACGTGCAGAGGT |
| | ACCCTCTGTATACCCAGATCACCGTGGATATCGGCACCCCTAGCTGACTCGA |
| | GTCTAGAGGGCCCGTTTAAACCCGCTGATCAGCCTCGACTGTGCCTTCTAGT |
| | TGCCAGCCATCTGTTGTTTGCCCCTCCCCCGTGCCTTCCTTGACCCTGGAAG |
| | GTGCCACTCCCACTGTCCTTTCCTAATAAAATGAGGAAATTGCATCGCATTG |
| | TCTGAGTAGGTGTCATTCTATTCTGGGGGGTGGGGTGGGGCAGGACAGCAAG |
| | GGGGAGGATTGGGAAGACAATAGCAGGCATGCTGGGGATGCGGTGGGCTCTA |
| | TGGCTTCTGAGGCGGAAAGAACCAGCTGGGGCTCTAGGGGGTATCCCCACGC |
| | GCCCTGTAGCGGCGCATTAAGCGCGGCGGGTGTGGTGGTTACGCGCAGCGTG |
| | ACCGCTACACTTGCCAGCGCCCTAGCGCCCGCTCCTTTCGCTTTCTTCCCTT |
| | CCTTTCTCGCCACGTTCGCCGGCTTTCCCCGTCAAGCTCTAAATCGGGGGCT |
| | CCCTTTAGGGTTCCGATTTAGTGCTTTACGGCACCTCGACCCCAAAAAACTT |
| | GATTAGGGTGATGGTTCACGTAGTGGGCCATCGCCCTGATAGACGGTTTTTC |
| | GCCCTTTGACGTTGGAGTCCACGTTCTTTAATAGTGGACTCTTGTTCCAAAC |
| | TGGAACAACACTCAACCCTATCTCGGTCTATTCTTTTGATTTATAAGGGATT |
| | TTGCCGATTTCGGCCTATTGGTTAAAAAATGAGCTGATTTAACAAAAATTTA |
| | ACGCGAATTAATTCTGTGGAATGTGTGTCAGTTAGGGTGTGGAAAGTCCCCA |
| | GGCTCCCCAGCAGGCAGAAGTATGCAAAGCATGCATCTCAATTAGTCAGCAA |
| | CCAGGTGTGGAAAGTCCCCAGGCTCCCCAGCAGGCAGAAGTATGCAAAGCAT |
| | GCATCTCAATTAGTCAGCAACCATAGTCCCGCCCCTAACTCCGCCCATCCCG |
| | CCCCTAACTCCGCCCAGTTCCGCCCATTCTCCGCCCCATGGCTGACTAATTT |
| | TTTTTATTTATGCAGAGGCCGAGGCCGCCTCTGCCTCTGAGCTATTCCAGAA |
| | GTAGTGAGGAGGCTTTTTTGGAGGCCTAGGCTTTTGCAAAAAGCTCCCGGGA |
| | GCTTGTATATCCATTTTCGGATCTGATCAAGAGACAGGATGAGGATCGTTTC |
| | GCATGATTGAACAAGATGGATTGCACGCAGGTTCTCCGGCCGCTTGGGTGGA |
| | GAGGCTATTCGGCTATGACTGGGCACAACAGACAATCGGCTGCTCTGATGCC |
| | GCCGTGTTCCGGCTGTCAGCGCAGGGGCGCCCGGTTCTTTTTGTCAAGACCG |
| | ACCTGTCCGGTGCCCTGAATGAACTGCAGGACGAGGCAGCGCGGCTATCGTG |
| | GCTGGCCACGACGGGCGTTCCTTGCGCAGCTGTGCTCGACGTTGTCACTGAA |
| | GCGGGAAGGGACTGGCTGCTATTGGGCGAAGTGCCGGGGCAGGATCTCCTGT |
| | CATCTCACCTTGCTCCTGCCGAGAAAGTATCCATCATGGCTGATGCAATGCG |
| | GCGGCTGCATACGCTTGATCCGGCTACCTGCCCATTCGACCACCAAGCGAAA |
| | CATCGCATCGAGCGAGCACGTACTCGGATGGAAGCCGGTCTTGTCGATCAGG |
| | ATGATCTGGACGAAGAGCATCAGGGGCTCGCGCCAGCCGAACTGTTCGCCAG |
| | GCTCAAGGCGCGCATGCCCGACGGCGAGGATCTCGTCGTGACCCATGGCGAT |
| | GCCTGCTTGCCGAATATCATGGTGGAAAATGGCCGCTTTTCTGGATTCATCG |
| | ACTGTGGCCGGCTGGGTGTGGCGGACCGCTATCAGGACATAGCGTTGGCTAC |
| | CCGTGATATTGCTGAAGAGCTTGGCGGCGAATGGGCTGACCGCTTCCTCGTG |
| | CTTTACGGTATCGCCGCTCCCGATTCGCAGCGCATCGCCTTCTATCGCCTTC |
| | TTGACGAGTTCTTCTGAGCGGGACTCTGGGGTTCGAAATGACCGACCAAGCG |
| | ACGCCCAACCTGCCATCACGAGATTTCGATTCCACCGCCGCCTTCTATGAAA |
| | GGTTGGGCTTCGGAATCGTTTTCCGGGACGCCGGCTGGATGATCCTCCAGCG |
| | CGGGGATCTCATGCTGGAGTTCTTCGCCCACCCCAACTTGTTTATTGCAGCT |
| | TATAATGGTTACAAATAAAGCAATAGCATCACAAATTTCACAAATAAAGCAT |
| | TTTTTTCACTGCATTCTAGTTGTGGTTTGTCCAAACTCATCAATGTATCTTA |
| | TCATGTCTGTATACCGTCGACCTCTAGCTAGAGCTTGGCGTAATCATGGTCA |
| | TAGCTGTTTCCTGTGTGAAATTGTTATCCGCTCACAATTCCACACAACATAC |
| | GAGCCGGAAGCATAAAGTGTAAAGCCTGGGGTGCCTAATGAGTGAGCTAACT |
| | CACATTAATTGCGTTGCGCTCACTGCCCGCTTTCCAGTCGGGAAACCTGTCG |
| | TGCCAGCTGCATTAATGAATCGGCCAACGCGCGGGGAGAGGCGGTTTGCGTA |
| | TTGGGCGCTCTTCCGCTTCCTCGCTCACTGACTCGCTGCGCTCGGTCGTTCG |
| | GCTGCGGCGAGCGGTATCAGCTCACTCAAAGGCGGTAATACGGTTATCCACA |
| | GAATCAGGGGATAACGCAGGAAAGAACATGTGAGCAAAAGGCCAGCAAAAGG |
| | CCAGGAACCGTAAAAAGGCCGCGTTGCTGGCGTTTTTCCATAGGCTCCGCCC |
| | CCCTGACGAGCATCACAAAAATCGACGCTCAAGTCAGAGGTGGCGAAACCCG |
| | ACAGGACTATAAAGATACCAGGCGTTTCCCCCTGGAAGCTCCCTCGTGCGCT |
| | CTCCTGTTCCGACCCTGCCGCTTACCGGATACCTGTCCGCCTTTCTCCCTTC |
| | GGGAAGCGTGGCGCTTTCTCATAGCTCACGCTGTAGGTATCTCAGTTCGGTG |
| | TAGGTCGTTCGCTCCAAGCTGGGCTGTGTGCACGAACCCCCCGTTCAGCCCG |
| | ACCGCTGCGCCTTATCCGGTAACTATCGTCTTGAGTCCAACCCGGTAAGACA |
| | CGACTTATCGCCACTGGCAGCAGCCACTGGTAACAGGATTAGCAGAGCGAGG |
| | TATGTAGGCGGTGCTACAGAGTTCTTGAAGTGGTGGCCTAACTACGGCTACA |
| | CTAGAAGAACAGTATTTGGTATCTGCGCTCTGCTGAAGCCAGTTACCTTCGG |
| | AAAAAGAGTTGGTAGCTCTTGATCCGGCAAACAAACCACCGCTGGTAGCGGT |
| | TTTTTTGTTTGCAAGCAGCAGATTACGCGCAGAAAAAAAGGATCTCAAGAAG |
| | ATCCTTTGATCTTTTCTACGGGGTCTGACGCTCAGTGGAACGAAAACTCACG |
| | TTAAGGGATTTTGGTCATGAGATTATCAAAAAGGATCTTCACCTAGATCCTT |
| | TTAAATTAAAAATGAAGTTTTAAATCAATCTAAAGTATATATGAGTAAACTT |
| | GGTCTGACAGTTACCAATGCTTAATCAGTGAGGCACCTATCTCAGCGATCTG |
| | TCTATTTCGTTCATCCATAGTTGCCTGACTCCCCGTCGTGTAGATAACTACG |
| | ATACGGGAGGGCTTACCATCTGGCCCCAGTGCTGCAATGATACCGCGAGACC |
| | CACGCTCACCGGCTCCAGATTTATCAGCAATAAACCAGCCAGCCGGAAGGGC |
| | CGAGCGCAGAAGTGGTCCTGCAACTTTATCCGCCTCCATCCAGTCTATTAAT |
| | TGTTGCCGGGAAGCTAGAGTAAGTAGTTCGCCAGTTAATAGTTTGCGCAACG |
| | TTGTTGCCATTGCTACAGGCATCGTGGTGTCACGCTCGTCGTTTGGTATGGC |
| | TTCATTCAGCTCCGGTTCCCAACGATCAAGGCGAGTTACATGATCCCCCATG |
| | TTGTGCAAAAAAGCGGTTAGCTCCTTCGGTCCTCCGATCGTTGTCAGAAGTA |
| | AGTTGGCCGCAGTGTTATCACTCATGGTTATGGCAGCACTGCATAATTCTCT |
| | TACTGTCATGCCATCCGTAAGATGCTTTTCTGTGACTGGTGAGTACTCAACC |
| | AAGTCATTCTGAGAATAGTGTATGCGGCGACCGAGTTGCTCTTGCCCGGCGT |
| | CAATACGGGATAATACCGCGCCACATAGCAGAACTTTAAAAGTGCTCATCAT |
| | TGGAAAACGTTCTTCGGGGCGAAAACTCTCAAGGATCTTACCGCTGTTGAGA |
| | TCCAGTTCGATGTAACCCACTCGTGCACCCAACTGATCTTCAGCATCTTTTA |
| | CTTTCACCAGCGTTTCTGGGTGAGCAAAAACAGGAAGGCAAAATGCCGCAAA |
| | AAAGGGAATAAGGGCGACACGGAAATGTTGAATACTCATACTCTTCCTTTTT |
| | CAATATTATTGAAGCATTTATCAGGGTTATTGTCTCATGAGCGGATACATAT |
| | TTGAATGTATTTAGAAAAATAAACAAATAGGGGTTCCGCGCACATTTCCCCG |
| | AAAAGTGCCACCTGACGTC |
| SEQ ID NO: 147 | GACGGATCGGGAGATCTCCCGATCCCCTATGGTGCACTCTCAGTACAATCTG |
| ST 6GAL1 | CTCGATGCCGCATAGTTAAGCCAGTATCTGCTCCCTGCTTGTGTGTTGGAGG |
| expression | TCGCTGAGTAGTGCGCGAGCAAAATTTAAGCTACAACAAGGCAAGGCTTGAC |
| vector | CGACAATTGCATGAAGAATCTGCTTAGGGTTAGGCGTTTTGCGCTGCTTCGC |
| | GATGTACGGGCCAGATATACGCGTTGACATTGATTATTGACTAGTTATTAAT |
| | AGTAATCAATTACGGGGTCATTAGTTCATAGCCCATATATGGAGTTCCGCGT |
| | TACATAACTTACGGTAAATGGCCCGCCTGGCTGACCGCCCAACGACCCCCGC |
| | CCATTGACGTCAATAATGACGTATGTTCCCATAGTAACGCCAATAGGGACTT |
| | TCCATTGACGTCAATGGGTGGAGTATTTACGGTAAACTGCCCACTTGGCAGT |
| | ACATCAAGTGTATCATATGCCAAGTACGCCCCCTATTGACGTCAATGACGGT |
| | AAATGGCCCGCCTGGCATTATGCCCAGTACATGACCTTATGGGACTTTCCTA |
| | CTTGGCAGTACATCTACGTATTAGTCATCGCTATTACCATGGTGATGCGGTT |
| | TTGGCAGTACATCAATGGGCGTGGATAGCGGTTTGACTCACGGGGATTTCCA |
| | AGTCTCCACCCCATTGACGTCAATGGGAGTTTGTTTTGGCACCAAAATCAAC |
| | GGGACTTTCCAAAATGTCGTAACAACTCCGCCCCATTGACGCAAATGGGCGG |
| | TAGGCGTGTACGGTGGGAGGTCTATATAAGCAGAGCTCTCTGGCTAACTAGA |
| | GAACCCACTGCTTACTGGCTTATCGAAATTAATACGACTCACTATAGGGAGA |
| | CCCAAGCTGGCTAGCGCCGCCACCATGATCCACACCAATCTGAAGAAGAAGT |
| | TTAGCTGTTGCGTGCTGGTGTTCCTGCTGTTCGCTGTGATCTGCGTGTGGAA |
| | GGAGAAGAAGAAGGGCAGCTATTATGACTCCTTTAAGCTGCAGACCAAGGAG |
| | TTCCAGGTGCTGAAGTCCCTGGGCAAGCTGGCCATGGGCAGCGACTCCCAGT |
| | CCGTGAGCTCCTCCAGCACCCAGGACCCCCACCGGGGAAGGCAGACCCTGGG |
| | ATCCCTGAGGGGCCTGGCTAAGGCCAAGCCTGAGGCTTCCTTCCAGGTGTGG |
| | AATAAGGATAGCTCCAGCAAGAACCTGATCCCTAGGCTGCAGAAGATCTGGA |
| | AGAACTATCTGAGCATGAACAAGTATAAGGTGAGCTATAAGGGCCCTGGCCC |
| | TGGCATCAAGTTCTCCGCCGAGGCCCTGAGGTGCCACCTGAGAGATCACGTG |
| | AATGTGAGCATGGTGGAGGTGACCGACTTCCCTTTTAACACCTCCGAGTGGG |
| | AGGGCTACCTGCCCAAGGAGAGCATCCGGACCAAGGCCGGCCCTTGGGGCAG |
| | ATGTGCCGTGGTGTCCAGCGCCGGCTCCCTGAAGAGCTCCCAGCTGGGCCGG |
| | GAGATCGATGACCACGATGCTGTGCTGAGGTTCAATGGCGCTCCTACCGCTA |
| | ACTTTCAGCAGGATGTGGGCACCAAGACCACCATCCGGCTGATGAATAGCCA |
| | GCTGGTGACCACCGAGAAGCGGTTTCTGAAGGACTCCCTGTACAACGAGGGC |
| | ATCCTGATCGTGTGGGACCCCTCCGTGTACCACAGCGACATCCCCAAGTGGT |
| | ATCAGAACCCCGACTACAACTTCTTCAATAATTACAAGACCTACCGGAAGCT |
| | GCACCCCAACCAGCCCTTTTATATCCTGAAGCCCCAGATGCCTTGGGAGCTG |
| | TGGGACATCCTGCAGGAGATCTCCCCCGAGGAGATCCAGCCTAACCCCCCTA |
| | GCAGCGGCATGCTGGGCATCATCATCATGATGACCCTGTGCGATCAGGTGGA |
| | TATCTACGAGTTCCTGCCCAGCAAGCGGAAGACCGACGTGTGTTATTATTAT |
| | CAGAAGTTCTTCGATTCCGCCTGCACCATGGGCGCCTATCACCCTCTGCTGT |
| | ACGAGAAGAATCTGGTGAAGCACCTGAACCAGGGCACCGACGAGGACATCTA |
| | TCTGCTGGGCAAGGCCACCCTGCCCGGCTTTAGGACCATCCACTGCTGACTC |
| | GAGTCTAGAGGGCCCGTTTAAACCCGCTGATCAGCCTCGACTGTGCCTTCTA |
| | GTTGCCAGCCATCTGTTGTTTGCCCCTCCCCCGTGCCTTCCTTGACCCTGGA |
| | AGGTGCCACTCCCACTGTCCTTTCCTAATAAAATGAGGAAATTGCATCGCAT |
| | TGTCTGAGTAGGTGTCATTCTATTCTGGGGGGTGGGGTGGGGCAGGACAGCA |
| | AGGGGGAGGATTGGGAAGACAATAGCAGGCATGCTGGGGATGCGGTGGGCTC |
| | TATGGCTTCTGAGGCGGAAAGAACCAGCTGGGGCTCTAGGGGGTATCCCCAC |
| | GCGCCCTGTAGCGGCGCATTAAGCGCGGCGGGTGTGGTGGTTACGCGCAGCG |
| | TGACCGCTACACTTGCCAGCGCCCTAGCGCCCGCTCCTTTCGCTTTCTTCCC |
| | TTCCTTTCTCGCCACGTTCGCCGGCTTTCCCCGTCAAGCTCTAAATCGGGGG |
| | CTCCCTTTAGGGTTCCGATTTAGTGCTTTACGGCACCTCGACCCCAAAAAAC |
| | TTGATTAGGGTGATGGTTCACGTAGTGGGCCATCGCCCTGATAGACGGTTTT |
| | TCGCCCTTTGACGTTGGAGTCCACGTTCTTTAATAGTGGACTCTTGTTCCAA |
| | ACTGGAACAACACTCAACCCTATCTCGGTCTATTCTTTTGATTTATAAGGGA |
| | TTTTGCCGATTTCGGCCTATTGGTTAAAAAATGAGCTGATTTAACAAAAATT |
| | TAACGCGAATTAATTCTGTGGAATGTGTGTCAGTTAGGGTGTGGAAAGTCCC |
| | CAGGCTCCCCAGCAGGCAGAAGTATGCAAAGCATGCATCTCAATTAGTCAGC |
| | AACCAGGTGTGGAAAGTCCCCAGGCTCCCCAGCAGGCAGAAGTATGCAAAGC |
| | ATGCATCTCAATTAGTCAGCAACCATAGTCCCGCCCCTAACTCCGCCCATCC |
| | CGCCCCTAACTCCGCCCAGTTCCGCCCATTCTCCGCCCCATGGCTGACTAAT |
| | TTTTTTTATTTATGCAGAGGCCGAGGCCGCCTCTGCCTCTGAGCTATTCCAG |
| | AAGTAGTGAGGAGGCTTTTTTGGAGGCCTAGGCTTTTGCAAAAAGCTCCCGG |
| | GAGCTTGTATATCCATTTTCGGATCTGATCAAGAGACAGGATGAGGATCGTT |
| | TCGCATGATTGAACAAGATGGATTGCACGCAGGTTCTCCGGCCGCTTGGGTG |
| | GAGAGGCTATTCGGCTATGACTGGGCACAACAGACAATCGGCTGCTCTGATG |
| | CCGCCGTGTTCCGGCTGTCAGCGCAGGGGCGCCCGGTTCTTTTTGTCAAGAC |
| | CGACCTGTCCGGTGCCCTGAATGAACTGCAGGACGAGGCAGCGCGGCTATCG |
| | TGGCTGGCCACGACGGGCGTTCCTTGCGCAGCTGTGCTCGACGTTGTCACTG |
| | AAGCGGGAAGGGACTGGCTGCTATTGGGCGAAGTGCCGGGGCAGGATCTCCT |
| | GTCATCTCACCTTGCTCCTGCCGAGAAAGTATCCATCATGGCTGATGCAATG |
| | CGGCGGCTGCATACGCTTGATCCGGCTACCTGCCCATTCGACCACCAAGCGA |
| | AACATCGCATCGAGCGAGCACGTACTCGGATGGAAGCCGGTCTTGTCGATCA |
| | GGATGATCTGGACGAAGAGCATCAGGGGCTCGCGCCAGCCGAACTGTTCGCC |
| | AGGCTCAAGGCGCGCATGCCCGACGGCGAGGATCTCGTCGTGACCCATGGCG |
| | ATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCT |
| | ACCCGTGATATTGCTGAAGAGCTTGGCGGCGAATGGGCTGACCGCTTCCTCG |
| | TGCTTTACGGTATCGCCGCTCCCGATTCGCAGCGCATCGCCTTCTATCGCCT |
| | TCTTGACGAGTTCTTCTGAGCGGGACTCTGGGGTTCGAAATGACCGACCAAG |
| | CGACGCCCAACCTGCCATCACGAGATTTCGATTCCACCGCCGCCTTCTATGA |
| | AAGGTTGGGCTTCGGAATCGTTTTCCGGGACGCCGGCTGGATGATCCTCCAG |
| | CGCGGGGATCTCATGCTGGAGTTCTTCGCCCACCCCAACTTGTTTATTGCAG |
| | CTTATAATGGTTACAAATAAAGCAATAGCATCACAAATTTCACAAATAAAGC |
| | ATTTTTTTCACTGCATTCTAGTTGTGGTTTGTCCAAACTCATCAATGTATCT |
| | TATCATGTCTGTATACCGTCGACCTCTAGCTAGAGCTTGGCGTAATCATGGT |
| | CATAGCTGTTTCCTGTGTGAAATTGTTATCCGCTCACAATTCCACACAACAT |
| | ACGAGCCGGAAGCATAAAGTGTAAAGCCTGGGGTGCCTAATGAGTGAGCTAA |
| | CTCACATTAATTGCGTTGCGCTCACTGCCCGCTTTCCAGTCGGGAAACCTGT |
| | CGTGCCAGCTGCATTAATGAATCGGCCAACGCGCGGGGAGAGGCGGTTTGCG |
| | TATTGGGCGCTCTTCCGCTTCCTCGCTCACTGACTCGCTGCGCTCGGTCGTT |
| | CGGCTGCGGCGAGCGGTATCAGCTCACTCAAAGGCGGTAATACGGTTATCCA |
| | CAGAATCAGGGGATAACGCAGGAAAGAACATGTGAGCAAAAGGCCAGCAAAA |
| | GGCCAGGAACCGTAAAAAGGCCGCGTTGCTGGCGTTTTTCCATAGGCTCCGC |
| | CCCCCTGACGAGCATCACAAAAATCGACGCTCAAGTCAGAGGTGGCGAAACC |
| | CGACAGGACTATAAAGATACCAGGCGTTTCCCCCTGGAAGCTCCCTCGTGCG |
| | CTCTCCTGTTCCGACCCTGCCGCTTACCGGATACCTGTCCGCCTTTCTCCCT |
| | TCGGGAAGCGTGGCGCTTTCTCATAGCTCACGCTGTAGGTATCTCAGTTCGG |
| | TGTAGGTCGTTCGCTCCAAGCTGGGCTGTGTGCACGAACCCCCCGTTCAGCC |
| | CGACCGCTGCGCCTTATCCGGTAACTATCGTCTTGAGTCCAACCCGGTAAGA |
| | CACGACTTATCGCCACTGGCAGCAGCCACTGGTAACAGGATTAGCAGAGCGA |
| | GGTATGTAGGCGGTGCTACAGAGTTCTTGAAGTGGTGGCCTAACTACGGCTA |
| | CACTAGAAGAACAGTATTTGGTATCTGCGCTCTGCTGAAGCCAGTTACCTTC |
| | GGAAAAAGAGTTGGTAGCTCTTGATCCGGCAAACAAACCACCGCTGGTAGCG |
| | GTTTTTTTGTTTGCAAGCAGCAGATTACGCGCAGAAAAAAAGGATCTCAAGA |
| | AGATCCTTTGATCTTTTCTACGGGGTCTGACGCTCAGTGGAACGAAAACTCA |
| | CGTTAAGGGATTTTGGTCATGAGATTATCAAAAAGGATCTTCACCTAGATCC |
| | TTTTAAATTAAAAATGAAGTTTTAAATCAATCTAAAGTATATATGAGTAAAC |
| | TTGGTCTGACAGTTACCAATGCTTAATCAGTGAGGCACCTATCTCAGCGATC |
| | TGTCTATTTCGTTCATCCATAGTTGCCTGACTCCCCGTCGTGTAGATAACTA |
| | CGATACGGGAGGGCTTACCATCTGGCCCCAGTGCTGCAATGATACCGCGAGA |
| | CCCACGCTCACCGGCTCCAGATTTATCAGCAATAAACCAGCCAGCCGGAAGG |
| | GCCGAGCGCAGAAGTGGTCCTGCAACTTTATCCGCCTCCATCCAGTCTATTA |
| | ATTGTTGCCGGGAAGCTAGAGTAAGTAGTTCGCCAGTTAATAGTTTGCGCAA |
| | CGTTGTTGCCATTGCTACAGGCATCGTGGTGTCACGCTCGTCGTTTGGTATG |
| | GCTTCATTCAGCTCCGGTTCCCAACGATCAAGGCGAGTTACATGATCCCCCA |
| | TGTTGTGCAAAAAAGCGGTTAGCTCCTTCGGTCCTCCGATCGTTGTCAGAAG |
| | TAAGTTGGCCGCAGTGTTATCACTCATGGTTATGGCAGCACTGCATAATTCT |
| | CTTACTGTCATGCCATCCGTAAGATGCTTTTCTGTGACTGGTGAGTACTCAA |
| | CCAAGTCATTCTGAGAATAGTGTATGCGGCGACCGAGTTGCTCTTGCCCGGC |
| | GTCAATACGGGATAATACCGCGCCACATAGCAGAACTTTAAAAGTGCTCATC |
| | ATTGGAAAACGTTCTTCGGGGCGAAAACTCTCAAGGATCTTACCGCTGTTGA |
| | GATCCAGTTCGATGTAACCCACTCGTGCACCCAACTGATCTTCAGCATCTTT |
| | TACTTTCACCAGCGTTTCTGGGTGAGCAAAAACAGGAAGGCAAAATGCCGCA |
| | AAAAAGGGAATAAGGGCGACACGGAAATGTTGAATACTCATACTCTTCCTTT |
| | TTCAATATTATTGAAGCATTTATCAGGGTTATTGTCTCATGAGCGGATACAT |
| | ATTTGAATGTATTTAGAAAAATAAACAAATAGGGGTTCCGCGCACATTTCCC |
| | CGAAAAGTGCCACCTGACGTC |
| SEQ ID NO: 148 | GACGGATCGGGAGATCTCCCGATCCCCTATGGTGCACTCTCAGTACAATCTG |
| B4GALT1 | CTCTGATGCCGCATAGTTAAGCCAGTATCTGCTCCCTGCTTGTGTGTTGGAG |
| expression | GTCGCTGAGTAGTGCGCGAGCAAAATTTAAGCTACAACAAGGCAAGGCTTGA |
| vector | CCGACAATTGCATGAAGAATCTGCTTAGGGTTAGGCGTTTTGCGCTGCTTCG |
| | CGATGTACGGGCCAGATATACGCGTTGACATTGATTATTGACTAGTTATTAA |
| | TAGTAATCAATTACGGGGTCATTAGTTCATAGCCCATATATGGAGTTCCGCG |
| | TTACATAACTTACGGTAAATGGCCCGCCTGGCTGACCGCCCAACGACCCCCG |
| | CCCATTGACGTCAATAATGACGTATGTTCCCATAGTAACGCCAATAGGGACT |
| | TTCCATTGACGTCAATGGGTGGAGTATTTACGGTAAACTGCCCACTTGGCAG |
| | TACATCAAGTGTATCATATGCCAAGTACGCCCCCTATTGACGTCAATGACGG |
| | TAAATGGCCCGCCTGGCATTATGCCCAGTACATGACCTTATGGGACTTTCCT |
| | ACTTGGCAGTACATCTACGTATTAGTCATCGCTATTACCATGGTGATGCGGT |
| | TTTGGCAGTACATCAATGGGCGTGGATAGCGGTTTGACTCACGGGGATTTCC |
| | AAGTCTCCACCCCATTGACGTCAATGGGAGTTTGTTTTGGCACCAAAATCAA |
| | CGGGACTTTCCAAAATGTCGTAACAACTCCGCCCCATTGACGCAAATGGGCG |
| | GTAGGCGTGTACGGTGGGAGGTCTATATAAGCAGAGCTCTCTGGCTAACTAG |
| | AGAACCCACTGCTTACTGGCTTATCGAAATTAATACGACTCACTATAGGGAG |
| | ACCCAAGCTGGCTAGCGCCGCCACCATGAGGCTGCGGGAGCCATTGCTGAGC |
| | GGCTCCGCTGCCATGCCTGGCGCTTCTCTGCAGAGGGCTTGTAGGCTGCTGG |
| | TGGCCGTGTGCGCCCTGCACCTGGGAGTGACCCTGGTGTACTACCTGGCTGG |
| | CCGGGACCTGTCCCGGCTGCCTCAGTTGGTGGGCGTGTCCACCCCTCTGCAG |
| | GGCGGAAGCAATAGCGCCGCTGCCATCGGCCAGTCCTCCGGAGAGCTGCGGA |
| | CCGGAGGCGCTAGGCCACCTCCACCTCTGGGCGCTTCCTCCCAGCCTCGGCC |
| | TGGAGGAGATAGCAGCCCCGTGGTGGATAGCGGCCCTGGCCCAGCTTCTAAC |
| | CTGACCAGCGTGCCTGTGCCCCACACCACCGCCCTGAGCCTGCCTGCTTGCC |
| | CCGAGGAGTCCCCCCTGCTGGTGGGACCAATGCTGATCGAGTTCAATATGCC |
| | TGTGGATCTGGAGCTGGTGGCCAAGCAGAATCCCAATGTGAAGATGGGCGGC |
| | CGGTACGCTCCCCGGGATTGTGTGTCCCCTCACAAGGTGGCTATCATCATCC |
| | CTTTCCGGAACCGGCAGGAGCACCTGAAGTACTGGCTGTATTACCTGCACCC |
| | CGTGCTGCAGAGGCAGCAGCTGGACTATGGCATCTACGTGATCAATCAGGCT |
| | GGCGACACCATCTTCAATCGGGCTAAGCTGCTGAACGTGGGCTTTCAGGAGG |
| | CTCTGAAGGACTACGACTACACCTGCTTTGTGTTCAGCGATGTGGACCTGAT |
| | CCCCATGAACGACCACAACGCTTATAGGTGCTTCTCCCAGCCTAGGCACATC |
| | TCCGTGGCTATGGACAAGTTTGGCTTCTCCCTGCCCTACGTGCAGTATTTTG |
| | GCGGCGTGTCCGCCCTGAGCAAGCAGCAGTTTCTGACCATCAATGGCTTTCC |
| | TAACAATTACTGGGGCTGGGGCGGCGAGGATGATGACATCTTCAACCGGCTG |
| | GTGTTCAGGGGCATGTCCATCAGCAGGCCTAACGCTGTGGTGGGCCGGTGTA |
| | GGATGATCAGGCACTCCCGGGACAAGAAGAATGAGCCCAATCCTCAGCGGTT |
| | TGACCGGATCGCCCACACCAAGGAGACCATGCTGTCCGACGGCCTGAATTCC |
| | CTGACCTACCAGGTGCTGGACGTGCAGAGGTATCCTCTGTACACCCAGATCA |
| | CCGTGGATATCGGCACCCCTTCCTGACTCGAGTCTAGAGGGCCCGTTTAAAC |
| | CCGCTGATCAGCCTCGACTGTGCCTTCTAGTTGCCAGCCATCTGTTGTTTGC |
| | CCCTCCCCCGTGCCTTCCTTGACCCTGGAAGGTGCCACTCCCACTGTCCTTT |
| | CCTAATAAAATGAGGAAATTGCATCGCATTGTCTGAGTAGGTGTCATTCTAT |
| | TCTGGGGGGTGGGGTGGGGCAGGACAGCAAGGGGGAGGATTGGGAAGACAAT |
| | AGCAGGCATGCTGGGGATGCGGTGGGCTCTATGGCTTCTGAGGCGGAAAGAA |
| | CCAGCTGGGGCTCTAGGGGGTATCCCCACGCGCCCTGTAGCGGCGCATTAAG |
| | CGCGGCGGGTGTGGTGGTTACGCGCAGCGTGACCGCTACACTTGCCAGCGCC |
| | CTAGCGCCCGCTCCTTTCGCTTTCTTCCCTTCCTTTCTCGCCACGTTCGCCG |
| | GCTTTCCCCGTCAAGCTCTAAATCGGGGGCTCCCTTTAGGGTTCCGATTTAG |
| | TGCTTTACGGCACCTCGACCCCAAAAAACTTGATTAGGGTGATGGTTCACGT |
| | AGTGGGCCATCGCCCTGATAGACGGTTTTTCGCCCTTTGACGTTGGAGTCCA |
| | CGTTCTTTAATAGTGGACTCTTGTTCCAAACTGGAACAACACTCAACCCTAT |
| | CTCGGTCTATTCTTTTGATTTATAAGGGATTTTGCCGATTTCGGCCTATTGG |
| | TTAAAAAATGAGCTGATTTAACAAAAATTTAACGCGAATTAATTCTGTGGAA |
| | TGTGTGTCAGTTAGGGTGTGGAAAGTCCCCAGGCTCCCCAGCAGGCAGAAGT |
| | ATGCAAAGCATGCATCTCAATTAGTCAGCAACCAGGTGTGGAAAGTCCCCAG |
| | GCTCCCCAGCAGGCAGAAGTATGCAAAGCATGCATCTCAATTAGTCAGCAAC |
| | CATAGTCCCGCCCCTAACTCCGCCCATCCCGCCCCTAACTCCGCCCAGTTCC |
| | GCCCATTCTCCGCCCCATGGCTGACTAATTTTTTTTATTTATGCAGAGGCCG |
| | AGGCCGCCTCTGCCTCTGAGCTATTCCAGAAGTAGTGAGGAGGCTTTTTTGG |
| | AGGCCTAGGCTTTTGCAAAAAGCTCCCGGGAGCTTGTATATCCATTTTCGGA |
| | TCTGATCAAGAGACAGGATGAGGATCGTTTCGCATGATTGAACAAGATGGAT |
| | TGCACGCAGGTTCTCCGGCCGCTTGGGTGGAGAGGCTATTCGGCTATGACTG |
| | GGCACAACAGACAATCGGCTGCTCTGATGCCGCCGTGTTCCGGCTGTCAGCG |
| | CAGGGGCGCCCGGTTCTTTTTGTCAAGACCGACCTGTCCGGTGCCCTGAATG |
| | AACTGCAGGACGAGGCAGCGCGGCTATCGTGGCTGGCCACGACGGGCGTTCC |
| | TTGCGCAGCTGTGCTCGACGTTGTCACTGAAGCGGGAAGGGACTGGCTGCTA |
| | TTGGGCGAAGTGCCGGGGCAGGATCTCCTGTCATCTCACCTTGCTCCTGCCG |
| | AGAAAGTATCCATCATGGCTGATGCAATGCGGCGGCTGCATACGCTTGATCC |
| | GGCTACCTGCCCATTCGACCACCAAGCGAAACATCGCATCGAGCGAGCACGT |
| | ACTCGGATGGAAGCCGGTCTTGTCGATCAGGATGATCTGGACGAAGAGCATC |
| | AGGGGCTCGCGCCAGCCGAACTGTTCGCCAGGCTCAAGGCGCGCATGCCCGA |
| | CGGCGAGGATCTCGTCGTGACCCATGGCGATGCCTGCTTGCCGAATATCATG |
| | GTGGAAAATGGCCGCTTTTCTGGATTCATCGACTGTGGCCGGCTGGGTGTGG |
| | CGGACCGCTATCAGGACATAGCGTTGGCTACCCGTGATATTGCTGAAGAGCT |
| | TGGCGGCGAATGGGCTGACCGCTTCCTCGTGCTTTACGGTATCGCCGCTCCC |
| | GATTCGCAGCGCATCGCCTTCTATCGCCTTCTTGACGAGTTCTTCTGAGCGG |
| | GACTCTGGGGTTCGAAATGACCGACCAAGCGACGCCCAACCTGCCATCACGA |
| | GATTTCGATTCCACCGCCGCCTTCTATGAAAGGTTGGGCTTCGGAATCGTTT |
| | TCCGGGACGCCGGCTGGATGATCCTCCAGCGCGGGGATCTCATGCTGGAGTT |
| | CTTCGCCCACCCCAACTTGTTTATTGCAGCTTATAATGGTTACAAATAAAGC |
| | AATAGCATCACAAATTTCACAAATAAAGCATTTTTTTCACTGCATTCTAGTT |
| | GTGGTTTGTCCAAACTCATCAATGTATCTTATCATGTCTGTATACCGTCGAC |
| | CTCTAGCTAGAGCTTGGCGTAATCATGGTCATAGCTGTTTCCTGTGTGAAAT |
| | TGTTATCCGCTCACAATTCCACACAACATACGAGCCGGAAGCATAAAGTGTA |
| | AAGCCTGGGGTGCCTAATGAGTGAGCTAACTCACATTAATTGCGTTGCGCTC |
| | ACTGCCCGCTTTCCAGTCGGGAAACCTGTCGTGCCAGCTGCATTAATGAATC |
| | GGCCAACGCGCGGGGAGAGGCGGTTTGCGTATTGGGCGCTCTTCCGCTTCCT |
| | CGCTCACTGACTCGCTGCGCTCGGTCGTTCGGCTGCGGCGAGCGGTATCAGC |
| | TCACTCAAAGGCGGTAATACGGTTATCCACAGAATCAGGGGATAACGCAGGA |
| | AAGAACATGTGAGCAAAAGGCCAGCAAAAGGCCAGGAACCGTAAAAAGGCCG |
| | CGTTGCTGGCGTTTTTCCATAGGCTCCGCCCCCCTGACGAGCATCACAAAAA |
| | TCGACGCTCAAGTCAGAGGTGGCGAAACCCGACAGGACTATAAAGATACCAG |
| | GCGTTTCCCCCTGGAAGCTCCCTCGTGCGCTCTCCTGTTCCGACCCTGCCGC |
| | TTACCGGATACCTGTCCGCCTTTCTCCCTTCGGGAAGCGTGGCGCTTTCTCA |
| | TAGCTCACGCTGTAGGTATCTCAGTTCGGTGTAGGTCGTTCGCTCCAAGCTG |
| | GGCTGTGTGCACGAACCCCCCGTTCAGCCCGACCGCTGCGCCTTATCCGGTA |
| | ACTATCGTCTTGAGTCCAACCCGGTAAGACACGACTTATCGCCACTGGCAGC |
| | AGCCACTGGTAACAGGATTAGCAGAGCGAGGTATGTAGGCGGTGCTACAGAG |
| | TTCTTGAAGTGGTGGCCTAACTACGGCTACACTAGAAGAACAGTATTTGGTA |
| | TCTGCGCTCTGCTGAAGCCAGTTACCTTCGGAAAAAGAGTTGGTAGCTCTTG |
| | ATCCGGCAAACAAACCACCGCTGGTAGCGGTTTTTTTGTTTGCAAGCAGCAG |
| | ATTACGCGCAGAAAAAAAGGATCTCAAGAAGATCCTTTGATCTTTTCTACGG |
| | GGTCTGACGCTCAGTGGAACGAAAACTCACGTTAAGGGATTTTGGTCATGAG |
| | ATTATCAAAAAGGATCTTCACCTAGATCCTTTTAAATTAAAAATGAAGTTTT |
| | AAATCAATCTAAAGTATATATGAGTAAACTTGGTCTGACAGTTACCAATGCT |
| | TAATCAGTGAGGCACCTATCTCAGCGATCTGTCTATTTCGTTCATCCATAGT |
| | TGCCTGACTCCCCGTCGTGTAGATAACTACGATACGGGAGGGCTTACCATCT |
| | GGCCCCAGTGCTGCAATGATACCGCGAGACCCACGCTCACCGGCTCCAGATT |
| | TATCAGCAATAAACCAGCCAGCCGGAAGGGCCGAGCGCAGAAGTGGTCCTGC |
| | AACTTTATCCGCCTCCATCCAGTCTATTAATTGTTGCCGGGAAGCTAGAGTA |
| | AGTAGTTCGCCAGTTAATAGTTTGCGCAACGTTGTTGCCATTGCTACAGGCA |
| | TCGTGGTGTCACGCTCGTCGTTTGGTATGGCTTCATTCAGCTCCGGTTCCCA |
| | ACGATCAAGGCGAGTTACATGATCCCCCATGTTGTGCAAAAAAGCGGTTAGC |
| | TCCTTCGGTCCTCCGATCGTTGTCAGAAGTAAGTTGGCCGCAGTGTTATCAC |
| | TCATGGTTATGGCAGCACTGCATAATTCTCTTACTGTCATGCCATCCGTAAG |
| | ATGCTTTTCTGTGACTGGTGAGTACTCAACCAAGTCATTCTGAGAATAGTGT |
| | ATGCGGCGACCGAGTTGCTCTTGCCCGGCGTCAATACGGGATAATACCGCGC |
| | CACATAGCAGAACTTTAAAAGTGCTCATCATTGGAAAACGTTCTTCGGGGCG |
| | AAAACTCTCAAGGATCTTACCGCTGTTGAGATCCAGTTCGATGTAACCCACT |
| | CGTGCACCCAACTGATCTTCAGCATCTTTTACTTTCACCAGCGTTTCTGGGT |
| | GAGCAAAAACAGGAAGGCAAAATGCCGCAAAAAAGGGAATAAGGGCGACACG |
| | GAAATGTTGAATACTCATACTCTTCCTTTTTCAATATTATTGAAGCATTTAT |
| | CAGGGTTATTGTCTCATGAGCGGATACATATTTGAATGTATTTAGAAAAATA |
| | AACAAATAGGGGTTCCGCGCACATTTCCCCGAAAAGTGCCACCTGACGTC |
| SEQ ID NO: 149 | GACGGATCGGGAGATCTCCCGATCCCCTATGGTGCACTCTCAGTACAATCTG |
| pcDNA2 TADA | CTCTGATGCCGCATAGTTAAGCCAGTATCTGCTCCCTGCTTGTGTGTTGGAG |
| (Adalimumab), | GTCGCTGAGTAGTGCGCGAGCAAAATTTAAGCTACAACAAGGCAAGGCTTGA |
| nt | CCGACAATTGCATGAAGAATCTGCTTAGGGTTAGGCGTTTTGCGCTGCTTCG |
| | CGATGTACGGGCCAGATATACGCGTTGACATTGATTATTGACTAGTTATTAA |
| | TAGTAATCAATTACGGGGTCATTAGTTCATAGCCCATATATGGAGTTCCGCG |
| | TTACATAACTTACGGTAAATGGCCCGCCTGGCTGACCGCCCAACGACCCCCG |
| | CCCATTGACGTCAATAATGACGTATGTTCCCATAGTAACGCCAATAGGGACT |
| | TTCCATTGACGTCAATGGGTGGAGTATTTACGGTAAACTGCCCACTTGGCAG |
| | TACATCAAGTGTATCATATGCCAAGTACGCCCCCTATTGACGTCAATGACGG |
| | TAAATGGCCCGCCTGGCATTATGCCCAGTACATGACCTTATGGGACTTTCCT |
| | ACTTGGCAGTACATCTACGTATTAGTCATCGCTATTACCATGGTGATGCGGT |
| | TTTGGCAGTACATCAATGGGCGTGGATAGCGGTTTGACTCACGGGGATTTCC |
| | AAGTCTCCACCCCATTGACGTCAATGGGAGTTTGTTTTGGCACCAAAATCAA |
| | CGGGACTTTCCAAAATGTCGTAACAACTCCGCCCCATTGACGCAAATGGGCG |
| | GTAGGCGTGTACGGTGGGAGGTCTATATAAGCAGAGCTCTCTGGCTAACTAG |
| | AGAACCCACTGCTTACTGGCTTATCGAAATTAATACGACTCACTATAGGGAG |
| | ACCCAAGCTAAGCTTGCCGCCACCATGAGGGTCCCCGCTCAGCTCCTGGGGC |
| | TCCTGCTGCTCTGGCTCCCAGGTGCACGATGTGATATCCAAATGACTCAAAG |
| | TCCAAGTAGTCTGTCCGCAAGCGTCGGCGATCGCGTGACCATCACATGTAGA |
| | GCTTCTCAAGGCATCCGGAACTACCTGGCTTGGTACCAGCAGAAGCCTGGCA |
| | AGGCCCCCAAGCTGCTGATCTATGCCGCTTCTACCCTCCAGTCCGGCGTGCC |
| | TAGCAGATTTTCCGGCTCCGGATCTGGAACAGACTTCACCCTGACCATCTCC |
| | TCTCTGCAGCCTGAGGACGTGGCCACCTACTACTGCCAGCGGTACAACAGAG |
| | CCCCATACACCTTCGGCCAGGGCACCAAGGTGGAAATCAAACGTACGGTGGC |
| | TGCACCATCTGTCTTCATCTTCCCGCCATCTGATGAGCAGTTGAAATCTGGA |
| | ACTGCCTCTGTTGTGTGCCTGCTGAATAACTTCTATCCCAGAGAGGCCAAAG |
| | TACAGTGGAAGGTGGATAACGCCCTCCAATCGGGTAACTCCCAGGAGAGTGT |
| | CACAGAGCAGGACAGCAAGGACAGCACCTACAGCCTCAGCAGCACCCTGACG |
| | CTGAGCAAAGCAGACTACGAGAAACACAAAGTCTACGCCTGCGAAGTCACCC |
| | ATCAGGGCCTGAGCTCGCCCGTCACAAAGAGCTTCAACAGGGGAGAGTGTTG |
| | AGGATCCACTAGTCCAGTGTGGTGGAATTCAGATCCGTTAACGGTTACCAAC |
| | TACCTAGACTGGATTCGTGACAACATGCGGCCGTGATATCTACGTATGATCA |
| | GCCTCGACTGTGCCTTCTAGTTGCCAGCCATCTGTTGTTTGCCCCTCCCCCG |
| | TGCCTTCCTTGACCCTGGAAGGTGCCACTCCCACTGTCCTTTCCTAATAAAA |
| | TGAGGAAATTGCATCGCATTGTCTGAGTAGGTGTCATTCTATTCTGGGGGGT |
| | GGGGTGGGGCAGGACAGCAAGGGGGAGGATTGGGAAGACAATAGCAGGCATG |
| | CTGGGGATGCGGTGGGCTCTATGGAACCAGCTGGGGCTCGACAGCTATGCCA |
| | AGTACGCCCCCTATTGACGTCAATGACGGTAAATGGCCCGCCTGGCATTATG |
| | CCCAGTACATGACCTTATGGGACTTTCCTACTTGGCAGTACATCTACGTATT |
| | AGTCATCGCTATTACCATGGTGATGCGGTTTTGGCAGTACATCAATGGGCGT |
| | GGATAGCGGTTTGACTCACGGGGATTTCCAAGTCTCCACCCCATTGACGTCA |
| | ATGGGAGTTTGTTTTGGCACCAAAATCAACGGGACTTTCCAAAATGTCGTAA |
| | CAACTCCGCCCCATTGACGCAAATGGGCGGTAGGCGTGTACGGTGGGAGGTC |
| | TATATAAGCAGAGCTGGGTACGTCCTCACATTCAGTGATCAGCACTGAACAC |
| | AGACCCGCGGCCGCTCGAGGCCGCCACCATGGGTTGGAGCCTCATCTTGCTC |
| | TTCCTTGTCGCTGTTGCTACGCGTGTCGAAGTGCAACTAGTGGAAAGTGGTG |
| | GTGGTCTCGTGCAGCCCGGAAGGTCTCTGCGGCTGTCCTGTGCTGCTTCCGG |
| | CTTCACCTTCGACGATTATGCCATGCACTGGGTCCGGCAAGCTCCTGGCAAG |
| | GGCCTGGAGTGGGTCTCTGCCATCACCTGGAATTCTGGCCACATCGACTACG |
| | CCGACTCCGTGGAAGGCAGATTTACCATCTCCAGAGACAACGCCAAGAACAG |
| | CCTGTACCTGCAGATGAACTCCCTGAGAGCCGAGGATACAGCCGTGTACTAC |
| | TGCGCCAAAGTGTCCTACCTGTCTACCGCTTCTAGCCTGGACTACTGGGGCC |
| | AGGGCACCCTGGTGACCGTGTCCAGCGCTAGCACCAAGGGCCCATCGGTCTT |
| | CCCCCTGGCACCCTCCTCCAAGAGCACCTCTGGGGGCACAGCGGCCCTGGGC |
| | TGCCTGGTCAAGGACTACTTCCCCGAACCGGTGACGGTGTCGTGGAACTCAG |
| | GCGCCCTGACCAGCGGCGTGCACACCTTCCCGGCTGTCCTACAGTCCTCAGG |
| | ACTCTACTCCCTCAGCAGCGTGGTGACCGTGCCCTCCAGCAGCTTGGGCACC |
| | CAGACCTACATCTGCAACGTGAATCACAAGCCCAGCAACACCAAGGTGGACA |
| | AGAAAGTAGAGCCCAAATCTTGTGACAAAACTCACACATGCCCACCGTGCCC |
| | AGCACCTGAACTCCTGGGGGGACCGTCAGTCTTCCTCTTC |
| | CCCCCAAAACCCAAGGACACCCTCATGATCTCCCGGACCCCTGAGGTCACAT |
| | GCGTGGTGGTGGACGTGAGCCACGAAGACCCTGAGGTCAAGTTCAACTGGTA |
| | CGTGGACGGCGTGGAGGTGCATAATGCCAAGACAAAGCCGCGGGAGGAGCAG |
| | TACAACAGCACGTACCGTGTGGTCAGCGTCCTCACCGTCCTGCACCAGGACT |
| | GGCTGAATGGCAAGGAGTACAAGTGCAAGGTCTCCAACAAAGCCCTCCCAGC |
| | CCCCATCGAGAAAACCATCTCCAAAGCCAAAGGGCAGCCCCGAGAACCACAG |
| | GTGTACACCCTGCCCCCATCCCGGGATGAGCTGACCAAGAACCAGGTCAGCC |
| | TGACCTGCCTGGTCAAAGGCTTCTATCCCAGCGACATCGCCGTGGAGTGGGA |
| | GAGCAATGGGCAGCCGGAGAACAACTACAAGACCACGCCTCCCGTGCTGGAC |
| | TCCGACGGCTCCTTCTTCCTCTACAGCAAGCTCACCGTGGACAAGAGCAGGT |
| | GGCAGCAGGGGAACGTCTTCTCATGCTCCGTGATGCATGAGGCTCTGCACAA |
| | CCACTACACGCAGAAGAGCCTCTCCCTGTCTCCGGGTAAATGAGTTTAAACC |
| | CGCTGATCAGCCTCGACTGTGCCTTCTAGTTGCCAGCCATCTGTTGTTTGCC |
| | CCTCCCCCGTGCCTTCCTTGACCCTGGAAGGTGCCACTCCCACTGTCCTTTC |
| | CTAATAAAATGAGGAAATTGCATCGCATTGTCTGAGTAGGTGTCATTCTATT |
| | CTGGGGGGTGGGGTGGGGCAGGACAGCAAGGGGGAGGATTGGGAAGACAATA |
| | GCAGGCATGCTGGGGATGCGGTGGGCTCTATGGCTTCTGAGGCGGAAAGAAC |
| | CAGCTGGGGCTCTAGGGGGTATCCCCACGCGCCCTGTAGCGGCGCATTAAGC |
| | GCGGCGGGTGTGGTGGTTACGCGCAGCGTGACCGCTACACTTGCCAGCGCCC |
| | TAGCGCCCGCTCCTTTCGCTTTCTTCCCTTCCTTTCTCGCCACGTTCGCCGG |
| | CTTTCCCCGTCAAGCTCTAAATCGGGGGCTCCCTTTAGGGTTCCGATTTAGT |
| | GCTTTACGGCACCTCGACCCCAAAAAACTTGATTAGGGTGATGGTTCACGTA |
| | GTGGGCCATCGCCCTGATAGACGGTTTTTCGCCCTTTGACGTTGGAGTCCAC |
| | GTTCTTTAATAGTGGACTCTTGTTCCAAACTGGAACAACACTCAACCCTATC |
| | TCGGTCTATTCTTTTGATTTATAAGGGATTTTGCCGATTTCGGCCTATTGGT |
| | TAAAAAATGAGCTGATTTAACAAAAATTTAACGCGAATTAATTCTGTGGAAT |
| | GTGTGTCAGTTAGGGTGTGGAAAGTCCCCAGGCTCCCCAGCAGGCAGAAGTA |
| | TGCAAAGCATGCATCTCAATTAGTCAGCAACCAGGTGTGGAAAGTCCCCAGG |
| | CTCCCCAGCAGGCAGAAGTATGCAAAGCATGCATCTCAATTAGTCAGCAACC |
| | ATAGTCCCGCCCCTAACTCCGCCCATCCCGCCCCTAACTCCGCCCAGTTCCG |
| | CCCATTCTCCGCCCCATGGCTGACTAATTTTTTTTATTTATGCAGAGGCCGA |
| | GGCCGCCTCTGCCTCTGAGCTATTCCAGAAGTAGTGAGGAGGCTTTTTTGGA |
| | GGCCTAGGCTTTTGCAAAAAGCTCCCGGGAGCTTGTATATCCATTTTCGGAT |
| | CTGATCAAGAGACAGGATGAGGATCGTTTCGCATGATTGAACAAGATGGATT |
| | GCACGCAGGTTCTCCGGCCGCTTGGGTGGAGAGGCTATTCGGCTATGACTGG |
| | GCACAACAGACAATCGGCTGCTCTGATGCCGCCGTGTTCCGGCTGTCAGCGC |
| | AGGGGCGCCCGGTTCTTTTTGTCAAGACCGACCTGTCCGGTGCCCTGAATGA |
| | ACTGCAGGACGAGGCAGCGCGGCTATCGTGGCTGGCCACGACGGGCGTTCCT |
| | TGCGCAGCTGTGCTCGACGTTGTCACTGAAGCGGGAAGGGACTGGCTGCTAT |
| | TGGGCGAAGTGCCGGGGCAGGATCTCCTGTCATCTCACCTTGCTCCTGCCGA |
| | GAAAGTATCCATCATGGCTGATGCAATGCGGCGGCTGCATACGCTTGATCCG |
| | GCTACCTGCCCATTCGACCACCAAGCGAAACATCGCATCGAGCGAGCACGTA |
| | CTCGGATGGAAGCCGGTCTTGTCGATCAGGATGATCTGGACGAAGAGCATCA |
| | GGGGCTCGCGCCAGCCGAACTGTTCGCCAGGCTCAAGGCGCGCATGCCCGAC |
| | GGCGAGGATCTCGTCGTGACCCATGGCGATGCCTGCTTGCCGAATATCATGG |
| | TGGAAAATGGCCGCTTTTCTGGATTCATCGACTGTGGCCGGCTGGGTGTGGC |
| | GGACCGCTATCAGGACATAGCGTTGGCTACCCGTGATATTGCTGAAGAGCTT |
| | GGCGGCGAATGGGCTGACCGCTTCCTCGTGCTTTACGGTATCGCCGCTCCCG |
| | ATTCGCAGCGCATCGCCTTCTATCGCCTTCTTGACGAGTTCTTCTGAGCGGG |
| | ACTCTGGGGTTCGAAATGACCGACCAAGCGACGCCCAACCTGCCATCACGAG |
| | ATTTCGATTCCACCGCCGCCTTCTATGAAAGGTTGGGCTTCGGAATCGTTTT |
| | CCGGGACGCCGGCTGGATGATCCTCCAGCGCGGGGATCTCATGCTGGAGTTC |
| | TTCGCCCACCCCAACTTGTTTATTGCAGCTTATAATGGTTACAAATAAAGCA |
| | ATAGCATCACAAATTTCACAAATAAAGCATTTTTTTCACTGCATTCTAGTTG |
| | TGGTTTGTCCAAACTCATCAATGTATCTTATCATGTCTGTATACCGTCGACC |
| | TCTAGCTAGAGCTTGGCGTAATCATGGTCATAGCTGTTTCCTGTGTGAAATT |
| | GTTATCCGCTCACAATTCCACACAACATACGAGCCGGAAGCATAAAGTGTAA |
| | AGCCTGGGGTGCCTAATGAGTGAGCTAACTCACATTAATTGCGTTGCGCTCA |
| | CTGCCCGCTTTCCAGTCGGGAAACCTGTCGTGCCAGCTGCATTAATGAATCG |
| | GCCAACGCGCGGGGAGAGGCGGTTTGCGTATTGGGCGCTCTTCCGCTTCCTC |
| | GCTCACTGACTCGCTGCGCTCGGTCGTTCGGCTGCGGCGAGCGGTATCAGCT |
| | CACTCAAAGGCGGTAATACGGTTATCCACAGAATCAGGGGATAACGCAGGAA |
| | AGAACATGTGAGCAAAAGGCCAGCAAAAGGCCAGGAACCGTAAAAAGGCCGC |
| | GTTGCTGGCGTTTTTCCATAGGCTCCGCCCCCCTGACGAGCATCACAAAAAT |
| | CGACGCTCAAGTCAGAGGTGGCGAAACCCGACAGGACTATAAAGATACCAGG |
| | CGTTTCCCCCTGGAAGCTCCCTCGTGCGCTCTCCTGTTCCGACCCTGCCGCT |
| | TACCGGATACCTGTCCGCCTTTCTCCCTTCGGGAAGCGTGGCGCTTTCTCAT |
| | AGCTCACGCTGTAGGTATCTCAGTTCGGTGTAGGTCGTTCGCTCCAAGCTGG |
| | GCTGTGTGCACGAACCCCCCGTTCAGCCCGACCGCTGCGCCTTATCCGGTAA |
| | CTATCGTCTTGAGTCCAACCCGGTAAGACACGACTTATCGCCACTGGCAGCA |
| | GCCACTGGTAACAGGATTAGCAGAGCGAGGTATGTAGGCGGTGCTACAGAGT |
| | TCTTGAAGTGGTGGCCTAACTACGGCTACACTAGAAGAACAGTATTTGGTAT |
| | CTGCGCTCTGCTGAAGCCAGTTACCTTCGGAAAAAGAGTTGGTAGCTCTTGA |
| SEQ ID NO: 150 | TCCGGCAAACAAACCACCGCTGGTAGCGGTTTTTTTGTTTGCAAGCAGCAGA |
| | TTACGCGCAGAAAAAAAGGATCTCAAGAAGATCCTTTGATCTTTTCTACGGG |
| | GTCTGACGCTCAGTGGAACGAAAACTCACGTTAAGGGATTTTGGTCATGAGA |
| | TTATCAAAAAGGATCTTCACCTAGATCCTTTTAAATTAAAAATGAAGTTTTA |
| | AATCAATCTAAAGTATATATGAGTAAACTTGGTCTGACAGTTACCAATGCTT |
| | AATCAGTGAGGCACCTATCTCAGCGATCTGTCTATTTCGTTCATCCATAGTT |
| | GCCTGACTCCCCGTCGTGTAGATAACTACGATACGGGAGGGCTTACCATCTG |
| | GCCCCAGTGCTGCAATGATACCGCGAGACCCACGCTCACCGGCTCCAGATTT |
| | ATCAGCAATAAACCAGCCAGCCGGAAGGGCCGAGCGCAGAAGTGGTCCTGCA |
| | ACTTTATCCGCCTCCATCCAGTCTATTAATTGTTGCCGGGAAGCTAGAGTAA |
| | GTAGTTCGCCAGTTAATAGTTTGCGCAACGTTGTTGCCATTGCTACAGGCAT |
| | CGTGGTGTCACGCTCGTCGTTTGGTATGGCTTCATTCAGCTCCGGTTCCCAA |
| | CGATCAAGGCGAGTTACATGATCCCCCATGTTGTGCAAAAAAGCGGTTAGCT |
| | CCTTCGGTCCTCCGATCGTTGTCAGAAGTAAGTTGGCCGCAGTGTTATCACT |
| | CATGGTTATGGCAGCACTGCATAATTCTCTTACTGTCATGCCATCCGTAAGA |
| | TGCTTTTCTGTGACTGGTGAGTACTCAACCAAGTCATTCTGAGAATAGTGTA |
| | TGCGGCGACCGAGTTGCTCTTGCCCGGCGTCAATACGGGATAATACCGCGCC |
| | ACATAGCAGAACTTTAAAAGTGCTCATCATTGGAAAACGTTCTTCGGGGCGA |
| | AAACTCTCAAGGATCTTACCGCTGTTGAGATCCAGTTCGATGTAACCCACTC |
| | GTGCACCCAACTGATCTTCAGCATCTTTTACTTTCACCAGCGTTTCTGGGTG |
| | AGCAAAAACAGGAAGGCAAAATGCCGCAAAAAAGGGAATAAGGGCGACACGG |
| | AAATGTTGAATACTCATACTCTTCCTTTTTCAATATTATTGAAGCATTTATC |
| | AGGGTTATTGTCTCATGAGCGGATACATATTTGAATGTATTTAGAAAAATAA |
| | ACAAATAGGGGTTCCGCGCACATTTCCCCGAAAAGTGCCACCTGACGTC |
| | CATGGACTGG TTCCTGGCGT TGG |
| CERNA | |
| RNA | |
| SEQ ID NO: 151 | CTCCAGCTTT GCAAGAATCT TGG |
| CERNA | |
| RNA | |
| SEQ ID NO: 152 | ATAAAACAAT AAGGTCCCCC AGG |
| CERNA | |
| RNA | |
| SEQ ID NO: 153 | GACAAGAAGTACAGCATCGGCCTGGACATCGGCACCAACTCTGTGGGCTGGGCCGTGATCA |
| Cas9 | CCGACGAGTACAAGGTGCCCAGCAAGAAATTCAAGGTGCTGGGCAACACCGACCGGCACAG |
| RNA | CATCAAGAAGAACCTGATCGGAGCCCTGCTGTTCGACAGCGGCGAAACAGCCGAGGCCACC |
| | CGGCTGAAGAGAACCGCCAGAAGAAGATACACCAGACGGAAGAACCGGATCTGCTATCTGC |
| | AAGAGATCTTCAGCAACGAGATGGCCAAGGTGGACGACAGCTTCTTCCACAGACTGGAAGA |
| | GTCCTTCCTGGTGGAAGAGGATAAGAAGCACGAGCGGCACCCCATCTTCGGCAACATCGTG |
| | GACGAGGTGGCCTACCACGAGAAGTACCCCACCATCTACCACCTGAGAAAGAAACTGGTGG |
| | ACAGCACCGACAAGGCCGACCTGCGGCTGATCTATCTGGCCCTGGCCCACATGATCAAGTT |
| | CCGGGGCCACTTCCTGATCGAGGGCGACCTGAACCCCGACAACAGCGACGTGGACAAGCTG |
| | TTCATCCAGCTGGTGCAGACCTACAACCAGCTGTTCGAGGAAAACCCCATCAACGCCAGCG |
| | GCGTGGACGCCAAGGCCATCCTGTCTGCCAGACTGAGCAAGAGCAGACGGCTGGAAAATCT |
| | GATCGCCCAGCTGCCCGGCGAGAAGAAGAATGGCCTGTTCGGAAACCTGATTGCCCTGAGC |
| | CTGGGCCTGACCCCCAACTTCAAGAGCAACTTCGACCTGGCCGAGGATGCCAAACTGCAGC |
| | TGAGCAAGGACACCTACGACGACGACCTGGACAACCTGCTGGCCCAGATCGGCGACCAGTA |
| | CGCCGACCTGTTTCTGGCCGCCAAGAACCTGTCCGACGCCATCCTGCTGAGCGACATCCTG |
| | AGAGTGAACACCGAGATCACCAAGGCCCCCCTGAGCGCCTCTATGATCAAGAGATACGACG |
| | AGCACCACCAGGACCTGACCCTGCTGAAAGCTCTCGTGCGGCAGCAGCTGCCTGAGAAGTA |
| | CAAAGAGATTTTCTTCGACCAGAGCAAGAACGGCTACGCCGGCTACATTGACGGCGGAGCC |
| | AGCCAGGAAGAGTTCTACAAGTTCATCAAGCCCATCCTGGAAAAGATGGACGGCACCGAGG |
| | AACTGCTCGTGAAGCTGAACAGAGAGGACCTGCTGCGGAAGCAGCGGACCTTCGACAACGG |
| | CAGCATCCCCCACCAGATCCACCTGGGAGAGCTGCACGCCATTCTGCGGCGGCAGGAAGAT |
| | TTTTACCCATTCCTGAAGGACAACCGGGAAAAGATCGAGAAGATCCTGACCTTCCGCATCC |
| | CCTACTACGTGGGCCCTCTGGCCAGGGGAAACAGCAGATTCGCCTGGATGACCAGAAAGAG |
| | CGAGGAAACCATCACCCCCTGGAACTTCGAGGAAGTGGTGGACAAGGGCGCTTCCGCCCAG |
| | AGCTTCATCGAGCGGATGACCAACTTCGATAAGAACCTGCCCAACGAGAAGGTGCTGCCCA |
| | AGCACAGCCTGCTGTACGAGTACTTCACCGTGTATAACGAGCTGACCAAAGTGAAATACGT |
| | GACCGAGGGAATGAGAAAGCCCGCCTTCCTGAGCGGCGAGCAGAAAAAGGCCATCGTGGAC |
| | CTGCTGTTCAAGACCAACCGGAAAGTGACCGTGAAGCAGCTGAAAGAGGACTACTTCAAGA |
| | AAATCGAGTGCTTCGACTCCGTGGAAATCTCCGGCGTGGAAGATCGGTTCAACGCCTCCCT |
| | GGGCACATACCACGATCTGCTGAAAATTATCAAGGACAAGGACTTCCTGGACAATGAGGAA |
| | AACGAGGACATTCTGGAAGATATCGTGCTGACCCTGACACTGTTTGAGGACAGAGAGATGA |
| | TCGAGGAACGGCTGAAAACCTATGCCCACCTGTTCGACGACAAAGTGATGAAGCAGCTGAA |
| | GCGGCGGAGATACACCGGCTGGGGCAGGCTGAGCCGGAAGCTGATCAACGGCATCCGGGAC |
| | AAGCAGTCCGGCAAGACAATCCTGGATTTCCTGAAGTCCGACGGCTTCGCCAACAGAAACT |
| | TCATGCAGCTGATCCACGACGACAGCCTGACCTTTAAAGAGGACATCCAGAAAGCCCAGGT |
| | GTCCGGCCAGGGCGATAGCCTGCACGAGCACATTGCCAATCTGGCCGGCAGCCCCGCCATT |
| | AAGAAGGGCATCCTGCAGACAGTGAAGGTGGTGGACGAGCTCGTGAAAGTGATGGGCCGGC |
| | ACAAGCCCGAGAACATCGTGATCGAAATGGCCAGAGAGAACCAGACCACCCAGAAGGGACA |
| | GAAGAACAGCCGCGAGAGAATGAAGCGGATCGAAGAGGGCATCAAAGAGCTGGGCAGCCAG |
| | ATCCTGAAAGAACACCCCGTGGAAAACACCCAGCTGCAGAACGAGAAGCTGTACCTGTACT |
| | ACCTGCAGAATGGGCGGGATATGTACGTGGACCAGGAACTGGACATCAACCGGCTGTCCGA |
| | CTACGATGTGGACCATATCGTGCCTCAGAGCTTTCTGAAGGACGACTCCATCGACAACAAG |
| | GTGCTGACCAGAAGCGACAAGAACCGGGGCAAGAGCGACAACGTGCCCTCCGAAGAGGTCG |
| | TGAAGAAGATGAAGAACTACTGGCGGCAGCTGCTGAACGCCAAGCTGATTACCCAGAGAAA |
| | GTTCGACAATCTGACCAAGGCCGAGAGAGGCGGCCTGAGCGAACTGGATAAGGCCGGCTTC |
| | ATCAAGAGACAGCTGGTGGAAACCCGGCAGATCACAAAGCACGTGGCACAGATCCTGGACT |
| | CCCGGATGAACACTAAGTACGACGAGAATGACAAGCTGATCCGGGAAGTGAAAGTGATCAC |
| | CCTGAAGTCCAAGCTGGTGTCCGATTTCCGGAAGGATTTCCAGTTTTACAAAGTGCGCGAG |
| | ATCAACAACTACCACCACGCCCACGACGCCTACCTGAACGCCGTCGTGGGAACCGCCCTGA |
| | TCAAAAAGTACCCTAAGCTGGAAAGCGAGTTCGTGTACGGCGACTACAAGGTGTACGACGT |
| | GCGGAAGATGATCGCCAAGAGCGAGCAGGAAATCGGCAAGGCTACCGCCAAGTACTICTTC |
| | TACAGCAACATCATGAACTTTTTCAAGACCGAGATTACCCTGGCCAACGGCGAGATCCGGA |
| | AGCGGCCTCTGATCGAGACAAACGGCGAAACCGGGGAGATCGTGTGGGATAAGGGCCGGGA |
| | TTTTGCCACCGTGCGGAAAGTGCTGAGCATGCCCCAAGTGAATATCGTGAAAAAGACCGAG |
| | GTGCAGACAGGCGGCTTCAGCAAAGAGTCTATCCTGCCCAAGAGGAACAGCGATAAGCTGA |
| | TCGCCAGAAAGAAGGACTGGGACCCTAAGAAGTACGGCGGCTTCGACAGCCCCACCGTGGC |
| | CTATTCTGTGCTGGTGGTGGCCAAAGTGGAAAAGGGCAAGTCCAAGAAACTGAAGAGTGTG |
| | AAAGAGCTGCTGGGGATCACCATCATGGAAAGAAGCAGCTTCGAGAAGAATCCCATCGACT |
| | TTCTGGAAGCCAAGGGCTACAAAGAAGTGAAAAAGGACCTGATCATCAAGCTGCCTAAGTA |
| | CTCCCTGTTCGAGCTGGAAAACGGCCGGAAGAGAATGCTGGCCTCTGCCGGCGAACTGCAG |
| | AAGGGAAACGAACTGGCCCTGCCCTCCAAATATGTGAACTTCCTGTACCTGGCCAGCCACT |
| | ATGAGAAGCTGAAGGGCTCCCCCGAGGATAATGAGCAGAAACAGCTGTTTGTGGAACAGCA |
| | CAAGCACTACCTGGACGAGATCATCGAGCAGATCAGCGAGTTCTCCAAGAGAGTGATCCTG |
| | GCCGACGCTAATCTGGACAAAGTGCTGTCCGCCTACAACAAGCACCGGGATAAGCCCATCA |
| | GAGAGCAGGCCGAGAATATCATCCACCTGTTTACCCTGACCAATCTGGGAGCCCCTGCCGC |
| | CTTCAAGTACTTTGACACCACCATCGACCGGAAGAGGTACACCAGCACCAAAGAGGTGCTG |
| | GACGCCACCCTGATCCACCAGAGCATCACCGGCCTGTACGAGACACGGATCGACCTGTCTC |
| | AGCTGGGAGGCGACAAAAGGCCGGCGGCCACGAAAAAGGCCGGCCAGGCAAAAAAGAAAAA |
| | GGGCTCCGGA |
| SEQ ID NO: 154 | GTTTTAGAGCTAGAAATAGCAAGTTAAAATAAGGCTAGTCCGTTATCAACTTGAAAAAGTG |
| tracrRNA | GCACCGAGTCGGTGCTTTT |
| RNA | |

What is claimed is:

1. An engineered glycoprotein population with a heterogeneous glycan profile, comprising a plurality of glycoproteins, wherein the heterogenous glycan profile comprises at least 20%, 25%, 30%, 35%, 40%, 45%, 50%, 55%, 60%, 65%, 70%, 75%, 80%, 85%, 86%, 88%, 90%, or 95% of sialylated complex type (SCT) glycans, and the SCT glycans comprises more than one kind of sialylated glycans.

2. The engineered glycoprotein population of claim 1, wherein the heterogeneous glycan profile comprises no more than 95% of SCT glycans.

3. The engineered glycoprotein population of claim 1, wherein the heterogenous glycan profile comprises 20% to 95% of SCT glycans.

4. The engineered glycoprotein population of claim 1, wherein the sialylated glycans comprises a terminal sialic acid, optionally a 7-fluoro sialic acid.

5. The engineered glycoprotein population of claim 4, wherein the terminal sialic acid is connected to a preceding sugar residue of the glycans via a a2,6 linkage or a2,3 linkage.

6. The engineered glycoprotein population of claim 1, wherein the heterogeneous glycan profile comprises (a) SiaGal₂GlcNAc₂Man₃GlcNAc₂ or SiaGal₂GlcNAc₂Man₃GlcNAc₂Fuc and (b) SiazGal₂GlcNAC₂Man₃GlcNAc₂ or SiazGal₂GlcNAc₂Man₃GlcNAc₂Fuc.

7. The engineered glycoprotein population of claim 6, wherein the heterogeneous glycan profile further comprises (c) SiaGalGlcNAcMansGlcNAc₂ or SiaGalGlcNAcMansGlcNAc₂Fuc, (d) SiaGalGlcNAc₂Man₃GlcNAc₂ or SiaGalGlcNAc₂Man₃GlcNAc₂Fuc, (e) SiaGalGlcNAcMan₄GlcNAc₂ or SiaGalGlcNAcMan₄GlcNAc₂Fuc, (f) SiaHexGal₂GlcNAc₂Man₃GlcNAc₂ or SiaHexGal₂GlcNAczMan₃GlcNAc₂Fuc, (g) SiaGalGlcNAc₂Man₃GlcNAc₂ or SiaGalGlcNAc₂Man₃GlcNAc₂Fuc, (h) SiaGal₃GlcNAc₃Man₃GlcNAc₂ or SiaGal₃GlcNAc₃Man₃GlcNAc₂Fuc, or a mixture thereof.

8. The engineered glycoprotein population of claim 1, wherein the SCT glycans do not have a core fucose.

9. The engineered glycoprotein population of claim 1, wherein the glycoprotein comprises a target-binding site and a glycosylation site.

10. The engineered glycoprotein population of claim 1, wherein the glycoprotein is an antibody or antigen-binding fragment thereof.

11. The engineered glycoprotein population of claim 1, wherein the glycoprotein is a therapeutic protein.

12. The engineered glycoprotein population of claim 1, wherein the glycoprotein is Adalimumab (Humira®), Adalimumab-atto (Amjevita®), Bevacizumab (Avastin®), Alemtuzumab (Campath®), Ipilimumab (Yervoy®), Avelumab (Bavencio®), Durvalumab (IMFINZI®), Pembrolizumab (Keytruda®), Nivolumab (Opdivo®), Etanercept (Enbrel®), Trastuzumab (Herceptin®), Pertuzumab (Perjeta®), Cetuximab (Erbitux®), Rituximab (Rituxan®), Rituximab-atto (Truxima®), Obinutuzumab (Gazyva®), Infliximab (Remicade®), Ofatumumab (Arzerra®), Golimumab (Simponi®), Atezolizumab (Tecentriq®), Ocrelizumab (OCREVUS®), Gazyvaro Infliximab (Remicade®), Zanidatamab (Ziihera®), Daratumumab (Darzalex®), or Trastuzumab emtansine (Kadcyla®);
optionally, provided that when the glycoprotein is Adalimumab (Humira®), Bevacizumab (Avastin®), Ipilimumab (Yervoy®), Avelumab (Bavencio®), Durvalumab (IMFINZI®), Pembrolizumab (Keytruda®), Nivolumab (Opdivo®), or Etanercept (Enbrel®), the heterogenous glycan profile comprises core fucose; and/or when the glycoprotein is Trastuzumab (Herceptin®), Pertuzumab (Perjeta®), Cetuximab (Erbitux®), Rituximab (Rituxan®), or Obinutuzumab (Gazyva®), the heterogenous glycan profile comprises no core fucose.

13. The engineered glycoprotein population of claim 1, being produced by a cell, wherein the cell constitutively and/or controllably expresses an exogenous sialyltransferase catalytic peptide and an exogenous galactosyltransferase catalytic peptide, wherein the exogenous sialyltransferase catalytic peptide and the exogenous galactosyltransferase catalytic peptide are translated in close proximity.

14. The engineered glycoprotein population of claim 13, wherein the cell comprises a vector comprising:
a nucleic acid encoding a Clustered Regularly Interspaced Short Palindromic Repeat (CRISPR) crRNA comprising a nucleotide sequence as set forth in SEQ ID NO: 150, SEQ ID NO: 151, or SEQ ID NO: 152;
a nucleic acid encoding a tracrRNA; and
a nucleic acid encoding a CRISPR-associated (Cas) protein.

15. A composition comprising an engineered glycoprotein population of claim 1 and a pharmaceutically acceptable excipient.

16. A method of increasing binding between a Fc gamma receptor (FcγR) and a glycoprotein, comprising: engineering the glycoprotein to obtain an engineered glycoprotein population, wherein the engineered glycoprotein population is according to the engineered glycoprotein population of claim 1.

17. A method of treating a disease caused by a dysfunctional cell, comprising administering a subject in need the engineered glycoprotein population of claim 1 at a pharmaceutically effective amount, wherein the glycoprotein is configured to target the dysfunctional cell.

18. A cell for expressing a sialylated glycoprotein, constitutively and/or controllably expressing an exogenous sialyltransferase catalytic peptide and an exogenous galactosyltransferase catalytic peptide, wherein the exogenous sialyltransferase catalytic peptide and the exogenous galactosyltransferase catalytic peptide are translated in close proximity; and
wherein the cell is deficient in endogenous fucosyltransferase activity, and deficiency in endogenous fucosyltransferase activity is made by introducing a vector into a parent cell of the cell, wherein the vector comprises:
a nucleic acid encoding a Clustered Regularly Interspaced Short Palindromic Repeat (CRISPR) crRNA comprising a nucleotide sequence as set forth in SEQ ID NO: 150, SEQ ID NO: 151, or SEQ ID NO: 152; and
a nucleic acid encoding a tracrRNA.

19. A cell for expressing a sialylated glycoprotein, comprising:
a first nucleic acid, encoding an exogenous sialyltransferase catalytic peptide;
a second nucleic acid, encoding an exogenous galactosyltransferase catalytic peptide; and
a vector comprising:
a nucleic acid encoding a Clustered Regularly Interspaced Short Palindromic Repeat (CRISPR) crRNA comprising a nucleotide sequence as set forth in SEQ ID NO: 150, SEQ ID NO: 151, or SEQ ID NO: 152; and
a nucleic acid encoding a tracrRNA.

20. A method of increasing ADCC, CDC, ADCP, vaccinal effect, and half-live of a glycoprotein, comprising: engineering the glycoprotein to obtain an engineered glycoprotein population, wherein the engineered glycoprotein population is according to the engineered glycoprotein population of claim 1.
